(19) United States
(12) Patent Application Publication
(10) Pub. No.: US 2024/0325570 A1
(43) Pub. Date: Oct. 3, 2024

(54) TREATING DISEASES AND IMPROVING NUCLEIC ACID DELIVERY

(71) Applicant: Mayo Foundation for Medical Education and Research, Rochester, MN (US)

(72) Inventors: Michael A. Barry, Rochester, MN (US); Christopher Y. Chen, Rochester, MN (US); Jeffrey D. Rubin, Palo Alto, CA (US); Vincente E. Torres, Rochester, MN (US)

(21) Appl. No.: 18/271,536

(22) PCT Filed: Jan. 14, 2022
(86) PCT No.: PCT/US2022/012461

§ 371 Date: Jul. 10, 2023

Related U.S. Application Data
(60) Provisional application No. 63/221,196, filed on Jul. 13, 2021.
(60) Provisional application No. 63/137,629, filed on Jan. 14, 2021.

(51) Int. Cl. A61K 48/00; A61K 38/17; A61P 13/12; C12N 9/22; C12N 15/11; C12N 15/86

(52) U.S. Cl. CPC A61K 48/005; A61K 38/1709; A61K 48/0083; A61P 13/12; C12N 9/22; C12N 15/11; C12N 15/86; C12N 2310/20; C12N 2710/10043; C12N 2750/14143

(57) ABSTRACT

This document relates to methods and materials for treating a mammal (e.g., a human) having, or at risk of developing, a polycystic disease (e.g., a polycystic kidney disease (PKD)). For example, methods and materials that can be used to increase a level of polycystin-1 (PC-1) polypeptides and/or polycystin-2 (PC-2) polypeptides within a mammal having, or at risk of developing, a polycystic disease) are provided. In some cases, nucleic acid designed to increase a level of PC-1 polypeptides and/or PC-2 polypeptides within a mammal can be administered to a mammal having, or at risk of developing, a polycystic disease to treat the mammal.

### CROSS-REFERENCE TO RELATED APPLICATIONS

This application claims the benefit of U.S. Patent Application Ser. No. 63/137,629, filed on Jan. 14, 2021, and U.S. Patent Application Ser. No. 63/221,196, filed on Jul. 13, 2021. The disclosures of the prior applications are considered part of (and are incorporated by reference in) the disclosure of this application.

### STATEMENT REGARDING FEDERAL FUNDING

This invention was made with government support under DK090728 and DK123858 awarded by the National Institutes of Health. The government has certain rights in the invention.

### SEQUENCE LISTING

This document includes a Sequence Listing that has been submitted electronically as an ASCII text file named 07039-2024WO1_ST25.txt. The ASCII text file, created on Jan. 14, 2022, is 269 kilobytes in size. The material in the ASCII text file is hereby incorporated by reference in its entirety.

### BACKGROUND

### 1. Technical Field

This document relates to methods and materials for treating a mammal (e.g., a human) having, or at risk of developing, a polycystic disease (e.g., a polycystic kidney disease (PKD)). For example, methods and materials provided herein can be used to increase a level of polycystin-1 (PC-1) polypeptides and/or polycystin-2 (PC-2) polypeptides within a mammal having, or at risk of developing, a polycystic disease. In some cases, nucleic acid designed to increase a level of PC-1 polypeptides and/or PC-2 polypeptides within a mammal can be administered to a mammal having, or at risk of developing, a polycystic disease to treat the mammal.

### 2. Background Information

Autosomal dominant polycystic kidney disease (ADPKD) is an inherited progressive disease with a prevalence of approximately one in one thousand live births in which patients develop fluid-filled cysts in their kidneys, losing kidney function, and which can end in kidney failure (see, e.g., Bergmann et al., Nat. Rev. Dis. Primers., 4(1):50 (2018)).

### SUMMARY

ADPKD can be caused by one or more mutations in the PKD1 gene (encoding the PC-1 polypeptide) and/or the PKD2 gene (encoding the PC-2 polypeptide). As such, ADPKD can be treated by gene therapy techniques that can deliver nucleic acid designed to increase a level of PC-1 polypeptides and/or PC-2 polypeptides within a mammal. However, while many gene therapy vectors can carry the 2.9 kilobase (kb) PKD2 cDNA, most gene therapy vectors and techniques cannot carry the extremely large 12.9 kb PKD1 cDNA.

This document is based, at least in part, on the development of vectors that can be used to deliver nucleic acid designed to increase a level of PC-1 polypeptides and/or PC-2 polypeptides within a mammal. In some cases, this document provides methods and materials for treating a mammal having, or at risk of developing, a polycystic disease (e.g., PKD). For example, nucleic acid designed to increase a level of PC-1 polypeptides and/or PC-2 polypeptides within a mammal can be administered to a mammal having, or at risk of developing, a polycystic disease to treat the mammal. As described herein, adeno-associated virus (AAV) vectors can be used to deliver nucleic acid designed to express a PC-2 polypeptide (e.g., a PKD2 cDNA) to increase the level of PC-2 polypeptides in cells, and helper-dependent adenovirus (HDAd) vectors can be used to deliver nucleic acid designed to express a PC-1 polypeptide (e.g., a PKD1 cDNA) and/or nucleic acid designed to express a PC-2 polypeptide (e.g., a PKD2 cDNA) to increase the level of PC-1 polypeptides and/or PC-2 polypeptides in cells. For example, vectors described herein containing nucleic acid designed to express a PC-1 polypeptide and/or nucleic acid designed to express a PC-2 polypeptide can be administered to a mammal (e.g., a human) having, or at risk of developing, a polycystic disease (e.g., a PKD) to increase a level of PC-1 polypeptides and/or PC-2 polypeptides within the mammal (e.g., to treat the mammal). Also as described herein, one or more AAV vectors can be used to deliver gene therapy components designed for targeted gene activation (e.g., designed for CRISPR-Cas9-based targeted gene activation) of the PKD1 gene and/or the PKD2 gene to upregulate transcription of the PKD1 gene and/or the PKD2 gene to increase the level of PC-1 polypeptides and/or PC-2 polypeptides in cells. For example, one or more nucleic acid molecules designed to express the components of a targeted gene activation system (or the components themselves) designed to activate transcription of a PKD1 gene and/or to activate transcription of a PKD2 gene can be administered to a mammal (e.g., a human) having, or at risk of developing, a polycystic disease (e.g., a PKD) to increase a level of PC-1 polypeptides and/or PC-2 polypeptides within the mammal (e.g., to treat the mammal).

This document also provides methods and materials for improving delivery of nucleic acid to a mammal. As described herein, inducing proteinuria in a mammal (e.g., prior to administering a nucleic acid molecule) can improve delivery of nucleic acid (e.g., nucleic acid designed to increase a level of PC-1 polypeptides and/or PC-2 polypeptides within a mammal) to the mammal (e.g., to one or more cells within the mammal). For example, one or more lipopolysaccharides (LPSs) can be administered to a mammal to induce proteinuria in the mammal to improve delivery of nucleic acid (e.g., nucleic acid designed to increase a level of PC-1 polypeptides and/or PC-2 polypeptides within a mammal) to cells (e.g., kidney cells) within the mammal.

Having the ability to increase a level of PC-1 polypeptides and/or PC-2 polypeptides within a mammal provides a unique and unrealized opportunity to treat a polycystic disease such as a PKD.

Having the ability to increase the delivery of nucleic acid to cells within a mammal as described herein can allow for more efficient gene therapy approaches.

In general, one aspect of this document features methods for treating a mammal having a PKD. The methods can include, or consist essentially of, administering to a mammal having a PKD nucleic acid encoding a PC-1 polypeptide or a variant of the PC-1 polypeptide, where the PC-1 polypeptide or the variant is expressed by kidney cells within the mammal. The nucleic acid encoding the PC-1 polypeptide or the variant can be administered to the mammal in the form of a viral vector (e.g., a helper-dependent adenovirus (HDAd) vector). The nucleic acid encoding the PC-1 polypeptide or the variant can be operably linked to a promoter sequence. The promoter sequence can be a human elongation factor 1α (EF1α) promoter sequence, a chicken ß-actin hybrid (CBh) promoter sequence, a PKD1 promoter sequence, a PKD2 promoter sequence, a cytomegalovirus (CMV) promoter sequence, a Rous sarcoma virus (RSV) promoter sequence, an aquaporin 2 (AQP2) promoter sequence, a gamma-glutamyltransferase 1 (Ggt1) promoter sequence, or a Ksp-cadherin promoter sequence. The method can include identifying the mammal as being in need of a treatment for the PKD. The mammal can be a human. The PKD can be an autosomal dominant PKD (ADPKD). The method also can include, prior to the administering the nucleic acid, administering a lipopolysaccharides (LPS) to the mammal. The LPS can be administered to the mammal at least 18 hours prior to the administering the nucleic acid. The LPS can be effective to deliver large nucleic acid to the kidney cells in the mammal.

In another aspect, this document features methods for treating a mammal having a PKD. The methods can include, or consist essentially of, administering to a mammal having a PKD nucleic acid encoding a PC-2 polypeptide or a variant of the PC-2 polypeptide, where the PC-2 polypeptide or the variant is expressed by kidney cells within the mammal. The nucleic acid encoding the PC-2 polypeptide or the variant can be administered to the mammal in the form of a viral vector (e.g., an adenovirus-associated virus (AAV) vector). The nucleic acid encoding the PC-2 polypeptide or the variant can be operably linked to a promoter sequence. The promoter sequence can be a EF1a promoter sequence, a CBh promoter sequence, a PKD1 promoter sequence, a PKD2 promoter sequence, a CMV promoter sequence, a RSV promoter sequence, an AQP2 promoter sequence, a Ggt1 promoter sequence, or a Ksp-cadherin promoter sequence. The method can include identifying the mammal as being in need of a treatment for the PKD. The mammal can be a human. The PKD can be an autosomal dominant PKD (ADPKD). The method also can include, prior to the administering the nucleic acid, administering a lipopolysaccharides (LPS) to the mammal. The LPS can be administered to the mammal at least 18 hours prior to the administering the nucleic acid. The LPS can be effective to deliver large nucleic acid to the kidney cells in the mammal.

In another aspect, this document features methods for treating a mammal having a PKD. The methods can include, or consist essentially of, administering to a mammal having a PKD: (a) nucleic acid encoding a PC-1 polypeptide or a variant of the PC-1 polypeptide, where the PC-1 polypeptide or the variant is expressed by kidney cells within the mammal; and (b) nucleic acid encoding a PC-2 polypeptide or a variant of the PC-2 polypeptide, where the PC-2 polypeptide or the variant is expressed by kidney cells within the mammal. The nucleic acid encoding the PC-1 polypeptide or the variant can be administered to the mammal in the form of a viral vector (e.g., a HDAd vector). The nucleic acid encoding the PC-1 polypeptide or the variant can be operably linked to a promoter sequence. The promoter sequence can be a EF1a promoter sequence, a CBh promoter sequence, a PKD1 promoter sequence, a PKD2 promoter sequence, a CMV promoter sequence, a RSV promoter sequence, an AQP2 promoter sequence, a Ggt1 promoter sequence, or a Ksp-cadherin promoter sequence. The nucleic acid encoding the PC-2 polypeptide or the variant can be administered to said mammal in the form of a viral vector (e.g., an AAV vector). The nucleic acid encoding the PC-2 polypeptide or the variant can be operably linked to a promoter sequence. The promoter sequence can be a a EF1a promoter sequence, a CBh promoter sequence, a PKD1 promoter sequence, a PKD2 promoter sequence, a CMV promoter sequence, a RSV promoter sequence, an AQP2 promoter sequence, a Ggt1 promoter sequence, or a Ksp-cadherin promoter sequence. The nucleic acid encoding the PC-1 polypeptide or the variant and the nucleic acid encoding the PC-2 polypeptide or the variant are administered to the mammal in the form of a viral vector (e.g., a HDAd vector). The nucleic acid encoding the PC-1 polypeptide or the variant can be operably linked to a first promoter sequence, and the nucleic acid encoding the PC-2 polypeptide or the variant can be operably linked to a second promoter sequence. The first promoter sequence and the second promoter sequence can each be independently selected from the group consisting of a EF1α promoter sequence, a CBh promoter sequence, a PKD1 promoter sequence, a PKD2 promoter sequence, a CMV promoter sequence, a RSV promoter sequence, an AQP2 promoter sequence, a Ggt1 promoter sequence, and a Ksp-cadherin promoter sequence. The method can include identifying the mammal as being in need of a treatment for the PKD. The mammal can be a human. The PKD can be an autosomal dominant PKD (ADPKD). The method also can include, prior to the administering the nucleic acid, administering a lipopolysaccharides (LPS) to the mammal. The LPS can be administered to the mammal at least 18 hours prior to the administering the nucleic acid. The LPS can be effective to deliver large nucleic acid to the kidney cells in the mammal. The method can include identifying the mammal as being in need of a treatment for the PKD. The mammal can be a human. The PKD can be an autosomal dominant PKD (ADPKD). The method also can include, prior to the administering the nucleic acid, administering a lipopolysaccharides (LPS) to the mammal. The LPS can be administered to the mammal at least 18 hours prior to the administering the nucleic acid. The LPS can be effective to deliver large nucleic acid to the kidney cells in the mammal.

In another aspect, this document features methods for treating a mammal having a PKD. The methods can include, or consist essentially of, administering to a mammal having a PKD: (a) nucleic acid encoding a fusion polypeptide including a deactivated Cas (dCas) polypeptide and a transcriptional activator polypeptide: (b) nucleic acid encoding a helper activator polypeptide; and (c) nucleic acid encoding a nucleic acid molecule including (i) a nucleic acid sequence that is complementary to a target sequence within a PKD1 gene, and (ii) a nucleic acid sequence that can bind the helper activator polypeptide. The dCas polypeptide can be a deactivated Cas9 (dCas9) polypeptide or a deactivated Cas phi (dCasΦ) polypeptide. The transcriptional activator polypeptide can be a VP64 polypeptide. The fusion polypeptide can be a dCas9-VP64 fusion polypeptide. The helper activator polypeptide can be a MS2 polypeptide, a p65 polypeptide, a HSF1 polypeptide, or a VP64 polypeptide. The helper activator polypeptide can include a MS2 polypeptide, a p65 polypeptide, and a HSF1 polypeptide. The nucleic acid (a), the nucleic acid (b), and the nucleic acid (c) can be administered to the mammal in the form of a viral vector. The viral vector can be a HDAd, a lentiviral vector, or an AAV vector. The nucleic acid (a) can be administered to the mammal in the form of a first viral vector, and the nucleic acid (b) and the nucleic acid (c) can be administered to the mammal in the form of a second viral vector. The first viral vector can be an AAV vector and the second viral vector can be an AAV vector. The nucleic acid (a) can be operably linked to a first promoter sequence, the nucleic acid (b) can be operably linked to a second promoter sequence, and the nucleic acid (c) can be operably linked to a third promoter sequence. The first promoter sequence, the second promoter sequence, and the third promoter sequence can each independently be selected from the group consisting of a EF1a promoter sequence, a CBh promoter sequence, a CMV promoter sequence, a RSV promoter sequence, a U6 promoter sequence, an AQP2 promoter sequence, a Ggt1 promoter sequence, and a Ksp-cadherin promoter sequence. The method also can include identifying the mammal as being in need of a treatment for the PKD. The mammal can be a human. The PKD can be an ADPKD. The also can include, prior to the administering the nucleic acid, administering a LPS to the mammal. The LPS can be administered to the mammal at least 18 hours prior to the administering the nucleic acid. The administering the LPS can be effective to deliver large nucleic acid to the kidney cells in the mammal.

In another aspect, this document features methods for delivering nucleic acid to a cell within a mammal. The methods can include, or consist essentially of, (a) administering a proteinuria-inducing agent to a mammal; and (b) administering nucleic acid to the mammal. The mammal can be a human. The proteinuria-inducing agent can be LPS, puromycin, adriamycin, protamine sulfate, cationic albumin, or polycations. The nucleic acid can be from about 0.15 kb to about 36 kb in size. The nucleic acid can have a mass of from about 10 kilodaltons (kDa) to about 50 kDa. The nucleic acid can have a diameter of from about 10 nm to about 26 nm. The method can include administering from about 7 milligrams per kilogram body weight (mg/kg) to about 9 mg/kg of the proteinuria-inducing agent to the mammal. The cell can be a kidney cell, a spleen cell, a lungs cell, or a brain cell. The proteinuria-inducing agent can be administered to the mammal at least 18 hours prior to the administering the nucleic acid. The administering the proteinuria-inducing agent can include intravenous injection. The administering the nucleic acid can include intravenous injection. The administering the proteinuria-inducing agent can include intravenous injection, and the administering the nucleic acid can include intravenous injection.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1D. Diagrams of exemplary in vivo vectors for delivery of PKD1 and PKD2 cDNAs. FIG. 1A shows a single HDAd vector including a PKD1 cDNA with additional space for cargo, denoted as “stuffer”. FIG. 1B shows an AAV vector including a PKD2 cDNA. FIG. 1C shows an HDAd vector including both a PKD1 cDNA and a PKD2 cDNA. ITR=inverted terminal repeat, EF1α=human elongation factor 1α promoter, CBh=chicken β-actin hybrid promoter. FIG. 1D shows alternative HDAd vectors including a PKD1 cDNA and/or a PKD2 cDNA.

FIG. 2. A schematic of an exemplary process used to generate triple transduced, stable cell lines expressing Cas9-SAM. LV=lentivirus, Bsd=blasticidin, Hyg=hygromycin, Zeo=zeocin.

FIG. 3. A graph showing fold PKD1 gene expression of human 293 cells transduced to express Cas9-SAM. qRT-PCR was performed with one biological replicate and three technical replicates (n=1). RQ=relative quantitation.

FIG. 4. A graph showing fold PKD1 gene expression of human RCTE cells transduced to express Cas9-SAM. qRT-PCR was performed with one biological replicate and three technical replicates (n=1). RQ=relative quantitation.

FIG. 5. A graph showing fold Pkd1 gene expression of mouse IMCD3 cells transduced to express Cas9-SAM. qRT-PCR was performed with one biological replicate and three technical replicates (n=1). RQ=relative quantitation.

FIGS. 6A-6D. Diagrams of exemplary vectors for in vivo delivery of Cas9-SAM. FIG. 6A shows a single HDAd vector delivering the entire Cas9-SAM system with additional space for cargo, denoted as “stuffer”. FIG. 6B shows a single lentiviral vector delivering the entire Cas9-SAM system. FIG. 6C shows a dual AAV vector system for delivering the Cas9-SAM system in two pieces. FIG. 6D shows a single AAV vector system for delivering the SAM system based on a newly discovered and smaller CasΦ protein. ITR=inverted terminal repeat, LTR=long terminal repeat, U6=U6 promoter, CMV=human cytomegalovirus promoter, EF1α=human elongation factor 1α promoter, CBh=chicken β-actin hybrid promoter, P2A=2A self-cleaving peptide.

FIG. 7. A western blot of dCas9VP64 protein from transfected viral vector expression cassettes. All three transfected AAV cassettes and the transfected Ad cassette produced dCas9VP64 protein, which is calculated to have a mass of 168.26 kilodaltons. EF1α=human elongation factor 1α promoter, CMV=human cytomegalovirus promoter, FpA=Ad5 Fiber polyadenylation signal, HGHpA=Human growth hormone polyadenylation signal.

FIG. 8. Ex vivo luminescent imaging of livers and kidneys after intravenous injection with AAV8, with or without induced proteinuria. Mice were administered either PBS or LPS by intraperitoneal (i.p.) injection or intravenous (i.v.) injection with 1.94e12 genome copies of self-complementary (sc) AAV8-Cre a day later (n=1). Six days after AAV injection, the mice were sacrificed and their livers and kidneys were imaged for luminescence ex vivo. While the liver signals remained consistent, the mouse injected with LPS exhibited greater luminescence from its kidneys than the PBS-injected mouse. LK=left kidney, RK=right kidney.

FIG. 9. Fluorescent imaging of liver and kidney sections after intravenous injection with AAV8, with or without induced proteinuria. The same liver and kidney tissues from FIG. 8 were sectioned to view transduced (EGFP⁺) cells. The livers from both mice appear to be almost entirely transduced after a high dose of the liver tropic AAV8. The kidneys of the LPS-injected mouse shows transduced glomeruli and proximal tubules whereas the kidneys of the PBS-injected mouse show only transduced glomeruli. Arrows point to transduced proximal tubules adjacent to glomeruli.

FIGS. 10A-10D. Ex vivo liver and kidney luminescence and flow cytometry with a lower dose of AAV8, with or without proteinuria. FIG. 10A contains a graph showing no significant difference in liver luminescent between PBS and LPS-injected mice (n=3; p=0.2000). FIG. 10B contains a graph showing that kidneys of LPS-injected mice exhibited significantly more luminescence than those of PBS-injected mice (n=; * p=0.0260). FIGS. 10C and 10D contain graphs showing the percent of GFP⁺ cells in kidneys from FIG. 10B that were homogenized, stained, and analyzed by flow cytometry. FIG. 10C shows that EpCAM⁺CD31⁻ (epithelial) cells had a significant increase in transduction (n=6: **p=0.0022). FIG. 10D shows that EpCAM⁻CD31⁺ (endothelial) cells showed no significant change in transduction between LPS and PBS-injected mice (n=6: p=0.6991).

FIGS. 11A-11C. Investigation of mice injected i.v. with Ad5-Cre, with or without induced proteinuria. FIG. 11A contains exemplary images of bisected kidneys of one PBS/Ad5-Cre mouse and one LPS/Ad5-Cre mouse. Mice were sacrificed and their kidneys were imaged ex vivo (n=3). LPS-injected mouse kidneys exhibiting increased luminescence. FIG. 11B contains a graph showing quantitation of ex vivo kidney luminescence. Luminescence significantly increased in LPS-injected mice from PBS-injected mice (n=6 kidneys: **p=0.0022). FIG. 11C contains exemplary fluorescent images of liver and kidney sections. Liver transduction decreased and kidney transduction increased, specifically in the glomeruli, in the LPS-injected mice. Arrows point to increased transduction in glomeruli.

FIGS. 12A-12B. PC-1 sequences. FIG. 12A is a representative nucleic acid sequence that can encode a human PC-1 polypeptide (SEQ ID NO:1). FIG. 12B is an amino acid sequence of a representative human PC-1 polypeptide (SEQ ID NO: 2).

FIGS. 13A-13B. PC-2 sequences. FIG. 13A is a representative nucleic acid sequence that can encode a human PC-2 polypeptide (SEQ ID NO:3). FIG. 13B is an amino acid sequence of a representative human PC-2 polypeptide (SEQ ID NO:4).

FIGS. 14A-14B. Intravenous delivery of AAV8 in a state of induced proteinuria enhances kidney transduction. FIG. 14A. Diagram of experimental scheme. Two month old male luciferase-mT/mG triple reporter mice were administered LPS intraperitoneally on Day −1 and scAAV intravenously on Day 0. In vivo bioluminescence was assessed daily until peak expression was observed at Day 6. FIG. 14B. In vivo bioluminescence at Day 6 followed by ex vivo luminescence of livers and kidneys. n=1 mouse per group.

FIG. 15. Intravenous delivery of multiple AAV serotypes enhances tubule epithelial cell transduction, but not necessarily proximal tubule cell transduction. The same kidneys from FIG. 14 were sectioned to examine endogenous mT and mG fluorescence. Arrows point to examples of transduced non-glomerular (tubular) cells. While some tubular cell transduction was observed in PBS-injected control mice (left panels), there were increased numbers of these cells in LPS-injected induced proteinuria mice (center panels). No instances of these transduced cells were observed to be counterstained by LTL, a marker of proximal tubule cells (right panels). n=1 mouse per group.

FIGS. 16A-16C. Intravenous delivery of scAAV8 in a state of induced proteinuria significantly increases transduction of renal epithelial cells. FIG. 16A. Three month old male mice were administered an i.p. injection of either PBS or LPS at Day −1 and an i.v. injection of 2.03e11 GC of scAAV8-Cre at Day 0. At Day 6, in vivo luminescence and ex vivo liver luminescence were not significantly different between PBS and LPS-injected groups, although brain luminescence was significantly increased in the LPS-injected group (p=0.0475 by Welch's t test). n=3 mice per group, except for control group where n=1: error bars are represented by mean with SD. FIG. 16B. Kidneys were bisected with a razor blade to reduce obstruction of luminescence and imaged ex vivo, with the LPS-injected group exhibiting increased luminescence compared to the PBS-injected group. FIG. 16C. Ex vivo luminescence from Panel B was quantified and kidneys were subsequently processed for flow cytometry. Overall, kidneys from LPS-injected mice showed significantly higher ex vivo luminescence and percentage of transduced epithelial cells, but not of transduced endothelial cells (p values obtained using Mann-Whitney test). n=6 kidneys per group, except for control group where n=1: error bars are represented by mean with SD.

FIGS. 17A-17B. AAVrh10 does not necessarily increase transduction of tubule epithelial cells during induced proteinuria. FIG. 17A. Eight month old female mice were administered an i.p. injection of either PBS or LPS at Day-1 and an i.v. injection of 1.76e11 GC of scAAVrh10-Cre at Day 0. At Day 5, kidneys were processed for flow cytometry. Although there was no difference in transduced CD45− (non-hematopoietic) kidney cells, kidneys of the LPS-injected group had a significant increase in CD45+ (hematopoietic) cells compared to the PBS-injected group (n=6 kidneys per group). FIG. 17B. Kidney CD45− (non-hematopoietic) cells were separately gated into EpCAM⁺ CD31− (all epithelial cells), EpCAM⁻ CD31⁺ (endothelial cells), and EpCAM⁺ LTL⁺ and EpCAM⁺ AQP1⁺ (two different markers of proximal tubule cells). None of the aforementioned gating strategies showed a significant difference in transduced cells between PBS-injected and LPS-injected groups. n=6 kidneys per group, except for control group where n=1: error bars are represented by mean with SD for all panels. p values determined using Mann-Whitney tests for all panels.

FIGS. 18A-18B. Examination of kidney transduction using a vector with low liver tropism. FIG. 18A. Four and a half month old female mice were administered an i.p. injection of PBS or LPS on Day-1 and an i.v. injection of 9.5e10 GC of scAAV1-Cre on Day 0. In vivo bioluminescence was assessed daily until peak expression was observed at Day 6. No significant difference was observed between groups, including a measurement of ex vivo liver luminescence (p values determined using Welch's t test). n=3 mice per group, except for control group where n=1. Error bars are represented by mean with error (top left) or mean with SD (top right). Ex vivo kidney luminescence showed that LPS-injected mice had an increased but insignificant amount of luminescence compared to PBS-injected mice as well as LPS and scAAV8-Cre injected mice (p values determined using Mann-Whitney test). n=6 kidneys per group, lower panels: error bars are represented by mean with SD: scAAV8-Cre data represents the same data shown in FIG. 16. FIG. 18B. The kidneys analyzed in Panel A were sectioned to observe endogenous mT and mG fluorescence. While mice treated with PBS and scAAV1-Cre showed transduction primarily in glomeruli (left), mice treated with LPS and scAAV1-Cre showed increased transduction in non-glomerular (tubular) cells (right). Arrows point to examples of transduced glomerular cells (left) or examples of transduced tubule cells (right).

FIGS. 19A-19C. Induced proteinuria increases adenovirus transduction of the kidney, but strictly in glomeruli. FIG. 19A. Four month old mice were administered an i.p. injection of PBS (male mice) or LPS (female mice) on Day-1 and an i.v. injection of 1e11 vp of Ad5-Cre on Day 0. In vivo bioluminescence was assessed daily until peak expression was observed at Day 5. Luminescence was significantly lower in LPS-injected mice compared to PBS-injected mice (p value determined using Welch's t test: n=3 mice per group: error bars are represented by mean with error, left), however, ex vivo kidney luminescence was significantly higher in LPS-injected mice compared to PBS-injected mice (p value determined using Mann-Whitney test: n=6 kidneys per group, except for control group where n=1: error bars are represented by mean with SD, right). FIG. 19B. Bioluminescent images of the kidneys quantified ex vivo in Panel A. While essentially no luminescence is visible in kidneys from mice injected with PBS, kidneys from mice injected with LPS showed luminescence localized to the renal pelvis region of the kidney. FIG. 19C. Kidneys shown in Panel B were sectioned to examine mT and mG endogenous fluorescence. Yellow arrows point to examples to transduced glomerular cells, which are present sparsely in mice injected with PBS and more frequently in mice injected with LPS. No instances of transduced tubular cells were observed in either group of mice.

FIGS. 20A-20B. Induced proteinuria increases AAV gene delivery to renal epithelial cells in mice with polycystic kidney disease. FIG. 20A. Male Pkd1RC/RC-mT/mG hybrid mice were generated, which have two hypomorphic Pkd1RC alleles and develop autosomal dominant polycystic kidney disease. Nine month old male mice were treated with PBS or LPS via i.p. injection at Day-1 and 1.64e11 GC of scAAV8-Cre via i.v. injection at Day 0. FIG. 20B. Mice were sacrificed at Day 6 and their kidneys were sectioned to examine mT and mG endogenous fluorescence. Arrows point to transduced cells. While transduced glomerular cells were observed in PBS-injected mice, transduced tubular cells were observed only in LPS-injected mice (n=1 mouse for each group).

FIG. 21. Diagram modeling vector pharmacokinetics in a state of induced proteinuria. LPS administration results in degradation of podocyte foot processes, effectively increasing the permselectivity of slit diaphragms to an unknown diameter above the natural 10 nm. This change in physiology allows the smaller AAV (25 nm i.d.) to penetrate into adjacent tubule cells while the larger Ad (90 nm i.d.) has increased penetration into glomerular cells but not tubular cells. It is also possible that AAV moved from the vasculature of the kidney to transduce cells of the macula densa.

FIG. 22. Example of proteinuria dipsticks used to assess induced proteinuria in mice. Mice administered LPS at Day-1 had a higher indicated level of proteinuria at Day 0 while mice administered PBS had a consistent level of proteinuria from Day-1 to Day 0. It was common for mice administered LPS to have a proteinuria level of greater than 2000 mg/dL the following day.

FIGS. 23A-23B. Administration of LPS to mice did not affect liver transduction by AAV but did result in renal medullar transduction across several serotypes of AAV. FIG. 23A. Quantification of in vivo luminescence images shown in FIG. 14. Mice that were administered i.p. injections of either PBS or LPS on Day −1 and i.v. injections of scAAV8-Cre, scAAV9-Cre, or scAAVrh10-Cre on Day 0 had levels of in vivo luminescence that varied by approximately two orders of magnitude on Day 1, but these signals reached approximately the same level by Day 6. FIG. 23B. Images of the medulla of kidneys of scAAV8 and scAAV9 injected mice from FIG. 15. Both images show that there is transduction in medullar cells in addition to the cortical tubular cells shown earlier.

FIGS. 24A-24B. Evidence of toxicity associated with combined LPS and AAV administration. FIG. 24A. Liver sections of mice injected with PBS or LPS followed by high-dose scAAV8-Cre. These sections are from the same mice injected with scAAV8-Cre in FIGS. 14 and 15. While both livers are entirely transduced by scAAV8-Cre, the liver of the LPS-injected mouse exhibited a globular cell phenotype indicative of toxicity. FIG. 24B. From the mice in FIG. 3, mice treated with LPS had significantly increased transduced levels of macrophages in the blood compared to mice treated PBS, indicating an overall increase in macrophage present in the blood after LPS treatment. (p=0.0475 by Welch's t test.)

FIGS. 25A-25D. Representative flow cytometry plots for mice administered scAAV8-Cre. Plots are from Left Kidney of Mouse #01 Left Kidney (treated with PBS followed by PBS, in a group of n=2 kidneys) and Mouse #07 (treated with LPS followed by scAAV8-Cre, in a group of n=6 kidneys).

FIGS. 26A-26C. Representative flow cytometry plots for mice administered scAAVrh10-Cre. Plots are from Left Kidney of Mouse #01 Left Kidney (treated with PBS followed by PBS, in a group of n=2 kidneys) and Mouse #02 (treated with LPS followed by scAAVrh10-Cre, in a group of n=6 kidneys).

FIGS. 27A-27B. Increased kidney transduction after administration of LPS and Ad5-Cre is negatively correlated with liver transduction. FIG. 27A. Example of liver sections of mice injected either with PBS followed by Ad5-Cre or LPS followed by Ad5-Cre. While the liver of the former is fully transduced, the liver of the latter is only partially transduced. FIG. 27.) The mice shown are the same mice from FIG. 19 injected with LPS followed by Ad5-Cre. In vivo imaging (top) is juxtaposed to corresponding liver section (middle) and ex vivo kidney imaging (bottom). Mice with weaker liver transduction exhibited stronger kidney transduction.

FIG. 28. Comparison of liver transduction across various vectors and doses. While scAAV8, scAAV9, and scAAVrh10 fully transduced the liver, scAAV1 only partially transduced the liver. Ad5-Cre fully transduced the liver, which was attenuated when LPS was administered prior to Ad5-Cre.

FIG. 29. Livers of mice with polycystic kidney disease were fully transduced by scAAV8-Cre. Livers of mice shown in FIG. 20. The livers of these mice were fully transduced when injected with scAAV8-Cre.

FIG. 30. A schematic representation of a cre recombinase activated reporter mouse model.

FIGS. 31A-31B. Representative images of mice showing luciferase expression. FIG. 31A. In vivo luminescent imaging. FIG. 31B. Fluorescent imaging of tissue sections.

FIGS. 32A-32E. FIG. 32A. Fluorescent imaging of kidney sections following transduction with different AAV serotypes. FIG. 32B. Immunostaining of smooth muscle and proximal tubules in kidneys of mice treated with AAV1. FIG. 32C. Immunostaining of smooth muscle and proximal tubules in kidneys of mice treated with AAV8. FIG. 32D. Immunostaining of podocytes, smooth muscle, and proximal tubules in kidneys of mice treated with AAV9. FIG. 32E. Immunostaining of endothelium, smooth muscle, and proximal tubules in kidneys of mice treated with AAVrh10.1.

FIG. 33. Immunostaining of endothelium in kidneys of mice treated with AAVrh10.1. mRFP indicates untransduced cells. mGFP indicates Cre-transduced cells. Violet-colored cells are cells detected with anti-CD31 antibody.

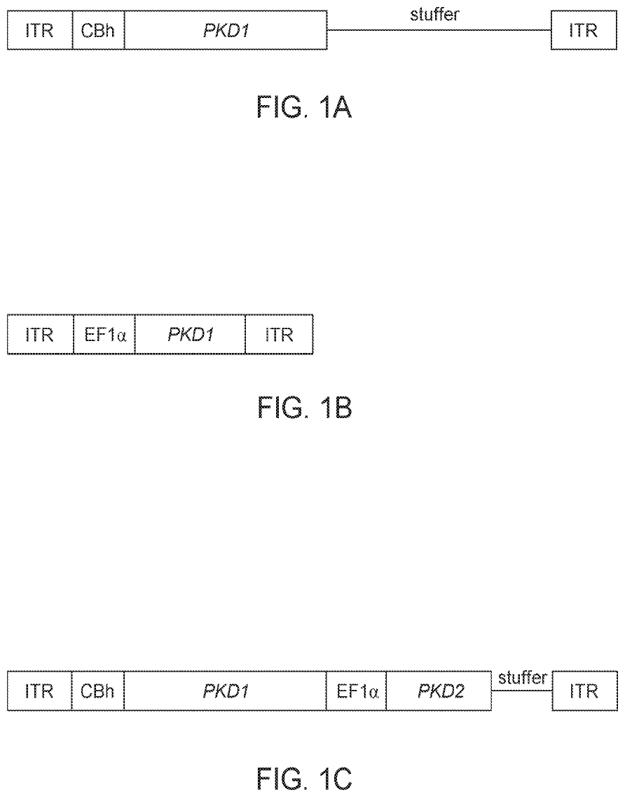

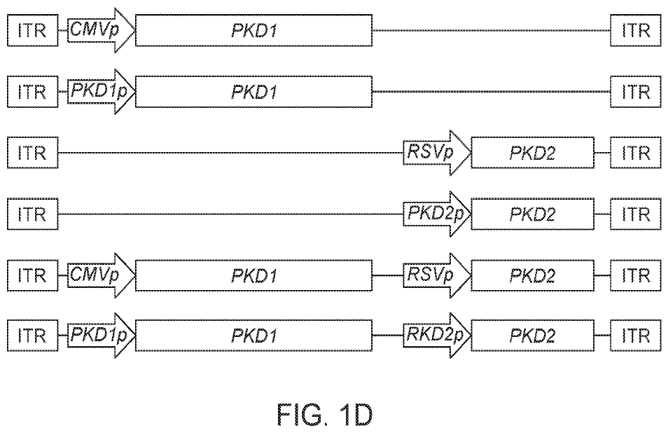

### DETAILED DESCRIPTION

This document provides methods and materials for treating a mammal (e.g., a human) having, or at risk of developing, a polycystic disease (e.g., a PKD). For example, methods and materials provided herein can be used to increase a level of PC-1 polypeptides and/or PC-2 polypeptides within a mammal having, or at risk of developing, a polycystic disease) to treat the mammal. In some cases, nucleic acid designed to increase a level of PC-1 polypeptides and/or PC-2 polypeptides within a mammal can be administered to a mammal (e.g., a human) having, or at risk of developing, a polycystic disease (e.g., a PKD) to treat the mammal. For example, nucleic acid designed to express a PC-1 polypeptide and/or nucleic acid designed to express a PC-2 polypeptide can be administered to a mammal (e.g., a human) having, or at risk of developing, a polycystic disease (e.g., a PKD) to increase a level of PC-1 polypeptides and/or PC-2 polypeptides within the mammal (e.g., to treat the mammal). For example, one or more nucleic acid molecules designed to express the components of a targeted gene activation system designed to activate transcription of a PKD1 gene (e.g., resulting in an increased level of PC-1 polypeptides) and/or to activate transcription of a PKD2 gene (e.g., resulting in an increased level of PC-2 polypeptides) can be administered to a mammal (e.g., a human) having, or at risk of developing, a polycystic disease (e.g., a PKD) to increase a level of PC-1 polypeptides and/or PC-2 polypeptides within the mammal (e.g., to treat the mammal).

As used herein, an “increased” level of PC-1 polypeptides and/or PC-2 polypeptides can be any level that is higher than a level of PC-1 polypeptides and/or PC-2 polypeptides in a mammal (e.g., human) that was observed prior to being treated as described herein (e.g., by administering nucleic acid designed to increase a level of PC-1 polypeptides and/or PC-2 polypeptides to the mammal). An increase in a level of PC-1 polypeptides and/or PC-2 polypeptides can be in any appropriate tissue and/or organ of a mammal (e.g., a human). Examples of tissues and/or organs in which a level of PC-1 polypeptides and/or PC-2 polypeptides can be increased as described herein (e.g., by administering nucleic acid designed to increase a level of PC-1 polypeptides and/or PC-2 polypeptides to the mammal) include, without limitation, kidneys, liver, spleen, lungs, and brain. In some cases, administering nucleic acid designed to increase a level of PC-1 polypeptides and/or PC-2 polypeptides to a mammal having a polycystic disease (e.g., a PKD) can be effective to increase a level of PC-1 polypeptides and/or PC-2 polypeptides in one or both kidneys in the mammal. For example, nucleic acid designed to express a PC-1 polypeptide and/or nucleic acid designed to express a PC-2 polypeptide can be administered to a mammal (e.g., a human) in need thereof (e.g., a human having, or at risk of developing, a polycystic disease such as PKD) as described herein to increase a level of PC-1 polypeptides and/or PC-2 polypeptides in the mammal by, for example, 10, 20, 30, 40, 50, 60, 70, 80, 90, 95, or more percent. In some cases, nucleic acid designed to express a PC-1 polypeptide and/or nucleic acid designed to express a PC-2 polypeptide can be administered to a mammal (e.g., a human) in need thereof (e.g., a human having, or at risk of developing, a polycystic disease such as PKD) as described herein to increase a level of PC-1 polypeptides and/or PC-2 polypeptides in the mammal by, for example, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, or more. For example, one or more nucleic acid molecules designed to express the components of a targeted gene activation system designed to activate transcription of a PKD1 gene and/or a PKD2 gene can be administered to a mammal (e.g., a human) in need thereof (e.g., a human having, or at risk of developing, a polycystic disease such as PKD) as described herein to increase a level of PC-1 polypeptides and/or PC-2 polypeptides in the mammal by, for example, 10, 20, 30, 40, 50, 60, 70, 80, 90, 95, or more percent. In some cases, one or more nucleic acid molecules designed to express the components of a targeted gene activation system designed to activate transcription of a PKD1 gene and/or a PKD2 gene can be administered to a mammal (e.g., a human) in need thereof (e.g., a human having, or at risk of developing, a polycystic disease such as PKD) as described herein to increase a level of PC-1 polypeptides and/or PC-2 polypeptides in the mammal by, for example, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, or more.

In some cases, a mammal (e.g., a human) having, or at risk of developing, a polycystic disease (e.g., a PKD) can be treated as described herein (e.g., by administering nucleic acid designed to increase a level of PC-1 polypeptides and/or PC-2 polypeptides within a mammal) to reduce or eliminate one or more symptoms of a polycystic disease (e.g., a PKD) and/or one or more complications associated with a polycystic disease (e.g., a PKD). Examples of symptoms of a polycystic disease (e.g., a PKD) and complications associated with a polycystic disease (e.g., a PKD) include, without limitation, back pain, side pain, headache, a feeling of fullness (e.g., in the abdomen), increased size of the abdomen (e.g., due to an enlarged kidney), blood in the urine, high blood pressure, loss of kidney function (e.g., kidney failure), heart valve abnormalities (e.g., mitral valve prolapse), colon problems (e.g., diverticulosis), development of an aneurysm (e.g., a brain aneurysm), and endothelial dysfunction (ED). For example, nucleic acid designed to express a PC-1 polypeptide and/or nucleic acid designed to express a PC-2 polypeptide can be administered to a mammal (e.g., a human) in need thereof (e.g., a human having, or at risk of developing, a PKD) as described herein to reduce the severity of one or more symptoms of a PDK and/or one or more complications associated with PKD by, for example, 10, 20, 30, 40, 50, 60, 70, 80, 90, 95, or more percent. For example, nucleic acid designed to express one or more gene therapy components (or the gene therapy components themselves) designed to activate transcription of a PKD1 gene and/or a PKD2 gene can be administered to a mammal (e.g., a human) in need thereof (e.g., a human having, or at risk of developing, a PKD) as described herein to reduce the severity of one or more symptoms of a PDK and/or one or more complications associated with PKD by, for example, 10, 20, 30, 40, 50, 60, 70, 80, 90, 95, or more percent.

In some cases, a mammal (e.g., a human) having, or at risk of developing, a polycystic disease (e.g., a PKD) can be treated as described herein (e.g., by administering nucleic acid designed to increase a level of PC-1 polypeptides and/or PC-2 polypeptides within a mammal) to reduce or eliminate one or more cysts (e.g., one or more renal cysts) within the mammal. For example, nucleic acid designed to express a PC-1 polypeptide and/or nucleic acid designed to express a PC-2 polypeptide can be administered to a mammal (e.g., a human) in need thereof (e.g., a human having one or more cysts associate with a polycystic disease such as PKD) as described herein to reduce the size (e.g., volume) of a cyst within the mammal by, for example, 10, 20, 30, 40, 50, 60, 70, 80, 90, 95, or more percent. For example, nucleic acid designed to express one or more gene therapy components (or the gene therapy components themselves) designed to activate transcription of a PKD1 gene and/or a PKD2 gene can be administered to a mammal (e.g., a human) in need thereof (e.g., a human having one or more cysts associated with a polycystic disease such as PKD) as described herein to reduce the cystic index (also referred to as a cystic burden; e.g., the percentage of an organ such as a kidney that is occupied by cysts) in the mammal by, for example, 10, 20, 30, 40, 50, 60, 70, 80, 90, 95, or more percent. Any appropriate method can be used to determine the size of a cyst (e.g., a renal cyst) and/or a cystic index within a mammal (e.g., a mammal having, or at risk of developing, a polycystic disease such as PKD). For example, ultrasound, computed tomography (CT) scanning, magnetic resonance imaging (MRI), and/or histological analysis can be used to determine the size of a cyst (e.g., a renal cyst) and/or a cystic index of a mammal (e.g., a mammal having, or at risk of developing, a polycystic disease such as PKD). In some cases, a cystic index can be determined as described elsewhere (see, e.g., Nieto et al., PLOS One, 11(10):e0163063 (2016)).

In some cases, a mammal (e.g., a human) having, or at risk of developing, a polycystic disease (e.g., a PKD) can be treated as described herein (e.g., by administering nucleic acid designed to increase a level of PC-1 polypeptides and/or PC-2 polypeptides within a mammal) to reduce the total kidney volume of one or both kidneys within the mammal and/or to reduce the body weight of the mammal. For example, nucleic acid designed to express a PC-1 polypeptide and/or nucleic acid designed to express a PC-2 polypeptide can be administered to a mammal (e.g., a human) in need thereof (e.g., a human having one or more cysts associate with a polycystic disease such as PKD) as described herein to reduce the total kidney volume of a kidney within the mammal and/or to reduce the body weight of the mammal by, for example, 10, 20, 30, 40, 50, 60, 70, 80, 90, 95, or more percent. For example, nucleic acid designed to express one or more gene therapy components (or the gene therapy components themselves) designed to activate transcription of a PKD1 gene and/or a PKD2 gene can be administered to a mammal (e.g., a human) in need thereof (e.g., a human having one or more cysts associate with a polycystic disease such as PKD) as described herein to reduce the total kidney volume of a kidney within the mammal and/or to reduce the body weight of the mammal by, for example, 10, 20, 30, 40, 50, 60, 70, 80, 90, 95, or more percent. Any appropriate method can be used to determine the total kidney volume of a kidney. For example, ultrasound, CT scanning, and/or MRI can be used to determine the weight of a kidney.

Any appropriate mammal having, or at risk of developing, a polycystic disease (e.g., a PKD) can be treated as described herein (e.g., by administering nucleic acid designed to increase a level of PC-1 polypeptides and/or PC-2 polypeptides within a mammal). Examples of mammals having, or at risk of developing, a polycystic disease (e.g., a PKD) that can be treated as described herein include, without limitation, humans, non-human primates (e.g., monkeys), dogs, cats, horses, cows, pigs, sheep, mice, rat, hamsters, camels, and llamas. In some cases, a human having, or at risk of developing, a polycystic disease (e.g., a PKD) can be treated by administering nucleic acid designed to express a PC-1 polypeptide and/or nucleic acid designed to express a PC-2 polypeptide to the human. In some cases, a human having, or at risk of developing, a polycystic disease (e.g., a PKD) can be treated by administering nucleic acid designed to express one or more gene therapy components (or the gene therapy components themselves) designed to activate transcription of a PKD1 gene and/or a PKD2 gene to the human.

Any appropriate polycystic disease can be treated as described herein (e.g., by administering nucleic acid designed to increase a level of PC-1 polypeptides and/or PC-2 polypeptides within a mammal). Examples of polycystic diseases that can be treated as described herein include, without limitation, PKDs such as ADPKD type 1 and ADPKD type 2. In some cases, a mammal (e.g., a human) having, or at risk of developing, PKD (e.g., ADPKD) can be treated by administering nucleic acid designed to express a PC-1 polypeptide and/or nucleic acid designed to express a PC-2 polypeptide to the mammal. In some cases, a mammal (e.g., a human) having, or at risk of developing, PKD (e.g., ADPKD) can be treated by administering nucleic acid designed to express one or more gene therapy components (or the gene therapy components themselves) designed to activate transcription of a PKD1 gene and/or a PKD2 gene to the mammal.

When treating a mammal having, or at risk of developing, a polycystic disease (e.g., a PKD) as described herein (e.g., by administering nucleic acid designed to increase a level of PC-1 polypeptides and/or PC-2 polypeptides within a mammal), the mammal can have one or more cysts present in and/or on any tissue or organ within the mammal. Examples of tissues and organs within a mammal having a polycystic disease (e.g., a PKD) that can have one or more cysts include, without limitation, the kidney, the liver, seminal vesicles, pancreas, and arachnoid membrane. For example, a mammal (e.g., a human) having a polycystic disease (e.g., a PKD) can have one or more renal cysts (e.g., one or more cysts present on or within one or both kidneys).

In some cases, methods for treating a mammal (e.g., a human) having, or at risk of developing, a polycystic disease (e.g., a PKD) also can include identifying a mammal as having, or as being at risk of developing, a polycystic disease (e.g., a PKD). Any appropriate method can be used to identify a mammal as having, or as being at risk of developing, a polycystic disease (e.g., a PKD). For example, imaging techniques (e.g., ultrasound, CT scan, and MRI), laboratory tests (e.g., genetic testing for mutation of one or both copies of the PKD1 gene and/or mutation of one or both copies of the PKD2 gene present in a mammal), and/or generation of family pedigrees can be used to identify a mammal as having, or as being at risk of developing, a polycystic disease (e.g., a PKD).

Once identified as having, or as being at risk of developing, a polycystic disease (e.g., a PKD), the mammal (e.g., the human) can be administered, or instructed to self-administer, nucleic acid designed to increase a level of PC-1 polypeptides and/or PC-2 polypeptides within a mammal as described herein.

In some cases, nucleic acid designed to increase a level of PC-1 polypeptides and/or PC-2 polypeptides within a mammal can include nucleic acid designed to express a PC-1 polypeptide and/or nucleic acid designed to express a PC-2 polypeptide. Nucleic acid designed to express PC-1 polypeptides and/or PC-2 polypeptides within a mammal can express any appropriate PC-1 polypeptide and/or any appropriate PC-2 polypeptide. In some cases, the methods and materials provided herein can include administering to a mammal (e.g., a human) having, or at risk of developing, a polycystic disease (e.g., a PKD) nucleic acid designed to express a PC-1 polypeptide. Examples of PC-1 polypeptides and nucleic acids encoding PC-1 polypeptides include, without limitation, those set forth in the National Center for Biotechnology Information (NCBI) databases at, for example, accession no. NM_001009944 (version NM_001009944.3), and accession no. AAC34211 (version AAC34211.1).

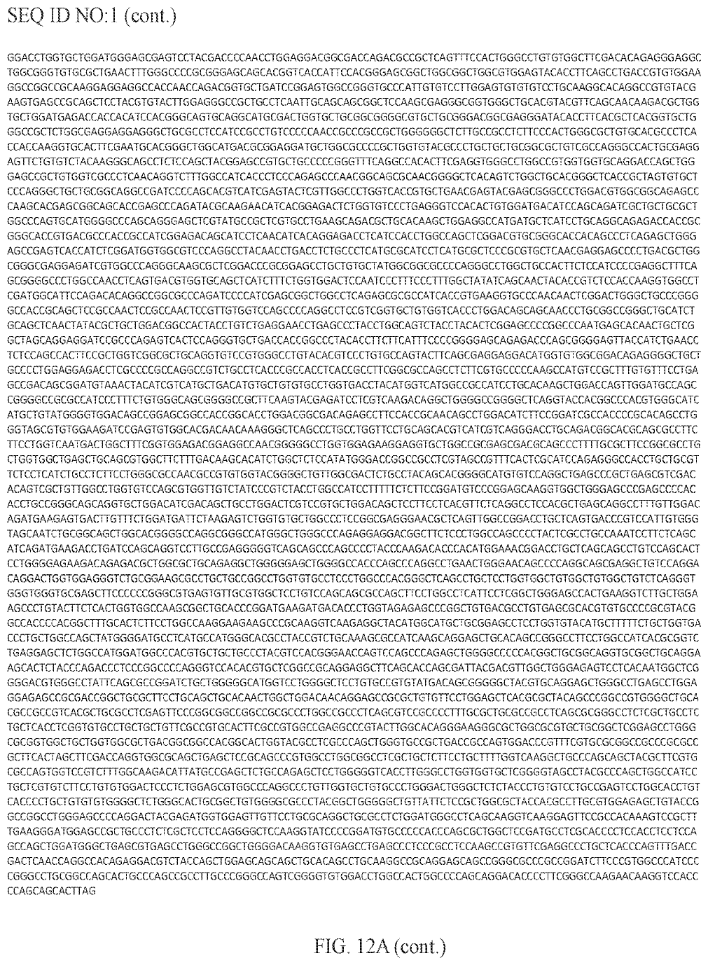

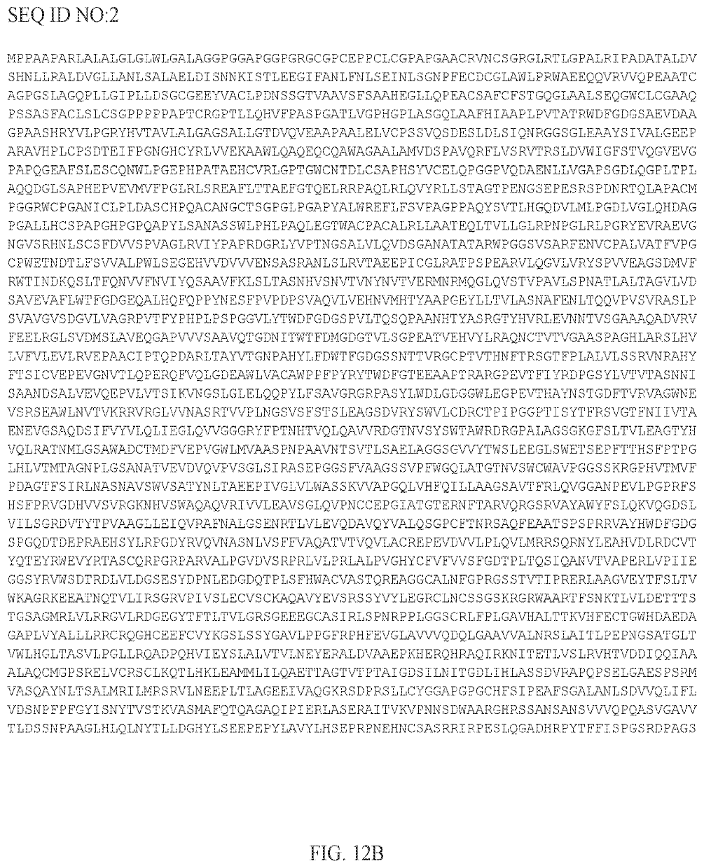

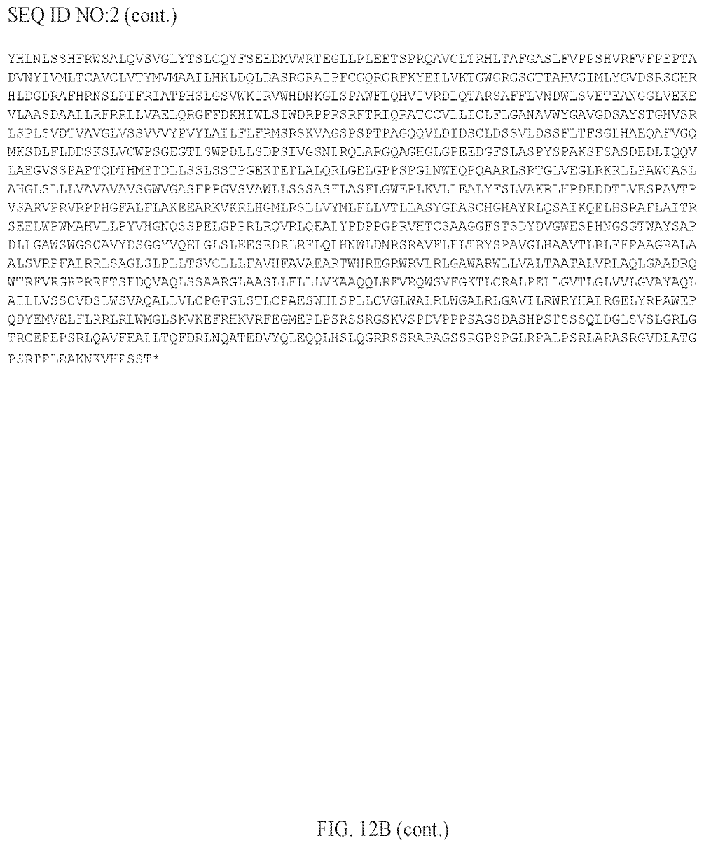

In some cases, a nucleic acid encoding a PC-1 polypeptide can have an nucleotide sequence set forth in SEQ ID NO: 1 (see, e.g., FIG. 12A). In some cases, a PC-1 polypeptide can have an amino acid sequence set forth in SEQ ID NO:2 (see, e.g., FIG. 12B).

In some cases, a variant of a PC-1 polypeptide can be used in place of or in addition to a PC-1 polypeptide. A variant of a PC-1 polypeptide can have the amino acid sequence of a naturally-occurring PC-1 polypeptide with one or more (e.g., e.g., one, two, three, four, five, six, seven, eight, nine, ten, or more) amino acid deletions, additions, substitutions, or combinations thereof, provided that the variant retains the function of a naturally-occurring PC-1 polypeptide.

In some cases, the methods and materials provided herein can include administering to a mammal (e.g., a human) having, or at risk of developing, a polycystic disease (e.g., a PKD) nucleic acid designed to express a PC-2 polypeptide. Examples of PC-2 polypeptides and nucleic acids encoding PC-2 polypeptides include, without limitation, those set forth in the National Center for Biotechnology Information (NCBI) databases at, for example, accession no. NR_156488 (version NR_156488.2), and accession no. Q13563 (version Q13563.3).

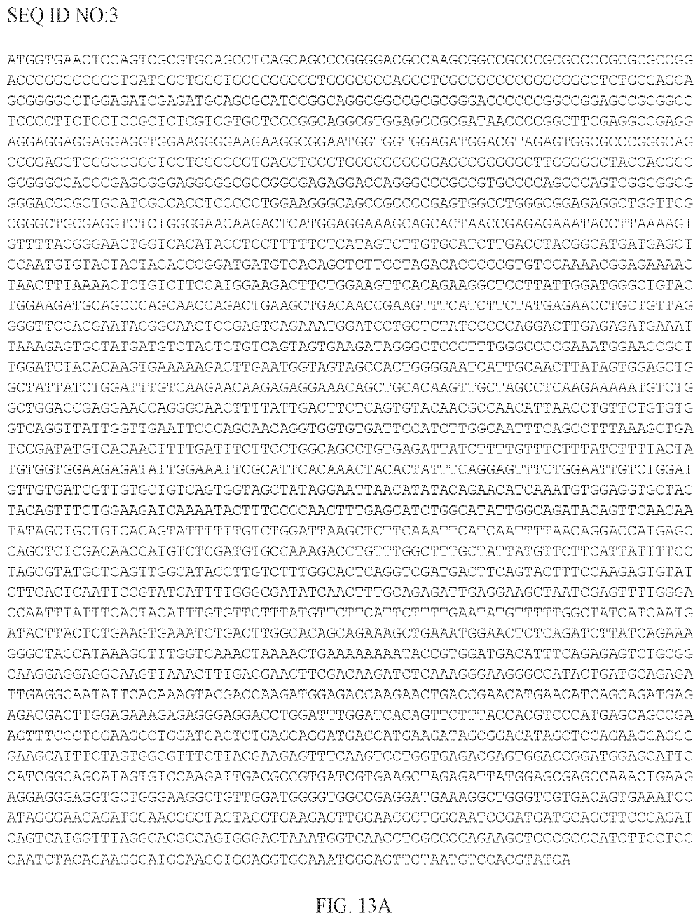

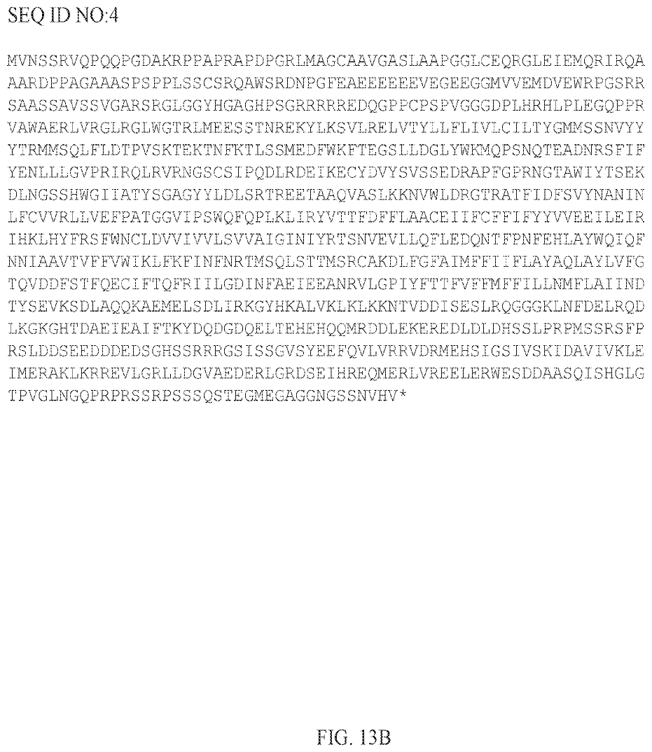

In some cases, a nucleic acid encoding a PC-2 polypeptide can have an nucleotide sequence set forth in SEQ ID NO:3 (see, e.g., FIG. 13A). In some cases, a PC-2 polypeptide can have an amino acid sequence set forth in SEQ ID NO:4 (see, e.g., FIG. 13B).

In some cases, a variant of a PC-2 polypeptide can be used in place of or in addition to a PC-2 polypeptide. A variant of a PC-2 polypeptide can have the amino acid sequence of a naturally-occurring PC-1 polypeptide with one or more (e.g., e.g., one, two, three, four, five, six, seven, eight, nine, ten, or more) amino acid deletions, additions, substitutions, or combinations thereof, provided that the variant retains the function of a naturally-occurring PC-2 polypeptide.

Any appropriate amino acid residue set forth in SEQ ID NO:2 and/or any appropriate amino acid residue set forth in SEQ ID NO:3 can be deleted, and any appropriate amino acid residue (e.g., any of the 20 conventional amino acid residues or any other type of amino acid such as ornithine or citrulline) can be added to or substituted within the sequence set forth in SEQ ID NO:2 and/or SEQ ID NO:4. The majority of naturally occurring amino acids are L-amino acids, and naturally occurring polypeptides are largely comprised of L-amino acids. D-amino acids are the enantiomers of L-amino acids. In some cases, a polypeptide provided herein can contain one or more D-amino acids. In some embodiments, a polypeptide can contain chemical structures such as ε-aminohexanoic acid: hydroxylated amino acids such as 3-hydroxyproline, 4-hydroxyproline, (5R)-5-hydroxy-L-lysine, allo-hydroxylysine, and 5-hydroxy-L-norvaline: or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine): (2) neutral hydrophilic amino acids (cysteine, serine, and threonine): (3) acidic amino acids (aspartic acid and glutamic acid): (4) basic amino acids (asparagine, glutamine, histidine, lysine, and arginine): (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for SEQ ID NO:2 and/or SEQ ID NO:4 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Further examples of conservative substitutions that can be made at any appropriate position within SEQ ID NO:2 and/or SEQ ID NO:4 are set forth in Table 1 below.

TABLE 1
Examples of conservative amino acid substitutions.

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some cases, a variant of a PC-1 polypeptide can be designed to include the amino acid sequence set forth in SEQ ID NO:2 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

In some cases, a variant of a PC-1 polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:2, provided that it includes at least one difference (e.g., at least one amino acid addition, deletion, or substitution) with respect to SEQ ID NO:2, can be used.

In some cases, a variant of a PC-2 polypeptide can be designed to include the amino acid sequence set forth in SEQ ID NO:4 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

In some cases, a variant of a PC-2 polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:4, provided that it includes at least one difference (e.g., at least one amino acid addition, deletion, or substitution) with respect to SEQ ID NO:4, can be used.

The percent sequence identity between a particular nucleic acid or amino acid sequence and a sequence referenced by a particular sequence identification number (e.g., SEQ ID NO:2 and/or SEQ ID NO:4) is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt): -j is set to a file containing the second nucleic acid sequence to be compared (e.g., C:\seq2.txt): -p is set to blastn: -o is set to any desired file name (e.g., C:\output.txt): -q is set to -l: -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -l -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology; then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:2 and/or SEQ ID NO:4), followed by multiplying the resulting value by 100. It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In some cases, nucleic acid designed to express a PC-1 polypeptide and/or nucleic acid designed to express a PC-2 polypeptide can be the form of a vector (e.g., a viral vector or a non-viral vector). In cases where the methods and materials provided herein include nucleic acid designed to express a PC-1 polypeptide and nucleic acid designed to express a PC-2 polypeptide, the nucleic acid designed to express a PC-1 polypeptide and the nucleic acid designed to express a PC-2 polypeptide can be present in the same vector or in separate vectors.

In some cases, nucleic acid designed to express a PC-1 polypeptide and/or nucleic acid designed to express a PC-2 polypeptide can be used for transient expression of a PC-1 polypeptide and/or a PC-2 polypeptide. In some cases, nucleic acid designed to express a PC-1 polypeptide and/or nucleic acid designed to express a PC-2 polypeptide can be used for stable expression of a PC-1 polypeptide and/or a PC-2 polypeptide. In cases where nucleic acid designed to express a PC-1 polypeptide and/or nucleic acid designed to express a PC-2 polypeptide is used for stable expression of a PC-1 polypeptide and/or a PC-2 polypeptide, the nucleic acid encoding a PC-1 polypeptide and/or the nucleic acid encoding a PC-2 polypeptide can be engineered to integrate into the genome of a cell. Nucleic acid can be engineered to integrate into the genome of a cell using any appropriate method. For example, gene editing techniques (e.g., CRISPR or TALEN gene editing) can be used to integrate nucleic acid designed to express a PC-1 polypeptide and/or nucleic acid designed to express a PC-2 polypeptide into the genome of a cell.

When a vector used to deliver nucleic acid designed to express a PC-1 polypeptide and/or nucleic acid designed to express a PC-2 polypeptide to a mammal is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. In some cases, a viral vector can be a helper dependent (HD) viral vector. Examples of virus-based vectors that can be used to deliver nucleic acid designed to express a PC-1 polypeptide and/or nucleic acid designed to express a PC-2 polypeptide to a mammal include, without limitation, virus-based vectors based on Ads (e.g., HDAds), AAVs, lentiviruses (LVs), measles viruses, Sendai viruses, herpes viruses, or vesicular stomatitis viruses (VSVs). In some cases, nucleic acid designed to express a PC-1 polypeptide and/or nucleic acid designed to express a PC-2 polypeptide can be delivered to a mammal using a HDAd vector. In some cases, nucleic acid designed to express a PC-2 polypeptide can be delivered to a mammal using an AAV vector. In some cases, a viral vector including nucleic acid designed to express a PC-1 polypeptide and/or nucleic acid designed to express a PC-2 polypeptide can have low seroprevalence in a mammal to be treated as described herein.

When a vector used to deliver nucleic acid designed to express a PC-1 polypeptide and/or nucleic acid designed to express a PC-2 polypeptide to a mammal (e.g., a human) is a non-viral vector, any appropriate non-viral vector can be used. In some cases, a non-viral vector can be an expression plasmid (e.g., a cDNA expression vector).

In some cases, nucleic acid designed to express a PC-1 polypeptide and/or nucleic acid designed to express a PC-2 polypeptide can be administered to a mammal complexed with lipids, polymers, nanoparticles (e.g., nanospheres), and/or lipid nanoparticles (LNPs). For example, nucleic acid designed to express a PC-1 polypeptide and/or nucleic acid designed to express a PC-2 polypeptide can be complexed to one or more LNPs.

In addition to nucleic acid designed to express a PC-1 polypeptide and/or nucleic acid designed to express a PC-2 polypeptide, nucleic acid designed to increase a level of PC-1 polypeptides and/or PC-2 polypeptides within a mammal can contain one or more regulatory elements operably linked to the nucleic acid encoding a PC-1 polypeptide and/or the nucleic acid encoding a PC-2 polypeptide. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a PC-1 polypeptide and/or nucleic acid encoding a PC-2 polypeptide. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner (e.g., a cadherin 16 (Cdh16 or Ksp-cadherin) promoter sequence such as a mouse Cdh16 promoter sequence). Examples of promoters that can be used to drive expression of a PC-1 polypeptide and/or PC-2 polypeptide include, without limitation, EF1α promoter sequences, CBh promoter sequences, PKD1 promoter sequences, PKD2 promoter sequences, cytomegalovirus (CMV) promoter sequences (e.g., human CMV promoter sequences), Rous sarcoma virus (RSV) promoter sequences, aquaporin 2 (AQP2) promoter sequences, gamma-glutamyltransferase 1 (Ggt1) promoter sequences, and Ksp-cadherin promoter sequences. As used herein, “operably linked” refers to positioning of a regulatory element in a vector relative to a nucleic acid encoding a polypeptide in such a way as to permit or facilitate expression of the encoded polypeptide. For example, a vector can contain a promoter and nucleic acid encoding a PC-1 polypeptide. In this case, the promoter is operably linked to a nucleic acid encoding a PC-1 polypeptide such that it drives expression of the PC-1 polypeptide in cells. In cases where a vector contains both nucleic acid designed to express a PC-1 polypeptide and nucleic acid designed to express a PC-2 polypeptide, the nucleic acid designed to express a PC-1 polypeptide and the nucleic acid designed to express a PC-2 polypeptide can be operably linked to the same promoter or different promoters.

In some cases, nucleic acid designed to express a PC-1 polypeptide and/or nucleic acid designed to express a PC-2 polypeptide can contain nucleic acid encoding a detectable label. For example, a vector can include nucleic acid designed to express a PC-1 polypeptide and nucleic acid encoding a detectable label positioned such that the encoded polypeptide is a fusion polypeptide that includes a PC-1 polypeptide fused to a detectable polypeptide. In some cases, a detectable label can be a peptide tag. Examples of detectable labels that can be used as described herein include, without limitation, HA tags, Myc-tags, FLAG-tags, fluorescent polypeptides (e.g., green fluorescent polypeptides (GFPs), and mCherry polypeptides), luciferase polypeptides, and sodium iodide symporter (NIS) polypeptides.

Nucleic acid designed to express a PC-1 polypeptide and/or nucleic acid designed to express a PC-2 polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a PC-1 polypeptide or a PC-2 polypeptide.

In some cases, a vector including nucleic acid designed to express a PC-1 polypeptide can be a HDAd vector including nucleic acid designed to express a PC-1 polypeptide that is operably linked to a CBh promoter sequence. An exemplary HDAd vector including nucleic acid encoding a PC-1 polypeptide that is operably linked to a CBh promoter sequence can include the nucleic acid sequence set forth in SEQ ID NO:5.

In some cases, a vector including nucleic acid designed to express a PC-2 polypeptide can be a AAV vector including nucleic acid designed to express a PC-2 polypeptide that is operably linked to a EF1a promoter sequence. An exemplary AAV vector including nucleic acid encoding a PC-2 polypeptide that is operably linked to a EF1α promoter sequence can include the nucleic acid sequence set forth in SEQ ID NO:6.

In some cases, a vector including nucleic acid designed to express a PC-1 polypeptide can be a HDAd vector including nucleic acid designed to express a PC-1 polypeptide that is operably linked to a CBh promoter sequence and include nucleic acid designed to express a PC-2 polypeptide that is operably linked to a EF1a promoter sequence. An exemplary HDAd vector including nucleic acid encoding a PC-1 polypeptide that is operably linked to a CBh promoter sequence and including nucleic acid encoding a PC-2 polypeptide that is operably linked to a EF1a promoter sequence can include the nucleic acid sequence set forth in SEQ ID NO:7.

In some cases, nucleic acid designed to increase a level of PC-1 polypeptides and/or PC-2 polypeptides within a mammal can include one or more nucleic acid molecules designed to express gene therapy components designed to activate transcription of a PKD1 gene (e.g., resulting in an increased level of PC-1 polypeptides) and/or to activate transcription of a PKD2 gene (e.g., resulting in an increased level of PC-2 polypeptides). For example, nucleic acid designed to increase a level of PC-1 polypeptides and/or PC-2 polypeptides within a mammal can include one or more nucleic acid molecules designed to express the components of a targeted gene activation system (e.g., designed for CRISPR-Cas9-based targeted gene activation system) designed to upregulate transcription of the PKD1 gene and/or the PKD2 gene to increase the level of PC-1 polypeptides and/or PC-2 polypeptides in cells. Any appropriate targeted gene activation system can be used (e.g., a synergistic activation mediators (SAM) system). In some cases, a targeted gene activation system can include (a) a fusion polypeptide including a deactivated Cas (dCas) polypeptide and a transcriptional activator polypeptide, (b) one or more helper activator polypeptides, and (c) a nucleic acid molecule including (i) a nucleic acid sequence that is complementary to a target sequence within a PKD1 gene and/or a PKD2 gene, and (ii) a nucleic acid sequence that can bind the one or more helper activator polypeptides. For example, nucleic acid designed to increase a level of PC-1 polypeptides and/or PC-2 polypeptides within a mammal can include (a) nucleic acid that can express a fusion polypeptide including a deactivated Cas (dCas) polypeptide and a transcriptional activator polypeptide, (b) nucleic acid that can express one or more helper activator polypeptides, and (c) nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides.

A fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can include any appropriate dCas polypeptide. Examples of dCas polypeptides that can be included in a fusion polypeptide including a dCas polypeptide and a transcriptional activator polypeptide that can be used as a targeted gene activation system (e.g., a SAM system) designed to activate transcription of a PKD1 gene and/or to activate transcription of a PKD2 gene can include, without limitation, deactivated Cas9 (dCas9) polypeptides (e.g., deactivated Streptococcus pyogenes Cas9 (dSpCas9), deactivated Staphylococcus aureus Cas9 (dSaCas9), and deactivated Campylobacter jejuni Cas9 (dCjCas9)), and deactivated Cas phi (dCasΦ) polypeptides. In some cases, a dCas polypeptide that can be included in a fusion polypeptide including a dCas polypeptide and a transcriptional activator polypeptide that can be used as a targeted gene activation system (e.g., a SAM system) designed to activate transcription of a PKD1 gene and/or to activate transcription of a PKD2 gene can be as described elsewhere (see, e.g., Konermann et al., Nature, January 29:517(7536):583-8 (2015) at, for example, the Supplementary Materials; Sajwan et al., Sci Rep., 9:18104 (2019) at, for example, Supplementary Materials; Jiang et al., Biosci. Rep., 39(8):BSR20191496 (2019) at, for example, Table 1). A dCas polypeptide in a fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be encoded by any appropriate nucleic acid sequence.

A fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can include any appropriate transcriptional activator polypeptide. In some cases, a transcriptional activator polypeptide can recruit an RNA polymerase. In some cases, a transcriptional activator polypeptide can recruit one or more transcription factors and/or transcription co-factors (e.g., RNA polymerase co-factors). Examples of transcriptional activator polypeptides that can be included in a fusion polypeptide including a dCas polypeptide and a transcriptional activator polypeptide that can be used in a targeted gene activation system (e.g., a SAM system) designed to activate transcription of a PKD1 gene and/or to activate transcription of a PKD2 gene can include, without limitation, polypeptides having four copies of viral protein 16 (VP64 polypeptides). In some cases, a transcriptional activator polypeptide that can be included in a fusion polypeptide including a dCas polypeptide and a transcriptional activator polypeptide that can be used in a targeted gene activation system (e.g., a SAM system) designed to activate transcription of a PKD1 gene and/or to activate transcription of a PKD2 gene can be as described elsewhere (see, e.g., Konermann et al., Nature, January 29:517(7536):583-8 (2015) at, for example, the Supplementary Materials; Sajwan et al., Sci Rep., 9:18104 (2019) at, for example, Supplementary Materials; Jiang et al., Biosci. Rep., 39(8):BSR20191496 (2019) at, for example, Table 1). A transcriptional activator polypeptide in a fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be encoded by any appropriate nucleic acid sequence.

A fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can include the dCas polypeptide and the transcriptional activator polypeptide in any orientation. In some cases, a transcriptional activator polypeptide can be fused to the N-terminus of a dCas polypeptide. In some cases, a transcriptional activator polypeptide can be fused to the C-terminus of a dCas polypeptide.

In some cases, a fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can include a dSpCas9 polypeptide and a VP64 polypeptide. For example, a fusion polypeptide including a dCas polypeptide and a transcriptional activator polypeptide that can be used in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be a dCas9-VP64 fusion polypeptide.

A fusion polypeptide including a dCas polypeptide and a transcriptional activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be encoded by any appropriate nucleic acid sequence.

A targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can include any appropriate helper activator polypeptide. Examples of helper activator polypeptides that can be used in a targeted gene activation system (e.g., a SAM system) designed to activate transcription of a PKD1 gene and/or to activate transcription of a PKD2 gene can include, without limitation, Escherichia virus MS2 coat protein (MS2) polypeptides, nuclear factor NF-kappa-B p65 subunit (p65) polypeptides, heat shock factor protein 1 (HSF1) polypeptides, VP64 polypeptides. In some cases, a helper activator polypeptide can include two or more (e.g., two, three, or more) helper activator polypeptides. For example, a helper activator polypeptide can be a fusion polypeptide including two or more helper activator polypeptides. For example, a helper activator polypeptide can be a complex including two or more helper activator polypeptide. In some cases, a helper activator polypeptide can include a MS2 polypeptide, a p65 polypeptide, and a HSF1 polypeptide (a MS2-P65-HSF1 (MPH) polypeptide). In some cases, a helper activator polypeptide that can be used in a targeted gene activation system (e.g., a SAM system) designed to activate transcription of a PKD1 gene and/or to activate transcription of a PKD2 gene can be as described elsewhere (see, e.g., Konermann et al., Nature, January 29:517(7536):583-8 (2015) at, for example, the Supplementary Materials; Sajwan et al., Sci Rep., 9:18104 (2019) at, for example, Supplementary Materials; Jiang et al., Biosci. Rep., 39(8):BSR20191496 (2019) at, for example, Table 1). A helper activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be encoded by any appropriate nucleic acid sequence.

A targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can include any appropriate nucleic acid molecule including (i) a nucleic acid sequence that is complementary to a target sequence within a PKD1 gene and/or a PKD2 gene, and (ii) a nucleic acid sequence that can bind the helper activator polypeptide. A nucleic acid sequence that is complementary to a target sequence within a PKD1 gene and/or a PKD2 gene can be any appropriate length. In some cases, a nucleic acid sequence that is complementary to a target sequence within a PKD1 gene and/or a PKD2 gene can include from 19 nucleotides to 21 nucleotides.

In some cases, a nucleic acid molecule including (i) a nucleic acid sequence that is complementary to a target sequence within a PKD1 gene and/or a PKD2 gene, and (ii) a nucleic acid sequence that can bind the helper activator polypeptide that can be used in a targeted gene activation system (e.g., a SAM system) designed to activate transcription of a PKD1 gene and/or to activate transcription of a PKD2 gene can include a nucleic acid sequence that is complementary to a target sequence within a PKD1 gene. A nucleic acid sequence that is complementary to a target sequence within a PKD1 gene can include any appropriate nucleic acid sequence. A nucleic acid sequence that is complementary to a target sequence within a PKD1 gene can be complementary to (e.g., can be designed to target) any target sequence within a PKD1 gene (e.g., can target any location within a PKD1 gene). In some cases, a nucleic acid sequence that is complementary to a target sequence within a PKD1 gene can be a single stranded nucleic acid sequence. In some cases, a target sequence within a PKD1 gene can be in a promoter sequence of the PKD1 gene. In some cases, a target sequence within a PKD1 gene can be from about 1 nucleotide to about 200 nucleotides away from a promoter sequence of the PKD1 gene. Examples of nucleic acid sequences that are complementary to a target sequence within a PKD1 gene include, without limitation, nucleic acid sequences that can be encoded by a nucleic acid sequence including the sequence TCGCGCTGTGGCGAAGGGGG (SEQ ID NO:13), a nucleic acid sequence including the sequence CCAGTCCCTCATCGCTGGCC (SEQ ID NO:14), and a nucleic acid sequence including the sequence GGAGCGGAGGGTGAAGCCTC (SEQ ID NO:15).

In some cases, a nucleic acid molecule including (i) a nucleic acid sequence that is complementary to a target sequence within a PKD1 gene and/or a PKD2 gene, and (ii) a nucleic acid sequence that can bind the helper activator polypeptide that can be used in a targeted gene activation system (e.g., a SAM system) designed to activate transcription of a PKD1 gene and/or to activate transcription of a PKD2 gene can include a nucleic acid sequence that is complementary to a target sequence within a PKD2 gene. A nucleic acid sequence that is complementary to a target sequence within a PKD2 gene can include any appropriate nucleic acid sequence. A nucleic acid sequence that is complementary to a target sequence within a PKD2 gene can be complementary to (e.g., can be designed to target) any target sequence within a PKD2 gene (e.g., can target any location within a PKD2 gene). In some cases, a nucleic acid sequence that is complementary to a target sequence within a PKD2 gene can be a single stranded nucleic acid sequence. In some cases, a target sequence within a PKD2 gene can be in a promoter sequence of the PKD2 gene. In some cases, a target sequence within a PKD2 gene can be from about 1 nucleotide to about 200 nucleotides away from a promoter sequence of the PKD2 gene. Examples of nucleic acid sequences that are complementary to a target sequence within a PKD2 gene include, without limitation, nucleic acid sequences that can be encoded by a nucleic acid sequence including the sequence ACGCGGACTCGGGAGCCGCC (SEQ ID NO:23), a nucleic acid sequence including the sequence ATCCGCCGCGGCGCGCTGAG (SEQ ID NO:24), and a nucleic acid sequence including the sequence GTGCGAGGGAGCCGCCCCCG (SEQ ID NO:25).

A nucleic acid sequence that is complementary to a target sequence within a PKD1 gene and/or a PKD2 gene that can be included in a nucleic acid molecule including (i) a nucleic acid sequence that is complementary to a target sequence within a PKD1 gene and/or a PKD2 gene, and (ii) a nucleic acid sequence that can bind the helper activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be encoded by any appropriate nucleic acid sequence. In some cases, nucleic acid sequences that encode a nucleic acid that is complementary to a target sequence within a PKD1 gene can be encoded by a nucleic acid sequence shown in Table 2 or Table 3.

In some cases, a nucleic acid molecule including (i) a nucleic acid sequence that is complementary to a target sequence within a PKD1 gene and/or a PKD2 gene, and (ii) a nucleic acid sequence that can bind the helper activator polypeptide that can be used in a targeted gene activation system (e.g., a SAM system) designed to activate transcription of a PKD1 gene and/or to activate transcription of a PKD2 gene can include any appropriate nucleic acid sequence that can bind the helper activator polypeptide. In some cases, a nucleic acid sequence that can bind the helper activator polypeptide can bind a MS2 polypeptide. Examples of nucleic acid sequences that can bind the helper activator polypeptide (e.g., a MS2 polypeptide) can include, without limitation, nucleic acid sequences that can be encoded by a nucleic acid sequence including the sequence ACATGAGGATCACCCATGT (SEQ ID NO:26). A nucleic acid sequence that can bind the helper activator polypeptide that can be included in a nucleic acid molecule including (i) a nucleic acid sequence that is complementary to a target sequence within a PKD1 gene and/or a PKD2 gene, and (ii) a nucleic acid sequence that can bind the helper activator polypeptide in a targeted gene activation system (e.g., a SAM system) designed to activate transcription of a PKD1 gene (e.g., resulting in an increased level of PC-1 polypeptides) and/or to activate transcription of a PKD2 gene (e.g., resulting in an increased level of PC-2 polypeptides) can be encoded by any appropriate nucleic acid sequence.

In addition to nucleic acid designed to express one or more gene therapy components designed to activate transcription of a PKD1 gene (e.g., resulting in an increased level of PC-1 polypeptides) and/or to activate transcription of a PKD2 gene (e.g., resulting in an increased level of PC-2 polypeptides), nucleic acid designed to increase a level of PC-1 polypeptides and/or PC-2 polypeptides within a mammal can contain one or more regulatory elements operably linked to nucleic acid that can express (a) a fusion polypeptide including a dCas polypeptide and a transcriptional activator polypeptide, (b) nucleic acid that can express one or more helper activator polypeptides, and/or (c) nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid that can express (a) a fusion polypeptide including a dCas polypeptide and a transcriptional activator polypeptide, (b) a nucleic acid that can express one or more helper activator polypeptides, and/or (c) a nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner (e.g., AQP2 promoter sequences, Ggt1 promoter sequences, and Ksp-cadherin promoter sequences). Examples of promoters that can be used to drive expression of (a) a fusion polypeptide including a dCas polypeptide and a transcriptional activator polypeptide, (b) one or more helper activator polypeptides. and/or (c) a nucleic acid molecule including (i) a nucleic acid sequence that is complementary to a target sequence within a PKD1 gene and/or a PKD2 gene, and (ii) a nucleic acid sequence that can bind the one or more helper activator polypeptides include, without limitation, EF1a promoter sequences, CBh promoter sequences, CMV promoter sequences (e.g., human CMV promoter sequences), RSV promoter sequences, U6 promoter sequences, AQP2 promoter sequences, Ggt1 promoter sequences, and Ksp-cadherin promoter sequences. As used herein, “operably linked” refers to positioning of a regulatory element in a vector relative to a nucleic acid encoding a polypeptide or a nucleic acid (e.g., an RNA) in such a way as to permit or facilitate expression of the encoded polypeptide or the transcribed nucleic acid. For example, a vector can contain a promoter and nucleic acid encoding a fusion polypeptide including a dCas polypeptide and a transcriptional activator polypeptide. In this case, the promoter is operably linked to a nucleic acid encoding a fusion polypeptide including a dCas polypeptide and a transcriptional activator polypeptide such that it drives expression of the fusion polypeptide including a dCas polypeptide and a transcriptional activator polypeptide in cells. In cases where a vector contains both a nucleic acid that can express one or more helper activator polypeptides and a nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides, the nucleic acid that can express one or more helper activator polypeptides and the nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides can be operably linked to the same promoter or different promoters. In cases where a vector contains each of a nucleic acid that can express (a) a fusion polypeptide including dCas polypeptide and a transcriptional activator polypeptide, (b) a nucleic acid that can express one or more helper activator polypeptides, and (c) a nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides, the nucleic acid that can express the fusion polypeptide including dCas polypeptide and a transcriptional activator polypeptide, the nucleic acid that can express the nucleic acid that can express one or more helper activator polypeptides, and the nucleic acid that can express a nucleic acid molecule including (i) a nucleic acid sequence that is complementary to a target sequence within a PKD1 gene and/or a PKD2 gene, and (ii) a nucleic acid sequence that can bind the one or more helper activator polypeptides can be operably linked to the same promoter or different promoters. In cases where two or more nucleic acid sequences are operably linked to a single promoter, the coding sequences of each nucleic acid sequence can be separated by a sequence encoding a cleavage signal (e.g., P2A cleavage signal).

In some cases, one or more nucleic acid molecules designed to express the components of a targeted gene activation system designed to activate transcription of a PKD1 gene (e.g., resulting in an increased level of PC-1 polypeptides) and/or to activate transcription of a PKD2 gene (e.g., resulting in an increased level of PC-2 polypeptides) can be the form of one or more vectors (e.g., viral vectors and/or non-viral vectors). In some cases, one or more nucleic acid molecules designed to express the components of a targeted gene activation system designed to activate transcription of a PKD1 gene and/or to activate transcription of a PKD2 gene can be present in the same vector or in separate vectors.

When a vector used to deliver one or more nucleic acid molecules designed to express the components of a targeted gene activation system designed to activate transcription of a PKD1 gene and/or to activate transcription of a PKD2 gene to a mammal is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples virus-based vectors that can be used to deliver nucleic acid designed to express a PC-1 polypeptide and/or nucleic acid designed to express a PC-2 polypeptide to a mammal include, without limitation, virus-based vectors based on Ads (e.g., HDAds), AAVs, LVs, measles viruses, Sendai viruses, herpes viruses, or VSVs.

When a vector used to deliver one or more nucleic acid molecules designed to express the components of a targeted gene activation system designed to activate transcription of a PKD1 gene and/or to activate transcription of a PKD2 gene to a mammal (e.g., a human) is a non-viral vector, any appropriate non-viral vector can be used. In some cases, a non-viral vector can be an expression plasmid (e.g., a cDNA expression vector).

In some cases, one or more nucleic acid molecules designed to express the components of a targeted gene activation system designed to activate transcription of a PKD1 gene and/or to activate transcription of a PKD2 gene can be administered to a mammal by direct injection of nucleic acid molecules complexed with lipids, polymers, nanoparticles (e.g., nanospheres), and/or LNPs. For example, nucleic acid designed to express a PC-1 polypeptide and/or nucleic acid designed to express a PC-2 polypeptide can be complexed to one or more LNPs.

In some cases, one or more nucleic acid molecules designed to express the components of a targeted gene activation system designed to activate transcription of a PKD1 gene (e.g., resulting in an increased level of PC-1 polypeptides) and/or to activate transcription of a PKD2 gene (e.g., resulting in an increased level of PC-2 polypeptides) can be in a HDAd vector (e.g., in a single HDAd vector) including (a) nucleic acid encoding a dCas9VP64 fusion polypeptide that is operably linked to a CMV promoter sequence, (b) nucleic acid encoding a MPH polypeptide that is operably linked to a EF1a promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence. Exemplary HDAd vectors including (a) nucleic acid encoding a dCas9VP64 fusion polypeptide that is operably linked to a CMV promoter sequence, (b) nucleic acid encoding a MPH polypeptide that is operably linked to a EF1a promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence can include, without limitation, the nucleic acid sequence set forth in SEQ ID NO:8, and the nucleic acid sequence set forth in SEQ ID NO:9.

In some cases, one or more nucleic acid molecules designed to express the components of a targeted gene activation system designed to activate transcription of a PKD1 gene (e.g., resulting in an increased level of PC-1 polypeptides) and/or to activate transcription of a PKD2 gene (e.g., resulting in an increased level of PC-2 polypeptides) can be in the form of two or more AAV vectors including (a) nucleic acid encoding a dCas9VP64 fusion polypeptide that is operably linked to a EF1a promoter sequence, (b) nucleic acid encoding a MPH polypeptide that is operably linked to a CMV promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence. For example, a first AAV vector can include (a) nucleic acid encoding a dCas9VP64 fusion polypeptide that is operably linked to a EF1a promoter sequence, and a second AAV vector can include (b) nucleic acid encoding a MPH polypeptide that is operably linked to a CMV promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence. An exemplary AAV vector including (a) nucleic acid encoding a dCas9VP64 fusion polypeptide that is operably linked to a EF1a promoter sequence can include the nucleic acid sequence set forth in SEQ ID NO: 10. An exemplary AAV vector including (b) nucleic acid encoding a MPH polypeptide that is operably linked to a CMV promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence can include the nucleic acid sequence set forth in SEQ ID NO:11.

In some cases, one or more nucleic acid molecules designed to express the components of a targeted gene activation system designed to activate transcription of a PKD1 gene (e.g., resulting in an increased level of PC-1 polypeptides) and/or to activate transcription of a PKD2 gene (e.g., resulting in an increased level of PC-2 polypeptides) can be in the form of an AAV vector (e.g., a single AAV vector) including (a) nucleic acid encoding a dCasΦ1 polypeptide that is operably linked to a CBh promoter sequence, (b) nucleic acid encoding a MPH polypeptide that is operably linked to the CBh promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence. An exemplary AAV vector including (a) nucleic acid encoding a dCasΦ1 polypeptide that is operably linked to a CBh promoter sequence. (b) nucleic acid encoding a MPH polypeptide that is operably linked to the CBh promoter sequence, and (c) nucleic acid encoding a nucleic acid molecule (e.g., gRNA) including (i) a nucleic acid sequence that is complementary to a target sequence within a PKD1 gene, and (ii) a nucleic acid sequence that can bind a MS2 polypeptide that is operably linked to a U6 promoter sequence can include the nucleic acid sequence set forth in SEQ ID NO:12.

Any appropriate method can be used to deliver nucleic acid designed to increase a level of PC-1 polypeptides and/or PC-2 polypeptides within a mammal to a mammal (e.g., a human). For example, nucleic acid designed to increase a level of PC-1 polypeptides and/or PC-2 polypeptides within a mammal can be administered locally or systemically. For example, nucleic acid designed to increase a level of PC-1 polypeptides and/or PC-2 polypeptides within a mammal can be administered locally by retro-ureter injection and/or subcapsular injection to a mammal (e.g., a human). For example, nucleic acid designed to increase a level of PC-1 polypeptides and/or PC-2 polypeptides within a mammal can be administered systemically by i.p. injection and/or i.v. injection to a mammal (e.g., a human).

Also provided herein are methods for improving delivery of nucleic acid (e.g., vectors such as viral vectors) to a mammal (e.g., to one or more cells within a mammal). For example, inducing proteinuria in a mammal prior to administering nucleic acid can be effective to improve delivery of nucleic acid to one or more cells (e.g., from blood within a mammal into one or more cells) within a mammal. In some cases, a mammal can first be administered one or more LPSs (e.g., to induce proteinuria in the mammal), and can subsequently be administered nucleic acid. For example, a mammal having, or at risk of developing, a polycystic disease (e.g., PKD) can first be administered one or more LPSs, and can subsequently be administered nucleic acid designed to increase a level of PC-1 polypeptides and/or PC-2 polypeptides within the mammal (e.g., to improve delivery of nucleic acid designed to increase a level of PC-1 polypeptides and/or PC-2 polypeptides to one or more cells within a mammal).

Any appropriate LPS having the ability to induce proteinuria in a mammal (e.g., a human) can be used to improve delivery of nucleic acid to cells within the mammal as described herein. In some cases, another agent (e.g., an agent that is not an LPS) that can induce proteinuria in a mammal (e.g., a human) can be used in place of or in addition to one or more LPSs to improve delivery of nucleic acid to a mammal (e.g., to one or more cells within a mammal). An agent that can induce proteinuria in a mammal can be any type of molecule (e.g., a polypeptide, and a small molecule). In some cases, an agent that can induce proteinuria in a mammal can be a cell-opening agent. Examples of agents that can induce proteinuria and be used as described herein include, without limitation, puromycin, adriamycin, protamine sulfate, cationic albumin, and polycations.

In some cases, administering one or more LPSs (and/or another agent or agents that can induce proteinuria in a mammal) prior to administering nucleic acid can be effective to improve delivery of the nucleic acid to the mammal (e.g., to one or more cells within a mammal) by, for example, 10, 20, 30, 40, 50, 60, 70, 80, 90, 95, or more percent (e.g., as compared to the amount of nucleic acid delivered to a mammal that has not been administered one or more LPSs and/or other agent(s) that can induce proteinuria in a mammal).

In some cases, administering one or more LPSs (and/or another agent or agents that can induce proteinuria in a mammal) prior to administering nucleic acid can be effective to deliver large nucleic acid to the mammal (e.g., to one or more cells within a mammal). For example, administering one or more LPSs (and/or another agent or agents that can induce proteinuria in a mammal) prior to administering nucleic acid can be effective to deliver nucleic acid having a size of from about 0.15 kb to about 36 kb (e.g., from about 0.15 kb to about 33 kb, from about 0.15 kb to about 30 kb, from about 0.15 kb to about 28 kb, from about 0.15 kb to about 25 kb, from about 0.15 kb to about 20 kb, from about 0.15 kb to about 17 kb, from about 0.15 kb to about 15 kb, from about 0.15 kb to about 12 kb, from about 0.15 kb to about 10 kb, from about 0.15 kb to about 8 kb, from about 0.15 kb to about 5 kb, from about 0.15 kb to about 3 kb, from about 0.15 kb to about 1 kb, from about 0.15 kb to about 0.5 kb, from about 0.5 kb to about 36 kb, from about 1 kb to about 36 kb, from about 5 kb to about 36 kb, from about 8 kb to about 36 kb, from about 10 kb to about 36 kb, from about 15 kb to about 36 kb, from about 20 kb to about 36 kb, from about 25 kb to about 36 kb, from about 30 kb to about 36 kb, from about 0.5 kb to about 30 kb, from about 1 kb to about 25 kb, from about 5 kb to about 20 kb, from about 10 kb to about 15 kb, from about 1 kb to about 5 kb, from about 5 kb to about 10 kb, from about 15 kb to about 20 kb, from about 20 kb to about 25 kb, from about 25 kb to about 30 kb, or from about 30 kb to about 35 kb) to the mammal (e.g., to one or more cells within a mammal). For example, administering one or more LPSs (and/or another agent or agents that can induce proteinuria in a mammal) prior to administering nucleic acid can be effective to deliver nucleic acid having a mass of from about 10 kilodaltons (kDa) to about 50 kDa (e.g., from about 10 kDa to about 50 kDa, from about 10 kDa to about 40 kDa, from about 10 kDa to about 30 kDa, from about 10 kDa to about 20 kDa, from about 20 kDa to about 40 kDa, from about 25 kDa to about 35 kDa, from about 15 kDa to about 20 kDa, from about 20 kDa to about 25 kDa, from about 25 kDa to about 30 kDa, from about 30 kDa to about 35 kDa, from about 35 kDa to about 40 kDa, from about 40 kDa to about 45 kDa, or from about 45 kDa to about 50 kDa) to the mammal (e.g., to one or more cells within a mammal). For example, administering one or more LPSs (and/or another agent or agents that can induce proteinuria in a mammal) prior to administering nucleic acid can be effective to deliver nucleic acid having a diameter of from about 10 nm to about 26 nm (e.g., from about 10 nm to about 25 nm, from about 10 nm to about 20 nm, from about 10 nm to about 17 nm, from about 10 nm to about 15 nm, from about 10 nm to about 12 nm, from about 12 nm to about 26 nm, from about 15 nm to about 26 nm, from about 18 nm to about 26 nm, from about 20 nm to about 26 nm, from about 22 nm to about 26 nm, from about 12 nm to about 20 nm, from about 15 nm to about 18 nm, from about 12 nm to about 15 nm, from about 18 nm to about 20 nm, or from about 20 nm to about 22 nm) to the mammal (e.g., to one or more cells within a mammal).

Any appropriate amount of one or more LPSs (and/or another agent or agents that can induce proteinuria in a mammal) can be administered to a mammal (e.g., a human) to improve delivery of nucleic acid to any type of cell within the mammal. For example, from about 7 milligrams per kilogram body weight (mg/kg) to about 9 mg/kg of one or more LPSs (and/or another agent or agents that can induce proteinuria in a mammal) can be administered to a mammal (e.g., a human) to improve delivery of nucleic acid to any type of cell within the mammal.

One or more LPSs (and/or another agent or agents that can induce proteinuria in a mammal) can improve delivery of nucleic acid to any type of cell within a mammal. Examples of types of cells that an agent that can induce proteinuria in a mammal can improve delivery of nucleic acid to include, without limitation, kidney cells (e.g., renal tubule epithelial cells and/or proximal tubule cells such as proximal tubule cells adjacent to glomeruli), spleen cells, lungs cells, and brain cells.

One or more LPSs (and/or another agent or agents that can induce proteinuria in a mammal) can be administered to a mammal (e.g., a human) at any appropriate time before nucleic acid is administered to the mammal. In some cases, one or more LPSs (and/or another agent or agents that can induce proteinuria in a mammal) can be administered to a mammal (e.g., a human) at least 18 hours prior to administering nucleic acid to the mammal. For example, one or more LPSs (and/or another agent or agents that can induce proteinuria in a mammal) can be administered to a mammal (e.g., a human) from about 18 hours to about 24 hours prior to administering nucleic acid to the mammal.

Any appropriate method can be used to deliver one or more LPSs (and/or another agent or agents that can induce proteinuria in a mammal) to a mammal (e.g., a human). For example, one or more LPSs (and/or another agent or agents that can induce proteinuria in a mammal) can be administered locally or systemically. For example, one or more LPSs (and/or another agent or agents that can induce proteinuria in a mammal) can be administered locally by retro-ureter injection and/or subcapsular injection to a mammal (e.g., a human). For example, one or more LPSs (and/or another agent or agents that can induce proteinuria in a mammal) can be administered systemically by i.p. injection and/or i.v. injection to a mammal (e.g., a human).

In some cases, methods for treating a mammal (e.g., a human) having, or at risk of developing, a polycystic disease (e.g., a PKD) can include administering to the mammal nucleic acid designed to increase a level of PC-1 polypeptides and/or PC-2 polypeptides within a mammal as the sole active ingredient to treat the mammal.

In some cases, methods for treating a mammal (e.g., a human) having, or at risk of developing, a polycystic disease (e.g., a PKD) as described herein (e.g., by administering nucleic acid designed to increase a level of PC-1 polypeptides and/or PC-2 polypeptides within a mammal) also can include administering to the mammal one or more (e.g., one, two, three, four, five or more) additional active agents (e.g., therapeutic agents) that are effective to treat one or more symptoms of a PKD and/or one or more complications associated with a polycystic disease (e.g., a PKD) to treat the mammal. Examples of additional active agents that can be used as described herein to treat one or more symptoms of a polycystic disease (e.g., a PKD) and/or one or more complications associated with a polycystic disease (e.g., a PKD) include, without limitation, an inhibitor of a vasopressin receptor (e.g., tolvaptan), angiotensin-converting enzyme (ACE) inhibitors, angiotensin II receptor blockers (ARBs), pain relievers (e.g., acetaminophen), antibiotics, pasireotide, and anti-miR-17 oligonucleotide RGLS4326. In some cases, the one or more additional active agents can be administered together with the administration of the nucleic acid designed to increase a level of PC-1 polypeptides and/or PC-2 polypeptides within a mammal. For example, a composition containing nucleic acid designed to increase a level of PC-1 polypeptides and/or PC-2 polypeptides within a mammal also can include one or more additional active agents that are effective to treat one or more symptoms of a polycystic disease (e.g., a PKD) and/or one or more complications associated with a polycystic disease (e.g., a PKD). In some cases, the one or more additional active agents that are effective to treat one or more symptoms of a polycystic disease (e.g., a PKD) and/or one or more complications associated with a polycystic disease (e.g., a PKD) can be administered independent of the administration of the nucleic acid designed to increase a level of PC-1 polypeptides and/or PC-2 polypeptides within a mammal. When the one or more additional active agents that are effective to treat one or more symptoms of a polycystic disease (e.g., a PKD) and/or one or more complications associated with a polycystic disease (e.g., a PKD) are administered independent of the administration of the nucleic acid designed to increase a level of PC-1 polypeptides and/or PC-2 polypeptides within a mammal, the nucleic acid designed to increase a level of PC-1 polypeptides and/or PC-2 polypeptides within a mammal can be administered first, and the one or more additional active agents that are effective to treat one or more symptoms of a polycystic disease (e.g., a PKD) and/or one or more complications associated with a polycystic disease (e.g., a PKD) performed second, or vice versa.

In some cases, methods for treating a mammal (e.g., a human) having, or at risk of developing, a polycystic disease (e.g., a PKD) as described herein (e.g., by administering nucleic acid designed to increase a level of PC-1 polypeptides and/or PC-2 polypeptides within a mammal) also can include subjecting the mammal one or more (e.g., one, two, three, four, five or more) additional treatments (e.g., therapeutic interventions) that are effective to treat one or more symptoms of a polycystic disease (e.g., a PKD) and/or one or more complications associated with a polycystic disease (e.g., a PKD) to treat the mammal. Examples of additional treatments that can be used as described herein to treat one or more symptoms of a polycystic disease (e.g., a PKD) and/or one or more complications associated with a polycystic disease (e.g., a PKD) include, without limitation, consuming a restricted diet (e.g., a diet low in methionine, high in choline, and/or high in betaine content), maintaining a healthy body weight, exercising regularly, undergoing dialysis, undergoing a kidney transplant, and dietary ketosis. In some cases, the one or more additional treatments that are effective to treat one or more symptoms of a polycystic disease (e.g., a PKD) and/or one or more complications associated with a polycystic disease (e.g., a PKD) can be performed at the same time as the administration of the nucleic acid designed to increase a level of PC-1 polypeptides and/or PC-2 polypeptides within a mammal. In some cases, the one or more additional treatments that are effective to treat one or more symptoms of a polycystic disease (e.g., a PKD) and/or one or more complications associated with a polycystic disease (e.g., a PKD) can be performed before and/or after the administration of the nucleic acid designed to increase a level of PC-1 polypeptides and/or PC-2 polypeptides within a mammal.

The invention will be further described in the following examples, which do not limit the scope of the invention described in the claims.

### EXAMPLES

### Example 1: Expression of PC-1 Polypeptides and or PC-2 Polypeptides to Treat ADPKD

This Example describes vectors that can be used as genetic therapies for treating ADPKD by delivering the cDNA of the PKD1 gene, the cDNA of the PKD2 gene, or both (e.g., simultaneously). Both viral and non-viral delivery methods are described.

### Results

A Helper-Dependent Adenoviral Vector that Expresses PKD1, PKD2, or Both

HDAds with all the Ad genome viral open reading frames removed has space for genetic cargo up to 35 kb. AAVs can deliver the 2.9 kb PKD2 cDNA while HDAds can deliver the 12.9 kb PKD1 cDNA or a combination of the PKD1 and PKD2 cDNAs.

### Materials and Methods

### HDAd Vectors

HD-Ad PKD1 vectors were generated that contained a PKD1 cDNA. GFP-Luciferase HDAd vectors were also generated for transduction testing.

A helper virus was used to provide the missing Ad genes and proteins for HDAd vectors. If a normal Ad was used as the helper virus, both the helper and the HDAd virus was packaged, producing a preparation that was contaminated by the helper virus. To avoid this contamination problem, the Ad helper virus has its packaging signal flanked by two LoxP sites.

When the HDAd vector and LoxP-modified helper virus are delivered into 116 cells that overexpress the Cre recombinase, Cre excises the helper virus' packaging signal, blocking its packaging, and significantly reducing helper virus contamination. This system routinely produces yields of HDAd of 10¹³ virus particles (vp) with helper virus contamination below 0.02%.

HDAd was passaged up to 6 times and then purified on 2 CsCl gradients. Once purified, each virus preparation was sequenced to verify identity, and the amount of vector and helper virus was measured by qPCR.

### Testing HDAd Vectors

Once produced, vectors are tested in vitro in 293 and RCTE human cells and IMCD mouse cells. The cells are infected at varied multiplicities of infection (MOI) of each vector. GFP fluorescence are analyzed by fluorescence microscopy and cell lysates will be prepared at the peak time of expression (usually day 2). Once GFPLuc expression is validated for each of the vectors, the vectors proceed to in vivo testing in RC mice. Groups of 5 male and 5 female mice are injected with each of the vectors by the retro-ureter route and sub-capsular routes. One group of male and female mice is injected with PBS as negative controls. Luciferase imaging is performed under isoflurane anesthesia on day 1 and 7. After luciferase imaging, all of the mice are euthanized using CO². Both kidneys are sectioned to identify the cells that are expressing GFP using antibodies against GFP and EpCAM as well as staining with biotinylated lotus tetragonolobus lectin (LTL) to label mature proximal tubules and papillary collecting ducts. The percent transgene protein positive tubule cells are quantified using ImageJ based on pixel counts. The level of gene delivery in the renal pelvis, distal and proximal tubule, and in the glomerulus are determined. ANOVA comparisons are used to compare injection methods and promoters.

Each vector is used to transduce PKD1 and PKD2 null mutant cells and PC-1 and PC-2 expression by the vectors is verified by western blot.

### Shorter Term In Vivo Therapeutic Testing

The vectors are injected into 1 month old RC/RC mice that are early in the PKD disease process. Each virus for injection is blinded. Mice are injected in the right kidney by the retro-ureter route in groups of 10 male and 10 female mice with PBS, HDAd-GFPLuc, HDAd-PKD1, or HDAd-PKD1 and PKD2. Cyst status for mice is established by MRI. The kidneys of the mice are monitored by MRI imaging bi-weekly to assess if vector injection into the right kidney delays cystogenesis progression relative to the uninjected kidneys. Serum creatinine and BUN are measured at varied times to assess kidney function.

Five animals from each group are sacrificed at one week and five animals from each group are sacrificed at one month. Luciferase imaging is performed in the GFP-Luciferase groups just prior to sacrifice to document the persistence of expression mediated by the HDAd vectors. The injected right kidney and the uninjected left kidney are weighed to determine kidney mass to body mass ratios. One half of each kidney is used for western blot and qPCR to determine whether PKD1 expression and PC-1 protein levels are increased. The remaining half is sectioned to identify the cells that are expressing exogenous human PC-1 and for histological examination to examine effects on cyst index, number and growth. Sections are stained by H&E to monitor changes in cyst sizes and infiltration of immune cells into the tissue.

HDAd-PKD1 or HDAd-PKD1 and PKD2 therapies can mediate changes in kidney size and cystic phenotypes relative to control vector and to PBS-injected controls. It is also examined if combined PKD1 and PKD2 provides better balanced expression than PKD1 alone.

### Longer Term In Vivo Therapeutic Testing

The Shorter Term testing described above is repeated, but over longer times with larger group sizes. Five animals from each group are sacrificed at one month, five animals from each group are sacrificed 3 months, five animals from each group are sacrificed 6 months, and five animals from each group are sacrificed at 9 months. Luciferase imaging is performed and gene expression, kidney size, creatinine, BUN, kidney mass, and cyst formation is evaluated to determine if HDAd-PKD1 therapy mediates changes in kidney size and cystic phenotypes relative to control vector and to PBS-injected controls and uninjected kidneys.

### Example 2: Targeted Gene Activation to Treat ADPKD

This Example describes gene activation machinery capable of increasing expression of the wild type PKD1 gene.

### Results

### Targeted Gene Activation of the PKD1 Allele in Human 293 (Adrenal-Derived) Cells

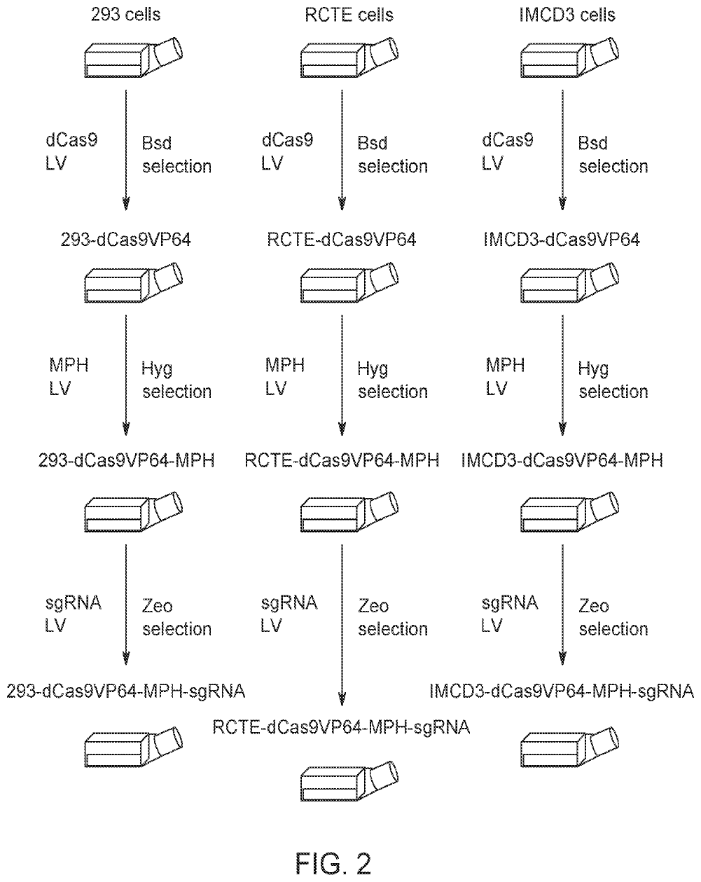

Three separate lentiviral vectors were produced, each of which expressed one of the three components of the Cas9-SAM system and a different selectable marker. Human 293 cells were transduced with the first lentivirus to express dCas9VP64 and selected for with blasticidin. Subsequently, cells were transduced with the second lentivirus to express MPH and selected for with hygromycin. Lastly, cells were transduced with the third lentivirus to express an sgRNA targeting the human PKD1 promoter and selected for with zeocin (FIG. 2).

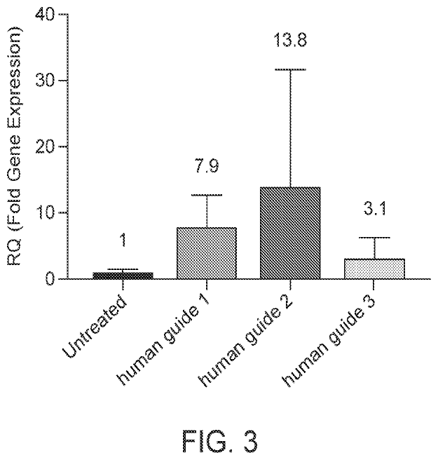

After this process produced a stable bulk population of modified 293 cells, RNA was purified from the cells. Quantitative reverse transcription polymerase chain reaction (qRT-PCR) was used to quantify the relative levels of PKD1 mRNA in the transduced cells versus untransduced cells (FIG. 3). Expression of human sgRNA1 brought PKD1 mRNA to a relative level of 7.9, human sgRNA2 brought it to 13.8, and human sgRNA3 brought it to 3.1. Therefore, each of these sgRNA's were effective at increasing the level of PKD1 mRNA, and also at different levels.

### Targeted Gene Activation of the PKD1 Allele in Human Renal Cortical Tubule Epithelial (RCTE) Cells

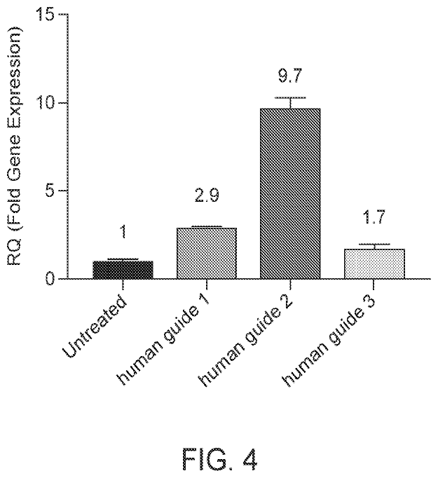

Human RCTE cells were subjected to the same process described above through the qRT-PCR step (FIG. 4). Expression of human sgRNA1 brought PKD1 mRNA to a relative level of 2.9, human sgRNA2 brought it to 9.7, and human sgRNA3 brought it to 1.7. The order of activation strength of these sgRNA's was conserved between 293 and RCTE cells, indicating that targeting particular promoter sequences may hold more inherent activation strength regardless of cell type.

### Targeted Gene Activation of the Pkd1 Allele in Mouse Inner Medullary Collecting Duct (IMCD3) Cells

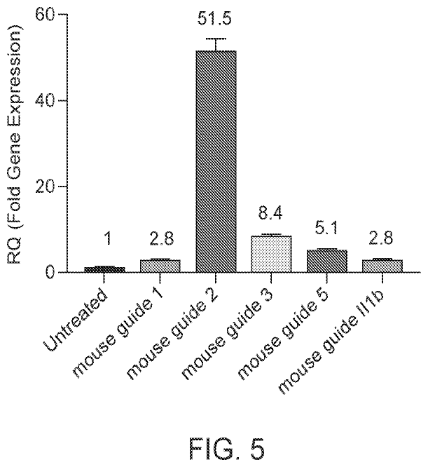

Mouse IMCD3 cells were subjected to the same process described above through the qRT-PCR step, with the exception that expressed sgRNA's were targeted to sequences in the mouse Pkd1 promoter rather than the human PKD1 promoter (FIG. 5). Expression of mouse sgRNA1 brought Pkd1 mRNA to a relative level of 2.8, mouse sgRNA2 brought it to 51.5, mouse sgRNA3 brought it to 8.4, and mouse sgRNA5 brought it to 5.1. In this case, a control sgRNA targeted to the promoter of the mouse Il1b gene was used as a control, which elevated the Pkd1 transcript to a level of 2.8, possibly due to dysregulation of cellular transcriptional networks.

Molecular Cloning of Dual AAV Vector SAM Plasmids and Verification of Protein Expression and sgRNA Sequences

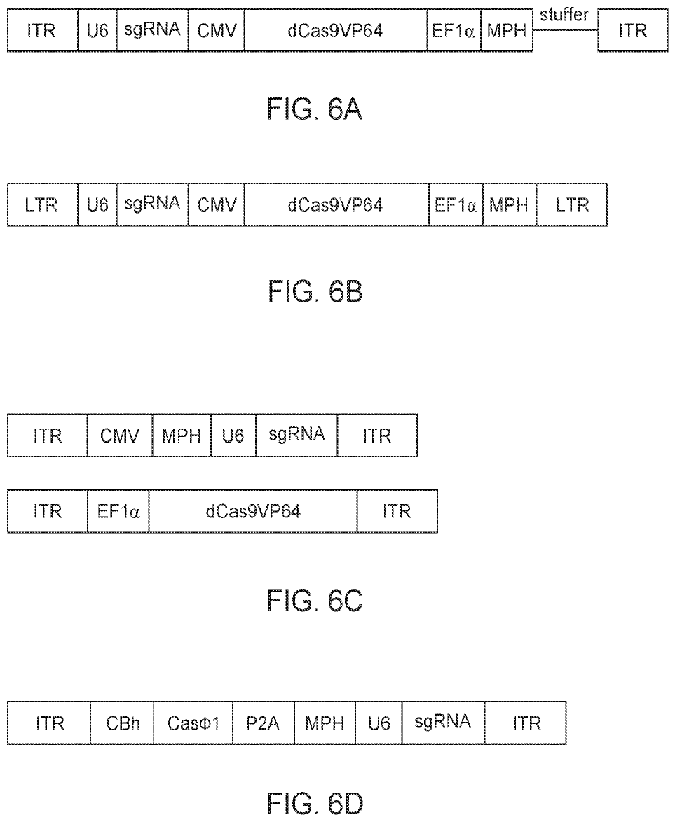

After sgRNAs compatible with activation of the human PKD1 and mouse Pkd1 genes were identified, construction of vectors for in vivo delivery of Cas9-SAM components began. One of these vectors is a HDAd capable of carrying all three components of the SAM system (FIG. 6A). Although less commonly used in vivo, a second option is a lentiviral vector carrying all components of the same system (FIG. 6B). The three components of the SAM system are too large to be packaged into a single AAV vector, so a third option is a dual AAV vector system, where the first AAV delivers MPH and the sgRNA and the second AAV delivers dCas9VP64 (FIG. 6C). While the Cas9-SAM system described thus far is too large to be packaged into a single AAV vector, the newly discovered CasΦ protein is small enough to make single AAV vector amenable to delivering CasΦ1, MPH, and an sgRNA (FIG. 6D).

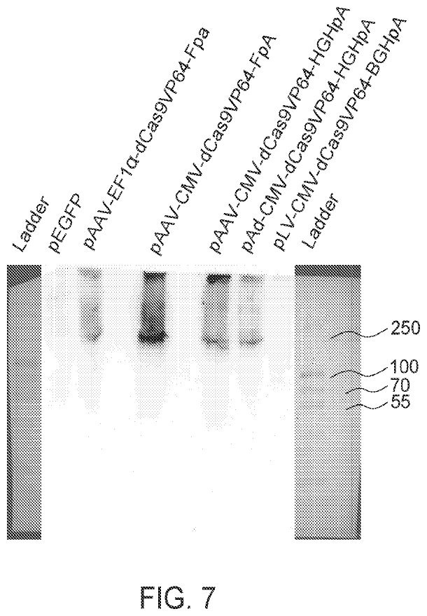

The first component of the SAM system, dCas9VP64, is 4.4 kb in length, which is already large for AAV. To ensure successful packaging, the transgene was flanked by relatively small expression elements in the AAV construct (FIG. 6C). To ensure robust dCas9VP64 expression from these expression cassettes, the vector production plasmids were transfected into 293 cells and dCas9VP64 protein was assayed three days later via western blot (FIG. 7). dCas9VP64 was detected in three different AAV expression cassettes with different combinations of promoters and polyadenylation signals as well as an adenoviral expression cassette. The lentiviral expression cassette transfected did not produce detectable dCas9VP64 protein. This assay confirmed that the first of two AAV's necessary for the dual vector system is expressing dCas9VP64. The second AAV, which must express MPH and an sgRNA, has been cloned to express one of three human PKD1 sgRNAs or one of seven mouse Pkd1 sgRNA's and sequence verified (Tables 2 and 3).

TABLE 2
sgRNA sequences used to target the human
PKD1 promoter.

| | | | | Nucleic | | | |
|---|---|---|---|---|---|---|---|
| | | | | acid | | | |
| | | | | encoding | | | |
| | Trans- | | TSS | the | SEQ | Se- | SEQ |
| Gene | cript | sgRNA | Dis- | Guide | ID | quencing | ID |
| Name | ID | ID | tance | Sequence | NO | result | NO |
| PKD1 | NM_ | Gen- | 84 | TCGCGCTG | 13 | TCGCGCTGTG | 13 |
| | 000296 | script3 | | TGGCGAAG | | GCGAAGGGGG | |
| | | | | GGGG | | | |
| PKD1 | NM_ | Gen- | 107 | CCAGTCCC | 14 | CCAGTCCCTC | 14 |
| | 000296 | script1 | | TCATCGCT | | ATCGCTGGCC | |
| | | | | GGCC | | | |
| PKD1 | NM_ | Gen- | 133 | GGAGCGGA | 15 | GGAGCGGAGG | 15 |
| | 000296 | script2 | | GGGTGAAG | | GTGAAGCCTC | |
| | | | | CCTC | | | |

TABLE 3
sgRNA sequences used to target the mouse
Pkd1 promoter.

| | | | | Nucleic | | | |
|---|---|---|---|---|---|---|---|
| | | | | acid | | | |
| | | | | encoding | | | |
| | Trans- | | TSS | the | SEQ | Se- | SEQ |
| Gene | cript | sgRNA | Dis- | Guide | ID | quencing | ID |
| Name | ID | ID | tance | Sequence | NO | result | NO |
| Pkd1 | NM_ | mouse | 13 | GAAGAGGG | 16 | GAAGAGGG | 16 |
| | 013630 | Pkd1 | | CGGAGCCT | | CGGAGCCT | |
| | | SAM | | GTGA | | GTGA | |
| | | sgRNA1 | | | | | |
| Pkd1 | NM_ | mouse | 34 | TTGCAGAT | 17 | TTGCAGAT | 17 |
| | 013630 | Pkd1 | | CCTGCAGT | | CCTGCAGT | |
| | | SAM | | AGGC | | AGGC | |
| | | sgRNA2 | | | | | |
| Pkd1 | NM_ | mouse | 60 | TGAAGGAA | 18 | TGAAGGAA | 18 |
| | 013630 | Pkd1 | | GGGCGCCC | | GGGCGCCC | |
| | | SAM | | TCAG | | TCAG | |
| | | sgRNA3 | | | | | |
| Pkd1 | NM_ | mouse | 86 | CGCCCAGT | 19 | CGCCCAGT | 19 |
| | 013630 | Pkd1 | | GAGCGTGA | | GAGCGTGA | |
| | | SAM | | GCCT | | GCCT | |
| | | sgRNA4 | | | | | |
| Pkd1 | NM_ | mouse | 107 | GGTGGGCG | 20 | GGTGGGCG | 20 |
| | 013630 | Pkd1 | | GGGTCTCA | | GGGTCTCA | |
| | | SAM | | CGGG | | CGGG | |
| | | sgRNA5 | | | | | |
| Pkd1 | NM_ | mouse | 138 | GCAGAAGG | 21 | GCAGAAGG | 21 |
| | 013630 | Pkd1 | | CGGGGCCT | | CGGGGCCT | |
| | | SAM | | CCGG | | CCGG | |
| | | sgRNA6 | | | | | |
| Pkd1 | NM_ | mouse | 160 | CGCTGGGT | 22 | CGCTGGGT | 22 |
| | 013630 | Pkd1 | | CTGCTGCA | | CTGCTGCA | |
| | | SAM | | GACC | | GACC | |
| | | sgRNA7 | | | | | |

### Non-Viral Delivery of Genetic Therapies for ADPKD

The same plasmids used for production of the viral vectors described above are complexed with lipid nanoparticles (LNPs) as a lower biosafety risk alternative to viral vectors. This plasmid DNA-LNP complexes is administered intravenously to transfect cells in vivo.

### Materials and Methods

### Generate and Test AAV, Lentiviral, and HDAd Vectors for TGA

A HDAd, a lentiviral vector, and two AAV vectors have been designed to carry the SAM system. Briefly, each expression cassette of dCas9-VP65; MS2-P65-HSF1; and the sgRNA cassette is amplified with oligonucleotides bearing large I-SceI or I-CeuI restriction sites. These products are inserted into unique I-SceI and I-CeuI restriction sites in the HDAd vector pDelta18, pAAV-SceCeu, and pLenti-SceCeu. dCas9-VP64 is amplified with I-SceI and I-CeuI sites, MS2-P65-HSF1 with I-SceI, and the mouse sgRNA cassettes with ICeuI. One AAV-dCas9-VP64 is used with three different AAVs expressing MS2-P65-HSF1 and one the one of three mouse sgRNAs. Similarly, there are three HDAds and three different lentiviruses carrying three mouse sgRNAs.

### In Vivo Transduction and Therapeutic Testing of Pkd1-TGA Vectors

Groups of 10 male and 10 female RC/RC mice are injected with PBS, HDAd-SAM (as a single vector), Lenti-SAM (as a single vector), or AAV-SAM (as a dual vector system). Retro-ureter or sub-capsular injection are used. 10¹¹ of HDAd-TGA gRNA vector is injected. 10⁶ transducing units (TU) of VSVg-pseudotyped lentivector with the entire SAM system is injected. AAV-Pkd1-TGA vectors can mediate therapy, even when they require co-infection of the cell by 2 vectors. AAVrh10 is used robustness and ability to transduce cells with high multiplicity. To maximize co-infection of the same renal cells with 2 AAVs, 10¹² vg of both AAVrh10-Pkd1-TGA vectors are delivered to the mice.

RC/RC mice are injected as described above. Each virus sample is blinded. MRI imaging, serum creatinine, and BUN are measured to assess kidney function. Five animals from each group are sacrificed at one week and five animals from each group are sacrificed at one month for western blot, qPCR, and histochemistry to determine whether Pkd1 expression and PC-1 protein levels are increased in the injected kidney and if there are positive or negative effects on cyst index, number and growth. Sections are stained by H&E to monitor changes in cyst sizes and immune infiltrates. Gene expression, kidney size, creatinine, BUN, kidney mass, and cyst formation are evaluated to determine if the HDAd, AAV, or lentivirus vectors mediate changes in kidney size and cystic phenotypes relative to controls.

### Example 3: Increasing Vector Penetration into Tissues from the Blood

Viral or non-viral gene therapy and cancer therapies use vectors that are many megaDaltons in size. These agents have a hard time entering into certain tissues like the kidney and brain after intravenous (i.v.) injections.

This Example describes methods that can loosen intracellular attachments to allow i.v. injected large vectors to penetrate into tissues such as the brain, lungs, spleen, liver, and kidney. For example, lipopolysaccharide (LPS) can be used to promote proteinurea and to increase leak of large vectors from the blood into tissues.

### Results

### Induced Proteinuria Increases Gene Delivery to Renal Tubule Epithelial Cells

Following intravenous administration of Ad or AAV, the vector appears to rarely penetrate past the glomerulus and further into the tubule of the nephron. The filtration properties of the glomerular barrier typically excludes solute in the blood that is greater than 10 kilodaltons (kDa) in mass or 10 nm in diameter. Ad and AAV are both significantly above these thresholds in size and thus are not generally expected to transduce renal tubule epithelial cells after intravenous injection. To overcome this limitation, proteinuria was induced in mice via effacement of podocyte foot processes in the glomerulus, which has been shown to structurally disrupt the glomerular filter and allow larger solute from the blood into the tubule of the nephron.

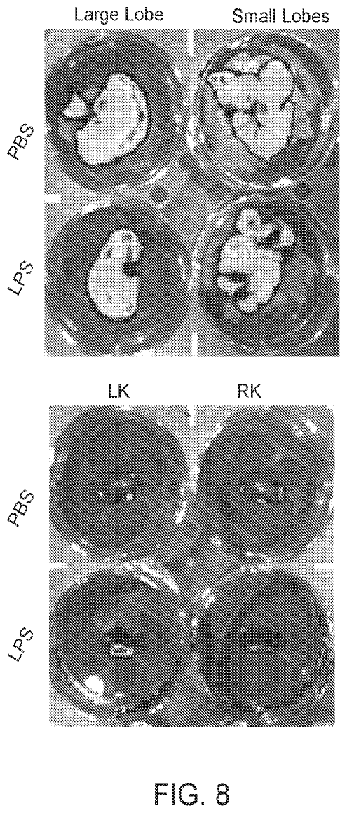

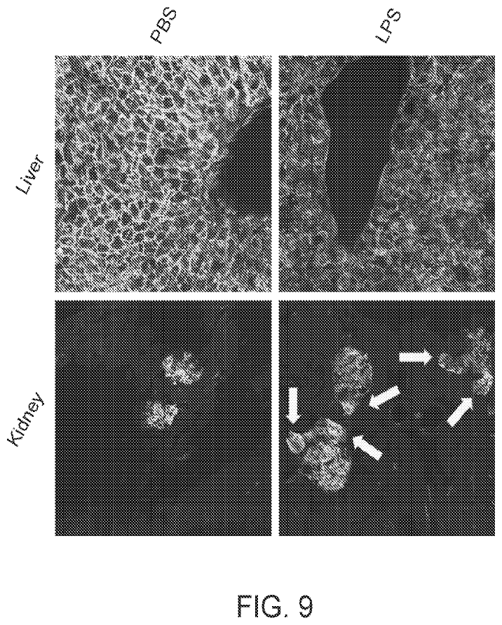

Luciferase/red-green hybrid reporter mice were intraperitoneally (i.p.) injected with 200 μg of lipopolysaccharides (LPS) to induce proteinuria. The next day, mice were given an intravenous injection of PBS, AAV8, AAV9, or AAVrh10 (n=1). In the case of AAV8, the mouse that had been administered LPS showed increase luminescence in its kidneys versus the PBS control (FIG. 8). When sectioning the kidneys of these mice, the LPS-injected mouse had consistently transduced (EGFP⁺) proximal tubules cell adjacent to glomeruli, while the PBS-injected mouse only had transduced cells in its glomeruli (FIG. 9).

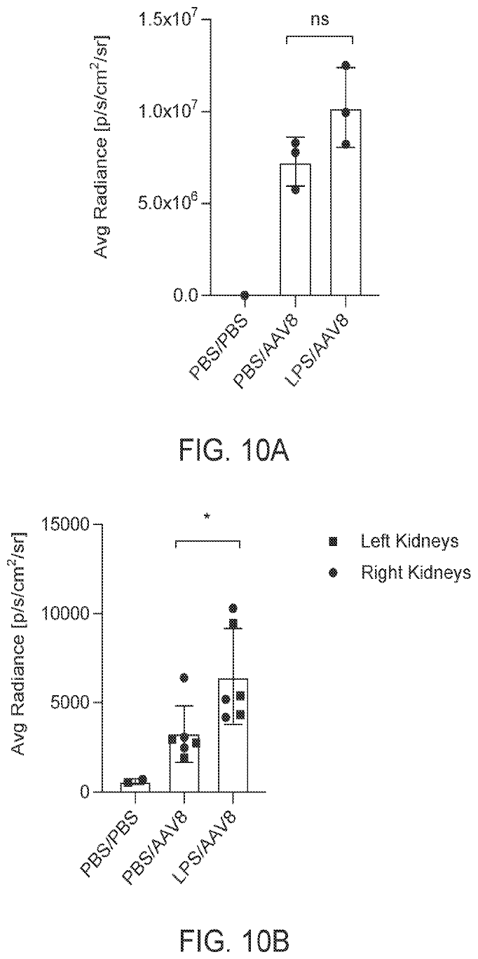

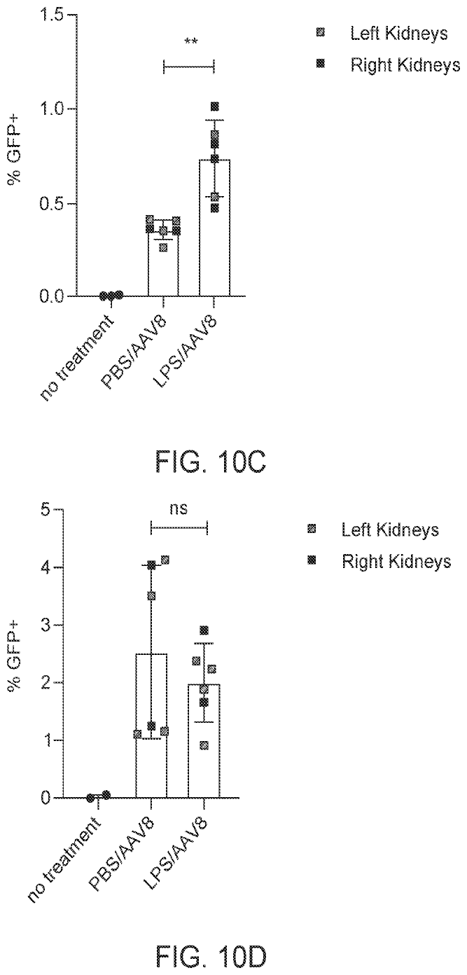

To quantify the extent to which tubule epithelial cells were being transduced during proteinuria, a larger scale experiment was performed using a lower dose of AAV (2e11 genome copies per mouse). Mice were injected with either PBS or LPS i.p., and were then injected with AAV8 the following day (n=3 mice for each group) or PBS as control (n=1 mouse for each group). Mice were sacrificed six days after AAV administration and tissues were imaged for luminescence ex vivo. Livers did not show a significant difference in luminescence between PBS and LPS-treated mice (FIG. 10A). However, ex vivo kidney luminescence showed a significance increase in LPS-treated mice versus PBS-treated mice (FIG. 10B). These kidneys were then homogenized and analyzed by flow cytometry. The cells were first gated into a CD45⁻ population, as to remove hematopoietic cells from the query. The EpCAM CD31 population, where EpCAM is a marker of epithelial cells and CD31 is a marker of endothelial cells, was then examined. In this population, the percentage of EGFP⁺ cells in LPS-treated mice was significantly increased from PBS-treated mice, indicating that induced proteinuria was transducing more epithelial cells (FIG. 10C). Transduced endothelial cells were also examined by analyzing the EpCAM⁻CD31⁺ population of cells and it was found that there was no significant difference between PBS and LPS-treated mice, indicating that induced proteinuria increased transduction of epithelial but not endothelial cells in the kidney (FIG. 10D). The ability to consistently target proximal tubule cells for transduction is useful for being able to treat ADPKD as well as other genetic kidney diseases.

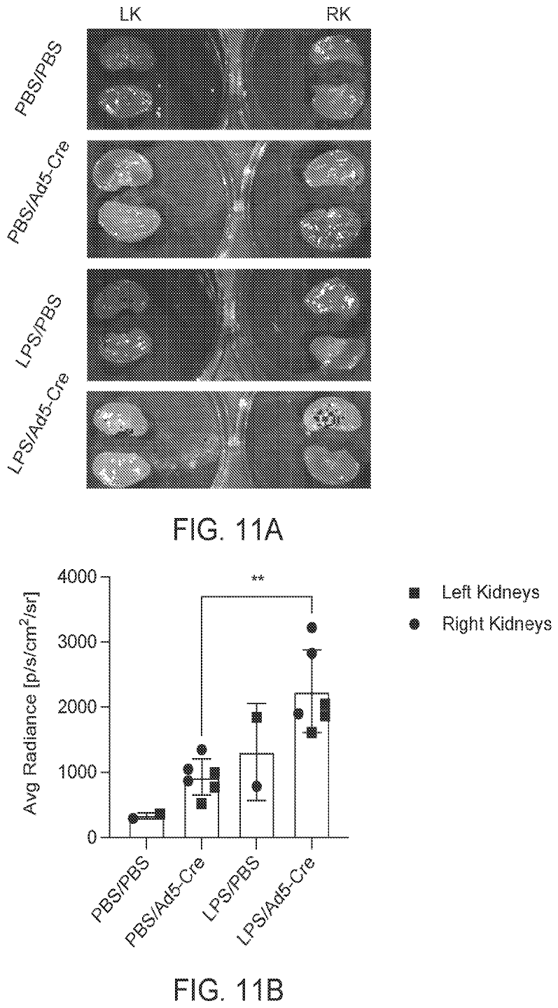

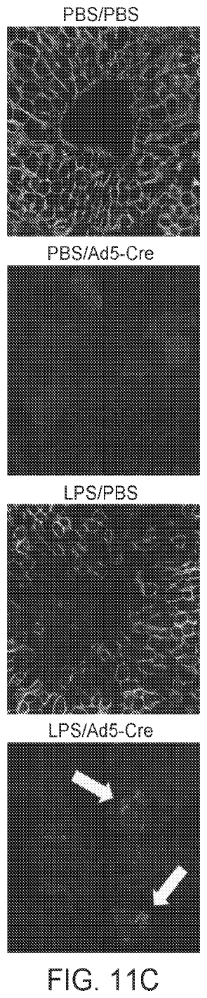

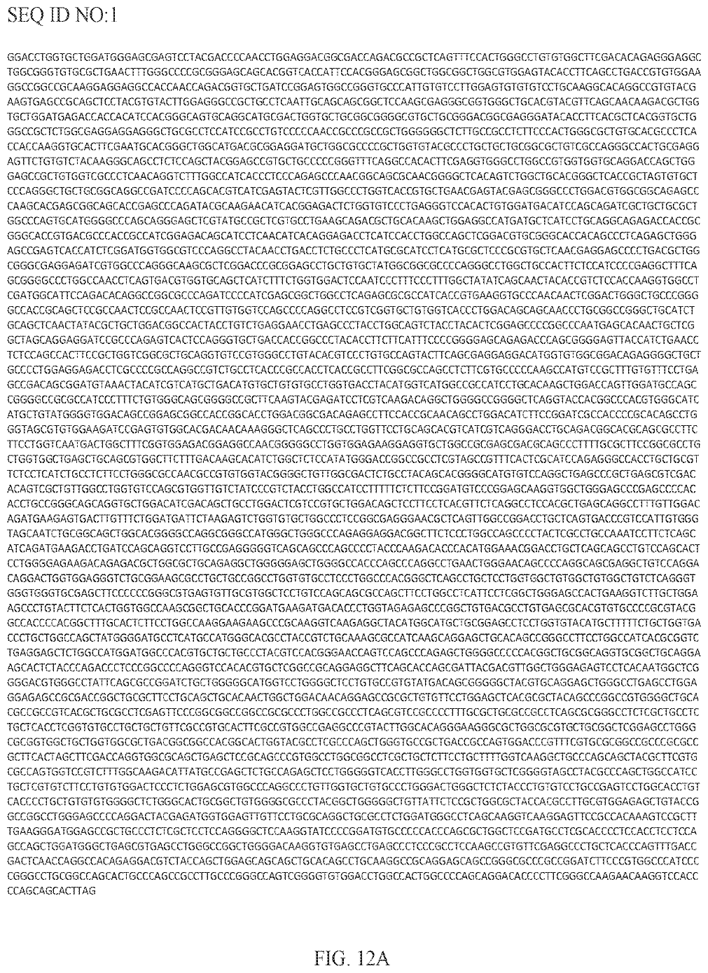

Since AAV showed promising results in renal tubule transduction when combined with induced proteinuria, it was investigated if the same effect could be achieved with a larger Ad vector. Mice were administered PBS or LPS followed by 1¹¹ viral particles of Ad5. Kidneys were imaged for luminescence ex vivo and some evidence of increased transduction in the LPS-treated mouse kidneys was observed (FIG. 11A). When the signals from these kidneys were quantified, it was found that the kidney luminescence had significantly increased in LPS-treated from PBS-treated (FIG. 11B). When livers and kidneys from these mice were sectioned for fluorescent histology, increased transduction was seen in the kidneys of LPS-injected mice, but only in the glomeruli (FIG. 11C). The LPS-treated mouse had reduced transduction in the liver compared to PBS-treated mice, possibly due to LPS interaction with the Kupffer cells in the liver.

### Materials and Methods

### Animals

Mice used in these experiments were F1 hybrids of loxP-STOP-loxP-Luciferase (LSL-Luc) mice (The Jackson Laboratory Stock No: 005125) and membrane-tomato/membrane-green (mT/mG) mice (The Jackson Laboratory Stock No: 007676). Thus, each mouse endogenously expressed tdTomato, and upon Cre-recombinase expression in a particular cell, has activated luciferase and EGFP genes.

### Proteinuria Induction in Mice

Urine was collected from mice of various ages and a baseline level of proteinuria was determined using Beyer Albustix. Mice were then injected with 200 μg of LPS (dissolved at 1 mg/mL in otherwise sterile PBS) intraperitoneally. Approximately 24 hours later, urine was collected and proteinuria levels were again determined. In most cases, administration of LPS versus a PBS control clearly caused an increased level of proteinuria in mice.

### Viral Vector Delivery

After induction of proteinuria via administration of LPS or a PBS control, mice were injected with adeno-associated virus serotype 8 (AAV8) expressing Cre recombinase or replication-defective adenovirus serotype 5 (RDAd5) expressing Cre recombinase intravenously via tail vein injection. Injection volumes were 100 μL. The dose of AAV8-Cre administered ranged from 2e11 to 1.94e12 genome copies while the dose of RDAd5-Cre administered was 1e11 viral particles.

### Luminescent Imaging

After viral vector injection, luminescent signals were monitored and quantified in vivo in mice until the signal peaked (observed to be six days) using Perkin Elmer IVIS Lumina and Living Image software. To do this, mice were anesthetized with isoflurane and injected intraperitoneally with luciferin, and imaged 10 minutes later. At the six day time point, mice were sacrificed and their tissues were dissected and placed in a six well plate to be imaged ex vivo and these signals were quantified. In some cases, the kidneys were laterally bisected to enhance the luminescent signal being emitted from within the tissue.

### Fluorescent Histology

The same tissues used for luminescent imaging were processed for fluorescent histology. Kidneys and liver were fixed in 4% paraformaldehyde overnight and then soaked in 15% sucrose/PBS followed by 30% sucrose/PBS until the tissues sank. Tissues were frozen in blocks in Optimal Cutting Temperature (OCT) medium. A Leica cryostat was used to section tissues at a thickness of 18 UM and mount them on glass slides. Mounting Medium with DAPI (Vector Labs) was then dropped on the sections and a glass coverslip was placed on top of the slide. Confocal microscopy was performed using a Zeiss LSM780 microscope with optimized settings to image tdTomato, EGFP, and DAPI.

### Flow Cytometry

Kidney samples were chopped into small pieces using scissors and put in Miltenyi tubes. 2.35 mL of DMEM was added. 100 μL of enzyme D, 50 μL of enzyme R, and 12.5 μL of enzyme A from the Miltenyi “Tumor Dissociation Kit” into were added to each sample. Program 37C_mTDK_1 or soft tissue dissociation was used on the OctoMACS machine. C-Tube was washed well by pouring DMEM, inverting, and passing through a 70 μM filter (15 mL volume). Cells were then spun at 400×g for 10 minutes. Samples were resuspended into 3.1 mL of cold DPBS and 900 μL of Miltenyi Debris removal solution was added and resuspended well. 4 mL of ice cold DPBS was carefully overlayed onto the samples. Samples were spun at 3000 g for 10 minutes with brakes on. 1 mL of ACK Lysis buffer was added for 1 minute and subsequently quenched by filling the tube to top (15 mL rol) with cold RPMI. All samples were processed and passed through filters and transferred to 5 mL flow tubes. Tubes were filled with PBS and spun at 400 g for 5 minutes. 500 μL of MasterMix was added to each sample to stain for flow cytometry, as follows: EpCAM PECy7 (1:250) (BioLegend, Cat #118216), CD31 AF647 32 (1:500) (BioLegend, Cat #102516), CD45 perCP (1:1000) (BioLegend, Cat #103130), Viability-ghost dye red 780 (1:2000) (Tonbo Biosciences, Cat #13-0865-T100), FC block (1:500) (BD Pharmingen, Cat #553141). Results were analyzed using FlowJo software.

### Example 4: Induced Proteinuria Enhances Adeno-Associated Virus Transduction of Renal Tubule Epithelial Cells after Intravenous Administration

There are a variety of genetic diseases of the kidney tubule that might be amenable to correction via gene therapy. However, gene delivery to renal tubule epithelial cells mediated by viral vectors via the blood is historically inefficient due to the permselectivity of the glomerular barrier, which typically will not allow molecules larger than 50 kilodaltons in mass or 10 nanometers in diameter to pass into the tubule of the nephron.

This Example demonstrates that AAV vectors can penetrate into the nephron and transduce tubule epithelial cells in a state of proteinuria.

### Results

### AAV8 Gene Delivery to the Kidney is Distinctly Enhanced in a State of Induced Proteinuria

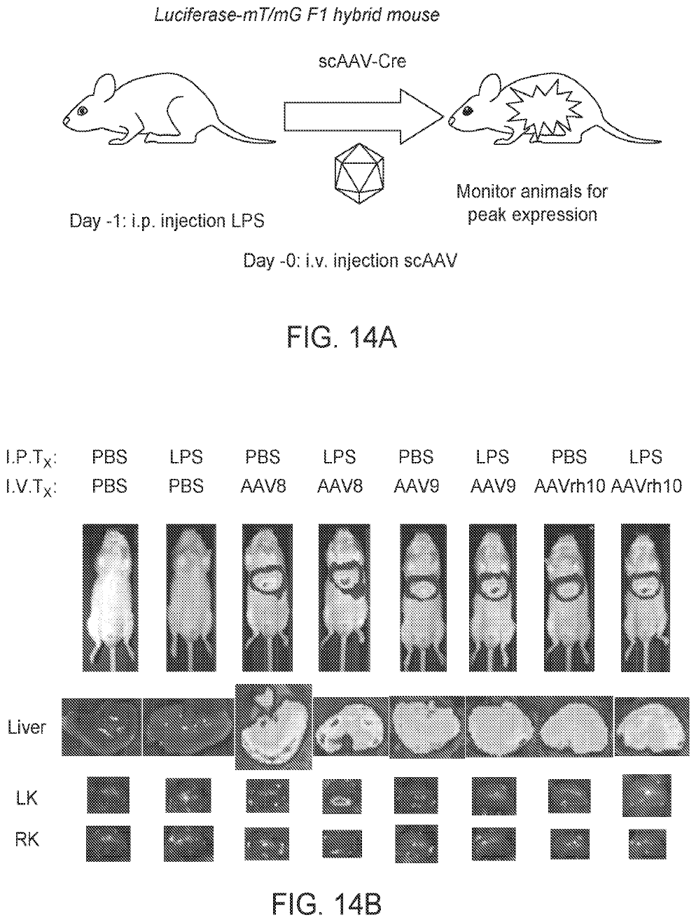

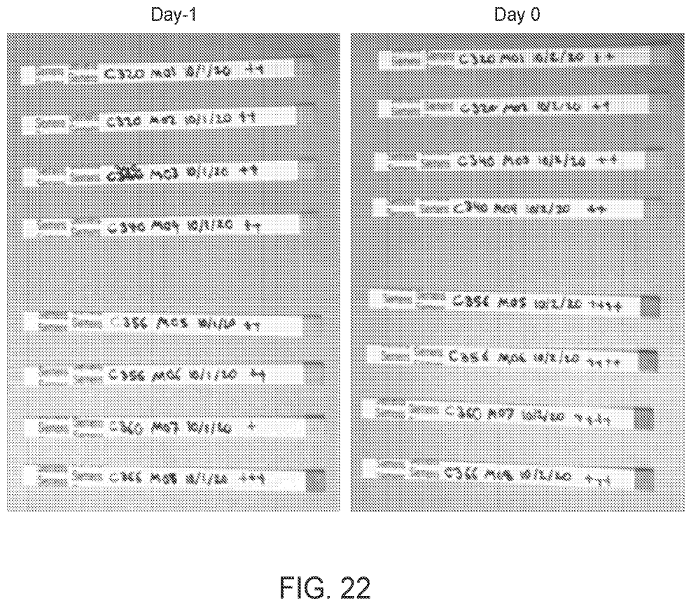

To begin to investigate the effects of induced proteinuria on viral vector gene delivery to the kidney, mice were administered an i.p. injection of 200 μg of LPS. The mode of delivery and dose were as described elsewhere (Reiser et al., J. Clin. Invest., 113:1390-1397 (2004)). The following morning, urine was collected from mice injected with either LPS or PBS as a control and assayed using a proteinuria dipstick to ascertain whether proteinuria had effectively been induced (example portrayed in FIG. 22). Subsequently, mice were administered i.v. injections of self-complementary AAV8-Cre (scAAV8-Cre), scAAV9-Cre, scAAVrh10-Cre, or PBS as control (n=1 for each combination of PBS or LPS and each vector). The mice used in this experiment are known as LSL-Luc-mT/mG F1 hybrid mice: each mouse has one LoxP-STOP-LoxP-Luciferase allele and one membrane-targeted tdTomato/membrane-targeted EGFP allele at the ROSA locus. Thus, each mouse has luciferase and mG genes activatable by Cre-expressing vectors, allowing for tracking of vector pharmacodynamics on both a cellular and tissue-specific level (FIG. 14A).

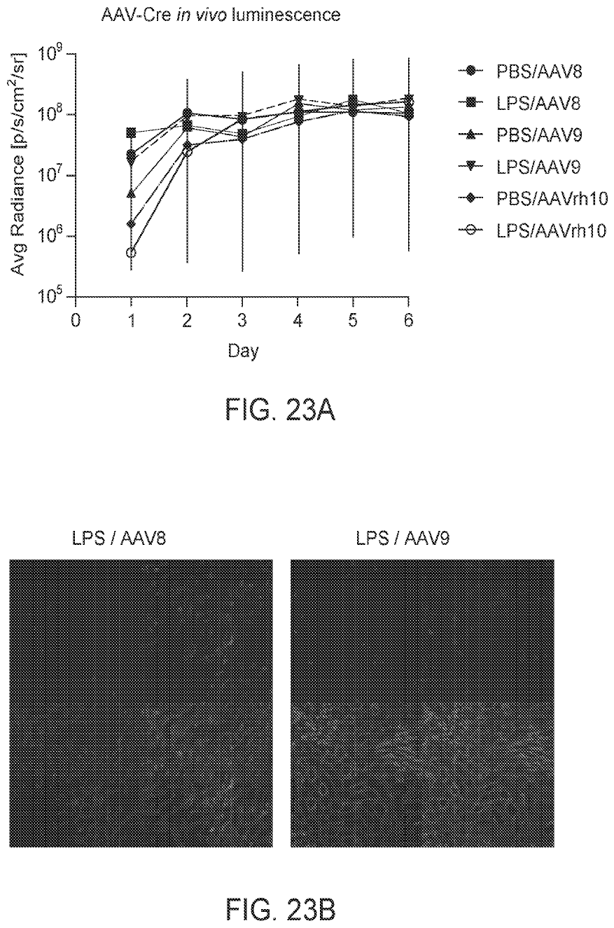

Luciferase activity in the mice was tracked daily via bioluminescent imaging until the signals reached an approximate plateau at day 6 (FIG. 23A). The signals measured in vivo almost were almost certainly emitted from luciferase activity in the livers of these, due to the high liver tropism of the three AAV serotypes used (FIG. 14B). To directly assess liver and kidney transduction of the injected mice, the mice were sacrificed and these organs were imaged ex vivo. While kidneys of the AAV9 and AAVrh10 injected mice with or without induced proteinuria exhibited minimal luminescence which was localized to the renal pelvis region of the kidney, the kidneys of the mouse with induced proteinuria injected with AAV8 had pervasive luciferase expression throughout the entire kidney (FIG. 14B). This observation of increased luciferase expression through the whole of the kidney tissue while in a state of proteinuria, as opposed to the luciferase activity seen exclusively on the edges of the kidney capsule of the control mouse, indicates a clear difference in vector pharmacodynamics between mice in states of induced proteinuria and not.

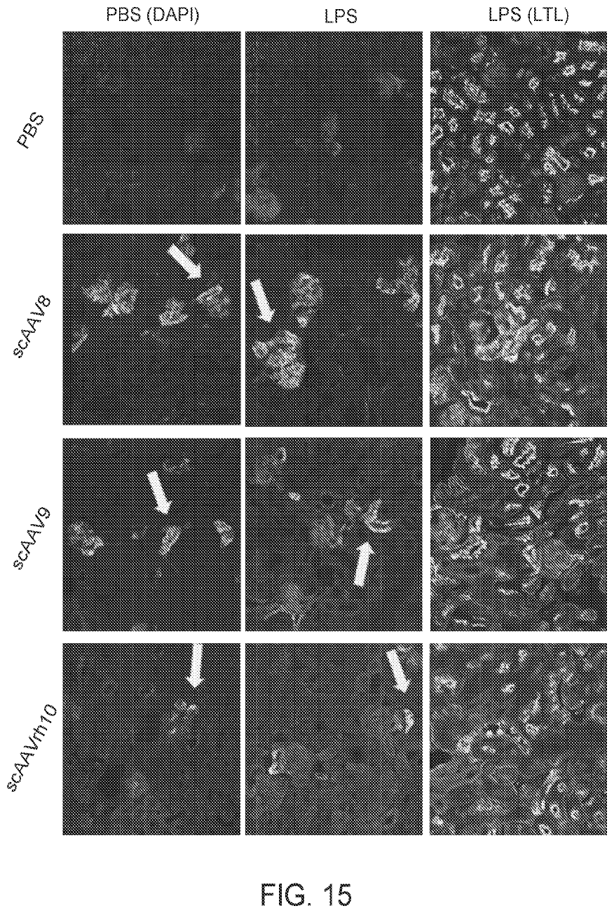

To assess kidney transduction on a cell-by-cell basis, the kidney and liver tissues were sectioned to view direct fluorescence via confocal microscopy. In the current reporter mouse model system, untransduced cells will endogenously express membrane-targeted tdTomato (mT), while Cre-expressing transduced cells will stop expressing tdTomato and begin to express membrane-targeted EGFP (mG). For each of the three AAV serotypes, it was observed that treating mice with LPS prior to AAV injection resulted in many instances of transduced cells with tubular morphology adjacent to glomeruli, as compared to control kidneys (FIG. 15). To determine if viral vectors might bypass the glomerulus and penetrate the most proximal part of the nephron, the proximal tubule, and to verify which additional cells AAV is transducing in an induced proteinuria state, kidney sections were counterstained with lotus tetragonolobus lectin (LTL), a marker of proximal tubule cells. No instances of EGFP⁺ transduced cells seemed to be double positive for the LTL stain. This indicates that although induced proteinuria seems to allow AAV to penetrate further into kidney tissue from the blood and transduce more tubule cells, these cells are not necessarily proximal tubule cells.

### AAV8 Significantly Increases Renal Epithelial Cell Transduction During Proteinuria

Data indicate that AAV serotypes 8, 9, and rh10 each potentially increase transduction of renal tubule epithelial cells when mice are in an induced state of proteinuria. In particular, AAV8 had the most striking effect in terms of increased transduction during induced proteinuria (FIG. 14B). To quantify this effect, and to determine if this effect could be achieved at a lower dose, new groups of mice were given an i.p. administration of either PBS or LPS at Day-1 and an i.v. administration of scAAV8-Cre at Day 0 at a dose of 2e11 genome copies (GC). Proteinuria dipsticks from these groups of mice at Day-1 (baseline) and Day 0 (post PBS or LPS) are shown as an example (FIG. 22).

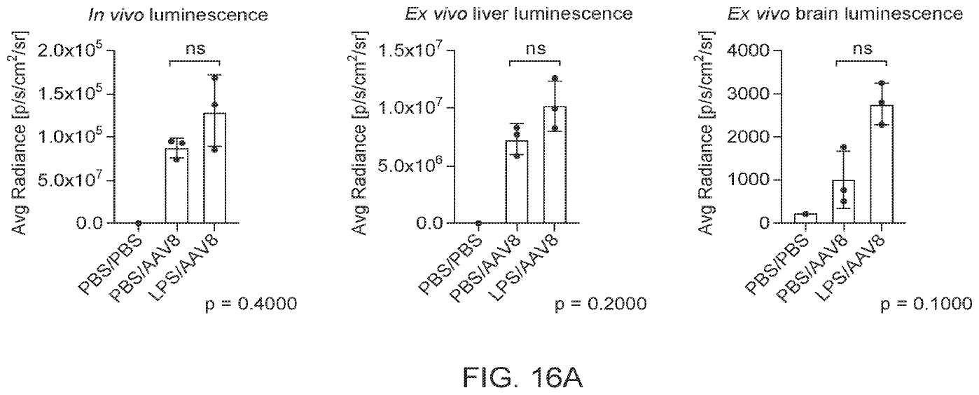

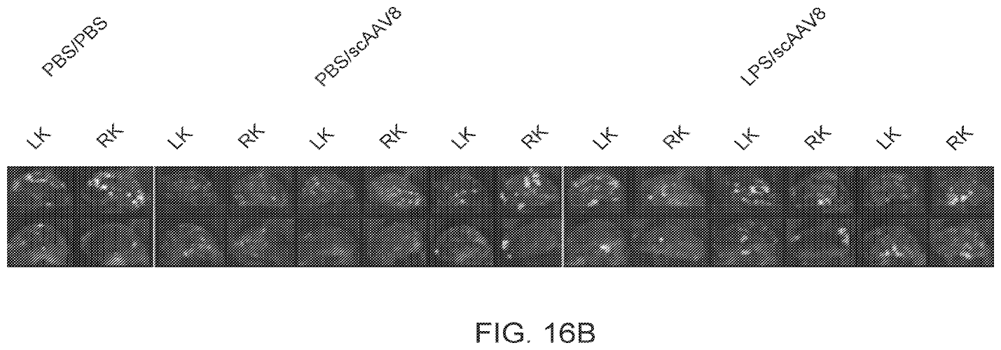

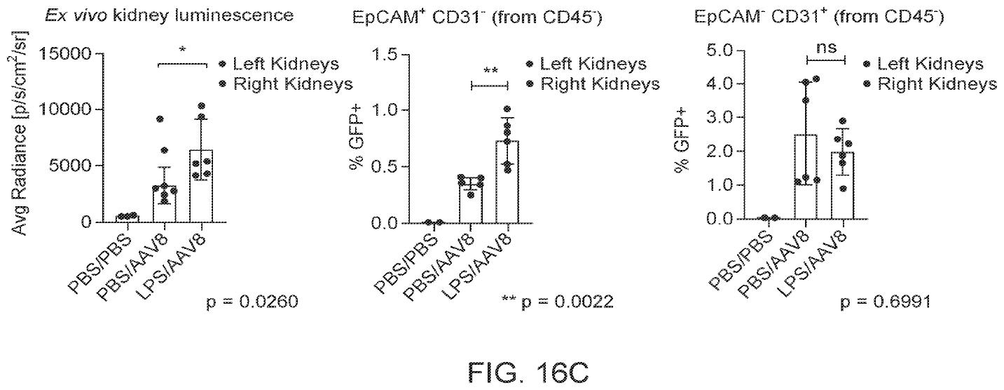

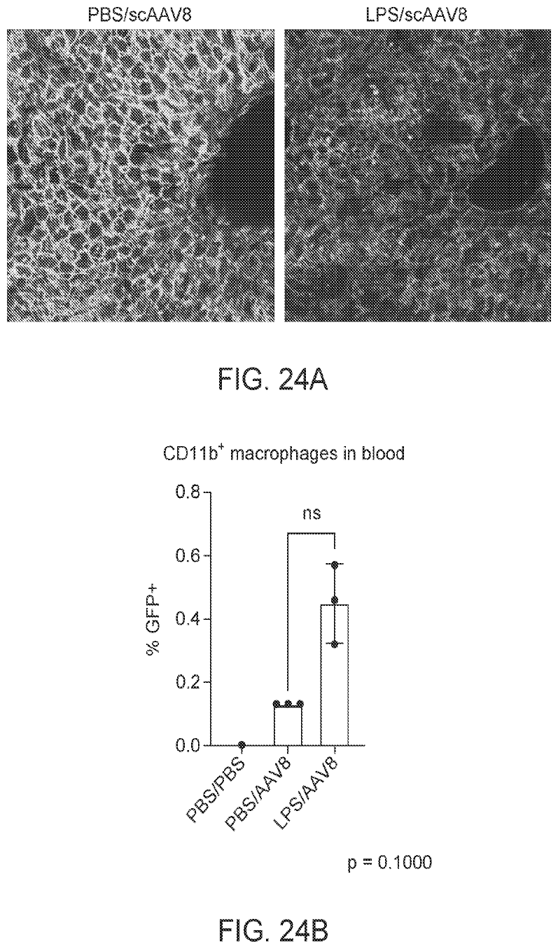

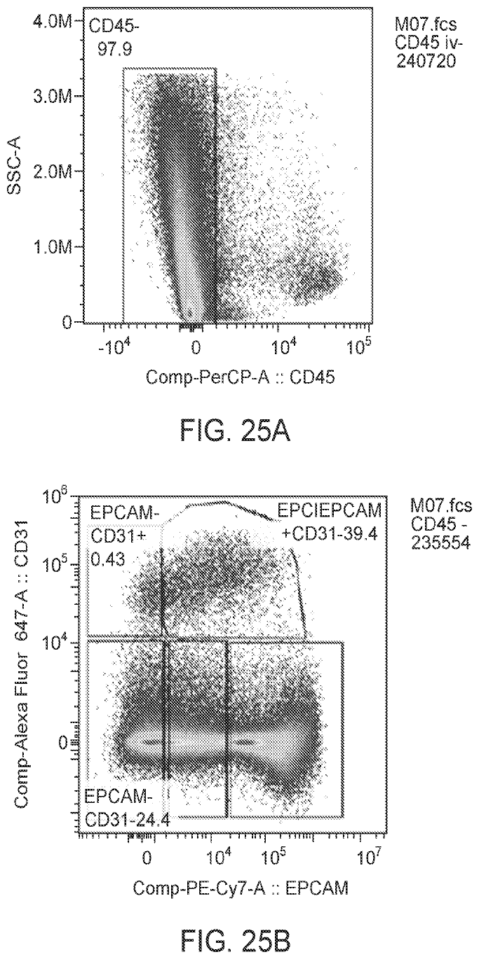

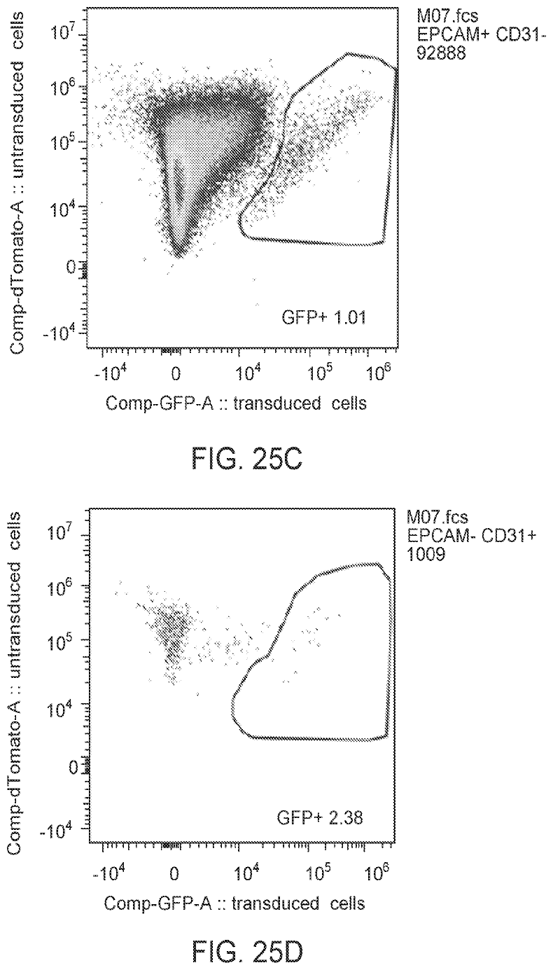

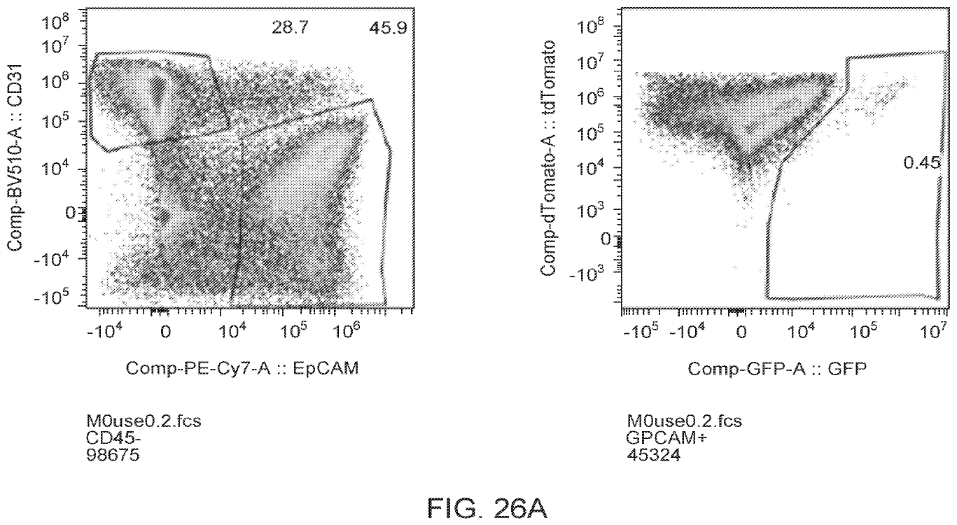

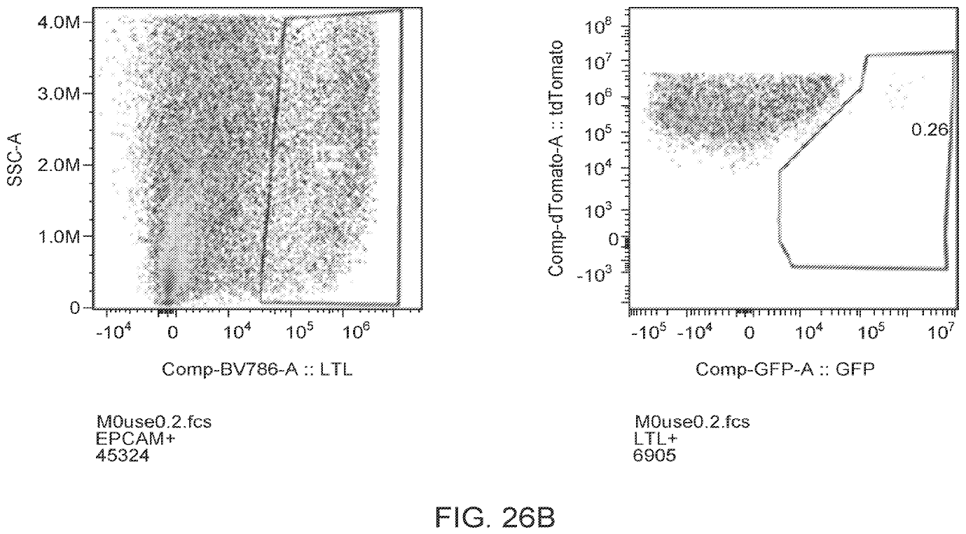

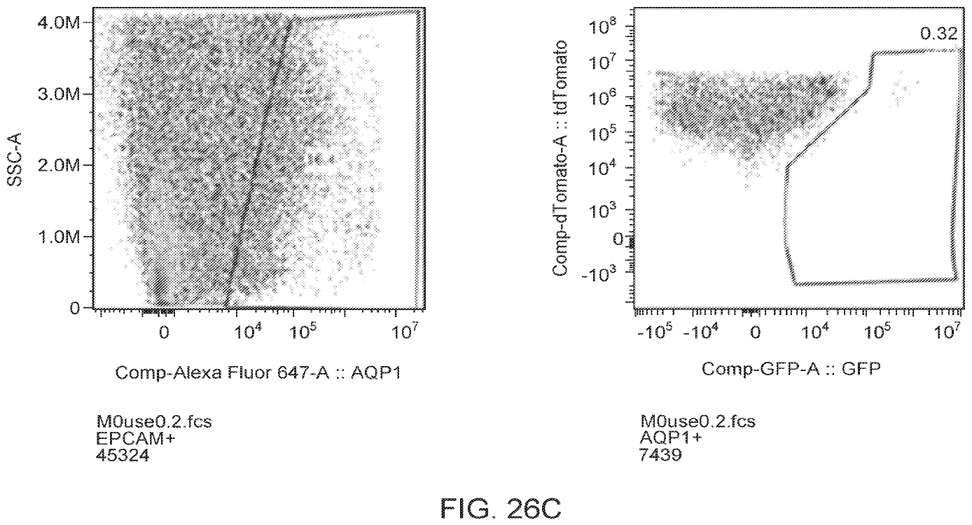

These mice were imaged for in vivo luminescence at Day 6 at which point the mice were sacrificed and their tissues imaged ex vivo. There was no significant difference observed between PBS and LPS-injected groups in vivo (indicative of liver transduction), liver ex vivo, or brain ex vivo (FIG. 16A). Although insignificant, brain luminescence was increased in all samples, indicating that LPS administration may induce some blood brain barrier disruption and increase transduction of cells in the brain. In contrast to the livers and brains, ex vivo kidney transduction visibly increased in LPS-injected mice versus PBS-injected mice (FIG. 16B). Upon quantitation of luminescence in these kidneys, the kidneys of the LPS-injected mice exhibited significantly higher luminescence than those of the PBS-injected mice (FIG. 16C). These kidneys were then processed for flow cytometry and labeled to detect epithelial cell adhesion molecule (EpCAM), a marker of epithelial cells, CD31, a marker of endothelial cells, and various other immune cell markers. Upon examination of the % EGFP⁺ (transduced) cells in EpCAM⁺ CD31⁻ and EpCAM-CD31⁺ populations, it was found that epithelial cells, but not endothelial cells, had a significant increase in transduction, indicating that the injected AAV8 did in fact have more access to epithelial cells during a state of induced proteinuria (FIG. 16C). In addition, an increased, albeit insignificant, % EGFP⁺ macrophages were found in the blood of LPS-injected mice as compared to PBS-injected mice, indicating that an increased presence of macrophages may have been induced by LPS administration and subsequently transduced by scAAV8-Cre (FIG. 24B). Representative flow plots and gating strategies are shown in FIG. 25.

### AAVrh10 Significantly Increases Hematopoietic Cell, but not Epithelial Cell Transduction, During LPS-Induced Proteinuria

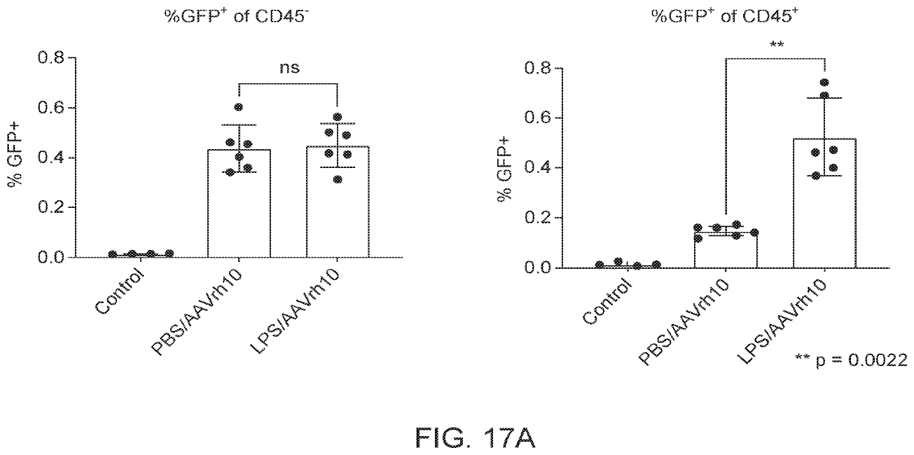

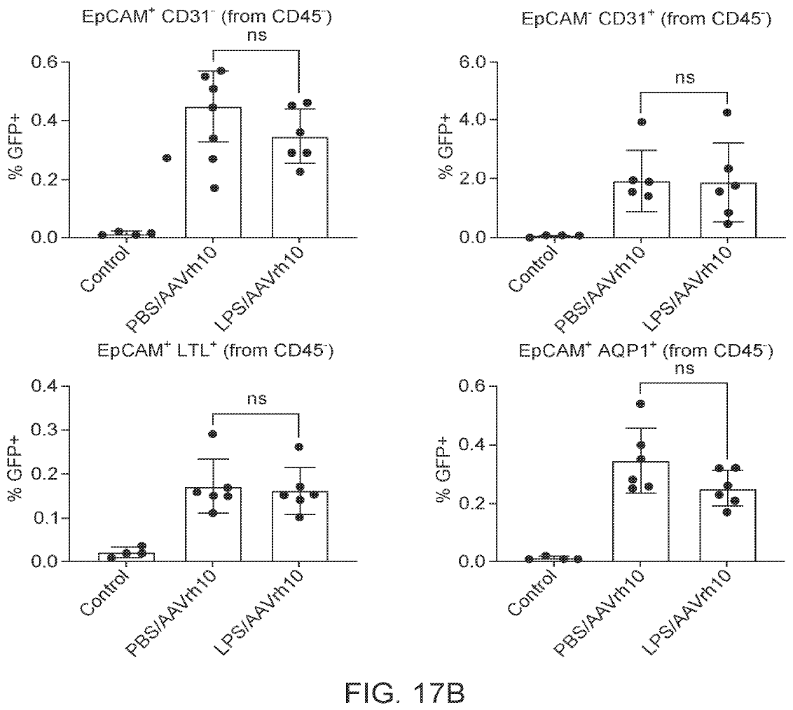

It was next sought to determine if a particular serotype of AAV could in fact result in a significantly increased number of epithelial cells in the kidney after i.v. injection in a state of induced proteinuria. In the initial experiment, AAV8 had stronger results than AAV9 or AAVrh10. To ascertain whether particular serotypes of AAV other than AAV8 might be able transduce significantly more renal epithelial cells in a state of induced proteinuria, the prior flow cytometry experiment was repeated using scAA Vrh10-Cre rather than scAAV8-Cre. The % EGFP⁺ (transduced) present among CD45⁻ (non-hematopoietic) and CD45⁺ (hematopoietic) cells in the kidneys was examined (FIG. 17A). There was no difference in transduced CD45⁻ cells between PBS and LPS-injected groups. However, there was a significant increase in transduced CD45⁻ cells. This effect may be due to an increased number of hematopoietic cells that infiltrated the kidney after LPS injection and were more susceptible to transduction the day after.

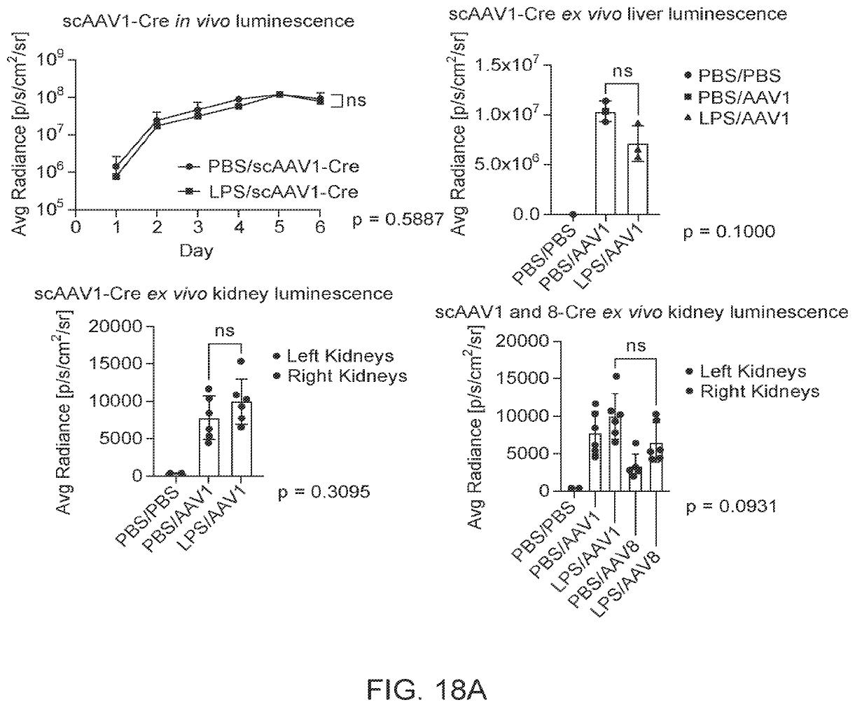

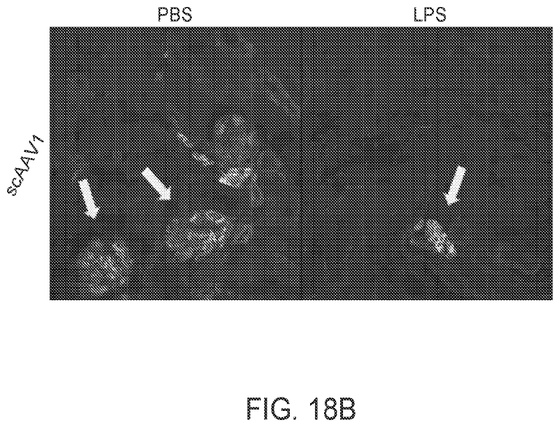

The transduction of epithelial cells in the kidney was examined. As with the previous experiment using scAAV8, the % EGFP+ cells amongst CD45⁻ EpCAM⁺ and CD45⁻ CD31⁺ populations, which represent transduced epithelial cells and transduced endothelial cells, respectively, was examined. When this experiment was performed using scAAV8 (FIG. 16), there was a significant increase in transduced epithelial cells, but not endothelial cells, between the LPS and PBS-injected mice. However, when this experiment was repeated using scAAVrh10, there was no significant difference between the LPS and PBS-injected groups of mice (FIG. 17B). To further drill down on proximal tubule cells, a specific subset of kidney tubule epithelial cells, samples were also labeled with LTL and aquaporin-1 (AQP1). In both cases, no significant difference was observed between transduced cells in LPS or PBS-injected mice. Overall, mice with or without induced proteinuria did not seem to have a change in transduced renal epithelial cells after intravenous injection of scAAVrh10-Cre. Representative flow plots and gating strategies are shown in Supplemental FIG. 18.

### A Naturally Liver-Detargeted Vector Enhances Kidney Transduction During Induced Proteinuria

Although increasing transduction in tubule cells in the kidney is an important goal for efficacy of gene therapy, detargeting vectors from off-target tissues is an important facet of gene therapy safety. While AAV8 showed efficacy in terms of increasing kidney transduction during a state of induced proteinuria, it also fully transduces the liver (FIG. 24A). To attempt to resolve the off-target tissue transduction, AAV1, a serotype known to have lower liver tropism than other serotypes, was tested in conjunction with induced proteinuria.

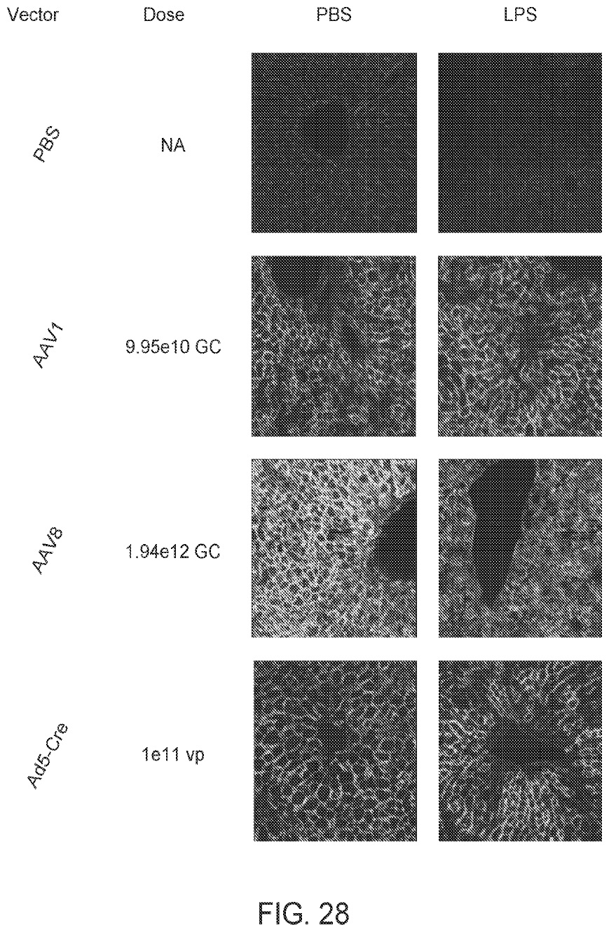

Mice were administered an i.p. injection of either PBS or LPS at Day-1 and an i.v. injection of scAAV1-Cre at Day 0 at a dose of 9.95e10 GC. Similar to previous experiments, in vivo luminescence signals peaked at Day 6, at which point mice were sacrificed and ex vivo liver luminescence was comparable between both groups of mice (FIG. 18A, top). Although mean kidney ex vivo luminescence was increased in LPS-injected mice versus PBS-injected mice, the difference was not significant. When comparing this data side-by-side with the ex vivo kidney luminescence data of the scAAV8-Cre injected mice from FIG. 16, both PBS-injected and LPS-injected groups of scAAV1-injected mice had higher signals than the LPS and scAAV8-injected mice, and at approximately half of the dose of scAAV8, indicating that scAAV1 may have a higher native kidney tropism both with and without induced proteinuria (FIG. 18A, lower). Upon sectioning of the kidneys of these mice to examine endogenous mT and mG fluorescence, mice injected with PBS followed by scAAV1 had many instances of transduced glomerular cells while mice injected with LPS followed by scAAV1 had increased instances of transduced tubular cells (FIG. 18B). Importantly, the livers of the mice injected with scAAV1 were only partially transduced, while the livers of the mice injected with scAAV8 were fully transduced (FIG. 28). These data indicate that scAAV1 may be an ideal vector for targeting renal tubule epithelial cells while avoiding unnecessary transduction of hepatocytes.

### Induced Proteinuria Enhances Ad5 Transduction of Glomerular, but not Epithelial Cells

Thus far, four different serotypes of AAV were tested in tandem with the LPS-induced proteinuria method. Between these serotypes, notable differences in the transduction profiles of kidney and liver cells were observed. The variation in transduction profiles is likely due to differences in receptor usage as well as capsid surface electromagnetic charges. To test other applications and potential limitations of the induced proteinuria method with respect to kidney transduction, physically larger gene delivery vector, replication-defective adenovirus serotype 5 expressing Cre recombinase (Ad5-Cre), was used.

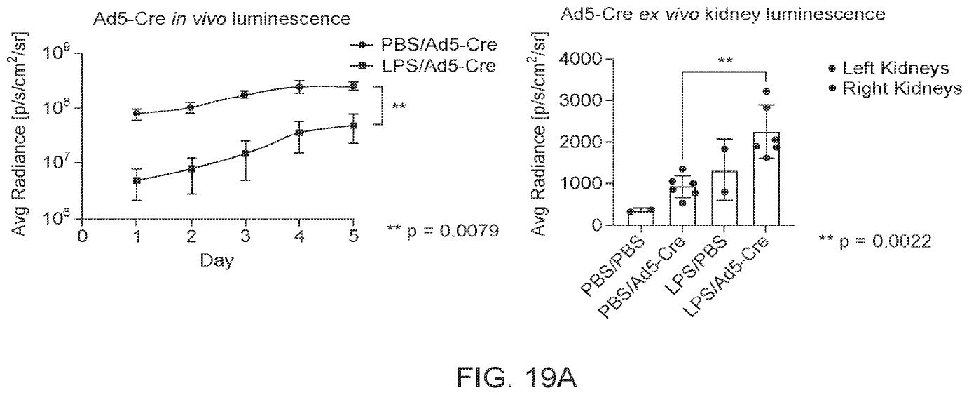

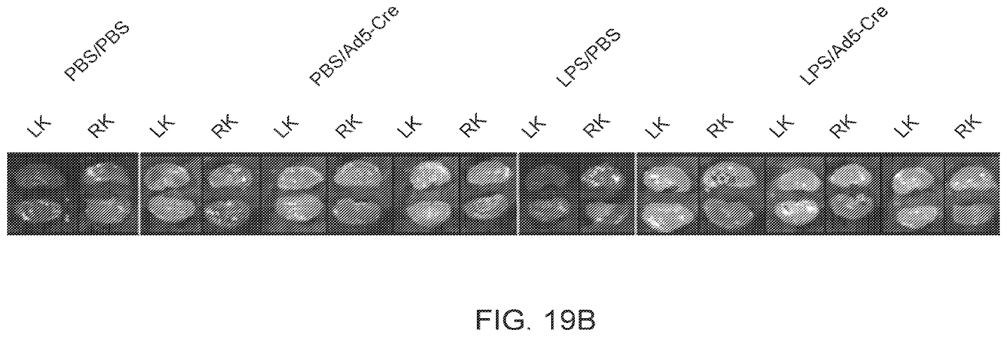

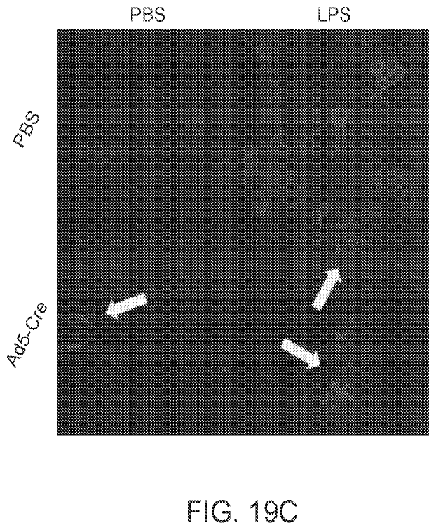

Mice were administered i.p. injections of either PBS or LPS on Day-1 and i.v. injections of Ad5-Cre on Day 0. In vivo luminescent signals (indicative of level of liver transduction) were monitored up to Day 5 until they peaked. In contrast to previous experiments using AAV, mice injected with LPS prior to Ad5-Cre had significantly reduced in vivo luminescence compared to PBS-injected mice (FIG. 19A, left). The mice were then sacrificed and their kidneys were imaged for ex vivo luminescence. As with the previous experiments performed with AAV, kidneys from mice administered LPS prior to Ad5-Cre had a significantly higher signal than those from mice administered PBS prior to Ad5-Cre (FIG. 19A, right). In the images of the kidneys of PBS and Ad5-Cre injected mice, little to no luminescence is visible, whereas in the kidneys of the LPS and Ad5-Cre injected mice, two out of three of the kidneys showed enhanced luminescence localized near the renal pelvis (FIG. 19B). This is in contrast to kidney images of mice injected with LPS and scAAV8, which showed more diffuse luminescence throughout the kidney (FIG. 16).

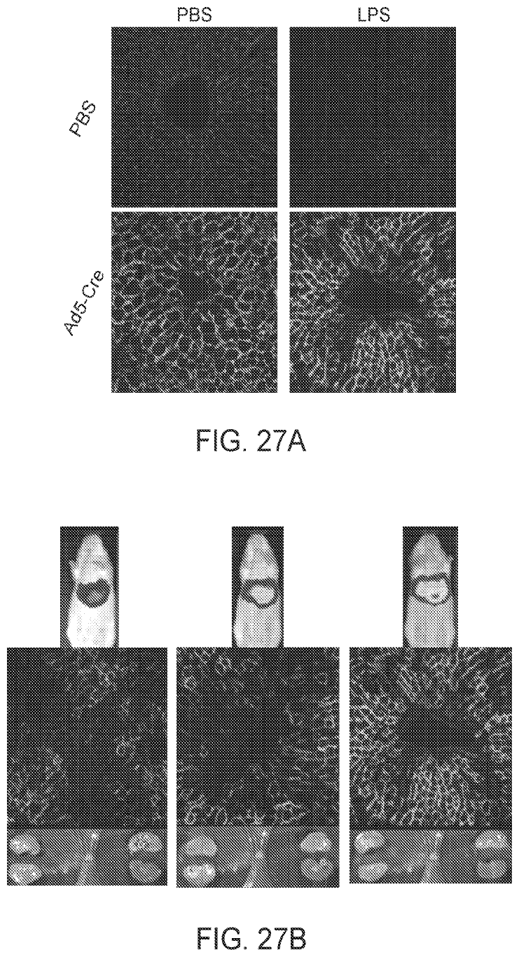

Kidneys from the Ad5-Cre injected mice were then sectioned to examine endogenous mT and mG fluorescence. Notably, in contrast to previous experiments using AAV, no instances of transduced tubule cells were observed in kidneys of PBS or LPS and Ad5-Cre injected mice. However, there were observed to be an increased number of glomerular cells transduced in the LPS and Ad5-Cre injected mice versus the PBS-injected mice, indicating that induced proteinuria did not enhance penetration of Ad5-Cre into renal epithelial tubular cells but may have aided penetration further into the glomerulus itself (FIG. 19C). In accordance with the in vivo luminescence signals from these mice, liver sectioning showed that mice injected with PBS followed by Ad5-Cre had fully transduced livers while mice injected with LPS followed by Ad5-Cre had only partially transduced livers, possibly as a result of LPS interactions with Kupffer cells (FIG. 27A). These data indicate that while the induced proteinuria method used in tandem with Ad may not necessarily be effective in treating genetic diseases of the tubule, such as polycystic kidney disease, it may be helpful in increasing gene delivery to the glomerulus.

### Induced Proteinuria Increases Epithelial Cell Transduction in a Mouse Model of ADPKD

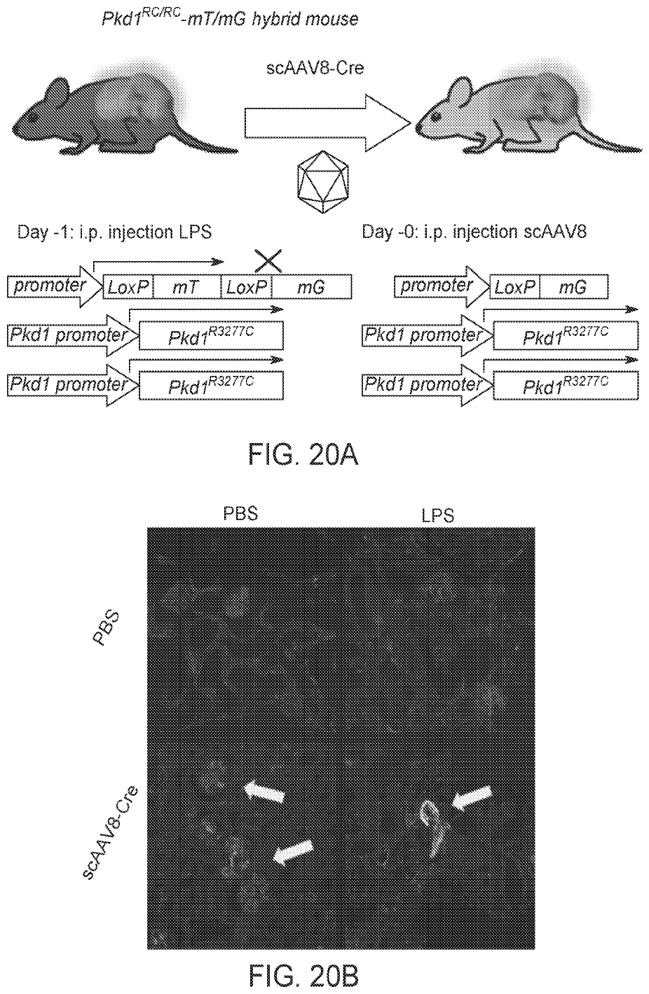

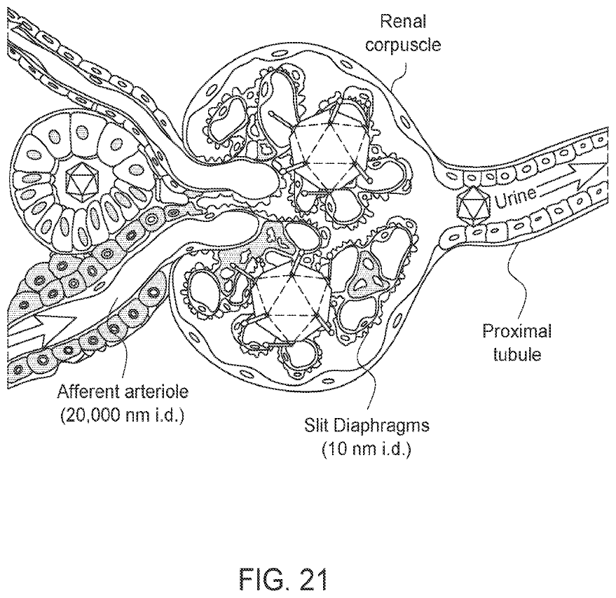

Thus far, out of a handful of gene therapy vectors tested, only particular vectors tended to increase transduction of renal tubule epithelial cells while mice were in a state of induced proteinuria: namely, AAV1 and AAV8. To test if the induced proteinuria method is amenable to enhancing renal tubule epithelial cell transduction in a mouse model of relevant human disease, this technique was employed on mice with ADPKD. Pkd1^{RC/RC} mice, which are homozygous for the hypomorphic Pkd1 allele p.R3277C and develop progressive ADPKD similar to human disease, were backcrossed to mT/mG mice until pups had exactly two Pkd1^{RC} alleles and at least one mT/mG allele. In essence, the newly generated mice are identical (give or take differences in genetic background due to a partial backcross) to the original mT/mG mice except they now develop ADPKD (FIG. 20A).

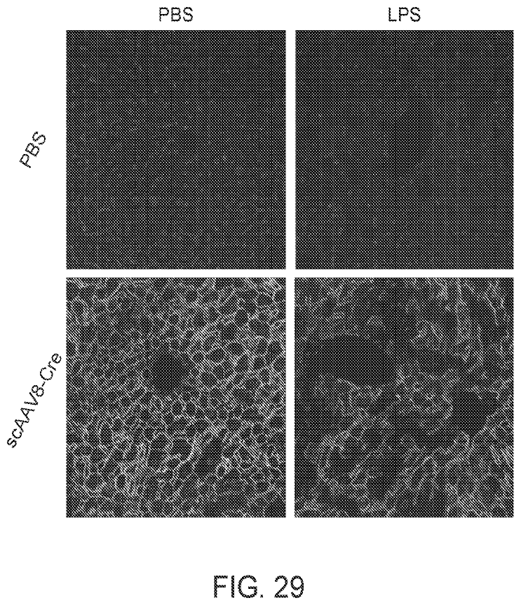

The Pkd1^{RC/RC}-mT/mG hybrid mice were administered i.p. injections of PBS or LPS on Day −1 and an i.v. injection of scAAV8-Cre at Day 0 at a dose of 2e11 GC. Under the assumption that vector pharmacodynamics would recapitulate those of the prior AAV experiments, mice were sacrificed at Day 6 and their tissues were sectioned. While evidence of glomerular transduction was apparent in the mouse injected with PBS followed by scAAV8-Cre, evidence of tubular cell transduction was observed only in the mouse injected with LPS followed by scAAV8-Cre (FIG. 20B). The livers of these mice were fully transduced by scAAV8-Cre, as expected (FIG. 29). Overall, these data support mice with ADPKD being amenable to increased tubule cell transduction and thereby enhanced potential for gene therapy by the induced proteinuria method.

### Materials and Methods

### Animal Studies

All experiments were carried out according to the provisions of the Animal Welfare Act, PHS Animal Welfare Policy, the principles of the NIH Guide for the Care and Use of Laboratory Animals.

### AAV Vectors

AAV vectors were produced using a standard triple transfection and iodixanol gradient purification method. Briefly, a vector plasmid (pTRS-CBh-Cre), a rep and cap plasmid (pRC), and a pHelper plasmid were transfected into 293T cells using polyethylenimine. Three days later, cells were harvested and lysed by successive freeze/thaw cycles. Cell lysate was overlayed onto an iodixanol gradient and ultracentrifuged for two hours. The banded AAV was extracted via needle and syringe and titrated via qPCR using SYBR™ Green. All AAV vectors used in this study were self-complementary (scAAV) with a cytomegalovirus chicken β-actin hybrid promoter (CBh) driving expression of the Cre recombinase gene.

### Ad Vectors

Replication-defective Ad vectors were produced in 293 cells and were purified by double banding on CsCl gradients. Cre expression is driven by the CMV promoter.

### Flow Cytometry

Kidney samples were chopped into small pieces using scissors and put in Miltenyi© tubes. 2.35 mL of Gibco DMEM (cat #11054001), 100 μL of enzyme D, 50 μL of enzyme R, and 12.5 μL of enzyme A from the Miltenyi “Tumor Dissociation Kit” were added into each sample. Samples were homogenized using soft tissue dissociation program on Miltenyi OctoMACS™ Separator. Samples were passed through 70 μM filters and spun at 400×g for 10 minutes. Pellets were resuspended in 3.1 mL of cold DPBS, treated with 900 μL of Miltenyi Debris removal solution, overlayed with 4 mL of ice cold DPBS, and spun at 3000×g for 10 minutes. The samples were washed with DPBS and red blood cells were lysed with 1 mL of ACK Lysis buffer for 1 minute. The samples were resuspended in 900 μL of RPMI and filtered using 35 μM flow tube filters.

Fluorescent staining occurred as follows: After all samples were processed and passed through filters, they were washed twice with PBS. Samples were stained with a master mix composed of EpCAM PECy7 (1:250) (BioLegend, Cat #118216), CD31 AF647 (1:500) (BioLegend, Cat #102516), CD45 perCP (1:1000) (BioLegend, Cat #103130), TCRβ BV421 (1:1000), CD4 BV510 32 μL (1:500), CD8 BV570 (1:500), CD11b BV650 (1:1000), Ghost Dye Red 780 (1:2000) (Tonbo Biosciences, Cat #13-0865-T100), and FC block (1:500) (BD Pharmingen, Cat #553141). Three minutes prior to experimental mice being sacrificed, 3 μg of CD45 BV711 was injected intravenously to be able to distinguish between circulating and tissue resident CD45+ cells. Samples were stained for 30 minutes at 4 C in the dark, washed twice with PBS and ran on Cytek™ Aurora spectral flow cytometer.

For the experiments also staining against α-Fucose, Lotus Tetragonolobus Lectin (LTL), Biotinylated (1:100) (Vector Laboratories, Cat #B-1325-2) was the primary stain and BV786 Streptavidin (1:2000) (BD Horizon, Cat #563858) was the secondary stain. For the experiments also staining against Aquaporin-1, Anti-Aquaporin-1 (1:100) (Boster Biological Technology, Cat #PB9473) was the primary stain and anti-rabbit AF647 (1:2000) (Invitrogen, Cat #A-21245) was the secondary stain. In these experiments, CD31 was stained using anti-CD31 BV510 (1:150) (BD Biosciences, Cat #740124).

### In Vivo Bioluminescent Imaging

Mice were anesthetized with isoflurane and injected intraperitoneally with 150 μL of D-Luciferin (20 mg/mL; RR Labs Inc., San Diego, CA). Images were taken using PerkinElmer IVIS® Lumina S5 Imaging System ten minutes after D-Luciferin administration and luminescence was quantified using Living Image software. During ex vivo tissue imaging, tissues were placed in either 6-well or 12-well tissue culture vessels and imaged. In all cases except for FIG. 14, kidneys were laterally bisected before imaging to prevent squelching of luminescence by the kidney capsule.

### Statistical Analyses

All statistical analyses were performed using GraphPad Prism 9. p-values were generated using Mann-Whitney tests unless otherwise noted.

### Tissue Sectioning and Confocal Microscopy

Tissues from mice with membrane-bound fluorescent proteins were fixed by overnight immersion in 4% paraformaldehyde (PFA)-PBS at 4° C., then cryoprotected overnight in 15% sucrose-PBS and 30% sucrose-PBS, successively, at 4° C. Trimmed tissues were then flash frozen by dry ice-cooled isopentane in optimal cutting temperature (O.C.T.) medium (Sakura Finetek). Cryosections (18 μm thickness) were prepared with a Leica CM1860 UV cryostat (Leica Biosystems) and mounted on slides (Superfrost Plus; Thermo Fisher Scientific, Waltham, MA) with VECTASHIELD with 4′,6-diamidino-2-phenylindole (DAPI) (Vector Laboratories, Burlingame, CA), and CytoSeal-60 coverslip sealant (Thermo Fisher Scientific). Confocal imaging was performed using a Zeiss LSM780 laser confocal microscope (Carl Zeiss Jena, Jena, Germany).

For tissue sections stained with lotus tetragonolobus lectin (LTL), the slides containing tissue sections were washed with PBS, treated with 5% normal goat serum (Abcam Catalog #ab7481) and 0.5% IGEPAL® CA-630 (Sigma 18896) dissolved in PBS blocking buffer for 1 hour at room temperature. The slides were then incubated with a 1:100 dilution of biotinylated LTL (Vector Laboratories Cat. No: B-1325) overnight at 4° C. The slides were washed and then incubated with a 1:200 dilution of streptavidin-Alexa Fluor 647 (Invitrogen Catalog #S21374) at room temperature for one hour. The slides were washed and coverslips were mounted using Vectashield (without DAPI).

### Transgenic Mice

LSL-Luc mice (Stock No: 005125) and mT/mG mice (Stock No: 007576) were originally purchased from The Jackson Laboratory. Pkd1^{RC/RC} mice of 129S6 genetic background, which develop polycystic kidney disease, were backcrossed with mT/mG mice until pups were acquired that had exactly two copies of the Pkd1RC allele and at least one copy of the mT/mG allele, which was confirmed via PCR genotyping.

### Example 5: AAV Serotypes and Transduction of Renal Tubule Epithelial Cells after Intravenous Administration

### Results

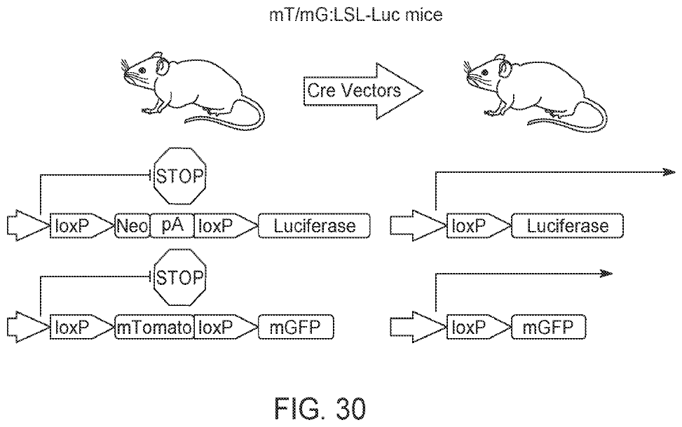

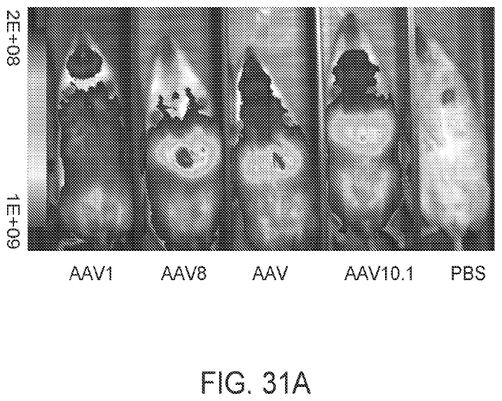

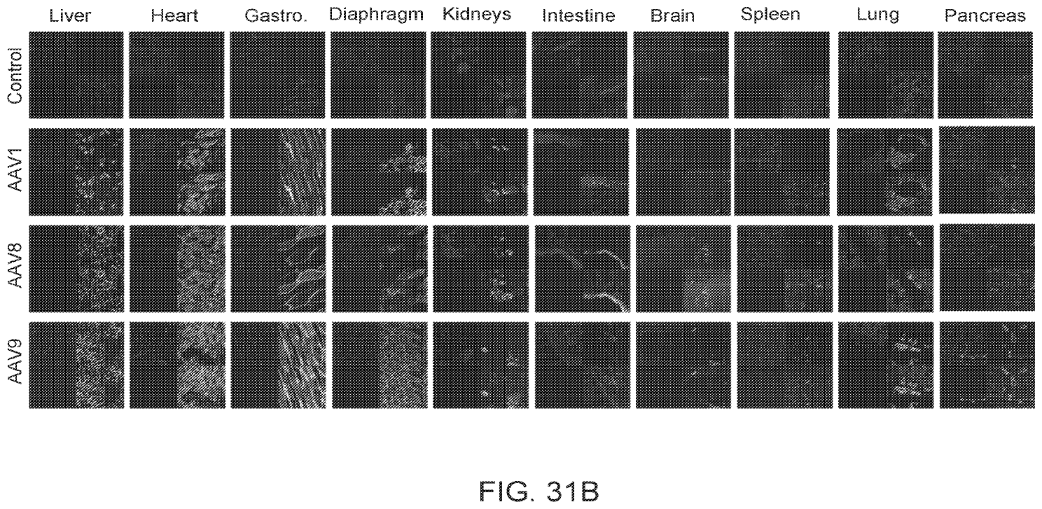

To examine the ability of AAV vectors to deliver genes into different tissues and the kidney, different AAV serotypes were used to package the Cre recombinase gene. These vectors were then used to infect cre-reporter luciferase and membrane-bound GFP (mGFP) mice by intravenous injection (FIG. 30). Luciferase imaging of living animals demonstrated the ability of different AAV-Cre serotypes to activate luciferase in the liver and other tissues (FIG. 31A).

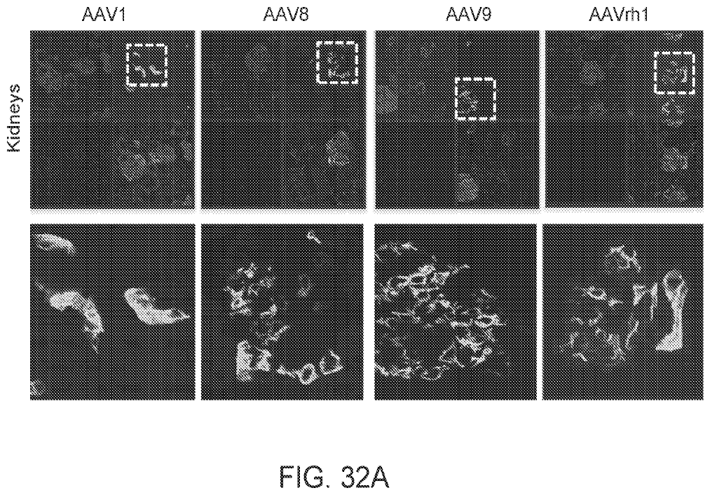

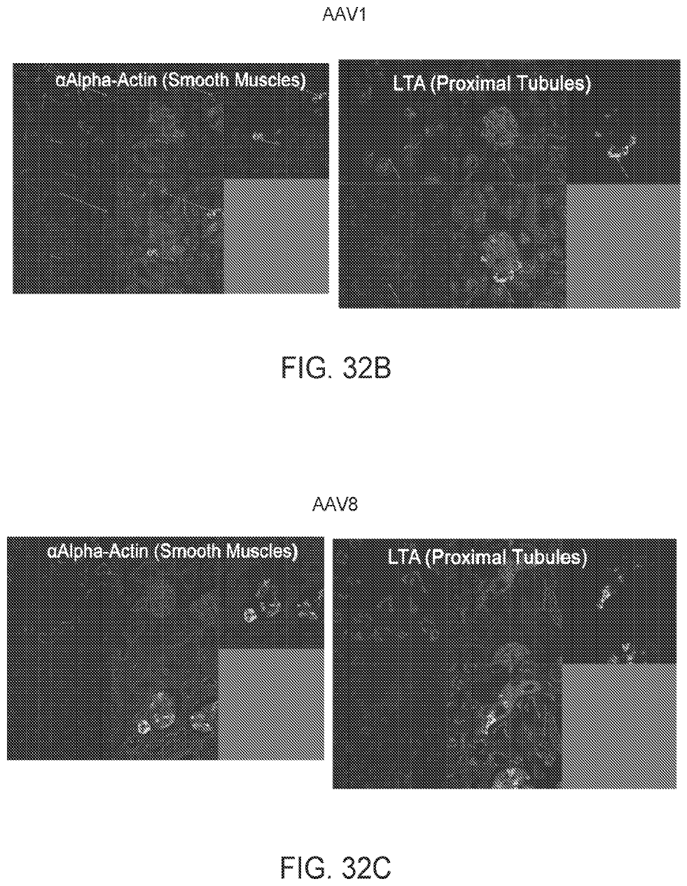

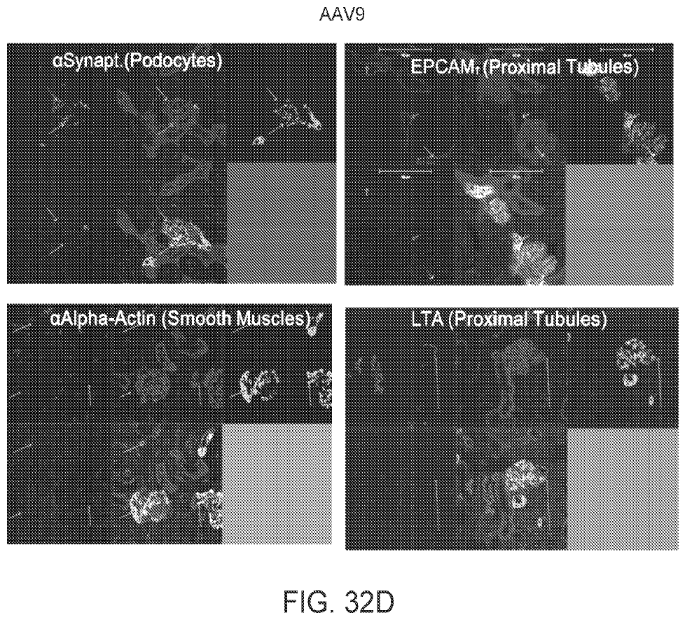

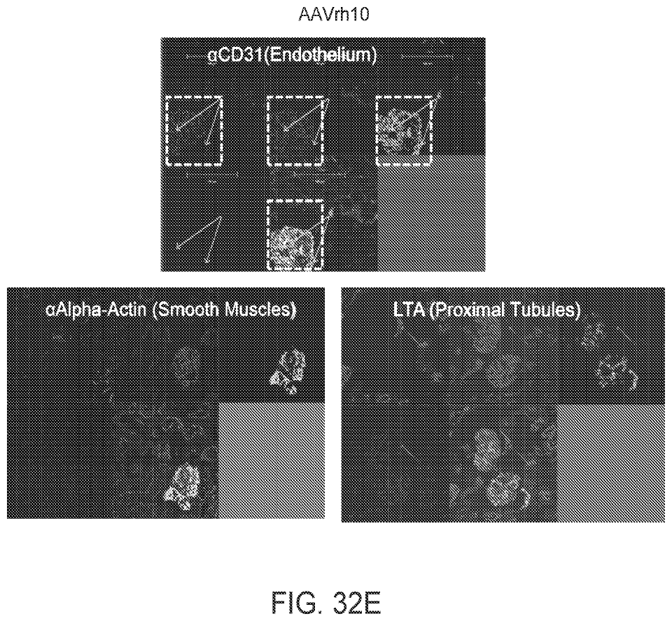

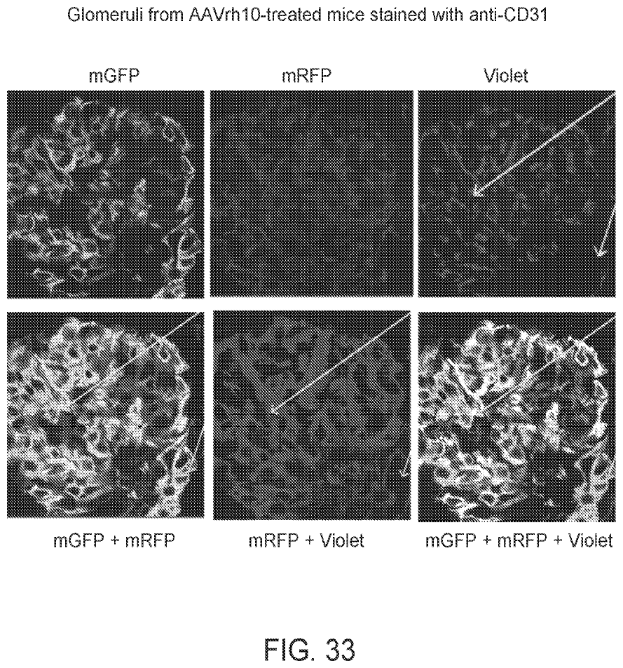

Tissues were collected from these animals and tissue- and cell-specific gene delivery was assessed by observing the conversion of membrane-targeted red fluorescent protein (mRFP)-positive cells that were converted to mGFP-positive cells by Cre by confocal microscopy of tissue sections (FIGS. 31B to 33). These data indicate that all AAVs have some level of transduction in multiple tissues, but with biases (FIG. 31B). When kidney sections were examined, the pattern of gene delivery as evidenced by mGFP localization was different by different serotypes (FIG. 32A). Globular patterns of mRFP-positive cells in the sections identify the glomerulus within these kidney sections (FIG. 32A-E). Observation of GFP-positive cells within these mRFP glomeruli demonstrates successful delivery of Cre recombinase to either endothelial cells or to podocytes within the glomerulus. GFP-positive cells outside of the mRFP-positive glomeruli indicate delivery to other renal cells.

When tissue sections were counterstained with cell-specific markers, AAV1 delivery localized with alpha-actin-positive smooth muscle cells in blood vessels rather than in glomerular cells. AAV1 also did not activate mGFP in Lotus Toxin Agglutin (LTA)-positive renal tubules cells (FIG. 32B).

AAV8 mediated Cre delivery to glomerular cells as well as macula densa cells, but not to alpha-actin positive smooth muscle cells and not to LTA-positive tubule cells (FIG. 32C).

AAV9 mediated Cre delivery to glomerular and macula densa cells, but not to alpha-actin positive smooth muscle cells, nor to alpha-synaptopodin (aSynapt)-positive podocytes, nor to LTA-positive tubule cells, but there was some delivery to EpCAM-positive proximal tubule cells (FIG. 32D).

AAVrh10 mediated Cre delivery to glomerular and macula densa cells including CD31-positive glomerular endothelial cells, but not to alpha-actin positive smooth muscle cells, nor to LTA-positive tubule cells (FIG. 32E). When CD31-stained glomeruli were examined at higher resolution, it was apparent that AAVrh10 was mediating equal transduction to CD31-positive endothelial cells and to CD31-negative podocytes within the glomerulus.

Together these results demonstrate that multiple serotypes of AAV can be used to deliver nucleic acid to cells within the kidneys. These results also demonstrate that different serotypes and different AAV capsids mediate delivery into different subsets of kidney cells.

### Methods

pAAV-Cre vectors were packaged an adenovirus helper plasmid with the indicated AAV Rep2/Cap1, 8, 9, or rh10 plasmids by triple transfection and AAV particles were purified. These were injected intravenously into Cre reporter mice by tail vein injection. Mice were anesthetized, injected with luciferin, and imaged for luciferase activity. Animals were sacrificed and frozen tissue sections were examined by confocal microscopy with and without counterstaining for cell-specific proteins using fluorescent antibodies.

SEQUENCES

PKD1 cDNA
SEQ ID NO: 1
ATGCCGCCCGCCGCGCCCGCCCGCCTGGCGCTGGCCCTGGGCCTGGGCCTGTGGCTCGGGGCGCTGGCGGGGGG
CCCCGGGGGCGCGCCGGGGGGCCCCGGGCGCGGCTGCGGGCCCTGCGAGCCCCCCTGCCTCTGCGGCCCAGCGC
CCGGCGCCGCCTGCCGCGTCAACTGCTCGGGCCGCGGGCTGCGGACGCTCGGTCCCGCGCTGCGCATCCCCGCG
GACGCCACAGCGCTAGACGTCTCCCACAACCTGCTCCGGGCGCTGGACGTTGGGCTCCTGGCGAACCTCTCGGC
GCTGGCAGAGCTGGATATAAGCAACAACAAGATTTCTACGTTAGAAGAAGGAATATTTGCTAATTTATTTAATT
TAAGTGAAATAAACCTGAGTGGGAACCCGTTTGAGTGTGACTGTGGCCTGGCGTGGCTGCCGCGATGGGCGGAG
GAGCAGCAGGTGCGGGTGGTGCAGCCCGAGGCAGCCACGTGTGCTGGGCCTGGCTCCCTGGCTGGCCAGCCTCT
GCTTGGCATCCCCTTGCTGGACAGTGGCTGTGGTGAGGAGTATGTCGCCTGCCTCCCTGACAACAGCTCAGGCA
CCGTGGCAGCAGTGTCCTTTTCAGCTGCCCACGAAGGCCTGCTTCAGCCAGAGGCCTGCAGCGCCTTCTGCTTC
TCCACCGGCCAGGGCCTCGCAGCCCTCTCGGAGCAGGGCTGGTGCCTGTGTGGGGCGGCCCAGCCCTCCAGTGC
CTCCTTTGCCTGCCTGTCCCTCTGCTCCGGCCCCCCGCCACCTCCTGCCCCCACCTGTAGGGGCCCCACCCTCC
TCCAGCACGTCTTCCCTGCCTCCCCAGGGGCCACCCTGGTGGGGCCCCACGGACCTCTGGCCTCTGGCCAGCTA
GCAGCCTTCCACATCGCTGCCCCGCTCCCTGTCACTGCCACACGCTGGGACTTCGGAGACGGCTCCGCCGAGGT
GGATGCCGCTGGGCCGGCTGCCTCGCATCGCTATGTGCTGCCTGGGCGCTATCACGTGACGGCCGTGCTGGCCC
TGGGGGCCGGCTCAGCCCTGCTGGGGACAGACGTGCAGGTGGAAGCGGCACCTGCCGCCCTGGAGCTCGTGTGC
CCGTCCTCGGTGCAGAGTGACGAGAGCCTTGACCTCAGCATCCAGAACCGCGGTGGTTCAGGCCTGGAGGCCGC
CTACAGCATCGTGGCCCTGGGCGAGGAGCCGGCCCGAGCGGTGCACCCGCTCTGCCCCTCGGACACGGAGATCT
TCCCTGGCAACGGGCACTGCTACCGCCTGGTGGTGGAGAAGGCGGCCTGGCTGCAGGCGCAGGAGCAGTGTCAG
GCCTGGGCCGGGGCCGCCCTGGCAATGGTGGACAGTCCCGCCGTGCAGCGCTTCCTGGTCTCCCGGGTCACCAG
GAGCCTAGACGTGTGGATCGGCTTCTCGACTGTGCAGGGGGTGGAGGTGGGCCCAGCGCCGCAGGGCGAGGCCT
TCAGCCTGGAGAGCTGCCAGAACTGGCTGCCCGGGGAGCCACACCCAGCCACAGCCGAGCACTGCGTCCGGCTC
GGGCCCACCGGGTGGTGTAACACCGACCTGTGCTCAGCGCCGCACAGCTACGTCTGCGAGCTGCAGCCCGGAGG
CCCAGTGCAGGATGCCGAGAACCTCCTCGTGGGAGCGCCCAGTGGGGACCTGCAGGGACCCCTGACGCCTCTGG
CACAGCAGGACGGCCTCTCAGCCCCGCACGAGCCCGTGGAGGTCATGGTATTCCCGGGCCTGCGTCTGAGCCGT
GAAGCCTTCCTCACCACGGCCGAATTTGGGACCCAGGAGCTCCGGCGGCCCGCCCAGCTGCGGCTGCAGGTGTA
CCGGCTCCTCAGCACAGCAGGGACCCCGGAGAACGGCAGCGAGCCTGAGAGCAGGTCCCCGGACAACAGGACCC
AGCTGGCCCCCGCGTGCATGCCAGGGGGACGCTGGTGCCCTGGAGCCAACATCTGCTTGCCGCTGGACGCCTCC
TGCCACCCCCAGGCCTGCGCCAATGGCTGCACGTCAGGGCCAGGGCTACCCGGGGCCCCCTATGCGCTATGGAG
AGAGTTCCTCTTCTCCGTTCCCGCGGGGCCCCCCGCGCAGTACTCGGTCACCCTCCACGGCCAGGATGTCCTCA
TGCTCCCTGGTGACCTCGTTGGCTTGCAGCACGACGCTGGCCCTGGCGCCCTCCTGCACTGCTCGCCGGCTCCC
GGCCACCCTGGTCCCCAGGCCCCGTACCTCTCCGCCAACGCCTCGTCATGGCTGCCCCACTTGCCAGCCCAGCT
GGAGGGCACTTGGGCCTGCCCTGCCTGTGCCCTGCGGCTGCTTGCAGCCACGGAACAGCTCACCGTGCTGCTGG
GCTTGAGGCCCAACCCTGGACTGCGGCTGCCTGGGCGCTATGAGGTCCGGGCAGAGGTGGGCAATGGCGTGTCC
AGGCACAACCTCTCCTGCAGCTTTGACGTGGTCTCCCCAGTGGCTGGGCTGCGGGTCATCTACCCTGCCCCCCG
CGACGGCCGCCTCTACGTGCCCACCAACGGCTCAGCCTTGGTGCTCCAGGTGGACTCTGGTGCCAACGCCACGG
CCACGGCTCGCTGGCCTGGGGGCAGTGTCAGCGCCCGCTTTGAGAATGTCTGCCCTGCCCTGGTGGCCACCTTC
GTGCCCGGCTGCCCCTGGGAGACCAACGATACCCTGTTCTCAGTGGTAGCACTGCCGTGGCTCAGTGAGGGGGA
GCACGTGGTGGACGTGGTGGTGGAAAACAGCGCCAGCCGGGCCAACCTCAGCCTGCGGGTGACGGCGGAGGAGC
CCATCTGTGGCCTCCGCGCCACGCCCAGCCCCGAGGCCCGTGTACTGCAGGGAGTCCTAGTGAGGTACAGCCCC
GTGGTGGAGGCCGGCTCGGACATGGTCTTCCGGTGGACCATCAACGACAAGCAGTCCCTGACCTTCCAGAACGT
GGTCTTCAATGTCATTTATCAGAGCGCGGCGGTCTTCAAGCTCTCACTGACGGCCTCCAACCACGTGAGCAACG
TCACCGTGAACTACAACGTAACCGTGGAGCGGATGAACAGGATGCAGGGTCTGCAGGTCTCCACAGTGCCGGCC
GTGGACCTTTGGGGATGGGGAGCAGGCCCTCCACCAGTTCCAGCCTCCGTACAACGAGTCCTTCCCGGTTCCAG
ACCCCTCGGTGGCCCAGGTGCTGGTGGAGCACAATGTCATGCACACCTACGCTGCCCCAGGTGAGTACCTCCTG
ACCGTGCTGGCATCTAATGCCTTCGAGAACCTGACGCAGCAGGTGCCTGTGAGCGTGCGCGCCTCCCTGCCCTC
CGTGGCTGTGGGTGTGAGTGACGGCGTCCTGGTGGCCGGCCGGCCCGTCACCTTCTACCCGCACCCGCTGCCCT
CGCCTGGGGGTGTTCTTTACACGTGGGACTTCGGGGACGGCTCCCCTGTCCTGACCCAGAGCCAGCCGGCTGCC
AACCACACCTATGCCTCGAGGGGCACCTACCACGTGCGCCTGGAGGTCAACAACACGGTGAGCGGTGCGGCGGC
CCAGGCGGATGTGCGCGTCTTTGAGGAGCTCCGCGGACTCAGCGTGGACATGAGCCTGGCCGTGGAGCAGGGCG
CCCCCGTGGTGGTCAGCGCCGCGGTGCAGACGGGCGACAACATCACGTGGACCTTCGACATGGGGGACGGCACC
GTGCTGTCGGGCCCGGAGGCAACAGTGGAGCATGTGTACCTGCGGGCACAGAACTGCACAGTGACCGTGGGTGC
GGCCAGCCCCGCCGGCCACCTGGCCCGGAGCCTGCACGTGCTGGTCTTCGTCCTGGAGGTGCTGCGCGTTGAAC
CCGCCGCCTGCATCCCCACGCAGCCTGACGCGCGGCTCACGGCCTACGTCACCGGGAACCCGGCCCACTACCTC
TTCGACTGGACCTTCGGGGATGGCTCCTCCAACACGACCGTGCGGGGGTGCCCGACGGTGACACACAACTTCAC
GCGGAGCGGCACGTTCCCCCTGGCGCTGGTGCTGTCCAGCCGCGTGAACAGGGCGCATTACTTCACCAGCATCT
GCGTGGAGCCAGAGGTGGGCAACGTCACCCTGCAGCCAGAGAGGCAGTTTGTGCAGCTCGGGGACGAGGCCTGG
CTGGTGGCATGTGCCTGGCCCCCGTTCCCCTACCGCTACACCTGGGACTTTGGCACCGAGGAAGCCGCCCCCAC
CCGTGCCAGGGGCCCTGAGGTGACGTTCATCTACCGAGACCCAGGCTCCTATCTTGTGACAGTCACCGCGTCCA
ACAACATCTCTGCTGCCAATGACTCAGCCCTGGTGGAGGTGCAGGAGCCCGTGCTGGTCACCAGCATCAAGGTC
AATGGCTCCCTTGGGCTGGAGCTGCAGCAGCCGTACCTGTTCTCTGCTGTGGGCCGTGGGCGCCCCGCCAGCTA
CCTGTGGGATCTGGGGGACGGTGGGTGGCTCGAGGGTCCGGAGGTCACCCACGCTTACAACAGCACAGGTGACT
TCACCGTTAGGGTGGCCGGCTGGAATGAGGTGAGCCGCAGCGAGGCCTGGCTCAATGTGACGGTGAAGCGGCGC
GTGCGGGGGCTCGTCGTCAATGCAAGCCGCACGGTGGTGCCCCTGAATGGGAGCGTGAGCTTCAGCACGTCGCT
GGAGGCCGGCAGTGATGTGCGCTATTCCTGGGTGCTCTGTGACCGCTGCACGCCCATCCCTGGGGGTCCTACCA
TCTCTTACACCTTCCGCTCCGTGGGCACCTTCAATATCATCGTCACGGCTGAGAACGAGGTGGGCTCCGCCCAG
GACAGCATCTTCGTCTATGTCCTGCAGCTCATAGAGGGGCTGCAGGTGGTGGGCGGTGGCCGCTACTTCCCCAC
CAACCACACGGTACAGCTGCAGGCCGTGGTTAGGGATGGCACCAACGTCTCCTACAGCTGGACTGCCTGGAGGG
ACAGGGGCCCGGCCCTGGCCGGCAGCGGCAAAGGCTTCTCGCTCACCGTGCTCGAGGCCGGCACCTACCATGTG
CAGCTGCGGGCCACCAACATGCTGGGCAGCGCCTGGGCCGACTGCACCATGGACTTCGTGGAGCCTGTGGGGTG
GCTGATGGTGGCCGCCTCCCCGAACCCAGCTGCCGTCAACACAAGCGTCACCCTCAGTGCCGAGCTGGCTGGTG
GCAGTGGTGTCGTATACACTTGGTCCTTGGAGGAGGGGCTGAGCTGGGAGACCTCCGAGCCATTTACCACCCAT
AGCTTCCCCACACCCGGCCTGCACTTGGTCACCATGACGGCAGGGAACCCGCTGGGCTCAGCCAACGCCACCGT
GGAAGTGGATGTGCAGGTGCCTGTGAGTGGCCTCAGCATCAGGGCCAGCGAGCCCGGAGGCAGCTTCGTGGCGG
CCGGGTCCTCTGTGCCCTTTTGGGGGCAGCTGGCCACGGGCACCAATGTGAGCTGGTGCTGGGCTGTGCCCGGC
GGCAGCAGCAAGCGTGGCCCTCATGTCACCATGGTCTTCCCGGATGCTGGCACCTTCTCCATCCGGCTCAATGC
CTCCAACGCAGTCAGCTGGGTCTCAGCCACGTACAACCTCACGGCGGAGGAGCCCATCGTGGGCCTGGTGCTGT
GGGCCAGCAGCAAGGTGGTGGCGCCCGGGCAGCTGGTCCATTTTCAGATCCTGCTGGCTGCCGGCTCAGCTGTC
ACCTTCCGCCTGCAGGTCGGCGGGGCCAACCCCGAGGTGCTCCCCGGGCCCCGTTTCTCCCACAGCTTCCCCCG
CGTCGGAGACCACGTGGTGAGCGTGCGGGGCAAAAACCACGTGAGCTGGGCCCAGGCGCAGGTGCGCATCGTGG
TGCTGGAGGCCGTGAGTGGGCTGCAGGTGCCCAACTGCTGCGAGCCTGGCATCGCCACGGGCACTGAGAGGAAC
TTCACAGCCCGCGTGCAGCGCGGCTCTCGGGTCGCCTACGCCTGGTACTTCTCGCTGCAGAAGGTCCAGGGCGA
CTCGCTGGTCATCCTGTCGGGCCGCGACGTCACCTACACGCCCGTGGCCGCGGGGCTGTTGGAGATCCAGGTGC
GCGCCTTCAACGCCCTGGGCAGTGAGAACCGCACGCTGGTGCTGGAGGTTCAGGACGCCGTCCAGTATGTGGCC
CTGCAGAGCGGCCCCTGCTTCACCAACCGCTCGGCGCAGTTTGAGGCCGCCACCAGCCCCAGCCCCCGGCGTGT
GGCCTACCACTGGGACTTTGGGGATGGGTCGCCAGGGCAGGACACAGATGAGCCCAGGGCCGAGCACTCCTACC
TGAGGCCTGGGGACTACCGCGTGCAGGTGAACGCCTCCAACCTGGTGAGCTTCTTCGTGGCGCAGGCCACGGTG
ACCGTCCAGGTGCTGGCCTGCCGGGAGCCGGAGGTGGACGTGGTCCTGCCCCTGCAGGTGCTGATGCGGCGATC
ACAGCGCAACTACTTGGAGGCCCACGTTGACCTGCGCGACTGCGTCACCTACCAGACTGAGTACCGCTGGGAGG
TGTATCGCACCGCCAGCTGCCAGCGGCCGGGGCGCCCAGCGCGTGTGGCCCTGCCCGGCGTGGACGTGAGCCGG
CCTCGGCTGGTGCTGCCGCGGCTGGCGCTGCCTGTGGGGCACTACTGCTTTGTGTTTGTCGTGTCATTTGGGGA
CACGCCACTGACACAGAGCATCCAGGCCAATGTGACGGTGGCCCCCGAGCGCCTGGTGCCCATCATTGAGGGTG
GCTCATACCGCGTGTGGTCAGACACACGGGACCTGGTGCTGGATGGGAGCGAGTCCTACGACCCCAACCTGGAG
GACGGCGACCAGACGCCGCTCAGTTTCCACTGGGCCTGTGTGGCTTCGACACAGAGGGAGGCTGGCGGGTGTGC
GCTGAACTTTGGGCCCCGCGGGAGCAGCACGGTCACCATTCCACGGGAGCGGCTGGCGGCTGGCGTGGAGTACA
CCTTCAGCCTGACCGTGTGGAAGGCCGGCCGCAAGGAGGAGGCCACCAACCAGACGGTGCTGATCCGGAGTGGC
CGGGTGCCCATTGTGTCCTTGGAGTGTGTGTCCTGCAAGGCACAGGCCGTGTACGAAGTGAGCCGCAGCTCCTA
CGTGTACTTGGAGGGCCGCTGCCTCAATTGCAGCAGCGGCTCCAAGCGAGGGCGGTGGGCTGCACGTACGTTCA
GCAACAAGACGCTGGTGCTGGATGAGACCACCACATCCACGGGCAGTGCAGGCATGCGACTGGTGCTGCGGCGG
GGCGTGCTGCGGGACGGCGAGGGATACACCTTCACGCTCACGGTGCTGGGCCGCTCTGGCGAGGAGGAGGGCTG
CGCCTCCATCCGCCTGTCCCCCAACCGCCCGCCGCTGGGGGGCTCTTGCCGCCTCTTCCCACTGGGCGCTGTGC
ACGCCCTCACCACCAAGGTGCACTTCGAATGCACGGGCTGGCATGACGCGGAGGATGCTGGCGCCCCGCTGGTG
TACGCCCTGCTGCTGCGGCGCTGTCGCCAGGGCCACTGCGAGGAGTTCTGTGTCTACAAGGGCAGCCTCTCCAG
CTACGGAGCCGTGCTGCCCCCGGGTTTCAGGCCACACTTCGAGGTGGGCCTGGCCGTGGTGGTGCAGGACCAGC
TGGGAGCCGCTGTGGTCGCCCTCAACAGGTCTTTGGCCATCACCCTCCCAGAGCCCAACGGCAGCGCAACGGGG
CTCACAGTCTGGCTGCACGGGCTCACCGCTAGTGTGCTCCCAGGGCTGCTGCGGCAGGCCGATCCCCAGCACGT
CATCGAGTACTCGTTGGCCCTGGTCACCGTGCTGAACGAGTACGAGCGGGCCCTGGACGTGGCGGCAGAGCCCA
AGCACGAGCGGCAGCACCGAGCCCAGATACGCAAGAACATCACGGAGACTCTGGTGTCCCTGAGGGTCCACACT
GTGGATGACATCCAGCAGATCGCTGCTGCGCTGGCCCAGTGCATGGGGCCCAGCAGGGAGCTCGTATGCCGCTC
GTGCCTGAAGCAGACGCTGCACAAGCTGGAGGCCATGATGCTCATCCTGCAGGCAGAGACCACCGCGGGCACCG
TGACGCCCACCGCCATCGGAGACAGCATCCTCAACATCACAGGAGACCTCATCCACCTGGCCAGCTCGGACGTG
CGGGCACCACAGCCCTCAGAGCTGGGAGCCGAGTCACCATCTCGGATGGTGGCGTCCCAGGCCTACAACCTGAC
CTCTGCCCTCATGCGCATCCTCATGCGCTCCCGCGTGCTCAACGAGGAGCCCCTGACGCTGGCGGGCGAGGAGA
TCGTGGCCCAGGGCAAGCGCTCGGACCCGCGGAGCCTGCTGTGCTATGGCGGCGCCCCAGGGCCTGGCTGCCAC
TTCTCCATCCCCGAGGCTTTCAGCGGGGCCCTGGCCAACCTCAGTGACGTGGTGCAGCTCATCTTTCTGGTGGA
CTCCAATCCCTTTCCCTTTGGCTATATCAGCAACTACACCGTCTCCACCAAGGTGGCCTCGATGGCATTCCAGA
CACAGGCCGGCGCCCAGATCCCCATCGAGCGGCTGGCCTCAGAGCGCGCCATCACCGTGAAGGTGCCCAACAAC
TCGGACTGGGCTGCCCGGGGCCACCGCAGCTCCGCCAACTCCGCCAACTCCGTTGTGGTCCAGCCCCAGGCCTC
CGTCGGTGCTGTGGTCACCCTGGACAGCAGCAACCCTGCGGCCGGGCTGCATCTGCAGCTCAACTATACGCTGC
TGGACGGCCACTACCTGTCTGAGGAACCTGAGCCCTACCTGGCAGTCTACCTACACTCGGAGCCCCGGCCCAAT
GAGCACAACTGCTCGGCTAGCAGGAGGATCCGCCCAGAGTCACTCCAGGGTGCTGACCACCGGCCCTACACCTT
CTTCATTTCCCCGGGGAGCAGAGACCCAGCGGGGAGTTACCATCTGAACCTCTCCAGCCACTTCCGCTGGTCGG
CGCTGCAGGTGTCCGTGGGCCTGTACACGTCCCTGTGCCAGTACTTCAGCGAGGAGGACATGGTGTGGCGGACA
GAGGGGCTGCTGCCCCTGGAGGAGACCTCGCCCCGCCAGGCCGTCTGCCTCACCCGCCACCTCACCGCCTTCGG
CGCCAGCCTCTTCGTGCCCCCAAGCCATGTCCGCTTTGTGTTTCCTGAGCCGACAGCGGATGTAAACTACATCG
TCATGCTGACATGTGCTGTGTGCCTGGTGACCTACATGGTCATGGCCGCCATCCTGCACAAGCTGGACCAGTTG
GATGCCAGCCGGGGCCGCGCCATCCCTTTCTGTGGGCAGCGGGGCCGCTTCAAGTACGAGATCCTCGTCAAGAC
AGGCTGGGGCCGGGGCTCAGGTACCACGGCCCACGTGGGCATCATGCTGTATGGGGTGGACAGCCGGAGCGGCC
ACCGGCACCTGGACGGCGACAGAGCCTTCCACCGCAACAGCCTGGACATCTTCCGGATCGCCACCCCGCACAGC
CTGGGTAGCGTGTGGAAGATCCGAGTGTGGCACGACAACAAAGGGCTCAGCCCTGCCTGGTTCCTGCAGCACGT
CATCGTCAGGGACCTGCAGACGGCACGCAGCGCCTTCTTCCTGGTCAATGACTGGCTTTCGGTGGAGACGGAGG
CCAACGGGGGCCTGGTGGAGAAGGAGGTGCTGGCCGCGAGCGACGCAGCCCTTTTGCGCTTCCGGCGCCTGCTG
GTGGCTGAGCTGCAGCGTGGCTTCTTTGACAAGCACATCTGGCTCTCCATATGGGACCGGCCGCCTCGTAGCCG
TTTCACTCGCATCCAGAGGGCCACCTGCTGCGTTCTCCTCATCTGCCTCTTCCTGGGCGCCAACGCCGTGTGGT
ACGGGGCTGTTGGCGACTCTGCCTACAGCACGGGGCATGTGTCCAGGCTGAGCCCGCTGAGCGTCGACACAGTC
GCTGTTGGCCTGGTGTCCAGCGTGGTTGTCTATCCCGTCTACCTGGCCATCCTTTTTCTCTTCCGGATGTCCCG
GAGCAAGGTGGCTGGGAGCCCGAGCCCCACACCTGCCGGGCAGCAGGTGCTGGACATCGACAGCTGCCTGGACT
CGTCCGTGCTGGACAGCTCCTTCCTCACGTTCTCAGGCCTCCACGCTGAGCAGGCCTTTGTTGGACAGATGAAG
AGTGACTTGTTTCTGGATGATTCTAAGAGTCTGGTGTGCTGGCCCTCCGGCGAGGGAACGCTCAGTTGGCCGGA
CCTGCTCAGTGACCCGTCCATTGTGGGTAGCAATCTGCGGCAGCTGGCACGGGGCCAGGCGGGCCATGGGCTGG
GCCCAGAGGAGGACGGCTTCTCCCTGGCCAGCCCCTACTCGCCTGCCAAATCCTTCTCAGCATCAGATGAAGAC
CTGATCCAGCAGGTCCTTGCCGAGGGGGTCAGCAGCCCAGCCCCTACCCAAGACACCCACATGGAAACGGACCT
GCTCAGCAGCCTGTCCAGCACTCCTGGGGAGAAGACAGAGACGCTGGCGCTGCAGAGGCTGGGGGAGCTGGGGC
CACCCAGCCCAGGCCTGAACTGGGAACAGCCCCAGGCAGCGAGGCTGTCCAGGACAGGACTGGTGGAGGGTCTG
CGGAAGCGCCTGCTGCCGGCCTGGTGTGCCTCCCTGGCCCACGGGCTCAGCCTGCTCCTGGTGGCTGTGGCTGT
GGCTGTCTCAGGGTGGGTGGGTGCGAGCTTCCCCCCGGGCGTGAGTGTTGCGTGGCTCCTGTCCAGCAGCGCCA
GCTTCCTGGCCTCATTCCTCGGCTGGGAGCCACTGAAGGTCTTGCTGGAAGCCCTGTACTTCTCACTGGTGGCC
AAGCGGCTGCACCCGGATGAAGATGACACCCTGGTAGAGAGCCCGGCTGTGACGCCTGTGAGCGCACGTGTGCC
CCGCGTACGGCCACCCCACGGCTTTGCACTCTTCCTGGCCAAGGAAGAAGCCCGCAAGGTCAAGAGGCTACATG
GCATGCTGCGGAGCCTCCTGGTGTACATGCTTTTTCTGCTGGTGACCCTGCTGGCCAGCTATGGGGATGCCTCA
TGCCATGGGCACGCCTACCGTCTGCAAAGCGCCATCAAGCAGGAGCTGCACAGCCGGGCCTTCCTGGCCATCAC
GCGGTCTGAGGAGCTCTGGCCATGGATGGCCCACGTGCTGCTGCCCTACGTCCACGGGAACCAGTCCAGCCCAG
AGCTGGGGCCCCCACGGCTGCGGCAGGTGCGGCTGCAGGAAGCACTCTACCCAGACCCTCCCGGCCCCAGGGTC
CACACGTGCTCGGCCGCAGGAGGCTTCAGCACCAGCGATTACGACGTTGGCTGGGAGAGTCCTCACAATGGCTC
GGGGACGTGGGCCTATTCAGCGCCGGATCTGCTGGGGGCATGGTCCTGGGGCTCCTGTGCCGTGTATGACAGCG
GGGGCTACGTGCAGGAGCTGGGCCTGAGCCTGGAGGAGAGCCGCGACCGGCTGCGCTTCCTGCAGCTGCACAAC
TGGCTGGACAACAGGAGCCGCGCTGTGTTCCTGGAGCTCACGCGCTACAGCCCGGCCGTGGGGCTGCACGCCGC
CGTCACGCTGCGCCTCGAGTTCCCGGCGGCCGGCCGCGCCCTGGCCGCCCTCAGCGTCCGCCCCTTTGCGCTGC
GCCGCCTCAGCGCGGGCCTCTCGCTGCCTCTGCTCACCTCGGTGTGCCTGCTGCTGTTCGCCGTGCACTTCGCC
GTGGCCGAGGCCCGTACTTGGCACAGGGAAGGGCGCTGGCGCGTGCTGCGGCTCGGAGCCTGGGCGCGGTGGCT
GCTGGTGGCGCTGACGGCGGCCACGGCACTGGTACGCCTCGCCCAGCTGGGTGCCGCTGACCGCCAGTGGACCC
GTTTCGTGCGCGGCCGCCCGCGCCGCTTCACTAGCTTCGACCAGGTGGCGCAGCTGAGCTCCGCAGCCCGTGGC
CTGGCGGCCTCGCTGCTCTTCCTGCTTTTGGTCAAGGCTGCCCAGCAGCTACGCTTCGTGCGCCAGTGGTCCGT
CTTTGGCAAGACATTATGCCGAGCTCTGCCAGAGCTCCTGGGGGTCACCTTGGGCCTGGTGGTGCTCGGGGTAG
CCTACGCCCAGCTGGCCATCCTGCTCGTGTCTTCCTGTGTGGACTCCCTCTGGAGCGTGGCCCAGGCCCTGTTG
GTGCTGTGCCCTGGGACTGGGCTCTCTACCCTGTGTCCTGCCGAGTCCTGGCACCTGTCACCCCTGCTGTGTGT
GGGGCTCTGGGCACTGCGGCTGTGGGGCGCCCTACGGCTGGGGGCTGTTATTCTCCGCTGGCGCTACCACGCCT
TGCGTGGAGAGCTGTACCGGCCGGCCTGGGAGCCCCAGGACTACGAGATGGTGGAGTTGTTCCTGCGCAGGCTG
CGCCTCTGGATGGGCCTCAGCAAGGTCAAGGAGTTCCGCCACAAAGTCCGCTTTGAAGGGATGGAGCCGCTGCC
CTCTCGCTCCTCCAGGGGCTCCAAGGTATCCCCGGATGTGCCCCCACCCAGCGCTGGCTCCGATGCCTCGCACC
CCTCCACCTCCTCCAGCCAGCTGGATGGGCTGAGCGTGAGCCTGGGCCGGCTGGGGACAAGGTGTGAGCCTGAG
CCCTCCCGCCTCCAAGCCGTGTTCGAGGCCCTGCTCACCCAGTTTGACCGACTCAACCAGGCCACAGAGGACGT
CTACCAGCTGGAGCAGCAGCTGCACAGCCTGCAAGGCCGCAGGAGCAGCCGGGCGCCCGCCGGATCTTCCCGTG
GCCCATCCCCGGGCCTGCGGCCAGCACTGCCCAGCCGCCTTGCCCGGGCCAGTCGGGGTGTGGACCTGGCCACT
GGCCCCAGCAGGACACCCCTTCGGGCCAAGAACAAGGTCCACCCCAGCAGCACTTAG
PC-1 polypeptide
SEQ ID NO: 2
MPPAAPARLALALGLGLWLGALAGGPGGAPGGPGRGCGPCEPPCLCGPAPGAACRVNCSGRGLRTLGPALRIPA
DATALDVSHNLLRALDVGLLANLSALAELDISNNKISTLEEGIFANLENLSEINLSGNPFECDCGLAWLPRWAE
EQQVRVVQPEAATCAGPGSLAGQPLLGIPLLDSGCGEEYVACLPDNSSGTVAAVSFSAAHEGLLQPEACSAFCF
STGQGLAALSEQGWCLCGAAQPSSASFACLSLCSGPPPPPAPTCRGPTLLQHVFPASPGATLVGPHGPLASGOL
AAFHIAAPLPVTATRWDFGDGSAEVDAAGPAASHRYVLPGRYHVTAVLALGAGSALLGTDVOVEAAPAALELVC
PSSVOSDESLDLSIQNRGGSGLEAAYSIVALGEEPARAVHPLCPSDTEIFPGNGHCYRLVVEKAAWLQAQEQCQ
AWAGAALAMVDSPAVQRFLVSRVTRSLDVWIGFSTVQGVEVGPAPQGEAFSLESCONWLPGEPHPATAEHCVRL
GPTGWCNTDLCSAPHSYVCELQPGGPVQDAENLLVGAPSGDLQGPLTPLAQQDGLSAPHEPVEVMVFPGIRLSR
EAFLTTAEFGTQELRRPAQLRLQVYRLLSTAGTPENGSEPESRSPDNRTQLAPACMPGGRWCPGANICLPLDAS
CHPQACANGCTSGPGLPGAPYALWREFLFSVPAGPPAQYSVTLHGQDVLMLPGDLVGLQHDAGPGALLHCSPAP
GHPGPQAPYLSANASSWLPHLPAQLEGTWACPACALRLLAATEQLTVLLGLRPNPGLRLPGRYEVRAEVGNGVS
RHNLSCSFDVVSPVAGLRVIYPAPRDGRLYVPTNGSALVLQVDSGANATATARWPGGSVSARFENVCPALVATF
VPGCPWETNDTLFSVVALPWLSEGEHVVDVVVENSASRANLSLRVTAEEPICGLRATPSPEARVLOGVLVRYSP
VVEAGSDMVFRWTINDKQSLTFQNVVENVIYQSAAVEKLSLTASNHVSNVTVNYNVTVERMNRMQGLQVSTVPA
VLSPNATLALTAGVLVDSAVEVAFLWTFGDGEQALHQFQPPYNESFPVPDPSVAQVLVEHNVMHTYAAPGEYLL
TVLASNAFENLTQQVPVSVRASLPSVAVGVSDGVLVAGRPVTFYPHPLPSPGGVLYTWDFGDGSPVLTQSQPAA
NHTYASRGTYHVRLEVNNTVSGAAAQADVRVFEELRGLSVDMSLAVEQGAPVVVSAAVQTGDNITWTFDMGDGT
VLSGPEATVEHVYLRAQNCTVTVGAASPAGHLARSLHVLVFVLEVLRVEPAACIPTQPDARLTAYVTGNPAHYL
FDWTFGDGSSNTTVRGCPTVTHNFTRSGTFPLALVLSSRVNRAHYFTSICVEPEVGNVTLQPERQFVOLGDEAW
LVACAWPPFPYRYTWDFGTEEAAPTRARGPEVTFIYRDPGSYLVTVTASNNISAANDSALVEVQEPVLVTSIKV
NGSLGLELQQPYLFSAVGRGRPASYLWDLGDGGWLEGPEVTHAYNSTGDFTVRVAGWNEVSRSEAWLNVTVKRR
VRGLVVNASRTVVPLNGSVSFSTSLEAGSDVRYSWVLCDRCTPIPGGPTISYTFRSVGTENIIVTAENEVGSAQ
DSIFVYVLQLIEGLQVVGGGRYFPTNHTVQLQAVVRDGTNVSYSWTAWRDRGPALAGSGKGFSLTVLEAGTYHV
QLRATNMLGSAWADCTMDFVEPVGWLMVAASPNPAAVNTSVTLSAELAGGSGVVYTWSLEEGLSWETSEPFTTH
SFPTPGLHLVTMTAGNPLGSANATVEVDVQVPVSGLSIRASEPGGSFVAAGSSVPFWGQLATGTNVSWCWAVPG
GSSKRGPHVTMVFPDAGTFSIRLNASNAVSWVSATYNLTAEEPIVGLVLWASSKVVAPGQLVHFQILLAAGSAV
TFRLQVGGANPEVLPGPRFSHSFPRVGDHVVSVRGKNHVSWAQAQVRIVVLEAVSGLQVPNCCEPGIATGTERN
FTARVQRGSRVAYAWYFSLQKVQGDSLVILSGRDVTYTPVAAGLLEIQVRAFNALGSENRTLVLEVODAVQYVA
LQSGPCFTNRSAQFEAATSPSPRRVAYHWDFGDGSPGODTDEPRAEHSYLRPGDYRVQVNASNLVSFFVAQATV
TVQVLACREPEVDVVLPLQVLMRRSQRNYLEAHVDLRDCVTYQTEYRWEVYRTASCQRPGRPARVALPGVDVSR
PRLVLPRLALPVGHYCFVFVVSFGDTPLTQSIQANVTVAPERLVPIIEGGSYRVWSDTRDLVLDGSESYDPNLE
DGDQTPLSFHWACVASTQREAGGCALNFGPRGSSTVTIPRERLAAGVEYTFSLTVWKAGRKEEATNOTVLIRSG
RVPIVSLECVSCKAQAVYEVSRSSYVYLEGRCLNCSSGSKRGRWAARTFSNKTLVLDETTTSTGSAGMRLVLRR
GVLRDGEGYTFTLTVLGRSGEEEGCASIRLSPNRPPLGGSCRLFPLGAVHALTTKVHFECTGWHDAEDAGAPLV
YALLLRRCROGHCEEFCVYKGSLSSYGAVLPPGFRPHFEVGLAVVVQDOLGAAVVALNRSLAITLPEPNGSATG
LTVWLHGLTASVLPGLLROADPQHVIEYSLALVTVLNEYERALDVAAEPKHERQHRAQIRKNITETLVSLRVHT
VDDIQQIAAALAQCMGPSRELVCRSCLKQTLHKLEAMMLILQAETTAGTVTPTAIGDSILNITGDLIHLASSDV
RAPQPSELGAESPSRMVASQAYNLTSALMRILMRSRVLNEEPLTLAGEEIVAQGKRSDPRSLLCYGGAPGPGCH
FSIPEAFSGALANLSDVVQLIFLVDSNPFPFGYISNYTVSTKVASMAFQTQAGAQIPIERLASERAITVKVPNN
SDWAARGHRSSANSANSVVVQPQASVGAVVTLDSSNPAAGLHLOLNYTLLDGHYLSEEPEPYLAVYLHSEPRPN
EHNCSASRRIRPESLOGADHRPYTFFISPGSRDPAGSYHLNLSSHFRWSALQVSVGLYTSLCQYFSEEDMVWRT
EGLLPLEETSPRQAVCLTRHLTAFGASLFVPPSHVRFVFPEPTADVNYIVMLTCAVCLVTYMVMAAILHKLDQL
DASRGRAIPFCGQRGRFKYEILVKTGWGRGSGTTAHVGIMLYGVDSRSGHRHLDGDRAFHRNSLDIFRIATPHS
LGSVWKIRVWHDNKGLSPAWFLOHVIVRDLQTARSAFFLVNDWLSVETEANGGLVEKEVLAASDAALLRERRLL
VAELQRGFFDKHIWLSIWDRPPRSRFTRIQRATCCVLLICLFLGANAVWYGAVGDSAYSTGHVSRLSPLSVDTV
AVGLVSSVVVYPVYLAILFLFRMSRSKVAGSPSPTPAGQQVLDIDSCLDSSVLDSSFLTFSGLHAEQAFVGQMK
SDLFLDDSKSLVCWPSGEGTLSWPDLLSDPSIVGSNLRQLARGOAGHGLGPEEDGFSLASPYSPAKSFSASDED
LIQQVLAEGVSSPAPTQDTHMETDLLSSLSSTPGEKTETLALQRLGELGPPSPGLNWEQPQAARLSRTGLVEGL
RKRLLPAWCASLAHGLSLLLVAVAVAVSGWVGASFPPGVSVAWLLSSSASFLASFLGWEPLKVLLEALYFSLVA
KRLHPDEDDTLVESPAVTPVSARVPRVRPPHGFALFLAKEEARKVKRLHGMLRSLLVYMLFLLVTLLASYGDAS
CHGHAYRLQSAIKQELHSRAFLAITRSEELWPWMAHVLLPYVHGNOSSPELGPPRLRQVRLOEALYPDPPGPRV
HTCSAAGGFSTSDYDVGWESPHNGSGTWAYSAPDLLGAWSWGSCAVYDSGGYVQELGLSLEESRDRLRFLQLHN
WLDNRSRAVFLELTRYSPAVGLHAAVTLRLEFPAAGRALAALSVRPFALRRLSAGLSLPLLTSVCLLLFAVHFA
VAEARTWHREGRWRVLRLGAWARWLLVALTAATALVRLAQLGAADRQWTRFVRGRPRRFTSFDQVAQLSSAARG
LAASLLFLLLVKAAQQLRFVROWSVFGKTLCRALPELLGVTLGLVVLGVAYAQLAILLVSSCVDSLWSVAQALL
VLCPGTGLSTLCPAESWHLSPLLCVGLWALRLWGALRLGAVILRWRYHALRGELYRPAWEPQDYEMVELFLRRL
RLWMGLSKVKEFRHKVRFEGMEPLPSRSSRGSKVSPDVPPPSAGSDASHPSTSSSQLDGLSVSLGRLGTRCEPE
PSRLQAVFEALLTQFDRLNQATEDVYQLEQQLHSLQGRRSSRAPAGSSRGPSPGLRPALPSRLARASRGVDLAT
GPSRTPLRAKNKVHPSST
PKD2 cDNA
SEQ ID NO: 3
ATGGTGAACTCCAGTCGCGTGCAGCCTCAGCAGCCCGGGGACGCCAAGCGGCCGCCCGCGCCCCGCGCGCCGGA
CCCGGGCCGGCTGATGGCTGGCTGCGCGGCCGTGGGCGCCAGCCTCGCCGCCCCGGGCGGCCTCTGCGAGCAGC
GGGGCCTGGAGATCGAGATGCAGCGCATCCGGCAGGCGGCCGCGCGGGACCCCCCGGCCGGAGCCGCGGCCTCC
CCTTCTCCTCCGCTCTCGTCGTGCTCCCGGCAGGCGTGGAGCCGCGATAACCCCGGCTTCGAGGCCGAGGAGGA
GGAGGAGGAGGTGGAAGGGGAAGAAGGCGGAATGGTGGTGGAGATGGACGTAGAGTGGCGCCCGGGCAGCCGGA
GGTCGGCCGCCTCCTCGGCCGTGAGCTCCGTGGGCGCGCGGAGCCGGGGGCTTGGGGGCTACCACGGCGCGGGC
CACCCGAGCGGGAGGCGGCGCCGGCGAGAGGACCAGGGCCCGCCGTGCCCCAGCCCAGTCGGCGGCGGGGACCC
GCTGCATCGCCACCTCCCCCTGGAAGGGCAGCCGCCCCGAGTGGCCTGGGCGGAGAGGCTGGTTCGCGGGCTGC
GAGGTCTCTGGGGAACAAGACTCATGGAGGAAAGCAGCACTAACCGAGAGAAATACCTTAAAAGTGTTTTACGG
GAACTGGTCACATACCTCCTTTTTCTCATAGTCTTGTGCATCTTGACCTACGGCATGATGAGCTCCAATGTGTA
CTACTACACCCGGATGATGTCACAGCTCTTCCTAGACACCCCCGTGTCCAAAACGGAGAAAACTAACTTTAAAA
CTCTGTCTTCCATGGAAGACTTCTGGAAGTTCACAGAAGGCTCCTTATTGGATGGGCTGTACTGGAAGATGCAG
CCCAGCAACCAGACTGAAGCTGACAACCGAAGTTTCATCTTCTATGAGAACCTGCTGTTAGGGGTTCCACGAAT
ACGGCAACTCCGAGTCAGAAATGGATCCTGCTCTATCCCCCAGGACTTGAGAGATGAAATTAAAGAGTGCTATG
ATGTCTACTCTGTCAGTAGTGAAGATAGGGCTCCCTTTGGGCCCCGAAATGGAACCGCTTGGATCTACACAAGT
GAAAAAGACTTGAATGGTAGTAGCCACTGGGGAATCATTGCAACTTATAGTGGAGCTGGCTATTATCTGGATTT
GTCAAGAACAAGAGAGGAAACAGCTGCACAAGTTGCTAGCCTCAAGAAAAATGTCTGGCTGGACCGAGGAACCA
GGGCAACTTTTATTGACTTCTCAGTGTACAACGCCAACATTAACCTGTTCTGTGTGGTCAGGTTATTGGTTGAA
TTCCCAGCAACAGGTGGTGTGATTCCATCTTGGCAATTTCAGCCTTTAAAGCTGATCCGATATGTCACAACTTT
TGATTTCTTCCTGGCAGCCTGTGAGATTATCTTTTGTTTCTTTATCTTTTACTATGTGGTGGAAGAGATATTGG
AAATTCGCATTCACAAACTACACTATTTCAGGAGTTTCTGGAATTGTCTGGATGTTGTGATCGTTGTGCTGTCA
GTGGTAGCTATAGGAATTAACATATACAGAACATCAAATGTGGAGGTGCTACTACAGTTTCTGGAAGATCAAAA
TACTTTCCCCAACTTTGAGCATCTGGCATATTGGCAGATACAGTTCAACAATATAGCTGCTGTCACAGTATTTT
TTGTCTGGATTAAGCTCTTCAAATTCATCAATTTTAACAGGACCATGAGCCAGCTCTCGACAACCATGTCTCGA
TGTGCCAAAGACCTGTTTGGCTTTGCTATTATGTTCTTCATTATTTTCCTAGCGTATGCTCAGTTGGCATACCT
TGTCTTTGGCACTCAGGTCGATGACTTCAGTACTTTCCAAGAGTGTATCTTCACTCAATTCCGTATCATTTTGG
GCGATATCAACTTTGCAGAGATTGAGGAAGCTAATCGAGTTTTGGGACCAATTTATTTCACTACATTTGTGTTC
TTTATGTTCTTCATTCTTTTGAATATGTTTTTGGCTATCATCAATGATACTTACTCTGAAGTGAAATCTGACTT
GGCACAGCAGAAAGCTGAAATGGAACTCTCAGATCTTATCAGAAAGGGCTACCATAAAGCTTTGGTCAAACTAA
AACTGAAAAAAAATACCGTGGATGACATTTCAGAGAGTCTGCGGCAAGGAGGAGGCAAGTTAAACTTTGACGAA
CTTCGACAAGATCTCAAAGGGAAGGGCCATACTGATGCAGAGATTGAGGCAATATTCACAAAGTACGACCAAGA
TGGAGACCAAGAACTGACCGAACATGAACATCAGCAGATGAGAGACGACTTGGAGAAAGAGAGGGAGGACCTGG
ATTTGGATCACAGTTCTTTACCACGTCCCATGAGCAGCCGAAGTTTCCCTCGAAGCCTGGATGACTCTGAGGAG
GATGACGATGAAGATAGCGGACATAGCTCCAGAAGGAGGGGAAGCATTTCTAGTGGCGTTTCTTACGAAGAGTT
TCAAGTCCTGGTGAGACGAGTGGACCGGATGGAGCATTCCATCGGCAGCATAGTGTCCAAGATTGACGCCGTGA
TCGTGAAGCTAGAGATTATGGAGCGAGCCAAACTGAAGAGGAGGGAGGTGCTGGGAAGGCTGTTGGATGGGGTG
GCCGAGGATGAAAGGCTGGGTCGTGACAGTGAAATCCATAGGGAACAGATGGAACGGCTAGTACGTGAAGAGTT
GGAACGCTGGGAATCCGATGATGCAGCTTCCCAGATCAGTCATGGTTTAGGCACGCCAGTGGGACTAAATGGTC
AACCTCGCCCCAGAAGCTCCCGCCCATCTTCCTCCCAATCTACAGAAGGCATGGAAGGTGCAGGTGGAAATGGG
AGTTCTAATGTCCACGTATGA
PC-2 polypeptide
SEQ ID NO: 4
MVNSSRVQPQQPGDAKRPPAPRAPDPGRLMAGCAAVGASLAAPGGLCEORGLEIEMORIRQAAARDPPAGAAAS
PSPPLSSCSRQAWSRDNPGFEAEEEEEEVEGEEGGMVVEMDVEWRPGSRRSAASSAVSSVGARSRGLGGYHGAG
HPSGRRRRREDQGPPCPSPVGGGDPLHRHLPLEGQPPRVAWAERLVRGLRGLWGTRLMEESSTNREKYLKSVLR
ELVTYLLFLIVLCILTYGMMSSNVYYYTRMMSQLFLDTPVSKTEKTNFKTLSSMEDFWKFTEGSLLDGLYWKMQ
PSNQTEADNRSFIFYENLLLGVPRIRQLRVRNGSCSIPQDLRDEIKECYDVYSVSSEDRAPFGPRNGTAWIYTS
EKDLNGSSHWGIIATYSGAGYYLDLSRTREETAAQVASLKKNVWLDRGTRATFIDESVYNANINLFCVVRLLVE
FPATGGVIPSWQFQPLKLIRYVTTFDFFLAACEIIFCFFIFYYVVEEILEIRIHKLHYFRSFWNCLDVVIVVLS
VVAIGINIYRTSNVEVLLQFLEDQNTFPNFEHLAYWQIQFNNIAAVTVFFVWIKLFKFINFNRTMSQLSTTMSR
CAKDLFGFAIMFFIIFLAYAQLAYLVFGTQVDDESTFQECIFTQFRIILGDINFAEIEEANRVLGPIYFTTFVF
FMFFILLNMFLAIINDTYSEVKSDLAQQKAEMELSDLIRKGYHKALVKLKLKKNTVDDISESLRQGGGKLNEDE
LRQDLKGKGHTDAEIEAIFTKYDQDGDQELTEHEHQQMRDDLEKEREDLDLDHSSLPRPMSSRSFPRSLDDSEE
DDDEDSGHSSRRRGSISSGVSYEEFQVLVRRVDRMEHSIGSIVSKIDAVIVKLEIMERAKLKRREVLGRLLDGV
AEDERLGRDSEIHREQMERLVREELERWESDDAASQISHGLGTPVGLNGQPRPRSSRPSSSQSTEGMEGAGGNG
SSNVHV
HDAd-PKD1
RightITR-CBh-mCherry: PKD1-HGHpA-PackagingSignal-LeftITR
SEQ ID NO: 5
CCCGTAGAAAAGATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCCGTAGTTAGGCCACCAC
TTCAAGAACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCAGCGGTCGG
GCTGAACGGGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCTGATACCGCTCGCC
GCAGCCGAACGACCGAGCGCAGCGAGTCAGTGAGCGAGGAAGCGGAAGAGCGCCTGATGCGGTATTTT
CTCCTTACGCATCTGTGCGGTATTTCACACCGCATATGGATCCATGCATGTTAAGTTTAAACATCATC
AATAATATACCTTATTTTGGATTGAAGCCAATATGATAATGAGGGGGTGGAGTTTGTGACGTGGCGCG
GGGCGTGGGAACGGGGCGGGTGACGTAGGTTTTAGGGCGGAGTAACTTGTATGTGTTGGGAATTGTAG
TTTTCTTAAAATGGGAAGTTACGTAACGTGGGAAAACGGAAGTGACGATTTGAGGAAGTTGTGGGTTT
TTTGGCTTTCGTTTCTGGGCGTAGGTTCGCGTGCGGTTTTCTGGGTGTTTTTTGTGGACTTTAACCGT
TACGTCATTTTTTAGTCCTATATATACTCGCTCTGCACTTGGCCCTTTTTTACACTGTGACTGATTGA
GCTGGTGCCGTGTCGAGTGGTGTTTTTTGATGCCCCCCCTCGAGGTTCGACGGTATCGATAAGCTTGA
TTTAATTAAGGCCGGCCCCTAGGGGCGCGCGCGGCCGCTAGGGATAACAGGGTAATTGTTGACAATTA
ATCATCGGCATAGTATATCGGCATAGTATAATACGACAAGGTGAGGAACTAAACCATGGCCAAGTTGA
CCAGTGCCGTTCCGGTGCTCACCGCGCGCGACGTCGCCGGAGCGGTCGAGTTCTGGACCGACCGGCTC
GGGTTCTCCCGGGACTTCGTGGAGGACGACTTCGCCGGTGTGGTCCGGGACGACGTGACCCTGTTCAT
CAGCGCGGTCCAGGACCAGGTGGTGCCGGACAACACCCTGGCCTGGGTGTGGGTGCGCGGCCTGGACG
AGCTGTACGCCGAGTGGTCGGAGGTCGTGTCCACGAACTTCCGGGACGCCTCCGGGCCGGCCATGACC
GAGATCGGCGAGCAGCCGTGGGGGCGGGAGTTCGCCCTGCGCGACCCGGCCGGCAACTGCGTGCACTT
CGTGGCCGAGGAGCAGGACTGAACGCGTCGTTACATAACTTACGGTAAATGGCCCGCCTGGCTGACCG
CCCAACGACCCCCGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGTCGAGGT
GAGCCCCACGTTCTGCTTCACTCTCCCCATCTCCCCCCCCTCCCCACCCCCAATTTTGTATTTATTTA
TTTTTTAATTATTTTGTGCAGCGATGGGGGCGGGGGGGGGGGGGGGGCGCGCGCCAGGCGGGGCGGGG
CGGGGCGAGGGGCGGGGCGGGGCGAGGCGGAGAGGTGCGGCGGCAGCCAATCAGAGCGGCGCGCTCCG
AAAGTTTCCTTTTATGGCGAGGCGGCGGCGGCGGCGGCCCTATAAAAAGCGAAGCGCGCGGCGGGCGG
GAGTCGCTGCGACGCTGCCTTCGCCCCGTGCCCCGCTCCGCCGCCGCCTCGCGCCGCCCGCCCCGGCT
CTGACTGACCGCGTTACTCCCACAGGTGAGCGGGCGGGACGGCCCTTCTCCTCCGGGCTGTAATTAGC
TGAGCAAGAGGTAAGGGTTTAAGGGATGGTTGGTTGGTGGGGTATTAATGTTTAATTACCTGGAGCAC
CTGTCCGGAGAATTCGCCACCATGCCGCCCGCCGCGCCCGCCCGCCTGGCGCTGGCCCTGGGCCTGGG
CCTGTGGCTCGGGGCGCTGGCGGGGGGCCCCGGGATGGTGAGCAAGGGCGAGGAGGATAACATGGCCA
TCATCAAGGAGTTCATGCGCTTCAAGGTGCACATGGAGGGCTCCGTGAACGGCCACGAGTTCGAGATC
GAGGGCGAGGGCGAGGGCCGCCCCTACGAGGGCACCCAGACCGCCAAGCTGAAGGTGACCAAGGGTGG
CCCCCTGCCCTTCGCCTGGGACATCCTGTCCCCTCAGTTCATGTACGGCTCCAAGGCCTACGTGAAGC
ACCCCGCCGACATCCCCGACTACTTGAAGCTGTCCTTCCCCGAGGGCTTCAAGTGGGAGCGCGTGATG
AACTTCGAGGACGGCGGCGTGGTGACCGTGACCCAGGACTCCTCCCTGCAGGACGGCGAGTTCATCTA
CAAGGTGAAGCTGCGCGGCACCAACTTCCCCTCCGACGGCCCCGTAATGCAGAAGAAGACCATGGGCT
GGGAGGCCTCCTCCGAGCGGATGTACCCCGAGGACGGCGCCCTGAAGGGCGAGATCAAGCAGAGGCTG
AAGCTGAAGGACGGCGGCCACTACGACGCTGAGGTCAAGACCACCTACAAGGCCAAGAAGCCCGTGCA
GCTGCCCGGCGCCTACAACGTCAACATCAAGTTGGACATCACCTCCCACAACGAGGACTACACCATCG
TGGAACAGTACGAACGCGCCGAGGGCCGCCACTCCACCGGCGGCATGGACGAGCTGTACAAGGGCGCG
CCGGGGGGCCCCGGGCGCGGCTGCGGGCCCTGCGAGCCCCCCTGCCTCTGCGGCCCAGCGCCCGGCGC
CGCCTGCCGCGTCAACTGCTCGGGCCGCGGGCTGCGGACGCTCGGTCCCGCGCTGCGCATCCCCGCGG
ACGCCACAGCGCTAGACGTCTCCCACAACCTGCTCCGGGCGCTGGACGTTGGGCTCCTGGCGAACCTC
TCGGCGCTGGCAGAGCTGGATATAAGCAACAACAAGATTTCTACGTTAGAAGAAGGAATATTTGCTAA
TTTATTTAATTTAAGTGAAATAAACCTGAGTGGGAACCCGTTTGAGTGTGACTGTGGCCTGGCGTGGC
TGCCGCGATGGGCGGAGGAGCAGCAGGTGCGGGTGGTGCAGCCCGAGGCAGCCACGTGTGCTGGGCCT
GGCTCCCTGGCTGGCCAGCCTCTGCTTGGCATCCCCTTGCTGGACAGTGGCTGTGGTGAGGAGTATGT
CGCCTGCCTCCCTGACAACAGCTCAGGCACCGTGGCAGCAGTGTCCTTTTCAGCTGCCCACGAAGGCC
TGCTTCAGCCAGAGGCCTGCAGCGCCTTCTGCTTCTCCACCGGCCAGGGCCTCGCAGCCCTCTCGGAG
CAGGGCTGGTGCCTGTGTGGGGCGGCCCAGCCCTCCAGTGCCTCCTTTGCCTGCCTGTCCCTCTGCTC
CGGCCCCCCGCCACCTCCTGCCCCCACCTGTAGGGGCCCCACCCTCCTCCAGCACGTCTTCCCTGCCT
CCCCAGGGGCCACCCTGGTGGGGCCCCACGGACCTCTGGCCTCTGGCCAGCTAGCAGCCTTCCACATC
GCTGCCCCGCTCCCTGTCACTGCCACACGCTGGGACTTCGGAGACGGCTCCGCCGAGGTGGATGCCGC
TGGGCCGGCTGCCTCGCATCGCTATGTGCTGCCTGGGCGCTATCACGTGACGGCCGTGCTGGCCCTGG
GGGCCGGCTCAGCCCTGCTGGGGACAGACGTGCAGGTGGAAGCGGCACCTGCCGCCCTGGAGCTCGTG
TGCCCGTCCTCGGTGCAGAGTGACGAGAGCCTTGACCTCAGCATCCAGAACCGCGGTGGTTCAGGCCT
GGAGGCCGCCTACAGCATCGTGGCCCTGGGCGAGGAGCCGGCCCGAGCGGTGCACCCGCTCTGCCCCT
CGGACACGGAGATCTTCCCTGGCAACGGGCACTGCTACCGCCTGGTGGTGGAGAAGGCGGCCTGGCTG
CAGGCGCAGGAGCAGTGTCAGGCCTGGGCCGGGGCCGCCCTGGCAATGGTGGACAGTCCCGCCGTGCA
GCGCTTCCTGGTCTCCCGGGTCACCAGGAGCCTAGACGTGTGGATCGGCTTCTCGACTGTGCAGGGGG
TGGAGGTGGGCCCAGCGCCGCAGGGCGAGGCCTTCAGCCTGGAGAGCTGCCAGAACTGGCTGCCCGGG
GAGCCACACCCAGCCACAGCCGAGCACTGCGTCCGGCTCGGGCCCACCGGGTGGTGTAACACCGACCT
GTGCTCAGCGCCGCACAGCTACGTCTGCGAGCTGCAGCCCGGAGGCCCAGTGCAGGATGCCGAGAACC
TCCTCGTGGGAGCGCCCAGTGGGGACCTGCAGGGACCCCTGACGCCTCTGGCACAGCAGGACGGCCTC
TCAGCCCCGCACGAGCCCGTGGAGGTCATGGTATTCCCGGGCCTGCGTCTGAGCCGTGAAGCCTTCCT
CACCACGGCCGAATTTGGGACCCAGGAGCTCCGGCGGCCCGCCCAGCTGCGGCTGCAGGTGTACCGGC
TCCTCAGCACAGCAGGGACCCCGGAGAACGGCAGCGAGCCTGAGAGCAGGTCCCCGGACAACAGGACC
CAGCTGGCCCCCGCGTGCATGCCAGGGGGACGCTGGTGCCCTGGAGCCAACATCTGCTTGCCGCTGGA
CGCCTCCTGCCACCCCCAGGCCTGCGCCAATGGCTGCACGTCAGGGCCAGGGCTACCCGGGGCCCCCT
ATGCGCTATGGAGAGAGTTCCTCTTCTCCGTTCCCGCGGGGCCCCCCGCGCAGTACTCGGTCACCCTC
CACGGCCAGGATGTCCTCATGCTCCCTGGTGACCTCGTTGGCTTGCAGCACGACGCTGGCCCTGGCGC
CCTCCTGCACTGCTCGCCGGCTCCCGGCCACCCTGGTCCCCAGGCCCCGTACCTCTCCGCCAACGCCT
CGTCATGGCTGCCCCACTTGCCAGCCCAGCTGGAGGGCACTTGGGCCTGCCCTGCCTGTGCCCTGCGG
CTGCTTGCAGCCACGGAACAGCTCACCGTGCTGCTGGGCTTGAGGCCCAACCCTGGACTGCGGCTGCC
TGGGCGCTATGAGGTCCGGGCAGAGGTGGGCAATGGCGTGTCCAGGCACAACCTCTCCTGCAGCTTTG
ACGTGGTCTCCCCAGTGGCTGGGCTGCGGGTCATCTACCCTGCCCCCCGCGACGGCCGCCTCTACGTG
CCCACCAACGGCTCAGCCTTGGTGCTCCAGGTGGACTCTGGTGCCAACGCCACGGCCACGGCTCGCTG
GCCTGGGGGCAGTGTCAGCGCCCGCTTTGAGAATGTCTGCCCTGCCCTGGTGGCCACCTTCGTGCCCG
GCTGCCCCTGGGAGACCAACGATACCCTGTTCTCAGTGGTAGCACTGCCGTGGCTCAGTGAGGGGGAG
CACGTGGTGGACGTGGTGGTGGAAAACAGCGCCAGCCGGGCCAACCTCAGCCTGCGGGTGACGGCGGA
GGAGCCCATCTGTGGCCTCCGCGCCACGCCCAGCCCCGAGGCCCGTGTACTGCAGGGAGTCCTAGTGA
GGTACAGCCCCGTGGTGGAGGCCGGCTCGGACATGGTCTTCCGGTGGACCATCAACGACAAGCAGTCC
CTGACCTTCCAGAACGTGGTCTTCAATGTCATTTATCAGAGCGCGGCGGTCTTCAAGCTCTCACTGAC
GGCCTCCAACCACGTGAGCAACGTCACCGTGAACTACAACGTAACCGTGGAGCGGATGAACAGGATGC
AGGGTCTGCAGGTCTCCACAGTGCCGGCCGTGCTGTCCCCCAATGCCACGCTAGCACTGACGGCGGGC
GTGCTGGTGGACTCGGCCGTGGAGGTGGCCTTCCTGTGGACCTTTGGGGATGGGGAGCAGGCCCTCCA
CCAGTTCCAGCCTCCGTACAACGAGTCCTTCCCGGTTCCAGACCCCTCGGTGGCCCAGGTGCTGGTGG
AGCACAATGTCATGCACACCTACGCTGCCCCAGGTGAGTACCTCCTGACCGTGCTGGCATCTAATGCC
TTCGAGAACCTGACGCAGCAGGTGCCTGTGAGCGTGCGCGCCTCCCTGCCCTCCGTGGCTGTGGGTGT
GAGTGACGGCGTCCTGGTGGCCGGCCGGCCCGTCACCTTCTACCCGCACCCGCTGCCCTCGCCTGGGG
GTGTTCTTTACACGTGGGACTTCGGGGACGGCTCCCCTGTCCTGACCCAGAGCCAGCCGGCTGCCAAC
CACACCTATGCCTCGAGGGGCACCTACCACGTGCGCCTGGAGGTCAACAACACGGTGAGCGGTGCGGC
GGCCCAGGCGGATGTGCGCGTCTTTGAGGAGCTCCGCGGACTCAGCGTGGACATGAGCCTGGCCGTGG
AGCAGGGCGCCCCCGTGGTGGTCAGCGCCGCGGTGCAGACGGGCGACAACATCACGTGGACCTTCGAC
ATGGGGGACGGCACCGTGCTGTCGGGCCCGGAGGCAACAGTGGAGCATGTGTACCTGCGGGCACAGAA
CTGCACAGTGACCGTGGGTGCGGCCAGCCCCGCCGGCCACCTGGCCCGGAGCCTGCACGTGCTGGTCT
TCGTCCTGGAGGTGCTGCGCGTTGAACCCGCCGCCTGCATCCCCACGCAGCCTGACGCGCGGCTCACG
GCCTACGTCACCGGGAACCCGGCCCACTACCTCTTCGACTGGACCTTCGGGGATGGCTCCTCCAACAC
GACCGTGCGGGGGTGCCCGACGGTGACACACAACTTCACGCGGAGCGGCACGTTCCCCCTGGCGCTGG
TGCTGTCCAGCCGCGTGAACAGGGCGCATTACTTCACCAGCATCTGCGTGGAGCCAGAGGTGGGCAAC
GTCACCCTGCAGCCAGAGAGGCAGTTTGTGCAGCTCGGGGACGAGGCCTGGCTGGTGGCATGTGCCTG
GCCCCCGTTCCCCTACCGCTACACCTGGGACTTTGGCACCGAGGAAGCCGCCCCCACCCGTGCCAGGG
GCCCTGAGGTGACGTTCATCTACCGAGACCCAGGCTCCTATCTTGTGACAGTCACCGCGTCCAACAAC
ATCTCTGCTGCCAATGACTCAGCCCTGGTGGAGGTGCAGGAGCCCGTGCTGGTCACCAGCATCAAGGT
CAATGGCTCCCTTGGGCTGGAGCTGCAGCAGCCGTACCTGTTCTCTGCTGTGGGCCGTGGGCGCCCCG
CCAGCTACCTGTGGGATCTGGGGGACGGTGGGTGGCTCGAGGGTCCGGAGGTCACCCACGCTTACAAC
AGCACAGGTGACTTCACCGTTAGGGTGGCCGGCTGGAATGAGGTGAGCCGCAGCGAGGCCTGGCTCAA
TGTGACGGTGAAGCGGCGCGTGCGGGGGCTCGTCGTCAATGCAAGCCGCACGGTGGTGCCCCTGAATG
GGAGCGTGAGCTTCAGCACGTCGCTGGAGGCCGGCAGTGATGTGCGCTATTCCTGGGTGCTCTGTGAC
CGCTGCACGCCCATCCCTGGGGGTCCTACCATCTCTTACACCTTCCGCTCCGTGGGCACCTTCAATAT
CATCGTCACGGCTGAGAACGAGGTGGGCTCCGCCCAGGACAGCATCTTCGTCTATGTCCTGCAGCTCA
TAGAGGGGCTGCAGGTGGTGGGCGGTGGCCGCTACTTCCCCACCAACCACACGGTACAGCTGCAGGCC
GTGGTTAGGGATGGCACCAACGTCTCCTACAGCTGGACTGCCTGGAGGGACAGGGGCCCGGCCCTGGC
CGGCAGCGGCAAAGGCTTCTCGCTCACCGTGCTCGAGGCCGGCACCTACCATGTGCAGCTGCGGGCCA
CCAACATGCTGGGCAGCGCCTGGGCCGACTGCACCATGGACTTCGTGGAGCCTGTGGGGTGGCTGATG
GTGGCCGCCTCCCCGAACCCAGCTGCCGTCAACACAAGCGTCACCCTCAGTGCCGAGCTGGCTGGTGG
CAGTGGTGTCGTATACACTTGGTCCTTGGAGGAGGGGCTGAGCTGGGAGACCTCCGAGCCATTTACCA
CCCATAGCTTCCCCACACCCGGCCTGCACTTGGTCACCATGACGGCAGGGAACCCGCTGGGCTCAGCC
AACGCCACCGTGGAAGTGGATGTGCAGGTGCCTGTGAGTGGCCTCAGCATCAGGGCCAGCGAGCCCGG
AGGCAGCTTCGTGGCGGCCGGGTCCTCTGTGCCCTTTTGGGGGCAGCTGGCCACGGGCACCAATGTGA
GCTGGTGCTGGGCTGTGCCCGGCGGCAGCAGCAAGCGTGGCCCTCATGTCACCATGGTCTTCCCGGAT
GCTGGCACCTTCTCCATCCGGCTCAATGCCTCCAACGCAGTCAGCTGGGTCTCAGCCACGTACAACCT
CACGGCGGAGGAGCCCATCGTGGGCCTGGTGCTGTGGGCCAGCAGCAAGGTGGTGGCGCCCGGGCAGC
TGGTCCATTTTCAGATCCTGCTGGCTGCCGGCTCAGCTGTCACCTTCCGCCTGCAGGTCGGCGGGGCC
AACCCCGAGGTGCTCCCCGGGCCCCGTTTCTCCCACAGCTTCCCCCGCGTCGGAGACCACGTGGTGAG
CGTGCGGGGCAAAAACCACGTGAGCTGGGCCCAGGCGCAGGTGCGCATCGTGGTGCTGGAGGCCGTGA
GTGGGCTGCAGGTGCCCAACTGCTGCGAGCCTGGCATCGCCACGGGCACTGAGAGGAACTTCACAGCC
CGCGTGCAGCGCGGCTCTCGGGTCGCCTACGCCTGGTACTTCTCGCTGCAGAAGGTCCAGGGCGACTC
GCTGGTCATCCTGTCGGGCCGCGACGTCACCTACACGCCCGTGGCCGCGGGGCTGTTGGAGATCCAGG
TGCGCGCCTTCAACGCCCTGGGCAGTGAGAACCGCACGCTGGTGCTGGAGGTTCAGGACGCCGTCCAG
TATGTGGCCCTGCAGAGCGGCCCCTGCTTCACCAACCGCTCGGCGCAGTTTGAGGCCGCCACCAGCCC
CAGCCCCCGGCGTGTGGCCTACCACTGGGACTTTGGGGATGGGTCGCCAGGGCAGGACACAGATGAGC
CCAGGGCCGAGCACTCCTACCTGAGGCCTGGGGACTACCGCGTGCAGGTGAACGCCTCCAACCTGGTG
AGCTTCTTCGTGGCGCAGGCCACGGTGACCGTCCAGGTGCTGGCCTGCCGGGAGCCGGAGGTGGACGT
GGTCCTGCCCCTGCAGGTGCTGATGCGGCGATCACAGCGCAACTACTTGGAGGCCCACGTTGACCTGC
GCGACTGCGTCACCTACCAGACTGAGTACCGCTGGGAGGTGTATCGCACCGCCAGCTGCCAGCGGCCG
GGGCGCCCAGCGCGTGTGGCCCTGCCCGGCGTGGACGTGAGCCGGCCTCGGCTGGTGCTGCCGCGGCT
GGCGCTGCCTGTGGGGCACTACTGCTTTGTGTTTGTCGTGTCATTTGGGGACACGCCACTGACACAGA
GCATCCAGGCCAATGTGACGGTGGCCCCCGAGCGCCTGGTGCCCATCATTGAGGGTGGCTCATACCGC
GTGTGGTCAGACACACGGGACCTGGTGCTGGATGGGAGCGAGTCCTACGACCCCAACCTGGAGGACGG
CGACCAGACGCCGCTCAGTTTCCACTGGGCCTGTGTGGCTTCGACACAGAGGGAGGCTGGCGGGTGTG
CGCTGAACTTTGGGCCCCGCGGGAGCAGCACGGTCACCATTCCACGGGAGCGGCTGGCGGCTGGCGTG
GAGTACACCTTCAGCCTGACCGTGTGGAAGGCCGGCCGCAAGGAGGAGGCCACCAACCAGACGGTGCT
GATCCGGAGTGGCCGGGTGCCCATTGTGTCCTTGGAGTGTGTGTCCTGCAAGGCACAGGCCGTGTACG
AAGTGAGCCGCAGCTCCTACGTGTACTTGGAGGGCCGCTGCCTCAATTGCAGCAGCGGCTCCAAGCGA
GGGCGGTGGGCTGCACGTACGTTCAGCAACAAGACGCTGGTGCTGGATGAGACCACCACATCCACGGG
CAGTGCAGGCATGCGACTGGTGCTGCGGCGGGGCGTGCTGCGGGACGGCGAGGGATACACCTTCACGC
TCACGGTGCTGGGCCGCTCTGGCGAGGAGGAGGGCTGCGCCTCCATCCGCCTGTCCCCCAACCGCCCG
CCGCTGGGGGGCTCTTGCCGCCTCTTCCCACTGGGCGCTGTGCACGCCCTCACCACCAAGGTGCACTT
CGAATGCACGGGCTGGCATGACGCGGAGGATGCTGGCGCCCCGCTGGTGTACGCCCTGCTGCTGCGGC
GCTGTCGCCAGGGCCACTGCGAGGAGTTCTGTGTCTACAAGGGCAGCCTCTCCAGCTACGGAGCCGTG
CTGCCCCCGGGTTTCAGGCCACACTTCGAGGTGGGCCTGGCCGTGGTGGTGCAGGACCAGCTGGGAGC
CGCTGTGGTCGCCCTCAACAGGTCTTTGGCCATCACCCTCCCAGAGCCCAACGGCAGCGCAACGGGGC
TCACAGTCTGGCTGCACGGGCTCACCGCTAGTGTGCTCCCAGGGCTGCTGCGGCAGGCCGATCCCCAG
CACGTCATCGAGTACTCGTTGGCCCTGGTCACCGTGCTGAACGAGTACGAGCGGGCCCTGGACGTGGC
GGCAGAGCCCAAGCACGAGCGGCAGCACCGAGCCCAGATACGCAAGAACATCACGGAGACTCTGGTGT
CCCTGAGGGTCCACACTGTGGATGACATCCAGCAGATCGCTGCTGCGCTGGCCCAGTGCATGGGGCCC
AGCAGGGAGCTCGTATGCCGCTCGTGCCTGAAGCAGACGCTGCACAAGCTGGAGGCCATGATGCTCAT
CCTGCAGGCAGAGACCACCGCGGGCACCGTGACGCCCACCGCCATCGGAGACAGCATCCTCAACATCA
CAGGAGACCTCATCCACCTGGCCAGCTCGGACGTGCGGGCACCACAGCCCTCAGAGCTGGGAGCCGAG
TCACCATCTCGGATGGTGGCGTCCCAGGCCTACAACCTGACCTCTGCCCTCATGCGCATCCTCATGCG
CTCCCGCGTGCTCAACGAGGAGCCCCTGACGCTGGCGGGCGAGGAGATCGTGGCCCAGGGCAAGCGCT
CGGACCCGCGGAGCCTGCTGTGCTATGGCGGCGCCCCAGGGCCTGGCTGCCACTTCTCCATCCCCGAG
GCTTTCAGCGGGGCCCTGGCCAACCTCAGTGACGTGGTGCAGCTCATCTTTCTGGTGGACTCCAATCC
CTTTCCCTTTGGCTATATCAGCAACTACACCGTCTCCACCAAGGTGGCCTCGATGGCATTCCAGACAC
AGGCCGGCGCCCAGATCCCCATCGAGCGGCTGGCCTCAGAGCGCGCCATCACCGTGAAGGTGCCCAAC
AACTCGGACTGGGCTGCCCGGGGCCACCGCAGCTCCGCCAACTCCGCCAACTCCGTTGTGGTCCAGCC
CCAGGCCTCCGTCGGTGCTGTGGTCACCCTGGACAGCAGCAACCCTGCGGCCGGGCTGCATCTGCAGC
TCAACTATACGCTGCTGGACGGCCACTACCTGTCTGAGGAACCTGAGCCCTACCTGGCAGTCTACCTA
CACTCGGAGCCCCGGCCCAATGAGCACAACTGCTCGGCTAGCAGGAGGATCCGCCCAGAGTCACTCCA
GGGTGCTGACCACCGGCCCTACACCTTCTTCATTTCCCCGGGGAGCAGAGACCCAGCGGGGAGTTACC
ATCTGAACCTCTCCAGCCACTTCCGCTGGTCGGCGCTGCAGGTGTCCGTGGGCCTGTACACGTCCCTG
TGCCAGTACTTCAGCGAGGAGGACATGGTGTGGCGGACAGAGGGGCTGCTGCCCCTGGAGGAGACCTC
GCCCCGCCAGGCCGTCTGCCTCACCCGCCACCTCACCGCCTTCGGCGCCAGCCTCTTCGTGCCCCCAA
GCCATGTCCGCTTTGTGTTTCCTGAGCCGACAGCGGATGTAAACTACATCGTCATGCTGACATGTGCT
GTGTGCCTGGTGACCTACATGGTCATGGCCGCCATCCTGCACAAGCTGGACCAGTTGGATGCCAGCCG
GGGCCGCGCCATCCCTTTCTGTGGGCAGCGGGGCCGCTTCAAGTACGAGATCCTCGTCAAGACAGGCT
GGGGCCGGGGCTCAGGTACCACGGCCCACGTGGGCATCATGCTGTATGGGGTGGACAGCCGGAGCGGC
CACCGGCACCTGGACGGCGACAGAGCCTTCCACCGCAACAGCCTGGACATCTTCCGGATCGCCACCCC
GCACAGCCTGGGTAGCGTGTGGAAGATCCGAGTGTGGCACGACAACAAAGGGCTCAGCCCTGCCTGGT
TCCTGCAGCACGTCATCGTCAGGGACCTGCAGACGGCACGCAGCGCCTTCTTCCTGGTCAATGACTGG
CTTTCGGTGGAGACGGAGGCCAACGGGGGCCTGGTGGAGAAGGAGGTGCTGGCCGCGAGCGACGCAGC
CCTTTTGCGCTTCCGGCGCCTGCTGGTGGCTGAGCTGCAGCGTGGCTTCTTTGACAAGCACATCTGGC
TCTCCATATGGGACCGGCCGCCTCGTAGCCGTTTCACTCGCATCCAGAGGGCCACCTGCTGCGTTCTC
CTCATCTGCCTCTTCCTGGGCGCCAACGCCGTGTGGTACGGGGCTGTTGGCGACTCTGCCTACAGCAC
GGGGCATGTGTCCAGGCTGAGCCCGCTGAGCGTCGACACAGTCGCTGTTGGCCTGGTGTCCAGCGTGG
TTGTCTATCCCGTCTACCTGGCCATCCTTTTTCTCTTCCGGATGTCCCGGAGCAAGGTGGCTGGGAGC
CCGAGCCCCACACCTGCCGGGCAGCAGGTGCTGGACATCGACAGCTGCCTGGACTCGTCCGTGCTGGA
CAGCTCCTTCCTCACGTTCTCAGGCCTCCACGCTGAGCAGGCCTTTGTTGGACAGATGAAGAGTGACT
TGTTTCTGGATGATTCTAAGAGTCTGGTGTGCTGGCCCTCCGGCGAGGGAACGCTCAGTTGGCCGGAC
CTGCTCAGTGACCCGTCCATTGTGGGTAGCAATCTGCGGCAGCTGGCACGGGGCCAGGCGGGCCATGG
GCTGGGCCCAGAGGAGGACGGCTTCTCCCTGGCCAGCCCCTACTCGCCTGCCAAATCCTTCTCAGCAT
CAGATGAAGACCTGATCCAGCAGGTCCTTGCCGAGGGGGTCAGCAGCCCAGCCCCTACCCAAGACACC
CACATGGAAACGGACCTGCTCAGCAGCCTGTCCAGCACTCCTGGGGAGAAGACAGAGACGCTGGCGCT
GCAGAGGCTGGGGGAGCTGGGGCCACCCAGCCCAGGCCTGAACTGGGAACAGCCCCAGGCAGCGAGGC
TGTCCAGGACAGGACTGGTGGAGGGTCTGCGGAAGCGCCTGCTGCCGGCCTGGTGTGCCTCCCTGGCC
CACGGGCTCAGCCTGCTCCTGGTGGCTGTGGCTGTGGCTGTCTCAGGGTGGGTGGGTGCGAGCTTCCC
CCCGGGCGTGAGTGTTGCGTGGCTCCTGTCCAGCAGCGCCAGCTTCCTGGCCTCATTCCTCGGCTGGG
AGCCACTGAAGGTCTTGCTGGAAGCCCTGTACTTCTCACTGGTGGCCAAGCGGCTGCACCCGGATGAA
GATGACACCCTGGTAGAGAGCCCGGCTGTGACGCCTGTGAGCGCACGTGTGCCCCGCGTACGGCCACC
CCACGGCTTTGCACTCTTCCTGGCCAAGGAAGAAGCCCGCAAGGTCAAGAGGCTACATGGCATGCTGC
GGAGCCTCCTGGTGTACATGCTTTTTCTGCTGGTGACCCTGCTGGCCAGCTATGGGGATGCCTCATGC
CATGGGCACGCCTACCGTCTGCAAAGCGCCATCAAGCAGGAGCTGCACAGCCGGGCCTTCCTGGCCAT
CACGCGGTCTGAGGAGCTCTGGCCATGGATGGCCCACGTGCTGCTGCCCTACGTCCACGGGAACCAGT
CCAGCCCAGAGCTGGGGCCCCCACGGCTGCGGCAGGTGCGGCTGCAGGAAGCACTCTACCCAGACCCT
CCCGGCCCCAGGGTCCACACGTGCTCGGCCGCAGGAGGCTTCAGCACCAGCGATTACGACGTTGGCTG
GGAGAGTCCTCACAATGGCTCGGGGACGTGGGCCTATTCAGCGCCGGATCTGCTGGGGGCATGGTCCT
GGGGCTCCTGTGCCGTGTATGACAGCGGGGGCTACGTGCAGGAGCTGGGCCTGAGCCTGGAGGAGAGC
CGCGACCGGCTGCGCTTCCTGCAGCTGCACAACTGGCTGGACAACAGGAGCCGCGCTGTGTTCCTGGA
GCTCACGCGCTACAGCCCGGCCGTGGGGCTGCACGCCGCCGTCACGCTGCGCCTCGAGTTCCCGGCGG
CCGGCCGCGCCCTGGCCGCCCTCAGCGTCCGCCCCTTTGCGCTGCGCCGCCTCAGCGCGGGCCTCTCG
CTGCCTCTGCTCACCTCGGTGTGCCTGCTGCTGTTCGCCGTGCACTTCGCCGTGGCCGAGGCCCGTAC
TTGGCACAGGGAAGGGCGCTGGCGCGTGCTGCGGCTCGGAGCCTGGGCGCGGTGGCTGCTGGTGGCGC
TGACGGCGGCCACGGCACTGGTACGCCTCGCCCAGCTGGGTGCCGCTGACCGCCAGTGGACCCGTTTC
GTGCGCGGCCGCCCGCGCCGCTTCACTAGCTTCGACCAGGTGGCGCAGCTGAGCTCCGCAGCCCGTGG
CCTGGCGGCCTCGCTGCTCTTCCTGCTTTTGGTCAAGGCTGCCCAGCAGCTACGCTTCGTGCGCCAGT
GGTCCGTCTTTGGCAAGACATTATGCCGAGCTCTGCCAGAGCTCCTGGGGGTCACCTTGGGCCTGGTG
GTGCTCGGGGTAGCCTACGCCCAGCTGGCCATCCTGCTCGTGTCTTCCTGTGTGGACTCCCTCTGGAG
CGTGGCCCAGGCCCTGTTGGTGCTGTGCCCTGGGACTGGGCTCTCTACCCTGTGTCCTGCCGAGTCCT
GGCACCTGTCACCCCTGCTGTGTGTGGGGCTCTGGGCACTGCGGCTGTGGGGCGCCCTACGGCTGGGG
GCTGTTATTCTCCGCTGGCGCTACCACGCCTTGCGTGGAGAGCTGTACCGGCCGGCCTGGGAGCCCCA
GGACTACGAGATGGTGGAGTTGTTCCTGCGCAGGCTGCGCCTCTGGATGGGCCTCAGCAAGGTCAAGG
AGTTCCGCCACAAAGTCCGCTTTGAAGGGATGGAGCCGCTGCCCTCTCGCTCCTCCAGGGGCTCCAAG
GTATCCCCGGATGTGCCCCCACCCAGCGCTGGCTCCGATGCCTCGCACCCCTCCACCTCCTCCAGCCA
GCTGGATGGGCTGAGCGTGAGCCTGGGCCGGCTGGGGACAAGGTGTGAGCCTGAGCCCTCCCGCCTCC
AAGCCGTGTTCGAGGCCCTGCTCACCCAGTTTGACCGACTCAACCAGGCCACAGAGGACGTCTACCAG
CTGGAGCAGCAGCTGCACAGCCTGCAAGGCCGCAGGAGCAGCCGGGCGCCCGCCGGATCTTCCCGTGG
CCCATCCCCGGGCCTGCGGCCAGCACTGCCCAGCCGCCTTGCCCGGGCCAGTCGGGGTGTGGACCTGG
CCACTGGCCCCAGCAGGACACCCCTTCGGGCCAAGAACAAGGTCCACCCCAGCAGCACTTAGTCCTCC
TTCCTGGCGGGGGTGGGCCGTGGAGTCGGAGTGGACACCGCTCAGTATTACTTTCTGCCGCTGTCAAG
GCCGAGGGCCAGGCAGAATGGCTGCACGTAGGTTCCCCAGAGAGCAGGCAGGGGCATCTGTCTGTCTG
TGGGCTTCAGCACTTTAAAGAGGCTGTGTGGCCAACCAGGACCCAGGGTCCCCTCCCCAGCTCCCTTG
GGAAGGACACAGCAGTATTGGACGGTTTCTAGCCTCTGAGATGCTAATTTATTTCCCCGAGTCCTCAG
GTACAGCGGGCTGTGCCCGGCCCCACCCCCTGGGCAGATGTCCCCCACTGCTAAGGCTGCTGGCTTCA
GGGAGGGTTAGCCTGCACCGCCGCCACCCTGCCCCTAAGTTATTACCTCTCCAGTTCCTACCGTACTC
CCTGCACCGTCTCACTGTGTGTCTCGTGTCAGTAATTTATATGGTGTTAAAATGTGTATATTTTTGTA
TGTCACTATTTTCACTAGGGCTGAGGGGCCTGCGCCCAGAGCTGGCCTCCCCCAACACCTGCTGCGCT
TGGTAGGTGTGGTGGCGTTATGGCAGCCCGGCTGCTGCTTGGATGCGAGCTTGGCCTTGGGCCGGTGC
TGGGGGCACAGCTGTCTGCCAGGCACTCTCATCACCCCAGAGGCCTTGTCATCCTCCCTTGCCCCAGG
CCAGGTAGCAAGAGAGCAGCGCCCAGGCCTGCTGGCATCAGGTCTGGGCAAGTAGCAGGACTAGGCAT
GTCAGAGGACCCCAGGGTGGTTAGAGGAAAAGACTCCTCCTGGGGGCTGGCTCCCAGGGTGGAGGAAG
GTGACTGTGTGTGTGTGTGTGTGCGCGCGCGCACGCGCGAGTGTGCTGTATGGCCCAGGCAGCCTCAA
GGCCCTCGGAGCTGGCTGTGCCTGCTTCTGTGTACCACTTCTGTGGGCATGGCCGCTTCTAGAACGGG
TGGCATCCCTGTGACCCCTCCCCAGTGCCTCTCCTGGCCCTGGAAGTTGCCACTCCAGTGCCCACCAG
CCTTGTCCTAATAAAATTAAGTTGCATCATTTTGTCTGACTAGGTGTCCTTCTATAATATTATGGGGT
GGAGGGGGGTGGTATGGAGCAAGGGGCAAGTTGGGAAGACAACCTGTAGGGCCTGCGGGGTCTATTGG
GAACCAAGCTGGAGTGCAGTGGCACAATCTTGGCTCACTGCAATCTCCGCCTCCTGGGTTCAAGCGAT
TCTCCTGCCTCAGCCTCCCGAGTTGTTGGGATTCCAGGCATGCATGACCAGGCTCAGCTAATTTTTGT
TTTTTTGGTAGAGACGGGGTTTCACCATATTGGCCAGGCTGGTCTCCAACTCCTAATCTCAGGTGATC
TACCCACCTTGGCCTCCCAAATTGCTGGGATTACAGGCGTGAACCACTGCTCCCTTCCCTGTCCTTTA
ACTATAACGGTCCTAAGGTAGCGAAGTCGACCGAATCGTTGTCCCTTGTCACAGCCATTGAGAATTTT
GGCAGGGAGCATGTTCTTAGAGCATTTTTAGGCTCTGCGGGACATAACAGCTCTGCCTCAGAGCACAT
GCCTTTCTCAGCTCCTGAAAGCCACTGATCAAATTGGAACATTTTGTACCTTAGGGATGAGGATATCA
ACTCTCCCAGCCACTTAGAGGGATAAATGTGATGATGCATTCAATTGTGACTACATCTGATCCCAACT
GTTGCTTCAGCTGCTCTCCTATAGCACATGGCGGGAGGCGTGCATCCCAGTAGCTACCTCCCCACTTT
TGGGGAGATGTGGTTCCATCCATGAAACCTGGGTACCCGCCTACCAGGTCCTGGCCTATCAGGTGGCA
GGGTCTGGTCAAAGAAGGGCATGTGTGGTCTTCAGCAAGGGAGACAGGACGGTGGTGCAGAGCGTCTA
GACCCTCAGGGCAAGTCTCCCCCACACCTGCTCCCGGGGCAGTTGTCTTTGTGACCTCCCATCCCCCT
CTGTTTCATCCTCTATAAAATGAGGGGCTGAGCCCCAAAATAACAGGCTTCTTTGCCATGATGCAAAA
CTGCTGAATCTTTCTTTCTGACACACAAGGCATCGAGCAGCCTCTGAAAGAACCAAAGCCACTAGCAG
GCTTCCTGACTTGGGTTTGTAGGTACTGAATACTCCCTTGAAAAATAAAAACATAGAGGCACTTTTCT
CCTGGCTGTTTATTACAGAACGAAGAAAAAACACACTGGCTTGAAACAGACGCCAGATTTCAAATGTA
GAGGTGAAATACGAGGTGGCAATTAAAATGTGATTACAGAAAGTCTGGACACTGAGAAAAGTTTACAG
GACAGTGGGTGTGGGTTTTCTATAACAGACACTTAAATATACATGACGATAATTGCAGATAGAAACCA
TCAAAGACAAACCCCAAATCAACTAATAATGTTTACAGATGTTCCCCCCCAAACCACAGAGCCTTACA
TCAAAACAAATACTGAAAGGCTTTAAACCAGGAACAGCTCGCCTTAACCCCACGAGGGTGCACACAAG
CTGGGCTTTTTCTCTCGGTCTGAATGGTAAAGGGAGGAGGATACTCTAGCTCCTCCAGGTGGATTGCT
GAGACAGGGCTCGGCTCACACACTGTCTCTGCGCCTCTCCCAAATCTGGAGAACTCTCCCAGCCTCCT
GGTAAAGTGTCTCTGTGGGGCACTTAACGATAAAACAGCTTCTGCTGTAAAGCTCATTAGGAAAGAGC
TAGCGGAGACTGAAAGGTTCGCAAAAGAGATTAAGAATCACACAAGGCAATAGGATTTTTAGTGAACA
TAGAAATAAATGGCCAAGTGGTTTTCTATTTGGCATTTGTCAACTTGCACAACAACTCTTGGTCATAT
CCACATTGCTCATTGCATTAAAACCATAAGCGACTCAGCCACCTAGCTTAACAAGGTATCACTGGAGC
AAACAACACGGTCTGCATATTTGTAACATTGTATAATAAACACAAAACAATGCATAGTAAACACAACT
CTACTGAAACAAAAGCCGTCGCTTTATTTACAAAGTCACAAAATGAAGTATAAATACTTCTGTCATTA
ATGTTTAGGAAAACCATTTACAAAATTTTCAAATATGTACACGTAGCTTGAAAAATCACCAGCTTTCC
ATTTTGTCACAGGTAGAGAGAGGGATAAGCATGGGCTGACAACACCACTCAAATTGTAACGGGAGACA
ACTGCGGGTATGGATCGACACCACTTCCTAGAGTGATGTCACCATGGGGGTTTCTATGGGCATCCTGC
TCAGATTTAAAGTGCCCCAGCATCCTGGGTGACTTGCCCAGAATTCTGGGCTGTGGCATTTTGAGCAG
CAGCATGCTGTTCCAAAATGTCGTCGATCAGCCTCAAGTTGCACACCCAGTCTTCATCTGGGCTCACA
CAGGAGCCTTTCAAGAGAGCTTCAATGAAATCTACCTCATTGCAGTCAGGTGACGAAATCAGATCATT
TAGTGGGGGTTGGGGCTGGCGCAAAAAGTCGGCAGGTGGCAGCTCAGGGGGAATATCCGTTCTGTCGA
ACGGACCTGGGAACTGGCTGGCAGCAACGGCAGAAGCAGCAGCAGCGGTGGCAGCAGCAGCCACATAG
CTTGGTGGCTCGATGCCCTGTATGGGGCTCAGGGGACTAAAGCTGGCCATACCCTGCTGGAGGAACTT
GGTGGTGTTTGCTACAGGCACCGGGCCCTGTACCGGGCTCTGCCTGAGGCTCTGGCTGCCCAGCAGGC
TGAAGCTGGGGTTGTTGGCCAGGGGCACTTGTGTTCCCATCGCAGCGGGCACTTGTGCCTCCCAATCA
GATGGCCTCTGAAGGCAGGCCTGGCCAGAAGGTGAGTGCTGCTGAACGCTATTATCCACTTGGCTGAG
GGGTGTTTTCCCCGAAACTGCTGTGGTCACAGCTGCTGCCGCTGTGACCCATGCAGCATTGTTGAACG
CAGTGGGCATTCTTGGCACACTAGGCCGTCTGAGCTGGTGGGGACTCAAGGACTGGGTGCCCAGGGAG
CTGGGACAGAACCCAGGCAGGGGCACTTCTGGTGGGGTGGCCTTGGGGCTCTGCATATGCTGGCAGAC
AGAGTCAAGTCTGCCCAGGGGAGTCTGGCCTGAGTGTGAGAGGATGGGACACTGGGGGCTGGAGGTGA
AAATTCCTTGCCGCTTCCCCAGAGTTGGTGAGATCACTCCCATGCCCTCGCAGCTCTGGTGCCTGGTG
AGTGGGATCATTCCTGGACTCAGATTGTTCTGAAGAAGCCCAGTTCTGGGTGGCATCAAGTGCTTGCT
AGATGGGGGGCTTGCCTTGATCCGGCTACACTTGGAGGTGACTTGTTCTTGGACGGCTACATACAGAA
AGAGAGAAGTGGGGATGAGTTCCAAAGGCATCCTCGACTTCGGCTGTGGCCACCGGAGGGTAGCTCCT
GGCCCAACACGGACTTCTCACCTCCCGCCCTTGGCTCTCTACTGAGCTCCCCCCTGCTCCCCAATTCC
TCGCCATTCCCCTCATTTCTCTGCCCTCAGCCTGGACTGCAGTTCTTCTGGGAAGCTGCCCCAACTCC
CTAGGTCTGTGCTCACCAAGAGCAGATCACACTGGACTGAAATGCCAGCTGATTTGTCTCTTCAAGAA
AATTGGAAGCTCCTGGAGGTCAGGGTCCATGTCTGCTTTTACACTCAGTGCTCTGTATGCAGGCCTGG
CACTGCCCACCCTTTGACAGGTGGTGCATATTTTGTAGAAGGAAGGAAGGGGCCAGGTGGGGTGGGCT
GGGCTGGTGGCGGGAGCTAGCTCAGCCTCTTAGATTCTCTACCCGATGGATGTGACCTGGGACAGCAA
GTGAGTGTGGTGAGTGAGTGCAGACGGTGCTTTGTTCCCCTCTTGTCTCATAGCCTAGATGGCCTCTG
AGCCCAGATCTGGGGCTCAGACAACATTTGTTCAACTGAACGGTAATGGGTTTCCTTTCTGAAGGCTG
AAATCTGGGAGCTGACATTCTGGACTCCCTGAGTTCTGAAGAGCCTGGGGATGGAGAGACACGGAGCA
GAAGATGGAAGGTAGAGTCCCAGGTGCCTAAGATGGGGAATACATCTCCCCTCATTGTCATGAGAGTC
CACTCTAGCTGATATCTACTGTGGCCAATATCTACCGGTACTTTTTTGGGGTGGACACTGAGTCATGC
AGCAGTCTTATGGTTTACCCAAGGTCAGGTAGGGGAGACAGTGCAGTCAGAGCACAAGCCCAGTGTGT
CTGACCCACCCAAGAATCCATGCTCGTATCTACAAAAATGATTTTTTCTCTTGTAATGGTGCCTAGGT
TCTTTTATTATCATGGCATGTGTATGTTTTTCAACTAGGTTACAATCTGGCCTTATAAGGTTAACCTC
CTGGAGGCCACCAGCCTTCCTGAAACTTGTCTGTGCTGTCCCTGCAACTGGAGTGTGCCTGATGTGGC
ACTCCAGCCTGGACAAGTGGGACACAGACTCCGCTGTTATCAGGCCCAAAGATGTCTTCCATAAGACC
AGAAGAGCAATGGTGTAGAGGTGTCATGGGCTACAATAAAGATGCTGACCTCCTGTCTGAGGGCAAGC
AGCCTCTTCTGGCCCTCAGACAAATGCTGAGTGTTCCCAAGACTACCCTCGGCCTGGTCCAATCTCAT
CCCACTGGTGCGTAAGGGTTGCTGAACTCATGACTTCTTGGCTAGCCTGCAACCTCCACGGAGTGGGA
ACTACATCAGGCATTTTGCTAACTGCTGTATCCTAGGCCAATAAATGTTGATCACATTTATAGCTGCC
ATGGTAGGGTGGGGACCCCTGCTATCTATCTGTGGAGGCTCTGGGAGCCCCTGACACAAACTTTCTGA
AGCAGAGCCTCCCCAACCCCTTTTCCATTCCCTATACCTGACAGATGGCCCAGGAACCCATTAGAAAT
GGAAGGTCACTGCAGCAGTATGTGAATGTGCGTGTGGGAGAAGGGCAGGATCAGAGCCCTGGGGGTGT
GGCAGCCCCCAAGTGATTCTAATCCAGATCCTAGGGTTGTTTCCCTGTCCCATTGAAATAGCTGCTTT
AAGGGGCCTGACTCAGGGAAATCAGTCTCTTGAATTAAGTGGTGATTTTGGAGTCATTTAGACCAGGC
CTTCAATTGGGATCCACTAGTTCTAGAGCGGCCGGGCCCAGGGAACCCCGCAGGCGGGGGCGGCCAGT
TTCCCGGGTTCGGCTTTACGTCACGCGAGGGCGGCAGGGAGGACGGAATGGCGGGGTTTGGGGTGGGT
CCCTCCTCGGGGGAGCCCTGGGAAAAGAGGACTGCGTGTGGGAAGAGAAGGTGGAAATGGCGTTTTGG
TTGACATGTGCCGCCTGCGAGCGTGCTGCGGGGAGGGGCCGAGGGCAGATTCGGGAATGATGGCGCGG
GGTGGGGGCGTGGGGGCTTTCTCGGGAGAGGCCCTTCCCTGGAAGTTTGGGGTGCGATGGTGAGGTTC
TCGGGGCACCTCTGGAGGGGCCTCGGCACGGAAAGCGACCACCTGGGAGGGCGTGTGGGGACCAGGTT
TTGCCTTTAGTTTTGCACACACTGTAGTTCATCTTTATGGAGATGCTCATGGCCTCATTGAAGCCCCA
CTACAGCTCTGGTAGCGGTAACCATGCGTATTTGACACACGAAGGAACTAGGGAAAAGGCATTAGGTC
ATTTCAAGCCGAAATTCACATGTGCTAGAATCCAGATTCCATGCTGACCGATGCCCCAGGATATAGAA
AATGAGAATCTGGTCCTTACCTTCAAGAACATTCTTAACCGTAATCAGCCTCTGGTATCTTAGCTCCA
CCCTCACTGGTTTTTTCTTGTTTGTTGAACCGGCCAAGCTGCTGGCCTCCCTCCTCAACCGTTCTGAT
CATGCTTGCTAAAATAGTCAAAACCCCGGCCAGTTAAATATGCTTTAGCCTGCTTTATTATGATTATT
TTTGTTGTTTTGGCAATGACCTGGTTACCTGTTGTTTCTCCCACTAAAACTTTTTAAGGGCAGGAATC
ACCGCCGTAACTCTAGCACTTAGCACAGTACTTGGCTTGTAAGAGGTCCTCGATGATGGTTTGTTGAA
TGAATACATTAAATAATTAACCACTTGAACCCTAAGAAAGAAGCGATTCTATTTCATATTAGGCATTG
TAATGACTTAAGGTAAAGAGCAGTGCTATTAACGGAGTCTAACTGGGAATCCAGCTTGTTTGGGCTAT
TTACTAGTTGTGTGGCTGTGGGCAACTTACTTCACCTCTCTGGGCTTAAGTCATTTTATGTATATCTG
AGGTGCTGGCTACCTCTTGGAGTTATTGAGAGGATTATAAGACAGTCTATGTGAATCAGCAACCCTTG
CATGGCCCCTGGCGGGGAACAGTAATAATAGCCATCATCATGTTTACTTACATAGTCCTAATTAGTCT
TCAAAACAGCCCTGTAGCAATGGTATGATTATTACCATTTTACAGATGAGGAACCTTTGAAGCCTCAG
AGAGGCTAACAGACATACCCTAGGTCATACAGTTATTAAGAGAAGGAGCTCTGTCTCGAACCTAGCTC
TCTCTCTCTCGAGTAATACCAGTTAAAAAATAGGCTACAAATAGGTACTCAAAAAAATGGTAGTGGCT
GTTGTTTTTATTCAGTTGCTGAGGAAAAAATGTTGATTTTTCATCTCTAAACATCAACTTACTTAATT
CTGCCAATTTCTTTTTTTTGAGACAGGGTCTCACTCTGTCACCTAGGATGGAGTGCAGTGGCACAATC
ACTGCTCACTGCAGCCTCGACTTCCCGGGCTCGGGTGATTCTCCCCAGGCTCAGGGGATTCTCCCACT
TCAGCCTCCCAAGTAGCTGGGACTACAGGTGCGCACCACCATCCCTGGCTAATATTTGTACTTTATTT
TATTTATTTATTTATTTATTTTTTGAGATGGAGTTTCGCTCTTGTTGCCCAAATGAATTGCCTCTTAT
TTAATTTCGTCTGATGATACATTTTGTTTTTATTTTGTAAAAAATTATTTTTTTTCTTTTTGGAGACA
GGGTCTTGCTCTGTTGCCCAGGCTGGTCACAAACTCCTGACCTCAAGCAATCCTCCTGCCTTAGCCTC
CCAAAATGCTGGGATTACAGGCGTGACGACCTCGCCCGGCCTTGTATTATGATACATTTTGAACAACT
ACAAGTAGACTTGGTATAATGAACCTGCACGTACCCATTGCCAAGTTCTGACAACTGTCTGTCTATAG
CCAATTATGCATTTCTTAAATTAGAACCCCCCCAATATACCCAAATATATATATATGTGTGCATATAT
ATAGTAAGTTGTAACAAAGTTGTGAATTCATACCTGAAGTATCTCAAGTGATGCAAGTTTTATGAATT
TTTGTTTATGCCTTTTGGGAAGAGTTGTATTGACAAATTTTTTATGCTTAAAGTAAACCATAAATCAA
AAAAATAAAATCTAGGATGCAATAAAACAAAACAACTTCTTGACATAAGTATGGTATGTAAATCTGTT
TTGATTGGAAATCAATTTGTTATATTGCCAGAATTCCTGTTTTAGAATACATCTCTGCTGATCTGTCT
GTATTCTTAGACTGCATATCTGGGATGAACTCTGGGCAGAATTCACATGGGCTTCCTTTGAAATAAAC
AAGACTTTTCAAATTCTTAGTCGATCTGCAGAACCTGTAGCCAGGCACTGAACCATTTTGATAGATGC
AGTAATCGTTGCAAGTGTATATTTCAAGGGAGTTCTGGCTGGGTCCTAGTTTATGCTTGTGGCAGAAG
CAGTGAGTAACTGGGAGGAAGTTGGTGAGTAAGCTTCAAGGAAGAAGTCATTTTTAGTACTCTGGATC
TTCCTGATTTTAAAGCACTACAAAATGGTGCATTTTCATTCTTGTCAAGTGATAACAGATATATTCTG
ATGAGCCTGAAATGAATATATATTGTATCATTTTTATAATATCTAGCAAGGTTTGTATTTTCCTAGAA
CTTGAACTAAATTTCAGTTCATAAAATTTATAAAATACTTAGTTGTTGTAAAATATTTTTGGAATGTT
CACATAGGTGACACACAAATGTCCCATTTTCATTCTTTCTATAGTAAATATGTTCTGATATGTGAAGG
TTTAGCAGATGCATCAGCATTTAATCCTAGAGGATCTGGCATAATCTTTTCCCCCAAGAATAGAAATT
TTTTCTGCTTATGAAAGTAGTACATGTTTCTTTAAAAACAAATCAATATTGACTTCTGCCTGCTGTAT
AGCACTATGCCTCCACCTGGCCATGACCAGGGGCATGTCCTGGTCCACCTACCTGAAAATGTTTGCAA
CCAGCCTCCTGGCCATGTGCACAGGGGCTGAAGTTGTCCCACAGGTATTACGGGCCAACCTGACAATA
CATGAAGTTCCACCAAAGTCTGAGAACTCAGAACTGAGCTTTGGGGACTGAAAGACAGCACAAACCTC
AAATTTCTCAGCACTGGAAACCTCAAAATATAACTGAATTCCATAAATAAGATTTTAAGTCTTAAATA
TGTATTTTTAAATGTATTAAAAGTCAAGCTGCTTGTATTTAAGCACCTAATACAATGCTTAGGTTGTA
AAAGGAGATGCTCAATAGGTACTAACTGATATATTGAGATTTAATTATGGTTTGACCAATATTTATTG
GAAACCGCCAAAGCTTAAATCATCAGCTTCTTGAATGTGATTTGAAAGGTAATTTAGTATTGAATAGC
ATGTGAGCTAGAGTATTTCATTCTTTCTGGTTTATTTCTTCAAATAGACTTTGAATATAATGGTGAAT
GGGTATTATAAATTAACTAATAAAAATGACATTGAAAATGAAAAAATATATATATTAAAGTGTAGAAA
GTGACCAGGCGTGGTGGCTCACACCTGTAATCCAAGCACCTTGGGAGGCTGAGGCAGGAGGATCTCTT
GATCCCAGGAGTTCAAGACCAGCCTGGGCAACATAGCGAGACTTCGTCTCTAAAAAAAAAAAAGAGAG
AGAAAAAAATTTTTTTTATTTAAAAAAAGTGTAGAAAGTGTCAAGACCCCACTTCTTACCATTATTTG
GTATATTTCTCTATACCCACCCACCCTTCCTCCTTACTCCCTCCCTCCCTTCCCAATCTTTTTATCTT
TTTGTATTCTGATTTTTTGTTTGTATATTTTGCTTTAATTTAATGTATCCTTTAAAAATTTCCCATAC
ATTTTATATGTATATATAAAAACGCATGCTGCCAAAGATAATTTATAAGAAAGACCATTGAATTTTTT
TAAAAGTGATATATATTCATTGAAAAAAATTTAGAATATATAGCAAAGCAATAAAGAACTAAATAAAA
TTGCTGTAACTCCTCTTTCAAAGATAAGTGCTTTTATGATTTTGTTGTATTTTTTTCTGTATATAGGT
ACATATATAGTATTTATAAAGCTGTACTCATAGTACATTTTCACATCACAGGTACCATATCAGTGTTA
TTAAATATTTTGTATGCCAGGGGCTAGACATACCAAGACAACCAATATGTGGTTCTACTTAAATAATA
TTAGAGTATCTTTTATGATGACACTTCATGAGTTGACTATAATAATCTTAGACTTCTAAGAGTTTGGG
TTTTCAAAAGATCACTTAGCTTTTTTGGGTGATTTTTCCCCCTTACTGTGAGATGAGAGAGGCTGTTT
GGATTTGGGATTGGGGTAGCGGGGACAGCAACTTTTCTTTTCTTTTTCTTTTTTATTTTGAGGTAGGG
TATTGCTGTGTCACCCAGGCTGGAGTGCAGTGGTGTGATCTCGGCTCACTGCAACCTCCACCTCCCGG
GCTCAGGTGATCCTCCTGCTTCAGCCTCCCAGTAACTGGGACTACAGGCGCGTGCCACATGCCTGGCT
AATTTTGTATTTTTAGTAGAGATGGGGTTTCACCATGTTGGCCAGGCTGGTCTCTAACTCCTGACCTC
AGGTGATACGCCCACCTGGGCCTCCCAAAATACTGGGATTACAGGCATGAGCCGCTGCATCAGCCAGC
AGTTTTTCTTGTGGTTTTTTTTGTTTGTTTTGTTTTGTTTTGTTTTTGAGATAGGGTCTTACTCTGTT
GTCCACGCTGGAGTGCTGTGGTATGATCGTAGCTCACTGCAGCCTCAAACTCCTGGGCTCAAGTGATT
CCTTCTGCCTCCGCCTCCCGAGTAGCTGGGACTACAGGTATGCACCACCATACCTGGCAAATTTTTAC
AAAGTTTTTTGTAGGGACGGGGTCTTGCTACATTCCCCATGTCGGTCTTGAACTCCTGGCCTCAAGCA
ACTCTCCTGTCTCAGCCTCCCAAAGCACTGGGATTACAAGTGTGAGCCACCACACCATGCCAGTTTTT
CCTGTTCAGTGTGATATTTTATCTTGTTAGACTACAGTGTGTTAAAACTTGTTTTACTAAATTTTCAA
ACATACTCAAAAGTGGAGAGAATAGTATAATGAATACCCGTATGTTCATCACCCATGTTTAGAATATT
ATTAAATATAAAGATTTTGCTGCGTTTGTCTTAGCTCTTTAAAATTTTTCTTTTTCTCTTTGTGACCT
AAAGGAAATTCCATATCTTATCACTTTACTTCTACATTCTTGACTAAGATGACTAAGACATATAGTTA
CATGGTTTTTTGTTTTGTTTTTGTTTTTTAAAGACGAAATCTCGCTCTTGTCCCCCAGGCTGGAGTGC
AATGGTGCCATCTCAGCTCAGTGCAACCTCTGCCTTCTGGGTACAAGCGATTCTCCTGCCTCAGCCTC
CCAAGTAGCTGGGATTACAGGCTCCTGCCACCACGCCTGGCTAATTTTTGTATTTTTAGTAGAGACGG
CGGGGGGAGGTTTCACCATGTTGACAAGGCTGGTCTGGAACTCCTGACCTCAGGTGATCCACCCGCCT
CGGCCTCCCAAAGTGCTGGGATTACAGGCGTGAGCCACCGCGCCCAGCCTGTTTTTTTGTTTGTGTGT
TTTGTTTTTTTTGAGACAGAGTCTTGCTCTGTTTCCCAGGCTGGAGTGAAGTGGTGCCATCTCAGCTC
AGAGACAGAGTCTTGCTCTGTTTCCCAGGCTGGAGTGAAGTGGTGCCATCTTGGCTCACTGCAACCTT
CACCTCCCAGGTTCAAGTGATTCTCCTGCCTCAGCCTCCCAAGTAGCTGGGACTACAGGCATGTGTCA
CCACACCCGGCTAATTTTTTTGTATTTTTAGTAGAGACGGGATTTCACCGTGTTGCCCAGGCTGGTCT
CGAACTCCTGAGCTCAGGCAGTCTGCCTGCCTCAGCCTCCCAAAGTGCTGGGATTACACGTGTGAACC
AACCCGCCCGGCCTGTTGTTTTCTTACATAATTCATTATCATACCTACAAAGTTAACAGTTACTAATA
TCATCTTACACCTAAATTTCTCTGATAGACTAAGGTTATTTTTTAACATCTTAATCCAATCAAATGTT
TGTATCCTGTAATGCTCTCATTGAAACAGCTATATTTCTTTTTCAGATTAGTGATGATGAACCAGGTT
ATGACCTTGATTTATTTTGCATACCTAATCATTATGCTGAGGATTTGGAAAGGGTGTTTATTCCTCAT
GGACTAATTATGGACAGGTAAGTAAGATCTTAAAATGAGGTTTTTTACTTTTTCTTGTGTTAATTTCA
AACATCAGCAGCTGTTCTGAGTACTTGCTATTTGAACATAAACTAGGCCAACTTATTAAATAACTGAT
GCTTTCTAAAATCTTCTTTATTAAAAATAAAAGAGGAGGGCCTTACTAATTACTTAGTATCAGTTGTG
GTATAGTGGGACTCTGTAGGGACCAGAACAAAGTAAACATTGAAGGGAGATGGAAGAAGGAACTCTAG
CCAGAGTCTTGCATTTCTCAGTCCTAAACAGGGTAATGGACTGGGGCTGAATCACATGAAGGCAAGGT
CAGATTTTTATTATTATGCACATCTAGCTTGAAAATTTTCTGTTAAGTCAATTACAGTGAAAAACCTT
ACCTGGTATTGAATGCTTGCATTGTATGTCTGGCTATTCTGTGTTTTTATTTTAAAATTATAATATCA
AAATATTTGTGTTATAAAATATTCTAACTATGGAGGCCATAAACAAGAAGACTAAAGTTCTCTCCTTT
CAGCCTTCTGTACACATTTCTTCTCAAGCACTGGCCTATGCATGTATACTATATGCAAAAGTACATAT
ATACATTTATATTTTAACGTATGAGTATAGTTTTAAATGTTATTGGACACTTTTAATATTAGTGTGTC
TAGAGCTATCTAATATATTTTAAAGGTTGCATAGCATTCTGTCTTATGGAGATACCATAACTGATTTA
ACCAGTCCACTATTGATAGACACTATTTTGTTCTTACCGACTGTACTAGAAGAAACATTCTTTTACAT
GTTTGGTACTTGTTCAGCTTTATTCAAGTGGAATTTCTGGGTCAAGGGGAAAGAGTTTATTGAATATT
TTGGTATTGCCAAATTTTCCTCTAAGAAGTTGAATCATTTTATACTCCTGATGTTATATGAGAGTACC
TTTCTCTTCACAATTTGTCTCTTTTTTTTTTTTTTTTGAGACAAGGTCTCTGTTGCCCAGGCTGGGGT
GCAGTGCAGCAGAATGATCACAGTTCACTGCAGTCTCAACCTCCTGGGTTCAAGCGATCCTTCCACCT
CAGCCTCCTGAGTAGCTGGGACTATAGGTGTGCGCCACCACTCCCAGCTAATATTTTTATTTTGTAGA
AACAGGGTTCGCCATGTTACCCAGCCTCCCAAAGTGCTGGGATTACAGGCATGAGCCACTGGCCCAGT
TTCTACAGTCTCTCTTAATATTGTATATTATCCAGAAAATTTCATTTAATCAGAACCTGCCAGTCTGA
TAGGTGAAAATGGTATCTTGTTTTTATTTGCATTTAAAAAAAATTATGATAGTGGTATGCTTGGTTTT
TTTGAAGGTATCAAATTTTTTACCTTATGAAACATGAGGGCAAAGGATGTGATACGTGGAAGATTTAA
AAAAAATTTTTAATGCATTTTTTTGAGACAAGGTCTTGCTCTATTGTCCAGGCTGGAGTGCAGTGGCA
CAATCACAGTTCACTCCAGCCTCAACATCCTGCACTAAAGTGATTTTCCCACCTCACCTCTCAAGTAG
CTGGGACTACAGGTACATGCTACCATGCCTGGCTAATTTTTTTTTTTTTGCAGGCATGGGGTCTCACT
ATATTGCCCAGGTTGGTGTGGAAGTTTAATGACTAAGAGGTGTTTGTTATAAAGTTTAATGTATGAAA
CTTTCTATTAAATTCCTGATTTTATTTCTGTAGGACTGAACGTCTTGCTCGAGATGTGATGAAGGAGA
TGGGAGGCCATCACATTGTAGCCCTCTGTGTGCTCAAGGGGGGCTATAAATTCTTTGCTGACCTGCTG
GATTACATCAAAGCACTGAATAGAAATAGTGATAGATCCATTCCTATGACTGTAGATTTTATCAGACT
GAAGAGCTATTGTGTGAGTATATTTAATATATGATTCTTTTTAGTGGCAACAGTAGGTTTTCTTATAT
TTTCTTTGAATCTCTGCAAACCATACTTGCTTTCATTTCACTTGGTTACAGTGAGATTTTTCTAACAT
ATTCACTAGTACTTTACATCAAAGCCAATACTGTTTTTTTAAAACTAGTCACCTTGGAGGATATATAC
TTATTTTACAGGTGTGTGTGGTTTTTTAAATAAACTCCTTTTAGGAATTGCTGTTGGGACTTGGGATA
CTTTTTTCACTATACATACTGGTGACAGATACCCTCTCTTGAGCTACATCGGTTTGTGGGGAGTCAAA
AGTCCTTTGGAGCTAGGTTTGACAAATAAGGTGGGTTAACACTTGTTTCCTAGAAAGCACATGGAGAG
CTAGAGTATTGGCGAATTGAAGAAATCCCCCTTTTTTTTTAACACACTTAAGAAAGGGGACTGCAGGT
ATACTCAAGAGAGTAAGTCGCACCAGAAACCACTTTTGATCCACAGTCTGCCTGTGTCACACAATTGA
AATGCATCACAACATTGACACTGTGGATGAAACAAAATCAGTGTGAATTTTAGTAGTGAATTTCATTC
ATAATTTGATCGTGCAAACGTTTGATTTTTATTACTTTAGACTATTGTTTCTGATTTTATGTTGGGTT
GGTATTTCCTGTGAGTTACTGTTTTACCTTTAAAATAGGAATTTTTCATACTCTTCAAAGATTAGAAC
AAATGTCCAGTTTTTGCTGTTTCATGAATGAGTCCTGTCCATCTTTGTAGAAACTCGCCTTATGTTCA
CATTTTTATTGAGAATAAGACCACTTATCTACATTTAACTATCAACCTCATCCTCTCCATTAATCATC
TATTTTAGTGACCCAAGTTTTTGACCTTTTCCATGTTTACATCAATCCTGTAGGTGATTGGGCAGCCA
TTTAAGTATTATTATAGACATTTTCACTATCCCATTAAAACCCTTTATGCCCATACATCATAACACTA
CTTCCTACCCATAAGCTCCTTTTAACTTGTTAAAGTCTTGCTTGAATTAAAGACTTGTTTACGGTATC
GATAAGCTTGATATCAAAACGCCAACTTTGACCCGGAACGCGGAAAACACCTGAGAAAAACACCTGGG
CGAGTCTCCACGTAAACGGTCAAAGTCCCCGCGGCCCTAGACAAATATTACGCGCTATGAGTAACACA
AAATTATTCAGATTTCACTTCCTCTTATTCAGTTTTCCCGCGAAAATGGCCAAATCTTACTCGGTTAC
GCCCAAATTTACTACAACATCCGCCTAAAACCGCGCGAAAATTGTCACTTCCTGTGTACACCGGCGCA
CACCAAAAACGTCACTTTTGCCACATCCGTCGCTTACATGTGTTCCGCCACACTTGCAACATCACACT
TCCGCCACACTACTACGTCACCCGCCCCGTTCCCACGCCCCGCGCCACGTCACAAACTCCACCCCCTC
ATTATCATATTGGCTTCAATCCAAAATAAGGTATATTATTGATGATGTTTAAACATTAAGAATTAATT
CGATCCTGAATGGCGAATGGACGCGCCCTGTAGCGGCGCATTAAGCGCGCGGGTGTGGTGGTTACGCG
CAGCGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAGAGCT
TTACGGCACCTCGACCGCAAAAAACTTGATTTGGGTGATGGTTCACGTAGTGGGCCATCGCCCTGATA
GACGGTTTTTCGCCCTTTGACGTTGGAGTCCACGTTCTTTAATAGTGGACTCTTGTTCCAAACTGGAA
CAACACTCAACCCTATCGCGGTCTATTCTTTTGATTTATAAGGGATGTTGCCGATTTCGGCCTATTGG
TTAAAAAATGAGCTGATTTAACAAAAATTTTAACAAAATTCAGAAGAACTCGTCAAGAAGGCGATAGA
AGGCGATGCGCTGCGAATCGGGAGCGGCGATACCGTAAAGCACGAGGAAGCGGTCAGCCCATTCGCCG
CCAAGCTCTTCAGCAATATCACGGGTAGCCAACGCTATGTCCTGATAGCGGTCCGCCACACCCAGCCG
GCCACAGTCGATGAATCCAGAAAAGCGGCCATTTTCCACCATGATATTCGGCAAGCAGGCATCGCCAT
GGGTCACGACGAGATCCTCGCCGTCGGGCATGCTCGCCTTGAGCCTGGCGAACAGTTCGGCTGGCGCG
AGCCCCTGATGCTCTTCGTCCAGATCATCCTGATCGACAAGACCGGCTTCCATCCGAGTACGTGCTCG
CTCGATGCGATGTTTCGCTTGGTGGTCGAATGGGCAGGTAGCCGGATCAAGCGTATGCAGCCGCCGCA
TTGCATCAGCCATGATGGATACTTTCTCGGCAGGAGCAAGGTGAGATGACAGGAGATCCTGCCCCGGC
ACTTCGCCCAATAGCAGCCAGTCCCTTCCCGCTTCAGTGACAACGTCGAGCACAGCTGCGCAAGGAAC
GCCCGTCGTGGCCAGCCACGATAGCCGCGCTGCCTCGTCTTGCAGTTCATTCAGGGCACCGGACAGGT
CGGTCTTGACAAAAAGAACCGGGCGCCCCTGCGCTGACAGCCGGAACACGGCGGCATCAGAGCAGCCG
ATTGTCTGTTGTGCCCAGTCATAGCCGAATAGCCTCTCCACCCAAGCGGCCGGAGAACCTGCGTGCAA
TCCATCTTGTTCAATCATGCGAAACGATCCTCATCCTGTCTCTTGATCAGAGCTTGATCCCCTGCGCC
ATCAGATCCTTGGCGGCAAGAAAGCCATCCAGTTTACTTTGCAGGGCTTCCCAACCTTACCAGAGGGC
GCCCCAGCTGGCAATTCCGGTTCGCTTGCTGTCCATAAAACCGCCCAGTCTAGCTATCGCCATGTAAG
CCCACTGCAAGCTACCTGCTTTCTCTTTGCGCTTGCGTTTTCCCTTGTCCAGATAGCCCAGTAGCTGA
CATTCATCCGGGGTCAGCACCGTTTCTGCGGACTGGCTTTCTACGTGAAAAGGATCTAGGTGAAGATC
CTTTTTGATAATCTCATGGCTGCAGCAATGGCAACAACGTTGCGCAAACTATTAACTGGCGAACTACT
TACTCTAGCTTCCCGGCAACAATTAATAGACTGGATGGAGGCGGATAAAGTTGCAGGACCACTTCTGC
GCTCGGCCCTTCCGGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGATTAAGCATTGGTAAC
TGTCAGACCAAGTTTACTCATATATACTTTAGATTGATTTAAAACTTCATTTTTAATTTAAAAGGATC
TAGGTGAAGATCCTTTTTGATAATCTCATGACCAAAATCCCTTAACGTGAGTTTTCGTTCCACTGAGC
GTCAGAC
RightITR = first underlined and bold sequence
CBh = first underlined sequence
mCherry: PKD1 = first bold sequence
HGHpA = second underlined sequence
Packaging Signal = second bold sequence
LeftITR = second underlined and bold sequence
AAV-PKD2
LeftITR-EFlα-PKD2-BGHpA-RightITR
SEQ ID NO: 6
AGATCAAAGGATCTTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGAAGGTAACTGGCTT
CAGCAGAGCGCAGATACCAAATACTGTCCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTCCTGCAGGCAGCTG
CGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCA
CGCGTTAACTATAACGGTCCTAAGGTAGCGAAGGCTCCGGTGCCCGTCAGTGGGCAGAGCGCACATCG
CCCACAGTCCCCGAGAAGTTGGGGGGAGGGGTCGGCAATTGAACCGGTGCCTAGAGAAGGTGGCGCGG
GGTAAACTGGGAAAGTGATGTCGTGTACTGGCTCCGCCTTTTTCCCGAGGGTGGGGGAGAACCGTATA
TAAGTGCAGTAGTCGCCGTGAACGTTCTTTTTCGCAACGGGTTTGCCGCCAGAACACAGGTAAGTGCC
GTGTGTGGTTCCCGCGGGCCTGGCCTCTTTACGGGTTATGGCCCTTGCGTGCCTTGAATTACTTCCAC
CTGGCTGCAGTACGTGATTCTTGATCCCGAGCTTCGGGTTGGAAGTGGGTGGGAGAGTTCGAGGCCTT
GCGCTTAAGGAGCCCCTTCGCCTCGTGCTTGAGTTGAGGCCTGGCCTGGGCGCTGGGGCCGCCGCGTG
CGAATCTGGTGGCACCTTCGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGGGCCAAGATCTGCACACTG
GTATTTCGGTTTTTGGGGCCGCGGGCGGCGACGGGGCCCGTGCGTCCCAGCGCACATGTTCGGCGAGG
CGGGGCCTGCGAGCGCGGCCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGGCCCGGTCGGCACCAGTTG
CGTGAGCGGAAAGATGGCCGCTTCCCGGCCCTGCTGCAGGGAGCTCAAAATGAAGGACGCGGCGCTCG
GGAGAGCGGGCGGGTGAGTCACCCACACAAAGGAAAAGGGCCTTTCCGTCCTCAGCCGTCGCTTCATG
TGACTCCACGGAGTACCGGGCGCCGTCCAGGCACCTCGATTAGTTCTCGAGCTTTTGGAGTACGTCGT
CTTTAGGTTGGGGGGAGGGGTTTTATGCGATGGAGTTTCCCCACACTGAGTGGGTGGAGACTGAAGTT
AGGCCAGCTTGGCACTTGATGTAATTCTCCTTGGAATTTGCCCTTTTTGAGTTTGGATCTTGGTTCAT
TCTCAAGCCTCAGACAGTGGTTCAAAGTTTTTTTCTTCCATTTCAGGTGTCGTGATCCGGAGGCGGCG
GCACGGGCGGCGGCAGCGGCGGCATGGTGAACTCCAGTCGCGTGCAGCCTCAGCAGCCCGGGGACGCC
AAGCGGCCGCCCGCGCCCCGCGCGCCGGACCCGGGCCGGCTGATGGCTGGCTGCGCGGCCGTGGGCGC
CAGCCTCGCCGCCCCGGGCGGCCTCTGCGAGCAGCGGGGCCTGGAGATCGAGATGCAGCGCATCCGGC
AGGCGGCCGCGCGGGACCCCCCGGCCGGAGCCGCGGCCTCCCCTTCTCCTCCGCTCTCGTCGTGCTCC
CGGCAGGCGTGGAGCCGCGATAACCCCGGCTTCGAGGCCGAGGAGGAGGAGGAGGAGGTGGAAGGGGA
AGAAGGCGGAATGGTGGTGGAGATGGACGTAGAGTGGCGCCCGGGCAGCCGGAGGTCGGCCGCCTCCT
CGGCCGTGAGCTCCGTGGGCGCGCGGAGCCGGGGGCTTGGGGGCTACCACGGCGCGGGCCACCCGAGC
GGGAGGCGGCGCCGGCGAGAGGACCAGGGCCCGCCGTGCCCCAGCCCAGTCGGCGGCGGGGACCCGCT
GCATCGCCACCTCCCCCTGGAAGGGCAGCCGCCCCGAGTGGCCTGGGCGGAGAGGCTGGTTCGCGGGC
TGCGAGGTCTCTGGGGAACAAGACTCATGGAGGAAAGCAGCACTAACCGAGAGAAATACCTTAAAAGT
GTTTTACGGGAACTGGTCACATACCTCCTTTTTCTCATAGTCTTGTGCATCTTGACCTACGGCATGAT
GAGCTCCAATGTGTACTACTACACCCGGATGATGTCACAGCTCTTCCTAGACACCCCCGTGTCCAAAA
CGGAGAAAACTAACTTTAAAACTCTGTCTTCCATGGAAGACTTCTGGAAGTTCACAGAAGGCTCCTTA
TTGGATGGGCTGTACTGGAAGATGCAGCCCAGCAACCAGACTGAAGCTGACAACCGAAGTTTCATCTT
CTATGAGAACCTGCTGTTAGGGGTTCCACGAATACGGCAACTCCGAGTCAGAAATGGATCCTGCTCTA
TCCCCCAGGACTTGAGAGATGAAATTAAAGAGTGCTATGATGTCTACTCTGTCAGTAGTGAAGATAGG
GCTCCCTTTGGGCCCCGAAATGGAACCGCTTGGATCTACACAAGTGAAAAAGACTTGAATGGTAGTAG
CCACTGGGGAATCATTGCAACTTATAGTGGAGCTGGCTATTATCTGGATTTGTCAAGAACAAGAGAGG
AAACAGCTGCACAAGTTGCTAGCCTCAAGAAAAATGTCTGGCTGGACCGAGGAACCAGGGCAACTTTT
ATTGACTTCTCAGTGTACAACGCCAACATTAACCTGTTCTGTGTGGTCAGGTTATTGGTTGAATTCCC
AGCAACAGGTGGTGTGATTCCATCTTGGCAATTTCAGCCTTTAAAGCTGATCCGATATGTCACAACTT
TTGATTTCTTCCTGGCAGCCTGTGAGATTATCTTTTGTTTCTTTATCTTTTACTATGTGGTGGAAGAG
ATATTGGAAATTCGCATTCACAAACTACACTATTTCAGGAGTTTCTGGAATTGTCTGGATGTTGTGAT
CGTTGTGCTGTCAGTGGTAGCTATAGGAATTAACATATACAGAACATCAAATGTGGAGGTGCTACTAC
AGTTTCTGGAAGATCAAAATACTTTCCCCAACTTTGAGCATCTGGCATATTGGCAGATACAGTTCAAC
AATATAGCTGCTGTCACAGTATTTTTTGTCTGGATTAAGCTCTTCAAATTCATCAATTTTAACAGGAC
CATGAGCCAGCTCTCGACAACCATGTCTCGATGTGCCAAAGACCTGTTTGGCTTTGCTATTATGTTCT
TCATTATTTTCCTAGCGTATGCTCAGTTGGCATACCTTGTCTTTGGCACTCAGGTCGATGACTTCAGT
ACTTTCCAAGAGTGTATCTTCACTCAATTCCGTATCATTTTGGGCGATATCAACTTTGCAGAGATTGA
GGAAGCTAATCGAGTTTTGGGACCAATTTATTTCACTACATTTGTGTTCTTTATGTTCTTCATTCTTT
TGAATATGTTTTTGGCTATCATCAATGATACTTACTCTGAAGTGAAATCTGACTTGGCACAGCAGAAA
GCTGAAATGGAACTCTCAGATCTTATCAGAAAGGGCTACCATAAAGCTTTGGTCAAACTAAAACTGAA
AAAAAATACCGTGGATGACATTTCAGAGAGTCTGCGGCAAGGAGGAGGCAAGTTAAACTTTGACGAAC
TTCGACAAGATCTCAAAGGGAAGGGCCATACTGATGCAGAGATTGAGGCAATATTCACAAAGTACGAC
CAAGATGGAGACCAAGAACTGACCGAACATGAACATCAGCAGATGAGAGACGACTTGGAGAAAGAGAG
GGAGGACCTGGATTTGGATCACAGTTCTTTACCACGTCCCATGAGCAGCCGAAGTTTCCCTCGAAGCC
TGGATGACTCTGAGGAGGATGACGATGAAGATAGCGGACATAGCTCCAGAAGGAGGGGAAGCATTTCT
AGTGGCGTTTCTTACGAAGAGTTTCAAGTCCTGGTGAGACGAGTGGACCGGATGGAGCATTCCATCGG
CAGCATAGTGTCCAAGATTGACGCCGTGATCGTGAAGCTAGAGATTATGGAGCGAGCCAAACTGAAGA
GGAGGGAGGTGCTGGGAAGGCTGTTGGATGGGGTGGCCGAGGATGAAAGGCTGGGTCGTGACAGTGAA
ATCCATAGGGAACAGATGGAACGGCTAGTACGTGAAGAGTTGGAACGCTGGGAATCCGATGATGCAGC
TTCCCAGATCAGTCATGGTTTAGGCACGCCAGTGGGACTAAATGGTCAACCTCGCCCCAGAAGCTCCC
GCCCATCTTCCTCCCAATCTACAGAAGGCATGGAAGGTGCAGGTGGAAATGGGAGTTCTAATGTCCAC
GTATGATTCTAGAGTCGACCTGCAGAAGCTTGCCTCGAGCCTGTGCCTTCTAGTTGCCAGCCATCTGT
TGTTTGCCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTCTATGGTAACTAT
AACGGTCCTAAGGTAGCGAACGGACCGAGCGGCCGCAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGGGGCGCCTGATGCGGTATTTTCTCCTTA
CGCATCTGTGCGGTATTTCACACCGCATACGTCAAAGCAACCATAGTACGCGCCCTGTAGCGGCGCAT
TAAGCGCGGCGGGTGTGGTGGTTACGCGCAGCGTGACCGCTACACTTGCCAGCGCCCTAGCGCCCGCT
CCTTTCGCTTTCTTCCCTTCCTTTCTCGCCACGTTCGCCGGCTTTCCCCGTCAAGCTCTAAATCGGGG
GCTCCCTTTAGGGTTCCGATTTAGTGCTTTACGGCACCTCGACCCCAAAAAACTTGATTTGGGTGATG
GTTCACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACCCTATCTCGGGCTATTCTTTTGATTTATA
AGGGATTTTGCCGATTTCGGCCTATTGGTTAAAAAATGAGCTGATTTAACAAAAATTTAACGCGAATT
TTAACAAAATATTAACGTTTACAATTTTATGGTGCACTCTCAGTACAATCTGCTCTGATGCCGCATAG
TTAAGCCAGCCCCGACACCCGCCAACACCCGCTGACGCGCCCTGACGGGCTTGTCTGCTCCCGGCATC
CGCTTACAGACAAGCTGTGACCGTCTCCGGGAGCTGCATGTGTCAGAGGTTTTCACCGTCATCACCGA
AACGCGCGAGACGAAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTGCCTTCCTGTT
TTTGCTCACCCAGAAACGCTGGTGAAAGTAAAAGATGCTGAAGATCAGTTGGGTGCACGAGTGGGTTA
CATCGAACTGGATCTCAACAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGACGCCGGGCAAGAGCAACTC
GGTCGCCGCATACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACATGGGGGATCATGTA
ACTCGCCTTGATCGTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTCTAGCTTCCCGGC
AACAATTAATAGACTGGATGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGCAACTATGGATGAAC
GAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGACCCCGTAGAAA
RightITR = first underlined and bold sequence
EF1α = first underlined sequence
PKD2 = bold sequence
BGHpA = second underlined sequence
LeftITR = second underlined and bold sequence
HDAd-PKD1-PKD2
RightITR-CBh-mCherry: PKD1-HGHpA-EF1α-PKD2-BGHpA-PackagingSignal-
LeftITR
SEQ ID NO: 7
CCCGTAGAAAAGATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCCGTAGTTAGGCCACCAC
TTCAAGAACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCAGCGGTCGG
GCTGAACGGGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCTGATACCGCTCGCC
GCAGCCGAACGACCGAGCGCAGCGAGTCAGTGAGCGAGGAAGCGGAAGAGCGCCTGATGCGGTATTTT
CTCCTTACGCATCTGTGCGGTATTTCACACCGCATATGGATCCATGCATGTTAAGTTTAAACATCATC
AATAATATACCTTATTTTGGATTGAAGCCAATATGATAATGAGGGGGTGGAGTTTGTGACGTGGCGCG
GGGCGTGGGAACGGGGCGGGTGACGTAGGTTTTAGGGCGGAGTAACTTGTATGTGTTGGGAATTGTAG
TTTTCTTAAAATGGGAAGTTACGTAACGTGGGAAAACGGAAGTGACGATTTGAGGAAGTTGTGGGTTT
TTTGGCTTTCGTTTCTGGGCGTAGGTTCGCGTGCGGTTTTCTGGGTGTTTTTTGTGGACTTTAACCGT
TACGTCATTTTTTAGTCCTATATATACTCGCTCTGCACTTGGCCCTTTTTTACACTGTGACTGATTGA
GCTGGTGCCGTGTCGAGTGGTGTTTTTTGATGCCCCCCCTCGAGGTTCGACGGTATCGATAAGCTTGA
TTTAATTAAGGCCGGCCCCTAGGGGCGCGCGCGGCCGCTAGGGATAACAGGGTAATTGTTGACAATTA
ATCATCGGCATAGTATATCGGCATAGTATAATACGACAAGGTGAGGAACTAAACCATGGCCAAGTTGA
CCAGTGCCGTTCCGGTGCTCACCGCGCGCGACGTCGCCGGAGCGGTCGAGTTCTGGACCGACCGGCTC
GGGTTCTCCCGGGACTTCGTGGAGGACGACTTCGCCGGTGTGGTCCGGGACGACGTGACCCTGTTCAT
CAGCGCGGTCCAGGACCAGGTGGTGCCGGACAACACCCTGGCCTGGGTGTGGGTGCGCGGCCTGGACG
AGCTGTACGCCGAGTGGTCGGAGGTCGTGTCCACGAACTTCCGGGACGCCTCCGGGCCGGCCATGACC
GAGATCGGCGAGCAGCCGTGGGGGCGGGAGTTCGCCCTGCGCGACCCGGCCGGCAACTGCGTGCACTT
CGTGGCCGAGGAGCAGGACTGAACGCGTCGTTACATAACTTACGGTAAATGGCCCGCCTGGCTGACCG
CCCAACGACCCCCGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGTCGAGGT
GAGCCCCACGTTCTGCTTCACTCTCCCCATCTCCCCCCCCTCCCCACCCCCAATTTTGTATTTATTTA
TTTTTTAATTATTTTGTGCAGCGATGGGGGCGGGGGGGGGGGGGGGGCGCGCGCCAGGCGGGGCGGGG
CGGGGCGAGGGGCGGGGCGGGGCGAGGCGGAGAGGTGCGGCGGCAGCCAATCAGAGCGGCGCGCTCCG
AAAGTTTCCTTTTATGGCGAGGCGGCGGCGGCGGCGGCCCTATAAAAAGCGAAGCGCGCGGCGGGCGG
GAGTCGCTGCGACGCTGCCTTCGCCCCGTGCCCCGCTCCGCCGCCGCCTCGCGCCGCCCGCCCCGGCT
CTGACTGACCGCGTTACTCCCACAGGTGAGCGGGCGGGACGGCCCTTCTCCTCCGGGCTGTAATTAGC
TGAGCAAGAGGTAAGGGTTTAAGGGATGGTTGGTTGGTGGGGTATTAATGTTTAATTACCTGGAGCAC
CTGTCCGGAGAATTCGCCACCATGCCGCCCGCCGCGCCCGCCCGCCTGGCGCTGGCCCTGGGCCTGGG
CCTGTGGCTCGGGGCGCTGGCGGGGGGCCCCGGGATGGTGAGCAAGGGCGAGGAGGATAACATGGCCA
TCATCAAGGAGTTCATGCGCTTCAAGGTGCACATGGAGGGCTCCGTGAACGGCCACGAGTTCGAGATC
GAGGGCGAGGGCGAGGGCCGCCCCTACGAGGGCACCCAGACCGCCAAGCTGAAGGTGACCAAGGGTGG
CCCCCTGCCCTTCGCCTGGGACATCCTGTCCCCTCAGTTCATGTACGGCTCCAAGGCCTACGTGAAGC
ACCCCGCCGACATCCCCGACTACTTGAAGCTGTCCTTCCCCGAGGGCTTCAAGTGGGAGCGCGTGATG
AACTTCGAGGACGGCGGCGTGGTGACCGTGACCCAGGACTCCTCCCTGCAGGACGGCGAGTTCATCTA
CAAGGTGAAGCTGCGCGGCACCAACTTCCCCTCCGACGGCCCCGTAATGCAGAAGAAGACCATGGGCT
GGGAGGCCTCCTCCGAGCGGATGTACCCCGAGGACGGCGCCCTGAAGGGCGAGATCAAGCAGAGGCTG
AAGCTGAAGGACGGCGGCCACTACGACGCTGAGGTCAAGACCACCTACAAGGCCAAGAAGCCCGTGCA
GCTGCCCGGCGCCTACAACGTCAACATCAAGTTGGACATCACCTCCCACAACGAGGACTACACCATCG
TGGAACAGTACGAACGCGCCGAGGGCCGCCACTCCACCGGCGGCATGGACGAGCTGTACAAGGGCGCG
CCGGGGGGCCCCGGGCGCGGCTGCGGGCCCTGCGAGCCCCCCTGCCTCTGCGGCCCAGCGCCCGGCGC
CGCCTGCCGCGTCAACTGCTCGGGCCGCGGGCTGCGGACGCTCGGTCCCGCGCTGCGCATCCCCGCGG
ACGCCACAGCGCTAGACGTCTCCCACAACCTGCTCCGGGCGCTGGACGTTGGGCTCCTGGCGAACCTC
TCGGCGCTGGCAGAGCTGGATATAAGCAACAACAAGATTTCTACGTTAGAAGAAGGAATATTTGCTAA
TTTATTTAATTTAAGTGAAATAAACCTGAGTGGGAACCCGTTTGAGTGTGACTGTGGCCTGGCGTGGC
TGCCGCGATGGGCGGAGGAGCAGCAGGTGCGGGTGGTGCAGCCCGAGGCAGCCACGTGTGCTGGGCCT
GGCTCCCTGGCTGGCCAGCCTCTGCTTGGCATCCCCTTGCTGGACAGTGGCTGTGGTGAGGAGTATGT
CGCCTGCCTCCCTGACAACAGCTCAGGCACCGTGGCAGCAGTGTCCTTTTCAGCTGCCCACGAAGGCC
TGCTTCAGCCAGAGGCCTGCAGCGCCTTCTGCTTCTCCACCGGCCAGGGCCTCGCAGCCCTCTCGGAG
CAGGGCTGGTGCCTGTGTGGGGCGGCCCAGCCCTCCAGTGCCTCCTTTGCCTGCCTGTCCCTCTGCTC
CGGCCCCCCGCCACCTCCTGCCCCCACCTGTAGGGGCCCCACCCTCCTCCAGCACGTCTTCCCTGCCT
CCCCAGGGGCCACCCTGGTGGGGCCCCACGGACCTCTGGCCTCTGGCCAGCTAGCAGCCTTCCACATC
GCTGCCCCGCTCCCTGTCACTGCCACACGCTGGGACTTCGGAGACGGCTCCGCCGAGGTGGATGCCGC
TGGGCCGGCTGCCTCGCATCGCTATGTGCTGCCTGGGCGCTATCACGTGACGGCCGTGCTGGCCCTGG
GGGCCGGCTCAGCCCTGCTGGGGACAGACGTGCAGGTGGAAGCGGCACCTGCCGCCCTGGAGCTCGTG
TGCCCGTCCTCGGTGCAGAGTGACGAGAGCCTTGACCTCAGCATCCAGAACCGCGGTGGTTCAGGCCT
GGAGGCCGCCTACAGCATCGTGGCCCTGGGCGAGGAGCCGGCCCGAGCGGTGCACCCGCTCTGCCCCT
CGGACACGGAGATCTTCCCTGGCAACGGGCACTGCTACCGCCTGGTGGTGGAGAAGGCGGCCTGGCTG
CAGGCGCAGGAGCAGTGTCAGGCCTGGGCCGGGGCCGCCCTGGCAATGGTGGACAGTCCCGCCGTGCA
GCGCTTCCTGGTCTCCCGGGTCACCAGGAGCCTAGACGTGTGGATCGGCTTCTCGACTGTGCAGGGGG
TGGAGGTGGGCCCAGCGCCGCAGGGCGAGGCCTTCAGCCTGGAGAGCTGCCAGAACTGGCTGCCCGGG
GAGCCACACCCAGCCACAGCCGAGCACTGCGTCCGGCTCGGGCCCACCGGGTGGTGTAACACCGACCT
GTGCTCAGCGCCGCACAGCTACGTCTGCGAGCTGCAGCCCGGAGGCCCAGTGCAGGATGCCGAGAACC
TCCTCGTGGGAGCGCCCAGTGGGGACCTGCAGGGACCCCTGACGCCTCTGGCACAGCAGGACGGCCTC
TCAGCCCCGCACGAGCCCGTGGAGGTCATGGTATTCCCGGGCCTGCGTCTGAGCCGTGAAGCCTTCCT
CACCACGGCCGAATTTGGGACCCAGGAGCTCCGGCGGCCCGCCCAGCTGCGGCTGCAGGTGTACCGGC
TCCTCAGCACAGCAGGGACCCCGGAGAACGGCAGCGAGCCTGAGAGCAGGTCCCCGGACAACAGGACC
CAGCTGGCCCCCGCGTGCATGCCAGGGGGACGCTGGTGCCCTGGAGCCAACATCTGCTTGCCGCTGGA
CGCCTCCTGCCACCCCCAGGCCTGCGCCAATGGCTGCACGTCAGGGCCAGGGCTACCCGGGGCCCCCT
ATGCGCTATGGAGAGAGTTCCTCTTCTCCGTTCCCGCGGGGCCCCCCGCGCAGTACTCGGTCACCCTC
CACGGCCAGGATGTCCTCATGCTCCCTGGTGACCTCGTTGGCTTGCAGCACGACGCTGGCCCTGGCGC
CCTCCTGCACTGCTCGCCGGCTCCCGGCCACCCTGGTCCCCAGGCCCCGTACCTCTCCGCCAACGCCT
CGTCATGGCTGCCCCACTTGCCAGCCCAGCTGGAGGGCACTTGGGCCTGCCCTGCCTGTGCCCTGCGG
CTGCTTGCAGCCACGGAACAGCTCACCGTGCTGCTGGGCTTGAGGCCCAACCCTGGACTGCGGCTGCC
TGGGCGCTATGAGGTCCGGGCAGAGGTGGGCAATGGCGTGTCCAGGCACAACCTCTCCTGCAGCTTTG
ACGTGGTCTCCCCAGTGGCTGGGCTGCGGGTCATCTACCCTGCCCCCCGCGACGGCCGCCTCTACGTG
CCCACCAACGGCTCAGCCTTGGTGCTCCAGGTGGACTCTGGTGCCAACGCCACGGCCACGGCTCGCTG
GCCTGGGGGCAGTGTCAGCGCCCGCTTTGAGAATGTCTGCCCTGCCCTGGTGGCCACCTTCGTGCCCG
GCTGCCCCTGGGAGACCAACGATACCCTGTTCTCAGTGGTAGCACTGCCGTGGCTCAGTGAGGGGGAG
CACGTGGTGGACGTGGTGGTGGAAAACAGCGCCAGCCGGGCCAACCTCAGCCTGCGGGTGACGGCGGA
GGAGCCCATCTGTGGCCTCCGCGCCACGCCCAGCCCCGAGGCCCGTGTACTGCAGGGAGTCCTAGTGA
GGTACAGCCCCGTGGTGGAGGCCGGCTCGGACATGGTCTTCCGGTGGACCATCAACGACAAGCAGTCC
CTGACCTTCCAGAACGTGGTCTTCAATGTCATTTATCAGAGCGCGGCGGTCTTCAAGCTCTCACTGAC
GGCCTCCAACCACGTGAGCAACGTCACCGTGAACTACAACGTAACCGTGGAGCGGATGAACAGGATGC
AGGGTCTGCAGGTCTCCACAGTGCCGGCCGTGCTGTCCCCCAATGCCACGCTAGCACTGACGGCGGGC
GTGCTGGTGGACTCGGCCGTGGAGGTGGCCTTCCTGTGGACCTTTGGGGATGGGGAGCAGGCCCTCCA
CCAGTTCCAGCCTCCGTACAACGAGTCCTTCCCGGTTCCAGACCCCTCGGTGGCCCAGGTGCTGGTGG
AGCACAATGTCATGCACACCTACGCTGCCCCAGGTGAGTACCTCCTGACCGTGCTGGCATCTAATGCC
TTCGAGAACCTGACGCAGCAGGTGCCTGTGAGCGTGCGCGCCTCCCTGCCCTCCGTGGCTGTGGGTGT
GAGTGACGGCGTCCTGGTGGCCGGCCGGCCCGTCACCTTCTACCCGCACCCGCTGCCCTCGCCTGGGG
GTGTTCTTTACACGTGGGACTTCGGGGACGGCTCCCCTGTCCTGACCCAGAGCCAGCCGGCTGCCAAC
CACACCTATGCCTCGAGGGGCACCTACCACGTGCGCCTGGAGGTCAACAACACGGTGAGCGGTGCGGC
GGCCCAGGCGGATGTGCGCGTCTTTGAGGAGCTCCGCGGACTCAGCGTGGACATGAGCCTGGCCGTGG
AGCAGGGCGCCCCCGTGGTGGTCAGCGCCGCGGTGCAGACGGGCGACAACATCACGTGGACCTTCGAC
ATGGGGGACGGCACCGTGCTGTCGGGCCCGGAGGCAACAGTGGAGCATGTGTACCTGCGGGCACAGAA
CTGCACAGTGACCGTGGGTGCGGCCAGCCCCGCCGGCCACCTGGCCCGGAGCCTGCACGTGCTGGTCT
TCGTCCTGGAGGTGCTGCGCGTTGAACCCGCCGCCTGCATCCCCACGCAGCCTGACGCGCGGCTCACG
GCCTACGTCACCGGGAACCCGGCCCACTACCTCTTCGACTGGACCTTCGGGGATGGCTCCTCCAACAC
GACCGTGCGGGGGTGCCCGACGGTGACACACAACTTCACGCGGAGCGGCACGTTCCCCCTGGCGCTGG
TGCTGTCCAGCCGCGTGAACAGGGCGCATTACTTCACCAGCATCTGCGTGGAGCCAGAGGTGGGCAAC
GTCACCCTGCAGCCAGAGAGGCAGTTTGTGCAGCTCGGGGACGAGGCCTGGCTGGTGGCATGTGCCTG
GCCCCCGTTCCCCTACCGCTACACCTGGGACTTTGGCACCGAGGAAGCCGCCCCCACCCGTGCCAGGG
GCCCTGAGGTGACGTTCATCTACCGAGACCCAGGCTCCTATCTTGTGACAGTCACCGCGTCCAACAAC
ATCTCTGCTGCCAATGACTCAGCCCTGGTGGAGGTGCAGGAGCCCGTGCTGGTCACCAGCATCAAGGT
CAATGGCTCCCTTGGGCTGGAGCTGCAGCAGCCGTACCTGTTCTCTGCTGTGGGCCGTGGGCGCCCCG
CCAGCTACCTGTGGGATCTGGGGGACGGTGGGTGGCTCGAGGGTCCGGAGGTCACCCACGCTTACAAC
AGCACAGGTGACTTCACCGTTAGGGTGGCCGGCTGGAATGAGGTGAGCCGCAGCGAGGCCTGGCTCAA
TGTGACGGTGAAGCGGCGCGTGCGGGGGCTCGTCGTCAATGCAAGCCGCACGGTGGTGCCCCTGAATG
GGAGCGTGAGCTTCAGCACGTCGCTGGAGGCCGGCAGTGATGTGCGCTATTCCTGGGTGCTCTGTGAC
CGCTGCACGCCCATCCCTGGGGGTCCTACCATCTCTTACACCTTCCGCTCCGTGGGCACCTTCAATAT
CATCGTCACGGCTGAGAACGAGGTGGGCTCCGCCCAGGACAGCATCTTCGTCTATGTCCTGCAGCTCA
TAGAGGGGCTGCAGGTGGTGGGCGGTGGCCGCTACTTCCCCACCAACCACACGGTACAGCTGCAGGCC
GTGGTTAGGGATGGCACCAACGTCTCCTACAGCTGGACTGCCTGGAGGGACAGGGGCCCGGCCCTGGC
CGGCAGCGGCAAAGGCTTCTCGCTCACCGTGCTCGAGGCCGGCACCTACCATGTGCAGCTGCGGGCCA
CCAACATGCTGGGCAGCGCCTGGGCCGACTGCACCATGGACTTCGTGGAGCCTGTGGGGTGGCTGATG
GTGGCCGCCTCCCCGAACCCAGCTGCCGTCAACACAAGCGTCACCCTCAGTGCCGAGCTGGCTGGTGG
CAGTGGTGTCGTATACACTTGGTCCTTGGAGGAGGGGCTGAGCTGGGAGACCTCCGAGCCATTTACCA
CCCATAGCTTCCCCACACCCGGCCTGCACTTGGTCACCATGACGGCAGGGAACCCGCTGGGCTCAGCC
AACGCCACCGTGGAAGTGGATGTGCAGGTGCCTGTGAGTGGCCTCAGCATCAGGGCCAGCGAGCCCGG
AGGCAGCTTCGTGGCGGCCGGGTCCTCTGTGCCCTTTTGGGGGCAGCTGGCCACGGGCACCAATGTGA
GCTGGTGCTGGGCTGTGCCCGGCGGCAGCAGCAAGCGTGGCCCTCATGTCACCATGGTCTTCCCGGAT
GCTGGCACCTTCTCCATCCGGCTCAATGCCTCCAACGCAGTCAGCTGGGTCTCAGCCACGTACAACCT
CACGGCGGAGGAGCCCATCGTGGGCCTGGTGCTGTGGGCCAGCAGCAAGGTGGTGGCGCCCGGGCAGC
TGGTCCATTTTCAGATCCTGCTGGCTGCCGGCTCAGCTGTCACCTTCCGCCTGCAGGTCGGCGGGGCC
AACCCCGAGGTGCTCCCCGGGCCCCGTTTCTCCCACAGCTTCCCCCGCGTCGGAGACCACGTGGTGAG
CGTGCGGGGCAAAAACCACGTGAGCTGGGCCCAGGCGCAGGTGCGCATCGTGGTGCTGGAGGCCGTGA
GTGGGCTGCAGGTGCCCAACTGCTGCGAGCCTGGCATCGCCACGGGCACTGAGAGGAACTTCACAGCC
CGCGTGCAGCGCGGCTCTCGGGTCGCCTACGCCTGGTACTTCTCGCTGCAGAAGGTCCAGGGCGACTC
GCTGGTCATCCTGTCGGGCCGCGACGTCACCTACACGCCCGTGGCCGCGGGGCTGTTGGAGATCCAGG
TGCGCGCCTTCAACGCCCTGGGCAGTGAGAACCGCACGCTGGTGCTGGAGGTTCAGGACGCCGTCCAG
TATGTGGCCCTGCAGAGCGGCCCCTGCTTCACCAACCGCTCGGCGCAGTTTGAGGCCGCCACCAGCCC
CAGCCCCCGGCGTGTGGCCTACCACTGGGACTTTGGGGATGGGTCGCCAGGGCAGGACACAGATGAGC
CCAGGGCCGAGCACTCCTACCTGAGGCCTGGGGACTACCGCGTGCAGGTGAACGCCTCCAACCTGGTG
AGCTTCTTCGTGGCGCAGGCCACGGTGACCGTCCAGGTGCTGGCCTGCCGGGAGCCGGAGGTGGACGT
GGTCCTGCCCCTGCAGGTGCTGATGCGGCGATCACAGCGCAACTACTTGGAGGCCCACGTTGACCTGC
GCGACTGCGTCACCTACCAGACTGAGTACCGCTGGGAGGTGTATCGCACCGCCAGCTGCCAGCGGCCG
GGGCGCCCAGCGCGTGTGGCCCTGCCCGGCGTGGACGTGAGCCGGCCTCGGCTGGTGCTGCCGCGGCT
GGCGCTGCCTGTGGGGCACTACTGCTTTGTGTTTGTCGTGTCATTTGGGGACACGCCACTGACACAGA
GCATCCAGGCCAATGTGACGGTGGCCCCCGAGCGCCTGGTGCCCATCATTGAGGGTGGCTCATACCGC
GTGTGGTCAGACACACGGGACCTGGTGCTGGATGGGAGCGAGTCCTACGACCCCAACCTGGAGGACGG
CGACCAGACGCCGCTCAGTTTCCACTGGGCCTGTGTGGCTTCGACACAGAGGGAGGCTGGCGGGTGTG
CGCTGAACTTTGGGCCCCGCGGGAGCAGCACGGTCACCATTCCACGGGAGCGGCTGGCGGCTGGCGTG
GAGTACACCTTCAGCCTGACCGTGTGGAAGGCCGGCCGCAAGGAGGAGGCCACCAACCAGACGGTGCT
GATCCGGAGTGGCCGGGTGCCCATTGTGTCCTTGGAGTGTGTGTCCTGCAAGGCACAGGCCGTGTACG
AAGTGAGCCGCAGCTCCTACGTGTACTTGGAGGGCCGCTGCCTCAATTGCAGCAGCGGCTCCAAGCGA
GGGCGGTGGGCTGCACGTACGTTCAGCAACAAGACGCTGGTGCTGGATGAGACCACCACATCCACGGG
CAGTGCAGGCATGCGACTGGTGCTGCGGCGGGGCGTGCTGCGGGACGGCGAGGGATACACCTTCACGC
TCACGGTGCTGGGCCGCTCTGGCGAGGAGGAGGGCTGCGCCTCCATCCGCCTGTCCCCCAACCGCCCG
CCGCTGGGGGGCTCTTGCCGCCTCTTCCCACTGGGCGCTGTGCACGCCCTCACCACCAAGGTGCACTT
CGAATGCACGGGCTGGCATGACGCGGAGGATGCTGGCGCCCCGCTGGTGTACGCCCTGCTGCTGCGGC
GCTGTCGCCAGGGCCACTGCGAGGAGTTCTGTGTCTACAAGGGCAGCCTCTCCAGCTACGGAGCCGTG
CTGCCCCCGGGTTTCAGGCCACACTTCGAGGTGGGCCTGGCCGTGGTGGTGCAGGACCAGCTGGGAGC
CGCTGTGGTCGCCCTCAACAGGTCTTTGGCCATCACCCTCCCAGAGCCCAACGGCAGCGCAACGGGGC
TCACAGTCTGGCTGCACGGGCTCACCGCTAGTGTGCTCCCAGGGCTGCTGCGGCAGGCCGATCCCCAG
CACGTCATCGAGTACTCGTTGGCCCTGGTCACCGTGCTGAACGAGTACGAGCGGGCCCTGGACGTGGC
GGCAGAGCCCAAGCACGAGCGGCAGCACCGAGCCCAGATACGCAAGAACATCACGGAGACTCTGGTGT
CCCTGAGGGTCCACACTGTGGATGACATCCAGCAGATCGCTGCTGCGCTGGCCCAGTGCATGGGGCCC
AGCAGGGAGCTCGTATGCCGCTCGTGCCTGAAGCAGACGCTGCACAAGCTGGAGGCCATGATGCTCAT
CCTGCAGGCAGAGACCACCGCGGGCACCGTGACGCCCACCGCCATCGGAGACAGCATCCTCAACATCA
CAGGAGACCTCATCCACCTGGCCAGCTCGGACGTGCGGGCACCACAGCCCTCAGAGCTGGGAGCCGAG
TCACCATCTCGGATGGTGGCGTCCCAGGCCTACAACCTGACCTCTGCCCTCATGCGCATCCTCATGCG
CTCCCGCGTGCTCAACGAGGAGCCCCTGACGCTGGCGGGCGAGGAGATCGTGGCCCAGGGCAAGCGCT
CGGACCCGCGGAGCCTGCTGTGCTATGGCGGCGCCCCAGGGCCTGGCTGCCACTTCTCCATCCCCGAG
GCTTTCAGCGGGGCCCTGGCCAACCTCAGTGACGTGGTGCAGCTCATCTTTCTGGTGGACTCCAATCC
CTTTCCCTTTGGCTATATCAGCAACTACACCGTCTCCACCAAGGTGGCCTCGATGGCATTCCAGACAC
AGGCCGGCGCCCAGATCCCCATCGAGCGGCTGGCCTCAGAGCGCGCCATCACCGTGAAGGTGCCCAAC
AACTCGGACTGGGCTGCCCGGGGCCACCGCAGCTCCGCCAACTCCGCCAACTCCGTTGTGGTCCAGCC
CCAGGCCTCCGTCGGTGCTGTGGTCACCCTGGACAGCAGCAACCCTGCGGCCGGGCTGCATCTGCAGC
TCAACTATACGCTGCTGGACGGCCACTACCTGTCTGAGGAACCTGAGCCCTACCTGGCAGTCTACCTA
CACTCGGAGCCCCGGCCCAATGAGCACAACTGCTCGGCTAGCAGGAGGATCCGCCCAGAGTCACTCCA
GGGTGCTGACCACCGGCCCTACACCTTCTTCATTTCCCCGGGGAGCAGAGACCCAGCGGGGAGTTACC
ATCTGAACCTCTCCAGCCACTTCCGCTGGTCGGCGCTGCAGGTGTCCGTGGGCCTGTACACGTCCCTG
TGCCAGTACTTCAGCGAGGAGGACATGGTGTGGCGGACAGAGGGGCTGCTGCCCCTGGAGGAGACCTC
GCCCCGCCAGGCCGTCTGCCTCACCCGCCACCTCACCGCCTTCGGCGCCAGCCTCTTCGTGCCCCCAA
GCCATGTCCGCTTTGTGTTTCCTGAGCCGACAGCGGATGTAAACTACATCGTCATGCTGACATGTGCT
GTGTGCCTGGTGACCTACATGGTCATGGCCGCCATCCTGCACAAGCTGGACCAGTTGGATGCCAGCCG
GGGCCGCGCCATCCCTTTCTGTGGGCAGCGGGGCCGCTTCAAGTACGAGATCCTCGTCAAGACAGGCT
GGGGCCGGGGCTCAGGTACCACGGCCCACGTGGGCATCATGCTGTATGGGGTGGACAGCCGGAGCGGC
CACCGGCACCTGGACGGCGACAGAGCCTTCCACCGCAACAGCCTGGACATCTTCCGGATCGCCACCCC
GCACAGCCTGGGTAGCGTGTGGAAGATCCGAGTGTGGCACGACAACAAAGGGCTCAGCCCTGCCTGGT
TCCTGCAGCACGTCATCGTCAGGGACCTGCAGACGGCACGCAGCGCCTTCTTCCTGGTCAATGACTGG
CTTTCGGTGGAGACGGAGGCCAACGGGGGCCTGGTGGAGAAGGAGGTGCTGGCCGCGAGCGACGCAGC
CCTTTTGCGCTTCCGGCGCCTGCTGGTGGCTGAGCTGCAGCGTGGCTTCTTTGACAAGCACATCTGGC
TCTCCATATGGGACCGGCCGCCTCGTAGCCGTTTCACTCGCATCCAGAGGGCCACCTGCTGCGTTCTC
CTCATCTGCCTCTTCCTGGGCGCCAACGCCGTGTGGTACGGGGCTGTTGGCGACTCTGCCTACAGCAC
GGGGCATGTGTCCAGGCTGAGCCCGCTGAGCGTCGACACAGTCGCTGTTGGCCTGGTGTCCAGCGTGG
TTGTCTATCCCGTCTACCTGGCCATCCTTTTTCTCTTCCGGATGTCCCGGAGCAAGGTGGCTGGGAGC
CCGAGCCCCACACCTGCCGGGCAGCAGGTGCTGGACATCGACAGCTGCCTGGACTCGTCCGTGCTGGA
CAGCTCCTTCCTCACGTTCTCAGGCCTCCACGCTGAGCAGGCCTTTGTTGGACAGATGAAGAGTGACT
TGTTTCTGGATGATTCTAAGAGTCTGGTGTGCTGGCCCTCCGGCGAGGGAACGCTCAGTTGGCCGGAC
CTGCTCAGTGACCCGTCCATTGTGGGTAGCAATCTGCGGCAGCTGGCACGGGGCCAGGCGGGCCATGG
GCTGGGCCCAGAGGAGGACGGCTTCTCCCTGGCCAGCCCCTACTCGCCTGCCAAATCCTTCTCAGCAT
CAGATGAAGACCTGATCCAGCAGGTCCTTGCCGAGGGGGTCAGCAGCCCAGCCCCTACCCAAGACACC
CACATGGAAACGGACCTGCTCAGCAGCCTGTCCAGCACTCCTGGGGAGAAGACAGAGACGCTGGCGCT
GCAGAGGCTGGGGGAGCTGGGGCCACCCAGCCCAGGCCTGAACTGGGAACAGCCCCAGGCAGCGAGGC
TGTCCAGGACAGGACTGGTGGAGGGTCTGCGGAAGCGCCTGCTGCCGGCCTGGTGTGCCTCCCTGGCC
CACGGGCTCAGCCTGCTCCTGGTGGCTGTGGCTGTGGCTGTCTCAGGGTGGGTGGGTGCGAGCTTCCC
CCCGGGCGTGAGTGTTGCGTGGCTCCTGTCCAGCAGCGCCAGCTTCCTGGCCTCATTCCTCGGCTGGG
AGCCACTGAAGGTCTTGCTGGAAGCCCTGTACTTCTCACTGGTGGCCAAGCGGCTGCACCCGGATGAA
GATGACACCCTGGTAGAGAGCCCGGCTGTGACGCCTGTGAGCGCACGTGTGCCCCGCGTACGGCCACC
CCACGGCTTTGCACTCTTCCTGGCCAAGGAAGAAGCCCGCAAGGTCAAGAGGCTACATGGCATGCTGC
GGAGCCTCCTGGTGTACATGCTTTTTCTGCTGGTGACCCTGCTGGCCAGCTATGGGGATGCCTCATGC
CATGGGCACGCCTACCGTCTGCAAAGCGCCATCAAGCAGGAGCTGCACAGCCGGGCCTTCCTGGCCAT
CACGCGGTCTGAGGAGCTCTGGCCATGGATGGCCCACGTGCTGCTGCCCTACGTCCACGGGAACCAGT
CCAGCCCAGAGCTGGGGCCCCCACGGCTGCGGCAGGTGCGGCTGCAGGAAGCACTCTACCCAGACCCT
CCCGGCCCCAGGGTCCACACGTGCTCGGCCGCAGGAGGCTTCAGCACCAGCGATTACGACGTTGGCTG
GGAGAGTCCTCACAATGGCTCGGGGACGTGGGCCTATTCAGCGCCGGATCTGCTGGGGGCATGGTCCT
GGGGCTCCTGTGCCGTGTATGACAGCGGGGGCTACGTGCAGGAGCTGGGCCTGAGCCTGGAGGAGAGC
CGCGACCGGCTGCGCTTCCTGCAGCTGCACAACTGGCTGGACAACAGGAGCCGCGCTGTGTTCCTGGA
GCTCACGCGCTACAGCCCGGCCGTGGGGCTGCACGCCGCCGTCACGCTGCGCCTCGAGTTCCCGGCGG
CCGGCCGCGCCCTGGCCGCCCTCAGCGTCCGCCCCTTTGCGCTGCGCCGCCTCAGCGCGGGCCTCTCG
CTGCCTCTGCTCACCTCGGTGTGCCTGCTGCTGTTCGCCGTGCACTTCGCCGTGGCCGAGGCCCGTAC
TTGGCACAGGGAAGGGCGCTGGCGCGTGCTGCGGCTCGGAGCCTGGGCGCGGTGGCTGCTGGTGGCGC
TGACGGCGGCCACGGCACTGGTACGCCTCGCCCAGCTGGGTGCCGCTGACCGCCAGTGGACCCGTTTC
GTGCGCGGCCGCCCGCGCCGCTTCACTAGCTTCGACCAGGTGGCGCAGCTGAGCTCCGCAGCCCGTGG
CCTGGCGGCCTCGCTGCTCTTCCTGCTTTTGGTCAAGGCTGCCCAGCAGCTACGCTTCGTGCGCCAGT
GGTCCGTCTTTGGCAAGACATTATGCCGAGCTCTGCCAGAGCTCCTGGGGGTCACCTTGGGCCTGGTG
GTGCTCGGGGTAGCCTACGCCCAGCTGGCCATCCTGCTCGTGTCTTCCTGTGTGGACTCCCTCTGGAG
CGTGGCCCAGGCCCTGTTGGTGCTGTGCCCTGGGACTGGGCTCTCTACCCTGTGTCCTGCCGAGTCCT
GGCACCTGTCACCCCTGCTGTGTGTGGGGCTCTGGGCACTGCGGCTGTGGGGCGCCCTACGGCTGGGG
GCTGTTATTCTCCGCTGGCGCTACCACGCCTTGCGTGGAGAGCTGTACCGGCCGGCCTGGGAGCCCCA
GGACTACGAGATGGTGGAGTTGTTCCTGCGCAGGCTGCGCCTCTGGATGGGCCTCAGCAAGGTCAAGG
AGTTCCGCCACAAAGTCCGCTTTGAAGGGATGGAGCCGCTGCCCTCTCGCTCCTCCAGGGGCTCCAAG
GTATCCCCGGATGTGCCCCCACCCAGCGCTGGCTCCGATGCCTCGCACCCCTCCACCTCCTCCAGCCA
GCTGGATGGGCTGAGCGTGAGCCTGGGCCGGCTGGGGACAAGGTGTGAGCCTGAGCCCTCCCGCCTCC
AAGCCGTGTTCGAGGCCCTGCTCACCCAGTTTGACCGACTCAACCAGGCCACAGAGGACGTCTACCAG
CTGGAGCAGCAGCTGCACAGCCTGCAAGGCCGCAGGAGCAGCCGGGCGCCCGCCGGATCTTCCCGTGG
CCCATCCCCGGGCCTGCGGCCAGCACTGCCCAGCCGCCTTGCCCGGGCCAGTCGGGGTGTGGACCTGG
CCACTGGCCCCAGCAGGACACCCCTTCGGGCCAAGAACAAGGTCCACCCCAGCAGCACTTAGTCCTCC
TTCCTGGCGGGGGTGGGCCGTGGAGTCGGAGTGGACACCGCTCAGTATTACTTTCTGCCGCTGTCAAG
GCCGAGGGCCAGGCAGAATGGCTGCACGTAGGTTCCCCAGAGAGCAGGCAGGGGCATCTGTCTGTCTG
TGGGCTTCAGCACTTTAAAGAGGCTGTGTGGCCAACCAGGACCCAGGGTCCCCTCCCCAGCTCCCTTG
GGAAGGACACAGCAGTATTGGACGGTTTCTAGCCTCTGAGATGCTAATTTATTTCCCCGAGTCCTCAG
GTACAGCGGGCTGTGCCCGGCCCCACCCCCTGGGCAGATGTCCCCCACTGCTAAGGCTGCTGGCTTCA
GGGAGGGTTAGCCTGCACCGCCGCCACCCTGCCCCTAAGTTATTACCTCTCCAGTTCCTACCGTACTC
CCTGCACCGTCTCACTGTGTGTCTCGTGTCAGTAATTTATATGGTGTTAAAATGTGTATATTTTTGTA
TGTCACTATTTTCACTAGGGCTGAGGGGCCTGCGCCCAGAGCTGGCCTCCCCCAACACCTGCTGCGCT
TGGTAGGTGTGGTGGCGTTATGGCAGCCCGGCTGCTGCTTGGATGCGAGCTTGGCCTTGGGCCGGTGC
TGGGGGCACAGCTGTCTGCCAGGCACTCTCATCACCCCAGAGGCCTTGTCATCCTCCCTTGCCCCAGG
CCAGGTAGCAAGAGAGCAGCGCCCAGGCCTGCTGGCATCAGGTCTGGGCAAGTAGCAGGACTAGGCAT
GTCAGAGGACCCCAGGGTGGTTAGAGGAAAAGACTCCTCCTGGGGGCTGGCTCCCAGGGTGGAGGAAG
GTGACTGTGTGTGTGTGTGTGTGCGCGCGCGCACGCGCGAGTGTGCTGTATGGCCCAGGCAGCCTCAA
GGCCCTCGGAGCTGGCTGTGCCTGCTTCTGTGTACCACTTCTGTGGGCATGGCCGCTTCTAGAACGGG
TGGCATCCCTGTGACCCCTCCCCAGTGCCTCTCCTGGCCCTGGAAGTTGCCACTCCAGTGCCCACCAG
CCTTGTCCTAATAAAATTAAGTTGCATCATTTTGTCTGACTAGGTGTCCTTCTATAATATTATGGGGT
GGAGGGGGGTGGTATGGAGCAAGGGGCAAGTTGGGAAGACAACCTGTAGGGCCTGCGGGGTCTATTGG
GAACCAAGCTGGAGTGCAGTGGCACAATCTTGGCTCACTGCAATCTCCGCCTCCTGGGTTCAAGCGAT
TCTCCTGCCTCAGCCTCCCGAGTTGTTGGGATTCCAGGCATGCATGACCAGGCTCAGCTAATTTTTGT
TTTTTTGGTAGAGACGGGGTTTCACCATATTGGCCAGGCTGGTCTCCAACTCCTAATCTCAGGTGATC
TACCCACCTTGGCCTCCCAAATTGCTGGGATTACAGGCGTGAACCACTGCTCCCTTCCCTGTCCTTTA
ACTATAACGGTCCTAAGGTAGCGAAGGCTCCGGTGCCCGTCAGTGGGCAGAGCGCACATCGCCCACAG
TCCCCGAGAAGTTGGGGGGAGGGGTCGGCAATTGAACCGGTGCCTAGAGAAGGTGGCGCGGGGTAAAC
TGGGAAAGTGATGTCGTGTACTGGCTCCGCCTTTTTCCCGAGGGTGGGGGAGAACCGTATATAAGTGC
AGTAGTCGCCGTGAACGTTCTTTTTCGCAACGGGTTTGCCGCCAGAACACAGGTAAGTGCCGTGTGTG
GTTCCCGCGGGCCTGGCCTCTTTACGGGTTATGGCCCTTGCGTGCCTTGAATTACTTCCACCTGGCTG
CAGTACGTGATTCTTGATCCCGAGCTTCGGGTTGGAAGTGGGTGGGAGAGTTCGAGGCCTTGCGCTTA
AGGAGCCCCTTCGCCTCGTGCTTGAGTTGAGGCCTGGCCTGGGCGCTGGGGCCGCCGCGTGCGAATCT
GGTGGCACCTTCGCGCCTGTCTCGCTGCTTTCGATAAGTCTCTAGCCATTTAAAATTTTTGATGACCT
GCTGCGACGCTTTTTTTCTGGCAAGATAGTCTTGTAAATGCGGGCCAAGATCTGCACACTGGTATTTC
GGTTTTTGGGGCCGCGGGCGGCGACGGGGCCCGTGCGTCCCAGCGCACATGTTCGGCGAGGCGGGGCC
TGCGAGCGCGGCCACCGAGAATCGGACGGGGGTAGTCTCAAGCTGGCCGGCCTGCTCTGGTGCCTGGC
CTCGCGCCGCCGTGTATCGCCCCGCCCTGGGCGGCAAGGCTGGCCCGGTCGGCACCAGTTGCGTGAGC
GGAAAGATGGCCGCTTCCCGGCCCTGCTGCAGGGAGCTCAAAATGAAGGACGCGGCGCTCGGGAGAGC
GGGCGGGTGAGTCACCCACACAAAGGAAAAGGGCCTTTCCGTCCTCAGCCGTCGCTTCATGTGACTCC
ACGGAGTACCGGGCGCCGTCCAGGCACCTCGATTAGTTCTCGAGCTTTTGGAGTACGTCGTCTTTAGG
TTGGGGGGAGGGGTTTTATGCGATGGAGTTTCCCCACACTGAGTGGGTGGAGACTGAAGTTAGGCCAG
CTTGGCACTTGATGTAATTCTCCTTGGAATTTGCCCTTTTTGAGTTTGGATCTTGGTTCATTCTCAAG
CCTCAGACAGTGGTTCAAAGTTTTTTTCTTCCATTTCAGGTGTCGTGATCCGGAGGCGGCGGCACGGG
CGGCGGCAGCGGCGGCATGGTGAACTCCAGTCGCGTGCAGCCTCAGCAGCCCGGGGACGCCAAGCGGC
CGCCCGCGCCCCGCGCGCCGGACCCGGGCCGGCTGATGGCTGGCTGCGCGGCCGTGGGCGCCAGCCTC
GCCGCCCCGGGCGGCCTCTGCGAGCAGCGGGGCCTGGAGATCGAGATGCAGCGCATCCGGCAGGCGGC
CGCGCGGGACCCCCCGGCCGGAGCCGCGGCCTCCCCTTCTCCTCCGCTCTCGTCGTGCTCCCGGCAGG
CGTGGAGCCGCGATAACCCCGGCTTCGAGGCCGAGGAGGAGGAGGAGGAGGTGGAAGGGGAAGAAGGC
GGAATGGTGGTGGAGATGGACGTAGAGTGGCGCCCGGGCAGCCGGAGGTCGGCCGCCTCCTCGGCCGT
GAGCTCCGTGGGCGCGCGGAGCCGGGGGCTTGGGGGCTACCACGGCGCGGGCCACCCGAGCGGGAGGC
GGCGCCGGCGAGAGGACCAGGGCCCGCCGTGCCCCAGCCCAGTCGGCGGCGGGGACCCGCTGCATCGC
CACCTCCCCCTGGAAGGGCAGCCGCCCCGAGTGGCCTGGGCGGAGAGGCTGGTTCGCGGGCTGCGAGG
TCTCTGGGGAACAAGACTCATGGAGGAAAGCAGCACTAACCGAGAGAAATACCTTAAAAGTGTTTTAC
GGGAACTGGTCACATACCTCCTTTTTCTCATAGTCTTGTGCATCTTGACCTACGGCATGATGAGCTCC
AATGTGTACTACTACACCCGGATGATGTCACAGCTCTTCCTAGACACCCCCGTGTCCAAAACGGAGAA
AACTAACTTTAAAACTCTGTCTTCCATGGAAGACTTCTGGAAGTTCACAGAAGGCTCCTTATTGGATG
GGCTGTACTGGAAGATGCAGCCCAGCAACCAGACTGAAGCTGACAACCGAAGTTTCATCTTCTATGAG
AACCTGCTGTTAGGGGTTCCACGAATACGGCAACTCCGAGTCAGAAATGGATCCTGCTCTATCCCCCA
GGACTTGAGAGATGAAATTAAAGAGTGCTATGATGTCTACTCTGTCAGTAGTGAAGATAGGGCTCCCT
TTGGGCCCCGAAATGGAACCGCTTGGATCTACACAAGTGAAAAAGACTTGAATGGTAGTAGCCACTGG
GGAATCATTGCAACTTATAGTGGAGCTGGCTATTATCTGGATTTGTCAAGAACAAGAGAGGAAACAGC
TGCACAAGTTGCTAGCCTCAAGAAAAATGTCTGGCTGGACCGAGGAACCAGGGCAACTTTTATTGACT
TCTCAGTGTACAACGCCAACATTAACCTGTTCTGTGTGGTCAGGTTATTGGTTGAATTCCCAGCAACA
GGTGGTGTGATTCCATCTTGGCAATTTCAGCCTTTAAAGCTGATCCGATATGTCACAACTTTTGATTT
CTTCCTGGCAGCCTGTGAGATTATCTTTTGTTTCTTTATCTTTTACTATGTGGTGGAAGAGATATTGG
AAATTCGCATTCACAAACTACACTATTTCAGGAGTTTCTGGAATTGTCTGGATGTTGTGATCGTTGTG
CTGTCAGTGGTAGCTATAGGAATTAACATATACAGAACATCAAATGTGGAGGTGCTACTACAGTTTCT
GGAAGATCAAAATACTTTCCCCAACTTTGAGCATCTGGCATATTGGCAGATACAGTTCAACAATATAG
CTGCTGTCACAGTATTTTTTGTCTGGATTAAGCTCTTCAAATTCATCAATTTTAACAGGACCATGAGC
CAGCTCTCGACAACCATGTCTCGATGTGCCAAAGACCTGTTTGGCTTTGCTATTATGTTCTTCATTAT
TTTCCTAGCGTATGCTCAGTTGGCATACCTTGTCTTTGGCACTCAGGTCGATGACTTCAGTACTTTCC
AAGAGTGTATCTTCACTCAATTCCGTATCATTTTGGGCGATATCAACTTTGCAGAGATTGAGGAAGCT
AATCGAGTTTTGGGACCAATTTATTTCACTACATTTGTGTTCTTTATGTTCTTCATTCTTTTGAATAT
GTTTTTGGCTATCATCAATGATACTTACTCTGAAGTGAAATCTGACTTGGCACAGCAGAAAGCTGAAA
TGGAACTCTCAGATCTTATCAGAAAGGGCTACCATAAAGCTTTGGTCAAACTAAAACTGAAAAAAAAT
ACCGTGGATGACATTTCAGAGAGTCTGCGGCAAGGAGGAGGCAAGTTAAACTTTGACGAACTTCGACA
AGATCTCAAAGGGAAGGGCCATACTGATGCAGAGATTGAGGCAATATTCACAAAGTACGACCAAGATG
GAGACCAAGAACTGACCGAACATGAACATCAGCAGATGAGAGACGACTTGGAGAAAGAGAGGGAGGAC
CTGGATTTGGATCACAGTTCTTTACCACGTCCCATGAGCAGCCGAAGTTTCCCTCGAAGCCTGGATGA
CTCTGAGGAGGATGACGATGAAGATAGCGGACATAGCTCCAGAAGGAGGGGAAGCATTTCTAGTGGCG
TTTCTTACGAAGAGTTTCAAGTCCTGGTGAGACGAGTGGACCGGATGGAGCATTCCATCGGCAGCATA
GTGTCCAAGATTGACGCCGTGATCGTGAAGCTAGAGATTATGGAGCGAGCCAAACTGAAGAGGAGGGA
GGTGCTGGGAAGGCTGTTGGATGGGGTGGCCGAGGATGAAAGGCTGGGTCGTGACAGTGAAATCCATA
GGGAACAGATGGAACGGCTAGTACGTGAAGAGTTGGAACGCTGGGAATCCGATGATGCAGCTTCCCAG
ATCAGTCATGGTTTAGGCACGCCAGTGGGACTAAATGGTCAACCTCGCCCCAGAAGCTCCCGCCCATC
TTCCTCCCAATCTACAGAAGGCATGGAAGGTGCAGGTGGAAATGGGAGTTCTAATGTCCACGTATGAT
TCTAGAGTCGACCTGCAGAAGCTTGCCTCGAGCCTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGTAACTATAACGGTC
CTAAGGTAGCGAAGTCGACCGAATCGTTGTCCCTTGTCACAGCCATTGAGAATTTTGGCAGGGAGCAT
GTTCTTAGAGCATTTTTAGGCTCTGCGGGACATAACAGCTCTGCCTCAGAGCACATGCCTTTCTCAGC
TCCTGAAAGCCACTGATCAAATTGGAACATTTTGTACCTTAGGGATGAGGATATCAACTCTCCCAGCC
ACTTAGAGGGATAAATGTGATGATGCATTCAATTGTGACTACATCTGATCCCAACTGTTGCTTCAGCT
GCTCTCCTATAGCACATGGCGGGAGGCGTGCATCCCAGTAGCTACCTCCCCACTTTTGGGGAGATGTG
GTTCCATCCATGAAACCTGGGTACCCGCCTACCAGGTCCTGGCCTATCAGGTGGCAGGGTCTGGTCAA
AGAAGGGCATGTGTGGTCTTCAGCAAGGGAGACAGGACGGTGGTGCAGAGCGTCTAGACCCTCAGGGC
AAGTCTCCCCCACACCTGCTCCCGGGGCAGTTGTCTTTGTGACCTCCCATCCCCCTCTGTTTCATCCT
CTATAAAATGAGGGGCTGAGCCCCAAAATAACAGGCTTCTTTGCCATGATGCAAAACTGCTGAATCTT
TCTTTCTGACACACAAGGCATCGAGCAGCCTCTGAAAGAACCAAAGCCACTAGCAGGCTTCCTGACTT
GGGTTTGTAGGTACTGAATACTCCCTTGAAAAATAAAAACATAGAGGCACTTTTCTCCTGGCTGTTTA
TTACAGAACGAAGAAAAAACACACTGGCTTGAAACAGACGCCAGATTTCAAATGTAGAGGTGAAATAC
GAGGTGGCAATTAAAATGTGATTACAGAAAGTCTGGACACTGAGAAAAGTTTACAGGACAGTGGGTGT
GGGTTTTCTATAACAGACACTTAAATATACATGACGATAATTGCAGATAGAAACCATCAAAGACAAAC
CCCAAATCAACTAATAATGTTTACAGATGTTCCCCCCCAAACCACAGAGCCTTACATCAAAACAAATA
CTGAAAGGCTTTAAACCAGGAACAGCTCGCCTTAACCCCACGAGGGTGCACACAAGCTGGGCTTTTTC
TCTCGGTCTGAATGGTAAAGGGAGGAGGATACTCTAGCTCCTCCAGGTGGATTGCTGAGACAGGGCTC
GGCTCACACACTGTCTCTGCGCCTCTCCCAAATCTGGAGAACTCTCCCAGCCTCCTGGTAAAGTGTCT
CTGTGGGGCACTTAACGATAAAACAGCTTCTGCTGTAAAGCTCATTAGGAAAGAGCTAGCGGAGACTG
AAAGGTTCGCAAAAGAGATTAAGAATCACACAAGGCAATAGGATTTTTAGTGAACATAGAAATAAATG
GCCAAGTGGTTTTCTATTTGGCATTTGTCAACTTGCACAACAACTCTTGGTCATATCCACATTGCTCA
TTGCATTAAAACCATAAGCGACTCAGCCACCTAGCTTAACAAGGTATCACTGGAGCAAACAACACGGT
CTGCATATTTGTAACATTGTATAATAAACACAAAACAATGCATAGTAAACACAACTCTACTGAAACAA
AAGCCGTCGCTTTATTTACAAAGTCACAAAATGAAGTATAAATACTTCTGTCATTAATGTTTAGGAAA
ACCATTTACAAAATTTTCAAATATGTACACGTAGCTTGAAAAATCACCAGCTTTCCATTTTGTCACAG
GTAGAGAGAGGGATAAGCATGGGCTGACAACACCACTCAAATTGTAACGGGAGACAACTGCGGGTATG
GATCGACACCACTTCCTAGAGTGATGTCACCATGGGGGTTTCTATGGGCATCCTGCTCAGATTTAAAG
TGCCCCAGCATCCTGGGTGACTTGCCCAGAATTCTGGGCTGTGGCATTTTGAGCAGCAGCATGCTGTT
CCAAAATGTCGTCGATCAGCCTCAAGTTGCACACCCAGTCTTCATCTGGGCTCACACAGGAGCCTTTC
AAGAGAGCTTCAATGAAATCTACCTCATTGCAGTCAGGTGACGAAATCAGATCATTTAGTGGGGGTTG
GGGCTGGCGCAAAAAGTCGGCAGGTGGCAGCTCAGGGGGAATATCCGTTCTGTCGAACGGACCTGGGA
ACTGGCTGGCAGCAACGGCAGAAGCAGCAGCAGCGGTGGCAGCAGCAGCCACATAGCTTGGTGGCTCG
ATGCCCTGTATGGGGCTCAGGGGACTAAAGCTGGCCATACCCTGCTGGAGGAACTTGGTGGTGTTTGC
TACAGGCACCGGGCCCTGTACCGGGCTCTGCCTGAGGCTCTGGCTGCCCAGCAGGCTGAAGCTGGGGT
TGTTGGCCAGGGGCACTTGTGTTCCCATCGCAGCGGGCACTTGTGCCTCCCAATCAGATGGCCTCTGA
AGGCAGGCCTGGCCAGAAGGTGAGTGCTGCTGAACGCTATTATCCACTTGGCTGAGGGGTGTTTTCCC
CGAAACTGCTGTGGTCACAGCTGCTGCCGCTGTGACCCATGCAGCATTGTTGAACGCAGTGGGCATTC
TTGGCACACTAGGCCGTCTGAGCTGGTGGGGACTCAAGGACTGGGTGCCCAGGGAGCTGGGACAGAAC
CCAGGCAGGGGCACTTCTGGTGGGGTGGCCTTGGGGCTCTGCATATGCTGGCAGACAGAGTCAAGTCT
GCCCAGGGGAGTCTGGCCTGAGTGTGAGAGGATGGGACACTGGGGGCTGGAGGTGAAAATTCCTTGCC
GCTTCCCCAGAGTTGGTGAGATCACTCCCATGCCCTCGCAGCTCTGGTGCCTGGTGAGTGGGATCATT
CCTGGACTCAGATTGTTCTGAAGAAGCCCAGTTCTGGGTGGCATCAAGTGCTTGCTAGATGGGGGGCT
TGCCTTGATCCGGCTACACTTGGAGGTGACTTGTTCTTGGACGGCTACATACAGAAAGAGAGAAGTGG
GGATGAGTTCCAAAGGCATCCTCGACTTCGGCTGTGGCCACCGGAGGGTAGCTCCTGGCCCAACACGG
ACTTCTCACCTCCCGCCCTTGGCTCTCTACTGAGCTCCCCCCTGCTCCCCAATTCCTCGCCATTCCCC
TCATTTCTCTGCCCTCAGCCTGGACTGCAGTTCTTCTGGGAAGCTGCCCCAACTCCCTAGGTCTGTGC
TCACCAAGAGCAGATCACACTGGACTGAAATGCCAGCTGATTTGTCTCTTCAAGAAAATTGGAAGCTC
CTGGAGGTCAGGGTCCATGTCTGCTTTTACACTCAGTGCTCTGTATGCAGGCCTGGCACTGCCCACCC
TTTGACAGGTGGTGCATATTTTGTAGAAGGAAGGAAGGGGCCAGGTGGGGTGGGCTGGGCTGGTGGCG
GGAGCTAGCTCAGCCTCTTAGATTCTCTACCCGATGGATGTGACCTGGGACAGCAAGTGAGTGTGGTG
AGTGAGTGCAGACGGTGCTTTGTTCCCCTCTTGTCTCATAGCCTAGATGGCCTCTGAGCCCAGATCTG
GGGCTCAGACAACATTTGTTCAACTGAACGGTAATGGGTTTCCTTTCTGAAGGCTGAAATCTGGGAGC
TGACATTCTGGACTCCCTGAGTTCTGAAGAGCCTGGGGATGGAGAGACACGGAGCAGAAGATGGAAGG
TAGAGTCCCAGGTGCCTAAGATGGGGAATACATCTCCCCTCATTGTCATGAGAGTCCACTCTAGCTGA
TATCTACTGTGGCCAATATCTACCGGTACTTTTTTGGGGTGGACACTGAGTCATGCAGCAGTCTTATG
GTTTACCCAAGGTCAGGTAGGGGAGACAGTGCAGTCAGAGCACAAGCCCAGTGTGTCTGACCCACCCA
AGAATCCATGCTCGTATCTACAAAAATGATTTTTTCTCTTGTAATGGTGCCTAGGTTCTTTTATTATC
ATGGCATGTGTATGTTTTTCAACTAGGTTACAATCTGGCCTTATAAGGTTAACCTCCTGGAGGCCACC
AGCCTTCCTGAAACTTGTCTGTGCTGTCCCTGCAACTGGAGTGTGCCTGATGTGGCACTCCAGCCTGG
ACAAGTGGGACACAGACTCCGCTGTTATCAGGCCCAAAGATGTCTTCCATAAGACCAGAAGAGCAATG
GTGTAGAGGTGTCATGGGCTACAATAAAGATGCTGACCTCCTGTCTGAGGGCAAGCAGCCTCTTCTGG
CCCTCAGACAAATGCTGAGTGTTCCCAAGACTACCCTCGGCCTGGTCCAATCTCATCCCACTGGTGCG
TAAGGGTTGCTGAACTCATGACTTCTTGGCTAGCCTGCAACCTCCACGGAGTGGGAACTACATCAGGC
ATTTTGCTAACTGCTGTATCCTAGGCCAATAAATGTTGATCACATTTATAGCTGCCATGGTAGGGTGG
GGACCCCTGCTATCTATCTGTGGAGGCTCTGGGAGCCCCTGACACAAACTTTCTGAAGCAGAGCCTCC
CCAACCCCTTTTCCATTCCCTATACCTGACAGATGGCCCAGGAACCCATTAGAAATGGAAGGTCACTG
CAGCAGTATGTGAATGTGCGTGTGGGAGAAGGGCAGGATCAGAGCCCTGGGGGTGTGGCAGCCCCCAA
GTGATTCTAATCCAGATCCTAGGGTTGTTTCCCTGTCCCATTGAAATAGCTGCTTTAAGGGGCCTGAC
TCAGGGAAATCAGTCTCTTGAATTAAGTGGTGATTTTGGAGTCATTTAGACCAGGCCTTCAATTGGGA
TCCACTAGTTCTAGAGCGGCCGGGCCCAGGGAACCCCGCAGGCGGGGGGGGCCAGTTTCCCGGGTTCG
GCTTTACGTCACGCGAGGGCGGCAGGGAGGACGGAATGGCGGGGTTTGGGGTGGGTCCCTCCTCGGGG
GAGCCCTGGGAAAAGAGGACTGCGTGTGGGAAGAGAAGGTGGAAATGGCGTTTTGGTTGACATGTGCC
GCCTGCGAGCGTGCTGCGGGGAGGGGCCGAGGGCAGATTCGGGAATGATGGCGCGGGGTGGGGGCGTG
GGGGCTTTCTCGGGAGAGGCCCTTCCCTGGAAGTTTGGGGTGCGATGGTGAGGTTCTCGGGGCACCTC
TGGAGGGGCCTCGGCACGGAAAGCGACCACCTGGGAGGGCGTGTGGGGACCAGGTTTTGCCTTTAGTT
TTGCACACACTGTAGTTCATCTTTATGGAGATGCTCATGGCCTCATTGAAGCCCCACTACAGCTCTGG
TAGCGGTAACCATGCGTATTTGACACACGAAGGAACTAGGGAAAAGGCATTAGGTCATTTCAAGCCGA
AATTCACATGTGCTAGAATCCAGATTCCATGCTGACCGATGCCCCAGGATATAGAAAATGAGAATCTG
GTCCTTACCTTCAAGAACATTCTTAACCGTAATCAGCCTCTGGTATCTTAGCTCCACCCTCACTGGTT
TTTTCTTGTTTGTTGAACCGGCCAAGCTGCTGGCCTCCCTCCTCAACCGTTCTGATCATGCTTGCTAA
AATAGTCAAAACCCCGGCCAGTTAAATATGCTTTAGCCTGCTTTATTATGATTATTTTTGTTGTTTTG
GCAATGACCTGGTTACCTGTTGTTTCTCCCACTAAAACTTTTTAAGGGCAGGAATCACCGCCGTAACT
CTAGCACTTAGCACAGTACTTGGCTTGTAAGAGGTCCTCGATGATGGTTTGTTGAATGAATACATTAA
ATAATTAACCACTTGAACCCTAAGAAAGAAGCGATTCTATTTCATATTAGGCATTGTAATGACTTAAG
GTAAAGAGCAGTGCTATTAACGGAGTCTAACTGGGAATCCAGCTTGTTTGGGCTATTTACTAGTTGTG
TGGCTGTGGGCAACTTACTTCACCTCTCTGGGCTTAAGTCATTTTATGTATATCTGAGGTGCTGGCTA
CCTCTTGGAGTTATTGAGAGGATTATAAGACAGTCTATGTGAATCAGCAACCCTTGCATGGCCCCTGG
CGGGGAACAGTAATAATAGCCATCATCATGTTTACTTACATAGTCCTAATTAGTCTTCAAAACAGCCC
TGTAGCAATGGTATGATTATTACCATTTTACAGATGAGGAACCTTTGAAGCCTCAGAGAGGCTAACAG
ACATACCCTAGGTCATACAGTTATTAAGAGAAGGAGCTCTGTCTCGAACCTAGCTCTCTCTCTCTCGA
GTAATACCAGTTAAAAAATAGGCTACAAATAGGTACTCAAAAAAATGGTAGTGGCTGTTGTTTTTATT
CAGTTGCTGAGGAAAAAATGTTGATTTTTCATCTCTAAACATCAACTTACTTAATTCTGCCAATTTCT
TTTTTTTGAGACAGGGTCTCACTCTGTCACCTAGGATGGAGTGCAGTGGCACAATCACTGCTCACTGC
AGCCTCGACTTCCCGGGCTCGGGTGATTCTCCCCAGGCTCAGGGGATTCTCCCACTTCAGCCTCCCAA
GTAGCTGGGACTACAGGTGCGCACCACCATCCCTGGCTAATATTTGTACTTTATTTTATTTATTTATT
TATTTATTTTTTGAGATGGAGTTTCGCTCTTGTTGCCCAAATGAATTGCCTCTTATTTAATTTCGTCT
GATGATACATTTTGTTTTTATTTTGTAAAAAATTATTTTTTTTCTTTTTGGAGACAGGGTCTTGCTCT
GTTGCCCAGGCTGGTCACAAACTCCTGACCTCAAGCAATCCTCCTGCCTTAGCCTCCCAAAATGCTGG
GATTACAGGCGTGACGACCTCGCCCGGCCTTGTATTATGATACATTTTGAACAACTACAAGTAGACTT
GGTATAATGAACCTGCACGTACCCATTGCCAAGTTCTGACAACTGTCTGTCTATAGCCAATTATGCAT
TTCTTAAATTAGAACCCCCCCAATATACCCAAATATATATATATGTGTGCATATATATAGTAAGTIGT
AACAAAGTTGTGAATTCATACCTGAAGTATCTCAAGTGATGCAAGTTTTATGAATTTTTGTTTATGCC
TTTTGGGAAGAGTTGTATTGACAAATTTTTTATGCTTAAAGTAAACCATAAATCAAAAAAATAAAATC
TAGGATGCAATAAAACAAAACAACTTCTTGACATAAGTATGGTATGTAAATCTGTTTTGATTGGAAAT
CAATTTGTTATATTGCCAGAATTCCTGTTTTAGAATACATCTCTGCTGATCTGTCTGTATTCTTAGAC
TGCATATCTGGGATGAACTCTGGGCAGAATTCACATGGGCTTCCTTTGAAATAAACAAGACTTTTCAA
ATTCTTAGTCGATCTGCAGAACCTGTAGCCAGGCACTGAACCATTTTGATAGATGCAGTAATCGTTGC
AAGTGTATATTTCAAGGGAGTTCTGGCTGGGTCCTAGTTTATGCTTGTGGCAGAAGCAGTGAGTAACT
GGGAGGAAGTTGGTGAGTAAGCTTCAAGGAAGAAGTCATTTTTAGTACTCTGGATCTTCCTGATTTTA
AAGCACTACAAAATGGTGCATTTTCATTCTTGTCAAGTGATAACAGATATATTCTGATGAGCCTGAAA
TGAATATATATTGTATCATTTTTATAATATCTAGCAAGGTTTGTATTTTCCTAGAACTTGAACTAAAT
TTCAGTTCATAAAATTTATAAAATACTTAGTTGTTGTAAAATATTTTTGGAATGTTCACATAGGTGAC
ACACAAATGTCCCATTTTCATTCTTTCTATAGTAAATATGTTCTGATATGTGAAGGTTTAGCAGATGC
ATCAGCATTTAATCCTAGAGGATCTGGCATAATCTTTTCCCCCAAGAATAGAAATTTTTTCTGCTTAT
GAAAGTAGTACATGTTTCTTTAAAAACAAATCAATATTGACTTCTGCCTGCTGTATAGCACTATGCCT
CCACCTGGCCATGACCAGGGGCATGTCCTGGTCCACCTACCTGAAAATGTTTGCAACCAGCCTCCTGG
CCATGTGCACAGGGGCTGAAGTTGTCCCACAGGTATTACGGGCCAACCTGACAATACATGAAGTTCCA
CCAAAGTCTGAGAACTCAGAACTGAGCTTTGGGGACTGAAAGACAGCACAAACCTCAAATTTCTCAGC
ACTGGAAACCTCAAAATATAACTGAATTCCATAAATAAGATTTTAAGTCTTAAATATGTATTTTTAAA
TGTATTAAAAGTCAAGCTGCTTGTATTTAAGCACCTAATACAATGCTTAGGTTGTAAAAGGAGATGCT
CAATAGGTACTAACTGATATATTGAGATTTAATTATGGTTTGACCAATATTTATTGGAAACCGCCAAA
GCTTAAATCATCAGCTTCTTGAATGTGATTTGAAAGGTAATTTAGTATTGAATAGCATGTGAGCTAGA
GTATTTCATTCTTTCTGGTTTATTTCTTCAAATAGACTTTGAATATAATGGTGAATGGGTATTATAAA
TTAACTAATAAAAATGACATTGAAAATGAAAAAATATATATATTAAAGTGTAGAAAGTGACCAGGCGT
GGTGGCTCACACCTGTAATCCAAGCACCTTGGGAGGCTGAGGCAGGAGGATCTCTTGATCCCAGGAGT
TCAAGACCAGCCTGGGCAACATAGCGAGACTTCGTCTCTAAAAAAAAAAAAGAGAGAGAAAAAAATTT
TTTTTATTTAAAAAAAGTGTAGAAAGTGTCAAGACCCCACTTCTTACCATTATTTGGTATATTTCTCT
ATACCCACCCACCCTTCCTCCTTACTCCCTCCCTCCCTTCCCAATCTTTTTATCTTTTTGTATTCTGA
TTTTTTGTTTGTATATTTTGCTTTAATTTAATGTATCCTTTAAAAATTTCCCATACATTTTATATGTA
TATATAAAAACGCATGCTGCCAAAGATAATTTATAAGAAAGACCATTGAATTTTTTTAAAAGTGATAT
ATATTCATTGAAAAAAATTTAGAATATATAGCAAAGCAATAAAGAACTAAATAAAATTGCTGTAACTC
CTCTTTCAAAGATAAGTGCTTTTATGATTTTGTTGTATTTTTTTCTGTATATAGGTACATATATAGTA
TTTATAAAGCTGTACTCATAGTACATTTTCACATCACAGGTACCATATCAGTGTTATTAAATATTTTG
TATGCCAGGGGCTAGACATACCAAGACAACCAATATGTGGTTCTACTTAAATAATATTAGAGTATCTT
TTATGATGACACTTCATGAGTTGACTATAATAATCTTAGACTTCTAAGAGTTTGGGTTTTCAAAAGAT
CACTTAGCTTTTTTGGGTGATTTTTCCCCCTTACTGTGAGATGAGAGAGGCTGTTTGGATTTGGGATT
GGGGTAGCGGGGACAGCAACTTTTCTTTTCTTTTTCTTTTTTATTTTGAGGTAGGGTATTGCTGTGTC
ACCCAGGCTGGAGTGCAGTGGTGTGATCTCGGCTCACTGCAACCTCCACCTCCCGGGCTCAGGTGATC
CTCCTGCTTCAGCCTCCCAGTAACTGGGACTACAGGCGCGTGCCACATGCCTGGCTAATTTTGTATTT
TTAGTAGAGATGGGGTTTCACCATGTTGGCCAGGCTGGTCTCTAACTCCTGACCTCAGGTGATACGCC
CACCTGGGCCTCCCAAAATACTGGGATTACAGGCATGAGCCGCTGCATCAGCCAGCAGTTTTTCTTGT
GGTTTTTTTTGTTTGTTTTGTTTTGTTTTGTTTTTGAGATAGGGTCTTACTCTGTTGTCCACGCTGGA
GTGCTGTGGTATGATCGTAGCTCACTGCAGCCTCAAACTCCTGGGCTCAAGTGATTCCTTCTGCCTCC
GCCTCCCGAGTAGCTGGGACTACAGGTATGCACCACCATACCTGGCAAATTTTTACAAAGTTTTTTGT
AGGGACGGGGTCTTGCTACATTCCCCATGTCGGTCTTGAACTCCTGGCCTCAAGCAACTCTCCTGTCT
CAGCCTCCCAAAGCACTGGGATTACAAGTGTGAGCCACCACACCATGCCAGTTTTTCCTGTTCAGTGT
GATATTTTATCTTGTTAGACTACAGTGTGTTAAAACTTGTTTTACTAAATTTTCAAACATACTCAAAA
GTGGAGAGAATAGTATAATGAATACCCGTATGTTCATCACCCATGTTTAGAATATTATTAAATATAAA
GATTTTGCTGCGTTTGTCTTAGCTCTTTAAAATTTTTCTTTTTCTCTTTGTGACCTAAAGGAAATTCC
ATATCTTATCACTTTACTTCTACATTCTTGACTAAGATGACTAAGACATATAGTTACATGGTTTTTTG
TTTTGTTTTTGTTTTTTAAAGACGAAATCTCGCTCTTGTCCCCCAGGCTGGAGTGCAATGGTGCCATC
TCAGCTCAGTGCAACCTCTGCCTTCTGGGTACAAGCGATTCTCCTGCCTCAGCCTCCCAAGTAGCTGG
GATTACAGGCTCCTGCCACCACGCCTGGCTAATTTTTGTATTTTTAGTAGAGACGGCGGGGGGAGGTT
TCACCATGTTGACAAGGCTGGTCTGGAACTCCTGACCTCAGGTGATCCACCCGCCTCGGCCTCCCAAA
GTGCTGGGATTACAGGCGTGAGCCACCGCGCCCAGCCTGTTTTTTTGTTTGTGTGTTTTGTTTTTTTT
GAGACAGAGTCTTGCTCTGTTTCCCAGGCTGGAGTGAAGTGGTGCCATCTCAGCTCAGAGACAGAGTC
TTGCTCTGTTTCCCAGGCTGGAGTGAAGTGGTGCCATCTTGGCTCACTGCAACCTTCACCTCCCAGGT
TCAAGTGATTCTCCTGCCTCAGCCTCCCAAGTAGCTGGGACTACAGGCATGTGTCACCACACCCGGCT
AATTTTTTTGTATTTTTAGTAGAGACGGGATTTCACCGTGTTGCCCAGGCTGGTCTCGAACTCCTGAG
CTCAGGCAGTCTGCCTGCCTCAGCCTCCCAAAGTGCTGGGATTACACGTGTGAACCAACCCGCCCGGC
CTGTTGTTTTCTTACATAATTCATTATCATACCTACAAAGTTAACAGTTACTAATATCATCTTACACC
TAAATTTCTCTGATAGACTAAGGTTATTTTTTAACATCTTAATCCAATCAAATGTTTGTATCCTGTAA
TGCTCTCATTGAAACAGCTATATTTCTTTTTCAGATTAGTGATGATGAACCAGGTTATGACCTTGATT
TATTTTGCATACCTAATCATTATGCTGAGGATTTGGAAAGGGTGTTTATTCCTCATGGACTAATTATG
GACAGGTAAGTAAGATCTTAAAATGAGGTTTTTTACTTTTTCTTGTGTTAATTTCAAACATCAGCAGC
TGTTCTGAGTACTTGCTATTTGAACATAAACTAGGCCAACTTATTAAATAACTGATGCTTTCTAAAAT
CTTCTTTATTAAAAATAAAAGAGGAGGGCCTTACTAATTACTTAGTATCAGTTGTGGTATAGTGGGAC
TCTGTAGGGACCAGAACAAAGTAAACATTGAAGGGAGATGGAAGAAGGAACTCTAGCCAGAGTCTTGC
ATTTCTCAGTCCTAAACAGGGTAATGGACTGGGGCTGAATCACATGAAGGCAAGGTCAGATTTTTATT
ATTATGCACATCTAGCTTGAAAATTTTCTGTTAAGTCAATTACAGTGAAAAACCTTACCTGGTATTGA
ATGCTTGCATTGTATGTCTGGCTATTCTGTGTTTTTATTTTAAAATTATAATATCAAAATATTTGTGT
TATAAAATATTCTAACTATGGAGGCCATAAACAAGAAGACTAAAGTTCTCTCCTTTCAGCCTTCTGTA
CACATTTCTTCTCAAGCACTGGCCTATGCATGTATACTATATGCAAAAGTACATATATACATTTATAT
TTTAACGTATGAGTATAGTTTTAAATGTTATTGGACACTTTTAATATTAGTGTGTCTAGAGCTATCTA
ATATATTTTAAAGGTTGCATAGCATTCTGTCTTATGGAGATACCATAACTGATTTAACCAGTCCACTA
TTGATAGACACTATTTTGTTCTTACCGACTGTACTAGAAGAAACATTCTTTTACATGTTTGGTACTTG
TTCAGCTTTATTCAAGTGGAATTTCTGGGTCAAGGGGAAAGAGTTTATTGAATATTTTGGTATTGCCA
AATTTTCCTCTAAGAAGTTGAATCATTTTATACTCCTGATGTTATATGAGAGTACCTTTCTCTTCACA
ATTTGTCTCTTTTTTTTTTTTTTTTGAGACAAGGTCTCTGTTGCCCAGGCTGGGGTGCAGTGCAGCAG
AATGATCACAGTTCACTGCAGTCTCAACCTCCTGGGTTCAAGCGATCCTTCCACCTCAGCCTCCTGAG
TAGCTGGGACTATAGGTGTGCGCCACCACTCCCAGCTAATATTTTTATTTTGTAGAAACAGGGTTCGC
CATGTTACCCAGCCTCCCAAAGTGCTGGGATTACAGGCATGAGCCACTGGCCCAGTTTCTACAGTCTC
TCTTAATATTGTATATTATCCAGAAAATTTCATTTAATCAGAACCTGCCAGTCTGATAGGTGAAAATG
GTATCTTGTTTTTATTTGCATTTAAAAAAAATTATGATAGTGGTATGCTTGGTTTTTTTGAAGGTATC
AAATTTTTTACCTTATGAAACATGAGGGCAAAGGATGTGATACGTGGAAGATTTAAAAAAAATTTTTA
ATGCATTTTTTTGAGACAAGGTCTTGCTCTATTGTCCAGGCTGGAGTGCAGTGGCACAATCACAGTTC
ACTCCAGCCTCAACATCCTGCACTAAAGTGATTTTCCCACCTCACCTCTCAAGTAGCTGGGACTACAG
GTACATGCTACCATGCCTGGCTAATTTTTTTTTTTTTGCAGGCATGGGGTCTCACTATATTGCCCAGG
TTGGTGTGGAAGTTTAATGACTAAGAGGTGTTTGTTATAAAGTTTAATGTATGAAACTTTCTATTAAA
TTCCTGATTTTATTTCTGTAGGACTGAACGTCTTGCTCGAGATGTGATGAAGGAGATGGGAGGCCATC
ACATTGTAGCCCTCTGTGTGCTCAAGGGGGGCTATAAATTCTTTGCTGACCTGCTGGATTACATCAAA
GCACTGAATAGAAATAGTGATAGATCCATTCCTATGACTGTAGATTTTATCAGACTGAAGAGCTATTG
TGTGAGTATATTTAATATATGATTCTTTTTAGTGGCAACAGTAGGTTTTCTTATATTTTCTTTGAATC
TCTGCAAACCATACTTGCTTTCATTTCACTTGGTTACAGTGAGATTTTTCTAACATATTCACTAGTAC
TTTACATCAAAGCCAATACTGTTTTTTTAAAACTAGTCACCTTGGAGGATATATACTTATTTTACAGG
TGTGTGTGGTTTTTTAAATAAACTCCTTTTAGGAATTGCTGTTGGGACTTGGGATACTTTTTTCACTA
TACATACTGGTGACAGATACCCTCTCTTGAGCTACATCGGTTTGTGGGGAGTCAAAAGTCCTTTGGAG
CTAGGTTTGACAAATAAGGTGGGTTAACACTTGTTTCCTAGAAAGCACATGGAGAGCTAGAGTATTGG
CGAATTGAAGAAATCCCCCTTTTTTTTTAACACACTTAAGAAAGGGGACTGCAGGTATACTCAAGAGA
GTAAGTCGCACCAGAAACCACTTTTGATCCACAGTCTGCCTGTGTCACACAATTGAAATGCATCACAA
CATTGACACTGTGGATGAAACAAAATCAGTGTGAATTTTAGTAGTGAATTTCATTCATAATTTGATCG
TGCAAACGTTTGATTTTTATTACTTTAGACTATTGTTTCTGATTTTATGTTGGGTTGGTATTTCCTGT
GAGTTACTGTTTTACCTTTAAAATAGGAATTTTTCATACTCTTCAAAGATTAGAACAAATGTCCAGTT
TTTGCTGTTTCATGAATGAGTCCTGTCCATCTTTGTAGAAACTCGCCTTATGTTCACATTTTTATTGA
GAATAAGACCACTTATCTACATTTAACTATCAACCTCATCCTCTCCATTAATCATCTATTTTAGTGAC
CCAAGTTTTTGACCTTTTCCATGTTTACATCAATCCTGTAGGTGATTGGGCAGCCATTTAAGTATTAT
TATAGACATTTTCACTATCCCATTAAAACCCTTTATGCCCATACATCATAACACTACTTCCTACCCAT
AAGCTCCTTTTAACTTGTTAAAGTCTTGCTTGAATTAAAGACTTGTTTACGGTATCGATAAGCTTGAT
ATCAAAACGCCAACTTTGACCCGGAACGCGGAAAACACCTGAGAAAAACACCTGGGCGAGTCTCCACG
TAAACGGTCAAAGTCCCCGCGGCCCTAGACAAATATTACGCGCTATGAGTAACACAAAATTATTCAGA
TTTCACTTCCTCTTATTCAGTTTTCCCGCGAAAATGGCCAAATCTTACTCGGTTACGCCCAAATTTAC
TACAACATCCGCCTAAAACCGCGCGAAAATTGTCACTTCCTGTGTACACCGGCGCACACCAAAAACGT
CACTTTTGCCACATCCGTCGCTTACATGTGTTCCGCCACACTTGCAACATCACACTTCCGCCACACTA
CTACGTCACCCGCCCCGTTCCCACGCCCCGCGCCACGTCACAAACTCCACCCCCTCATTATCATATTG
GCTTCAATCCAAAATAAGGTATATTATTGATGATGTTTAAACATTAAGAATTAATTCGATCCTGAATG
GCGAATGGACGCGCCCTGTAGCGGCGCATTAAGCGCGCGGGTGTGGTGGTTACGCGCAGCGTGACCGC
TACACTTGCCAGCGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGAGCTTTACGGCACCTC
GACCGCAAAAAACTTGATTTGGGTGATGGTTCACGTAGTGGGCCATCGCCCTGATAGACGGTTTTTCG
CCCTTTGACGTTGGAGTCCACGTTCTTTAATAGTGGACTCTTGTTCCAAACTGGAACAACACTCAACC
CTATCGCGGTCTATTCTTTTGATTTATAAGGGATGTTGCCGATTTCGGCCTATTGGTTAAAAAATGAG
CTGATTTAACAAAAATTTTAACAAAATTCAGAAGAACTCGTCAAGAAGGCGATAGAAGGCGATGCGCT
GCGAATCGGGAGCGGCGATACCGTAAAGCACGAGGAAGCGGTCAGCCCATTCGCCGCCAAGCTCTTCA
GCAATATCACGGGTAGCCAACGCTATGTCCTGATAGCGGTCCGCCACACCCAGCCGGCCACAGTCGAT
GAATCCAGAAAAGCGGCCATTTTCCACCATGATATTCGGCAAGCAGGCATCGCCATGGGTCACGACGA
GATCCTCGCCGTCGGGCATGCTCGCCTTGAGCCTGGCGAACAGTTCGGCTGGCGCGAGCCCCTGATGC
TCTTCGTCCAGATCATCCTGATCGACAAGACCGGCTTCCATCCGAGTACGTGCTCGCTCGATGCGATG
TTTCGCTTGGTGGTCGAATGGGCAGGTAGCCGGATCAAGCGTATGCAGCCGCCGCATTGCATCAGCCA
TGATGGATACTTTCTCGGCAGGAGCAAGGTGAGATGACAGGAGATCCTGCCCCGGCACTTCGCCCAAT
AGCAGCCAGTCCCTTCCCGCTTCAGTGACAACGTCGAGCACAGCTGCGCAAGGAACGCCCGTCGTGGC
CAGCCACGATAGCCGCGCTGCCTCGTCTTGCAGTTCATTCAGGGCACCGGACAGGTCGGTCTTGACAA
AAAGAACCGGGCGCCCCTGCGCTGACAGCCGGAACACGGCGGCATCAGAGCAGCCGATTGTCTGTTGT
GCCCAGTCATAGCCGAATAGCCTCTCCACCCAAGCGGCCGGAGAACCTGCGTGCAATCCATCTTGTTC
AATCATGCGAAACGATCCTCATCCTGTCTCTTGATCAGAGCTTGATCCCCTGCGCCATCAGATCCTTG
GCGGCAAGAAAGCCATCCAGTTTACTTTGCAGGGCTTCCCAACCTTACCAGAGGGCGCCCCAGCTGGC
AATTCCGGTTCGCTTGCTGTCCATAAAACCGCCCAGTCTAGCTATCGCCATGTAAGCCCACTGCAAGC
TACCTGCTTTCTCTTTGCGCTTGCGTTTTCCCTTGTCCAGATAGCCCAGTAGCTGACATTCATCCGGG
GTCAGCACCGTTTCTGCGGACTGGCTTTCTACGTGAAAAGGATCTAGGTGAAGATCCTTTTTGATAAT
CTCATGGCTGCAGCAATGGCAACAACGTTGCGCAAACTATTAACTGGCGAACTACTTACTCTAGCTTC
CCGGCAACAATTAATAGACTGGATGGAGGCGGATAAAGTTGCAGGACCACTTCTGCGCTCGGCCCTTC
CGGCTGGCTGGTTTATTGCTGATAAATCTGGAGCCGGTGAGCGTGGGTCTCGCGGTATCATTGCAGCA
CTGGGGCCAGATGGTAAGCCCTCCCGTATCGTAGTTATCTACACGACGGGGAGTCAGGCAACTATGGA
TGAACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGCGTCAGAC
RightITR = first underlined and bold sequence
CBh = first underlined sequence
mCherry: PKD1 = first bold sequence
HGHpA = second underlined sequence
EF1α = second bold sequence
PKD2 = third underlined sequence
BGHpA = third bold sequence
Packaging Signal = fourth underlined sequence
LeftITR = second underlined and bold sequence
HDAd-SAM
RightITR-U6-sgRNA-CMV-dCas9VP64-HGHpA-EFlα-MPH-HGHpA-
PackagingSignal-LeftITR
SEQ ID NO: 8
CCCGTAGAAAAGATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCCGTAGTTAGGCCACCAC
TTCAAGAACTCTGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGCGCAGCGGTCGG
GCTGAACGGGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCTGATACCGCTCGCC
GCAGCCGAACGACCGAGCGCAGCGAGTCAGTGAGCGAGGAAGCGGAAGAGCGCCTGATGCGGTATTTT
CTCCTTACGCATCTGTGCGGTATTTCACACCGCATATGGATCCATGCATGTTAAGTTTAAACATCATC
AATAATATACCTTATTTTGGATTGAAGCCAATATGATAATGAGGGGGTGGAGTTTGTGACGTGGCGCG
GGGCGTGGGAACGGGGCGGGTGACGTAGGTTTTAGGGCGGAGTAACTTGTATGTGTTGGGAATTGTAG
TTTTCTTAAAATGGGAAGTTACGTAACGTGGGAAAACGGAAGTGACGATTTGAGGAAGTTGTGGGTTT
TTTGGCTTTCGTTTCTGGGCGTAGGTTCGCGTGCGGTTTTCTGGGTGTTTTTTGTGGACTTTAACCGT
TACGTCATTTTTTAGTCCTATATATACTCGCTCTGCACTTGGCCCTTTTTTACACTGTGACTGATTGA
GCTGGTGCCGTGTCGAGTGGTGTTTTTTGATGCCCCCCCTCGAGGTTCGACGGTATCGATAAGCTTGA
TTTAATTAAGGCCGGCCCCTAGGGGCGCGCGCGGCCGCTAGGGATAACAGGGTAATGAGGGCCTATTT
CCCATGATTCCTTCATATTTGCATATACGATACAAGGCTGTTAGAGAGATAATTGGAATTAATTTGAC
TGTAAACACAAAGATATTAGTACAAAATACGTGACGTAGAAAGTAATAATTTCTTGGGTAGTTTGCAG
TTTTAAAATTATGTTTTAAAATGGACTATCATATGCTTACCGTAACTTGAAAGTATTTCGATTTCTTG

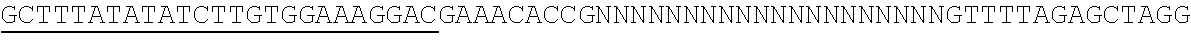

CCAACATGAGGATCACCCATGTCTGCAGGGCCTAGCAAGTTAAAATAAGGCTAGTCCGTTATCAACTT
GGCCAACATGAGGATCACCCATGTCTGCAGGGCCAAGTGGCACCGAGTCGGTGCTTTTTTTGGATCCT
GTTGACAATTAATCATCGGCATAGTATATCGGCATAGTATAATACGACAAGGTGAGGAACTAAACCAT
GGCCAAGTTGACCAGTGCCGTTCCGGTGCTCACCGCGCGCGACGTCGCCGGAGCGGTCGAGTTCTGGA
CCGACCGGCTCGGGTTCTCCCGGGACTTCGTGGAGGACGACTTCGCCGGTGTGGTCCGGGACGACGTG
ACCCTGTTCATCAGCGCGGTCCAGGACCAGGTGGTGCCGGACAACACCCTGGCCTGGGTGTGGGTGCG
CGGCCTGGACGAGCTGTACGCCGAGTGGTCGGAGGTCGTGTCCACGAACTTCCGGGACGCCTCCGGGC
CGGCCATGACCGAGATCGGCGAGCAGCCGTGGGGGCGGGAGTTCGCCCTGCGCGACCCGGCCGGCAAC
TGCGTGCACTTCGTGGCCGAGGAGCAGGACTGATAGGGATAACAGGGTAATGCTAGCATAGTAATCAA
TTACGGGGTCATTAGTTCATAGCCCATATATGGAGTTCCGCGTTACATAACTTACGGTAAATGGCCCG
CCTGGCTGACCGCCCAACGACCCCCGCCCATTGACGTCAATAATGACGTATGTTCCCATAGTAACGTC
AATAGGGACTTTCCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCCGCCTGGCATTAT
GCCCAGTACATGACCTTATGGGACTTTCCTACTTGGCAGTACATCTACGTATTAGTCATCGCTATTAC
CATGGTGATGCGGTTTTGGCAGTACATCAATGGGCGTGGATAGCGGTTTGACTCACGGGGATTTCCAA
GTCTCCACCCCATTGACGTCAATGGGAGTTTGTTTTGCACCAAAATCAACGGGACTTTCCAAAATGTC
GTAACAACTCCGCCCCATTGACGCAAATGGGCGGTAGGCGTGTACGGTGGGAGGTCTATATAAGCAGA
GCTCGTTTAGTGAACCGTCAGATCGCCTGGAGACGCCATCCACGCTGTTTTGACCTCCATAGAAGACA
CCGGGACCGATCCAGCCTCCGCGGATTCGAATCCCGGCCGGGAACGGTGCATTGGAACGCGGATTCCC
CGTGCCAAGAGTGACGTAAGTACCGCCTATAGAGTCTATAGGCCCACAAAAAATGCTTTCTTCTTTTA
ATATACTTTTTTGTTTATCTTATTTCTAATACTTTCCCTAATCTCTTTCTTTCAGGGCAATAATGATA
CAATGTATCATGCCTCTTTGCACCATTCTAAAGAATAACAGTGATAATTTCTGGGTTAAGGCAATAGC
AATATTTCTGCATATAAATATTTCTGCATATAAATTGTAACTGATGTAAGAGGTTTCATATTGCTAAT
AGCAGCTACAATCCAGCTACCATTCTGCTTTTATTTTATGGTTGGGATAAGGCTGGATTATTCTGAGT
CCAAGCTAGGCCCTTTTGCTAATCATGTTCATACCTCTTATCTTCCTCCCACAGCTCCTGGGCAACGT
GCTGGTCTGTGTGCTGGCCCATCACTTTGGCAAAGAATTGGGATCGTACGGCCACCATGAAAAGGCCG
GCGGCCACGAAAAAGGCCGGCCAGGCAAAAAAGAAAAAGGACAAGAAGTACAGCATCGGCCTGGCCAT
CGGCACCAACTCTGTGGGCTGGGCCGTGATCACCGACGAGTACAAGGTGCCCAGCAAGAAATTCAAGG
TGCTGGGCAACACCGACCGGCACAGCATCAAGAAGAACCTGATCGGAGCCCTGCTGTTCGACAGCGGC
GAAACAGCCGAGGCCACCCGGCTGAAGAGAACCGCCAGAAGAAGATACACCAGACGGAAGAACCGGAT
CTGCTATCTGCAAGAGATCTTCAGCAACGAGATGGCCAAGGTGGACGACAGCTTCTTCCACAGACTGG
AAGAGTCCTTCCTGGTGGAAGAGGATAAGAAGCACGAGCGGCACCCCATCTTCGGCAACATCGTGGAC
GAGGTGGCCTACCACGAGAAGTACCCCACCATCTACCACCTGAGAAAGAAACTGGTGGACAGCACCGA
CAAGGCCGACCTGCGGCTGATCTATCTGGCCCTGGCCCACATGATCAAGTTCCGGGGCCACTTCCTGA
TCGAGGGCGACCTGAACCCCGACAACAGCGACGTGGACAAGCTGTTCATCCAGCTGGTGCAGACCTAC
AACCAGCTGTTCGAGGAAAACCCCATCAACGCCAGCGGCGTGGACGCCAAGGCCATCCTGTCTGCCAG
ACTGAGCAAGAGCAGACGGCTGGAAAATCTGATCGCCCAGCTGCCCGGCGAGAAGAAGAATGGCCTGT
TCGGCAACCTGATTGCCCTGAGCCTGGGCCTGACCCCCAACTTCAAGAGCAACTTCGACCTGGCCGAG
GATGCCAAACTGCAGCTGAGCAAGGACACCTACGACGACGACCTGGACAACCTGCTGGCCCAGATCGG
CGACCAGTACGCCGACCTGTTTCTGGCCGCCAAGAACCTGTCCGACGCCATCCTGCTGAGCGACATCC
TGAGAGTGAACACCGAGATCACCAAGGCCCCCCTGAGCGCCTCTATGATCAAGAGATACGACGAGCAC
CACCAGGACCTGACCCTGCTGAAAGCTCTCGTGCGGCAGCAGCTGCCTGAGAAGTACAAAGAGATTTT
CTTCGACCAGAGCAAGAACGGCTACGCCGGCTACATTGACGGCGGAGCCAGCCAGGAAGAGTTCTACA
AGTTCATCAAGCCCATCCTGGAAAAGATGGACGGCACCGAGGAACTGCTCGTGAAGCTGAACAGAGAG
GACCTGCTGCGGAAGCAGCGGACCTTCGACAACGGCAGCATCCCCCACCAGATCCACCTGGGAGAGCT
GCACGCCATTCTGCGGCGGCAGGAAGATTTTTACCCATTCCTGAAGGACAACCGGGAAAAGATCGAGA
AGATCCTGACCTTCCGCATCCCCTACTACGTGGGCCCTCTGGCCAGGGGAAACAGCAGATTCGCCTGG
ATGACCAGAAAGAGCGAGGAAACCATCACCCCCTGGAACTTCGAGGAAGTGGTGGACAAGGGCGCTTC
CGCCCAGAGCTTCATCGAGCGGATGACCAACTTCGATAAGAACCTGCCCAACGAGAAGGTGCTGCCCA
AGCACAGCCTGCTGTACGAGTACTTCACCGTGTATAACGAGCTGACCAAAGTGAAATACGTGACCGAG
GGAATGAGAAAGCCCGCCTTCCTGAGCGGCGAGCAGAAAAAGGCCATCGTGGACCTGCTGTTCAAGAC
CAACCGGAAAGTGACCGTGAAGCAGCTGAAAGAGGACTACTTCAAGAAAATCGAGTGCTTCGACTCCG
TGGAAATCTCCGGCGTGGAAGATCGGTTCAACGCCTCCCTGGGCACATACCACGATCTGCTGAAAATT
ATCAAGGACAAGGACTTCCTGGACAATGAGGAAAACGAGGACATTCTGGAAGATATCGTGCTGACCCT
GACACTGTTTGAGGACAGAGAGATGATCGAGGAACGGCTGAAAACCTATGCCCACCTGTTCGACGACA
AAGTGATGAAGCAGCTGAAGCGGCGGAGATACACCGGCTGGGGCAGGCTGAGCCGGAAGCTGATCAAC
GGCATCCGGGACAAGCAGTCCGGCAAGACAATCCTGGATTTCCTGAAGTCCGACGGCTTCGCCAACAG
AAACTTCATGCAGCTGATCCACGACGACAGCCTGACCTTTAAAGAGGACATCCAGAAAGCCCAGGTGT
CCGGCCAGGGCGATAGCCTGCACGAGCACATTGCCAATCTGGCCGGCAGCCCCGCCATTAAGAAGGGC
ATCCTGCAGACAGTGAAGGTGGTGGACGAGCTCGTGAAAGTGATGGGCCGGCACAAGCCCGAGAACAT
CGTGATCGAAATGGCCAGAGAGAACCAGACCACCCAGAAGGGACAGAAGAACAGCCGCGAGAGAATGA
AGCGGATCGAAGAGGGCATCAAAGAGCTGGGCAGCCAGATCCTGAAAGAACACCCCGTGGAAAACACC
CAGCTGCAGAACGAGAAGCTGTACCTGTACTACCTGCAGAATGGGCGGGATATGTACGTGGACCAGGA
ACTGGACATCAACCGGCTGTCCGACTACGATGTGGACCACATCGTGCCTCAGAGCTTTCTGAAGGACG
ACTCCATCGACAACAAGGTGCTGACCAGAAGCGACAAGGCCCGGGGCAAGAGCGACAACGTGCCCTCC
GAAGAGGTCGTGAAGAAGATGAAGAACTACTGGCGGCAGCTGCTGAACGCCAAGCTGATTACCCAGAG
AAAGTTCGACAATCTGACCAAGGCCGAGAGAGGCGGCCTGAGCGAACTGGATAAGGCCGGCTTCATCA
AGAGACAGCTGGTGGAAACCCGGCAGATCACAAAGCACGTGGCACAGATCCTGGACTCCCGGATGAAC
ACTAAGTACGACGAGAATGACAAGCTGATCCGGGAAGTGAAAGTGATCACCCTGAAGTCCAAGCTGGT
GTCCGATTTCCGGAAGGATTTCCAGTTTTACAAAGTGCGCGAGATCAACAACTACCACCACGCCCACG
ACGCCTACCTGAACGCCGTCGTGGGAACCGCCCTGATCAAAAAGTACCCTAAGCTGGAAAGCGAGTTC
GTGTACGGCGACTACAAGGTGTACGACGTGCGGAAGATGATCGCCAAGAGCGAGCAGGAAATCGGCAA
GGCTACCGCCAAGTACTTCTTCTACAGCAACATCATGAACTTTTTCAAGACCGAGATTACCCTGGCCA
ACGGCGAGATCCGGAAGCGGCCTCTGATCGAGACAAACGGCGAAACCGGGGAGATCGTGTGGGATAAG
GGCCGGGATTTTGCCACCGTGCGGAAAGTGCTGAGCATGCCCCAAGTGAATATCGTGAAAAAGACCGA
GGTGCAGACAGGCGGCTTCAGCAAAGAGTCTATCCTGCCCAAGAGGAACAGCGATAAGCTGATCGCCA
GAAAGAAGGACTGGGACCCTAAGAAGTACGGCGGCTTCGACAGCCCCACCGTGGCCTATTCTGTGCTG
GTGGTGGCCAAAGTGGAAAAGGGCAAGTCCAAGAAACTGAAGAGTGTGAAAGAGCTGCTGGGGATCAC
CATCATGGAAAGAAGCAGCTTCGAGAAGAATCCCATCGACTTTCTGGAAGCCAAGGGCTACAAAGAAG
TGAAAAAGGACCTGATCATCAAGCTGCCTAAGTACTCCCTGTTCGAGCTGGAAAACGGCCGGAAGAGA
ATGCTGGCCTCTGCCGGCGAACTGCAGAAGGGAAACGAACTGGCCCTGCCCTCCAAATATGTGAACTT
CCTGTACCTGGCCAGCCACTATGAGAAGCTGAAGGGCTCCCCCGAGGATAATGAGCAGAAACAGCTGT
TTGTGGAACAGCACAAGCACTACCTGGACGAGATCATCGAGCAGATCAGCGAGTTCTCCAAGAGAGTG
ATCCTGGCCGACGCTAATCTGGACAAAGTGCTGTCCGCCTACAACAAGCACCGGGATAAGCCCATCAG
AGAGCAGGCCGAGAATATCATCCACCTGTTTACCCTGACCAATCTGGGAGCCCCTGCCGCCTTCAAGT
ACTTTGACACCACCATCGACCGGAAGAGGTACACCAGCACCAAAGAGGTGCTGGACGCCACCCTGATC
CACCAGAGCATCACCGGCCTGTACGAGACACGGATCGACCTGTCTCAGCTGGGAGGCGACAGCGCTGG
AGGAGGTGGAAGCGGAGGAGGAGGAAGCGGAGGAGGAGGTAGCGGACCTAAGAAAAAGAGGAAGGTGG
CGGCCGCTGGATCCGGACGGGCTGACGCATTGGACGATTTTGATCTGGATATGCTGGGAAGTGACGCC
CTCGATGATTTTGACCTTGACATGCTTGGTTCGGATGCCCTTGATGACTTTGACCTCGACATGCTCGG
CAGTGACGCCCTTGATGATTTCGACCTGGACATGCTGATTAACTGTACATAAACGGGTGGCATCCCTG
TGACCCCTCCCCAGTGCCTCTCCTGGCCCTGGAAGTTGCCACTCCAGTGCCCACCAGCCTTGTCCTAA
TAAAATTAAGTTGCATCATTTTGTCTGACTAGGTGTCCTTCTATAATATTATGGGGTGGAGGGGGGTG
GTATGGAGCAAGGGGCAAGTTGGGAAGACAACCTGTAGGGCCTGCGGGGTCTATTGGGAACCAAGCTG
GAGTGCAGTGGCACAATCTTGGCTCACTGCAATCTCCGCCTCCTGGGTTCAAGCGATTCTCCTGCCTC
AGCCTCCCGAGTTGTTGGGATTCCAGGCATGCATGACCAGGCTCAGCTAATTTTTGTTTTTTTGGTAG
AGACGGGGTTTCACCATATTGGCCAGGCTGGTCTCCAACTCCTAATCTCAGGTGATCTACCCACCTTG
GCCTCCCAAATTGCTGGGATTACAGGCGTGAACCACTGCTCCCTTCCCTGTCCTTGAATTCTAACTAT
AACGGTCCTAAGGTAGCGAAGCTAGCTGCAAAGATGGATAAAGTTTTAAACAGAGAGGAATCTTTGCA
GCTAATGGACCTTCTAGGTCTTGAAAGGAGTGGGAATTGGCTCCGGTGCCCGTCAGTGGGCAGAGCGC
ACATCGCCCACAGTCCCCGAGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTTGCCGCCAGAACACAGGTA
AGTGCCGTGTGTGGTTCCCGCGGGCCTGGCCTCTTTACGGGTTATGGCCCTTGCGTGCCTTGAATTAC
TTCCACCTGGCTGCAGTACGTGATTCTTGATCCCGAGCTTCGGGTTGGAAGTGGGTGGGAGAGTTCGA
GGCCTTGCGCTTAAGGAGCCCCTTCGCCTCGTGCTTGAGTTGAGGCCTGGCCTGGGCGCTGGGGCCGC
CGCGTGCGAATCTGGTGGCACCTTCGCGCCTGTCTCGCTGCTTTCGATAAGTCTCTAGCCATTTAAAA
TTTTTGATGACCTGCTGCGACGCTTTTTTTCTGGCAAGATAGTCTTGTAAATGCGGGCCAAGATCTGC
ACACTGGTATTTCGGTTTTTGGGGCCGCGGGCGGCGACGGGGCCCGTGCGTCCCAGCGCACATGTTCG
GCGAGGCGGGGCCTGCGAGCGCGGCCACCGAGAATCGGACGGGGGTAGTCTCAAGCTGGCCGGCCTGC
TCTGGTGCCTGGCCTCGCGCCGCCGTGTATCGCCCCGCCCTGGGCGGCAAGGCTGGCCCGGTCGGCAC
CAGTTGCGTGAGCGGAAAGATGGCCGCTTCCCGGCCCTGCTGCAGGGAGCTCAAAATGAAGGACGCGG
CGCTCGGGAGAGCGGGCGGGTGAGTCACCCACACAAAGGAAAAGGGCCTTTCCGTCCTCAGCCGTCGC
TTCATGTGACTCCACGGAGTACCGGGCGCCGTCCAGGCACCTCGATTAGTTCTCGAGCTTTTGGAGTA
CGTCGTCTTTAGGTTGGGGGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCTTTTTGAGTTTGGATCTTG
GTTCATTCTCAAGCCTCAGACAGTGGTTCAAAGTTTTTTTCTTCCATTTCAGGTGTCGTGACGTACGG
CCACCATGGCTTCAAACTTTACTCAGTTCGTGCTCGTGGACAATGGTGGGACAGGGGATGTGACAGTG
GCTCCTTCTAATTTCGCTAATGGGGTGGCAGAGTGGATCAGCTCCAACTCACGGAGCCAGGCCTACAA
GGTGACATGCAGCGTCAGGCAGTCTAGTGCCCAGAAGAGAAAGTATACCATCAAGGTGGAGGTCCCCA
AAGTGGCTACCCAGACAGTGGGCGGAGTCGAACTGCCTGTCGCCGCTTGGAGGTCCTACCTGAACATG
GAGCTCACTATCCCAATTTTCGCTACCAATTCTGACTGTGAACTCATCGTGAAGGCAATGCAGGGGCT
CCTCAAAGACGGTAATCCTATCCCTTCCGCCATCGCCGCTAACTCAGGTATCTACAGCGCTGGAGGAG
GTGGAAGCGGAGGAGGAGGAAGCGGAGGAGGAGGTAGCGGACCTAAGAAAAAGAGGAAGGTGGCGGCC
GCTGGATCCCCTTCAGGGCAGATCAGCAACCAGGCCCTGGCTCTGGCCCCTAGCTCCGCTCCAGTGCT
GGCCCAGACTATGGTGCCCTCTAGTGCTATGGTGCCTCTGGCCCAGCCACCTGCTCCAGCCCCTGTGC
TGACCCCAGGACCACCCCAGTCACTGAGCGCTCCAGTGCCCAAGTCTACACAGGCCGGCGAGGGGACT
CTGAGTGAAGCTCTGCTGCACCTGCAGTTCGACGCTGATGAGGACCTGGGAGCTCTGCTGGGGAACAG
CACCGATCCCGGAGTGTTCACAGATCTGGCCTCCGTGGACAACTCTGAGTTTCAGCAGCTGCTGAATC
AGGGCGTGTCCATGTCTCATAGTACAGCCGAACCAATGCTGATGGAGTACCCCGAAGCCATTACCCGG
CTGGTGACCGGCAGCCAGCGGCCCCCCGACCCCGCTCCAACTCCCCTGGGAACCAGCGGCCTGCCTAA
TGGGCTGTCCGGAGATGAAGACTTCTCAAGCATCGCTGATATGGACTTTAGTGCCCTGCTGTCACAGA
TTTCCTCTAGTGGGCAGGGAGGAGGTGGAAGCGGCTTCAGCGTGGACACCAGTGCCCTGCTGGACCTG
TTCAGCCCCTCGGTGACCGTGCCCGACATGAGCCTGCCTGACCTTGACAGCAGCCTGGCCAGTATCCA
AGAGCTCCTGTCTCCCCAGGAGCCCCCCAGGCCTCCCGAGGCAGAGAACAGCAGCCCGGATTCAGGGA
AGCAGCTGGTGCACTACACAGCGCAGCCGCTGTTCCTGCTGGACCCCGGCTCCGTGGACACCGGGAGC
AACGACCTGCCGGTGCTGTTTGAGCTGGGAGAGGGCTCCTACTTCTCCGAAGGGGACGGCTTCGCCGA
GGACCCCACCATCTCCCTGCTGACAGGCTCGGAGCCTCCCAAAGCCAAGGACCCCACTGTCTCCTGTA
CATAAACGGGTGGCATCCCTGTGACCCCTCCCCAGTGCCTCTCCTGGCCCTGGAAGTTGCCACTCCAG
TGCCCACCAGCCTTGTCCTAATAAAATTAAGTTGCATCATTTTGTCTGACTAGGTGTCCTTCTATAAT
ATTATGGGGTGGAGGGGGGTGGTATGGAGCAAGGGGCAAGTTGGGAAGACAACCTGTAGGGCCTGCGG
GGTCTATTGGGAACCAAGCTGGAGTGCAGTGGCACAATCTTGGCTCACTGCAATCTCCGCCTCCTGGG
TTCAAGCGATTCTCCTGCCTCAGCCTCCCGAGTTGTTGGGATTCCAGGCATGCATGACCAGGCTCAGC
TAATTTTTGTTTTTTTGGTAGAGACGGGGTTTCACCATATTGGCCAGGCTGGTCTCCAACTCCTAATC
TCAGGTGATCTACCCACCTTGGCCTCCCAAATTGCTGGGATTACAGGCGTGAACCACTGCTCCCTTCC
CTGTCCTTGAATTCTAACTATAACGGTCCTAAGGTAGCGAAGTCGACCGAATCGTTGTCCCTTGTCAC
AGCCATTGAGAATTTTGGCAGGGAGCATGTTCTTAGAGCATTTTTAGGCTCTGCGGGACATAACAGCT
CTGCCTCAGAGCACATGCCTTTCTCAGCTCCTGAAAGCCACTGATCAAATTGGAACATTTTGTACCTT
AGGGATGAGGATATCAACTCTCCCAGCCACTTAGAGGGATAAATGTGATGATGCATTCAATTGTGACT
ACATCTGATCCCAACTGTTGCTTCAGCTGCTCTCCTATAGCACATGGCGGGAGGCGTGCATCCCAGTA
GCTACCTCCCCACTTTTGGGGAGATGTGGTTCCATCCATGAAACCTGGGTACCCGCCTACCAGGTCCT
GGCCTATCAGGTGGCAGGGTCTGGTCAAAGAAGGGCATGTGTGGTCTTCAGCAAGGGAGACAGGACGG
TGGTGCAGAGCGTCTAGACCCTCAGGGCAAGTCTCCCCCACACCTGCTCCCGGGGCAGTTGTCTTTGT
GACCTCCCATCCCCCTCTGTTTCATCCTCTATAAAATGAGGGGCTGAGCCCCAAAATAACAGGCTTCT
TTGCCATGATGCAAAACTGCTGAATCTTTCTTTCTGACACACAAGGCATCGAGCAGCCTCTGAAAGAA
CCAAAGCCACTAGCAGGCTTCCTGACTTGGGTTTGTAGGTACTGAATACTCCCTTGAAAAATAAAAAC
ATAGAGGCACTTTTCTCCTGGCTGTTTATTACAGAACGAAGAAAAAACACACTGGCTTGAAACAGACG
CCAGATTTCAAATGTAGAGGTGAAATACGAGGTGGCAATTAAAATGTGATTACAGAAAGTCTGGACAC
TGAGAAAAGTTTACAGGACAGTGGGTGTGGGTTTTCTATAACAGACACTTAAATATACATGACGATAA
TTGCAGATAGAAACCATCAAAGACAAACCCCAAATCAACTAATAATGTTTACAGATGTTCCCCCCCAA
ACCACAGAGCCTTACATCAAAACAAATACTGAAAGGCTTTAAACCAGGAACAGCTCGCCTTAACCCCA
CGAGGGTGCACACAAGCTGGGCTTTTTCTCTCGGTCTGAATGGTAAAGGGAGGAGGATACTCTAGCTC
CTCCAGGTGGATTGCTGAGACAGGGCTCGGCTCACACACTGTCTCTGCGCCTCTCCCAAATCTGGAGA
ACTCTCCCAGCCTCCTGGTAAAGTGTCTCTGTGGGGCACTTAACGATAAAACAGCTTCTGCTGTAAAG
CTCATTAGGAAAGAGCTAGCGGAGACTGAAAGGTTCGCAAAAGAGATTAAGAATCACACAAGGCAATA
GGATTTTTAGTGAACATAGAAATAAATGGCCAAGTGGTTTTCTATTTGGCATTTGTCAACTTGCACAA
CAACTCTTGGTCATATCCACATTGCTCATTGCATTAAAACCATAAGCGACTCAGCCACCTAGCTTAAC
AAGGTATCACTGGAGCAAACAACACGGTCTGCATATTTGTAACATTGTATAATAAACACAAAACAATG
CATAGTAAACACAACTCTACTGAAACAAAAGCCGTCGCTTTATTTACAAAGTCACAAAATGAAGTATA
AATACTTCTGTCATTAATGTTTAGGAAAACCATTTACAAAATTTTCAAATATGTACACGTAGCTTGAA
AAATCACCAGCTTTCCATTTTGTCACAGGTAGAGAGAGGGATAAGCATGGGCTGACAACACCACTCAA
ATTGTAACGGGAGACAACTGCGGGTATGGATCGACACCACTTCCTAGAGTGATGTCACCATGGGGGTT
TCTATGGGCATCCTGCTCAGATTTAAAGTGCCCCAGCATCCTGGGTGACTTGCCCAGAATTCTGGGCT
GTGGCATTTTGAGCAGCAGCATGCTGTTCCAAAATGTCGTCGATCAGCCTCAAGTTGCACACCCAGTC
TTCATCTGGGCTCACACAGGAGCCTTTCAAGAGAGCTTCAATGAAATCTACCTCATTGCAGTCAGGTG
ACGAAATCAGATCATTTAGTGGGGGTTGGGGCTGGCGCAAAAAGTCGGCAGGTGGCAGCTCAGGGGGA
ATATCCGTTCTGTCGAACGGACCTGGGAACTGGCTGGCAGCAACGGCAGAAGCAGCAGCAGCGGTGGC
AGCAGCAGCCACATAGCTTGGTGGCTCGATGCCCTGTATGGGGCTCAGGGGACTAAAGCTGGCCATAC
CCTGCTGGAGGAACTTGGTGGTGTTTGCTACAGGCACCGGGCCCTGTACCGGGCTCTGCCTGAGGCTC
TGGCTGCCCAGCAGGCTGAAGCTGGGGTTGTTGGCCAGGGGCACTTGTGTTCCCATCGCAGCGGGCAC
TTGTGCCTCCCAATCAGATGGCCTCTGAAGGCAGGCCTGGCCAGAAGGTGAGTGCTGCTGAACGCTAT
TATCCACTTGGCTGAGGGGTGTTTTCCCCGAAACTGCTGTGGTCACAGCTGCTGCCGCTGTGACCCAT
GCAGCATTGTTGAACGCAGTGGGCATTCTTGGCACACTAGGCCGTCTGAGCTGGTGGGGACTCAAGGA
CTGGGTGCCCAGGGAGCTGGGACAGAACCCAGGCAGGGGCACTTCTGGTGGGGTGGCCTTGGGGCTCT
GCATATGCTGGCAGACAGAGTCAAGTCTGCCCAGGGGAGTCTGGCCTGAGTGTGAGAGGATGGGACAC
TGGGGGCTGGAGGTGAAAATTCCTTGCCGCTTCCCCAGAGTTGGTGAGATCACTCCCATGCCCTCGCA
GCTCTGGTGCCTGGTGAGTGGGATCATTCCTGGACTCAGATTGTTCTGAAGAAGCCCAGTTCTGGGTG
GCATCAAGTGCTTGCTAGATGGGGGGCTTGCCTTGATCCGGCTACACTTGGAGGTGACTTGTTCTTGG
ACGGCTACATACAGAAAGAGAGAAGTGGGGATGAGTTCCAAAGGCATCCTCGACTTCGGCTGTGGCCA
CCGGAGGGTAGCTCCTGGCCCAACACGGACTTCTCACCTCCCGCCCTTGGCTCTCTACTGAGCTCCCC
CCTGCTCCCCAATTCCTCGCCATTCCCCTCATTTCTCTGCCCTCAGCCTGGACTGCAGTTCTTCTGGG
AAGCTGCCCCAACTCCCTAGGTCTGTGCTCACCAAGAGCAGATCACACTGGACTGAAATGCCAGCTGA
TTTGTCTCTTCAAGAAAATTGGAAGCTCCTGGAGGTCAGGGTCCATGTCTGCTTTTACACTCAGTGCT
CTGTATGCAGGCCTGGCACTGCCCACCCTTTGACAGGTGGTGCATATTTTGTAGAAGGAAGGAAGGGG
CCAGGTGGGGTGGGCTGGGCTGGTGGCGGGAGCTAGCTCAGCCTCTTAGATTCTCTACCCGATGGATG
TGACCTGGGACAGCAAGTGAGTGTGGTGAGTGAGTGCAGACGGTGCTTTGTTCCCCTCTTGTCTCATA
GCCTAGATGGCCTCTGAGCCCAGATCTGGGGCTCAGACAACATTTGTTCAACTGAACGGTAATGGGTT
TCCTTTCTGAAGGCTGAAATCTGGGAGCTGACATTCTGGACTCCCTGAGTTCTGAAGAGCCTGGGGAT
GGAGAGACACGGAGCAGAAGATGGAAGGTAGAGTCCCAGGTGCCTAAGATGGGGAATACATCTCCCCT
CATTGTCATGAGAGTCCACTCTAGCTGATATCTACTGTGGCCAATATCTACCGGTACTTTTTTGGGGT
GGACACTGAGTCATGCAGCAGTCTTATGGTTTACCCAAGGTCAGGTAGGGGAGACAGTGCAGTCAGAG
CACAAGCCCAGTGTGTCTGACCCACCCAAGAATCCATGCTCGTATCTACAAAAATGATTTTTTCTCTT
GTAATGGTGCCTAGGTTCTTTTATTATCATGGCATGTGTATGTTTTTCAACTAGGTTACAATCTGGCC
TTATAAGGTTAACCTCCTGGAGGCCACCAGCCTTCCTGAAACTTGTCTGTGCTGTCCCTGCAACTGGA
GTGTGCCTGATGTGGCACTCCAGCCTGGACAAGTGGGACACAGACTCCGCTGTTATCAGGCCCAAAGA
TGTCTTCCATAAGACCAGAAGAGCAATGGTGTAGAGGTGTCATGGGCTACAATAAAGATGCTGACCTC
CTGTCTGAGGGCAAGCAGCCTCTTCTGGCCCTCAGACAAATGCTGAGTGTTCCCAAGACTACCCTCGG
CCTGGTCCAATCTCATCCCACTGGTGCGTAAGGGTTGCTGAACTCATGACTTCTTGGCTAGCCTGCAA
CCTCCACGGAGTGGGAACTACATCAGGCATTTTGCTAACTGCTGTATCCTAGGCCAATAAATGTTGAT
CACATTTATAGCTGCCATGGTAGGGTGGGGACCCCTGCTATCTATCTGTGGAGGCTCTGGGAGCCCCT
GACACAAACTTTCTGAAGCAGAGCCTCCCCAACCCCTTTTCCATTCCCTATACCTGACAGATGGCCCA
GGAACCCATTAGAAATGGAAGGTCACTGCAGCAGTATGTGAATGTGCGTGTGGGAGAAGGGCAGGATC
AGAGCCCTGGGGGTGTGGCAGCCCCCAAGTGATTCTAATCCAGATCCTAGGGTTGTTTCCCTGTCCCA
TTGAAATAGCTGCTTTAAGGGGCCTGACTCAGGGAAATCAGTCTCTTGAATTAAGTGGTGATTTTGGA
GTCATTTAGACCAGGCCTTCAATTGGGATCCACTAGTTCTAGAGCGGCCGGGCCCAGGGAACCCCGCA
GGCGGGGGCGGCCAGTTTCCCGGGTTCGGCTTTACGTCACGCGAGGGCGGCAGGGAGGACGGAATGGC
GGGGTTTGGGGTGGGTCCCTCCTCGGGGGAGCCCTGGGAAAAGAGGACTGCGTGTGGGAAGAGAAGGT
GGAAATGGCGTTTTGGTTGACATGTGCCGCCTGCGAGCGTGCTGCGGGGAGGGGCCGAGGGCAGATTC
GGGAATGATGGCGCGGGGTGGGGGCGTGGGGGCTTTCTCGGGAGAGGCCCTTCCCTGGAAGTTTGGGG
TGCGATGGTGAGGTTCTCGGGGCACCTCTGGAGGGGCCTCGGCACGGAAAGCGACCACCTGGGAGGGC
GTGTGGGGACCAGGTTTTGCCTTTAGTTTTGCACACACTGTAGTTCATCTTTATGGAGATGCTCATGG
CCTCATTGAAGCCCCACTACAGCTCTGGTAGCGGTAACCATGCGTATTTGACACACGAAGGAACTAGG
GAAAAGGCATTAGGTCATTTCAAGCCGAAATTCACATGTGCTAGAATCCAGATTCCATGCTGACCGAT
GCCCCAGGATATAGAAAATGAGAATCTGGTCCTTACCTTCAAGAACATTCTTAACCGTAATCAGCCTC
TGGTATCTTAGCTCCACCCTCACTGGTTTTTTCTTGTTTGTTGAACCGGCCAAGCTGCTGGCCTCCCT
CCTCAACCGTTCTGATCATGCTTGCTAAAATAGTCAAAACCCCGGCCAGTTAAATATGCTTTAGCCTG
CTTTATTATGATTATTTTTGTTGTTTTGGCAATGACCTGGTTACCTGTTGTTTCTCCCACTAAAACTT
TTTAAGGGCAGGAATCACCGCCGTAACTCTAGCACTTAGCACAGTACTTGGCTTGTAAGAGGTCCTCG
ATGATGGTTTGTTGAATGAATACATTAAATAATTAACCACTTGAACCCTAAGAAAGAAGCGATTCTAT
TTCATATTAGGCATTGTAATGACTTAAGGTAAAGAGCAGTGCTATTAACGGAGTCTAACTGGGAATCC
AGCTTGTTTGGGCTATTTACTAGTTGTGTGGCTGTGGGCAACTTACTTCACCTCTCTGGGCTTAAGTC
ATTTTATGTATATCTGAGGTGCTGGCTACCTCTTGGAGTTATTGAGAGGATTATAAGACAGTCTATGT
GAATCAGCAACCCTTGCATGGCCCCTGGCGGGGAACAGTAATAATAGCCATCATCATGTTTACTTACA
TAGTCCTAATTAGTCTTCAAAACAGCCCTGTAGCAATGGTATGATTATTACCATTTTACAGATGAGGA
ACCTTTGAAGCCTCAGAGAGGCTAACAGACATACCCTAGGTCATACAGTTATTAAGAGAAGGAGCTCT
GTCTCGAACCTAGCTCTCTCTCTCTCGAGTAATACCAGTTAAAAAATAGGCTACAAATAGGTACTCAA
AAAAATGGTAGTGGCTGTTGTTTTTATTCAGTTGCTGAGGAAAAAATGTTGATTTTTCATCTCTAAAC
ATCAACTTACTTAATTCTGCCAATTTCTTTTTTTTGAGACAGGGTCTCACTCTGTCACCTAGGATGGA
GTGCAGTGGCACAATCACTGCTCACTGCAGCCTCGACTTCCCGGGCTCGGGTGATTCTCCCCAGGCTC
AGGGGATTCTCCCACTTCAGCCTCCCAAGTAGCTGGGACTACAGGTGCGCACCACCATCCCTGGCTAA
TATTTGTACTTTATTTTATTTATTTATTTATTTATTTTTTGAGATGGAGTTTCGCTCTTGTTGCCCGG
GCTGGAGTACAGTGGCATGATCTCGGCTCAGTGCAACCTCTGCCTCCCGGGTTCAAGCGATTCTCCTA
CCTCATCCCCCTGAGTAGCTGGGATTACAGGCGCCTGCCACCATGCCTGGCTAATTTTTTGTATTTTT
AATAGAGACGAGGTTTCACCATGTTGGCCAGGCTACTCTCGAACTCCTGATCTCAGGTGATCCACCCG
CCTTGGCCTCCCAAAGTGCTGGGATTACAGGCGTGAGCCACTGCGCCCGGCCTAATATTTGTATTTTT
TGTAGAGATGGTGTTTTGCCATGTTGTCCAGGCTGGTCTTGAACTCCTGAGCTCAAGCGATCTGCCCG
CCTCTGCTTCCCAAAGTGCTGGGATTACAGGCATGAGCCACCGTGCCTGGCCTAGGTAGACGCTTTTA
GCTTTGGGGTGTGATGCCTGCCCCAGTATATAGTGAATTTAATTATTGCTAGAGCTGGCTGTTTGTTA
GTTTTCTTTGAACATAAGATACTCATTGTTTTTAGTTTGCAAATCCCTCTTCCTTTTTAAAAAATTTC
TTTCCCTTAAATTGTTTGCATGTTAGCAATAACAAATGCTTAAATGGTGCTATGTGCTAGATACTCTT
CTAAGCCCTGTTATGTATATTAACTAATTTTTTAAATTACACAAATCAGAGAGGTTAAGTAACTTGCC
CAAGATTACCCAACAATACTAGGATTTGAACCTAAGTTTGTCTCACCCCAGATTCTGCTCTTAATCTC
TAAACTTTTAAGTTAGTAGTGACAATAGTAGGTATTTATTGAATACTTAACTATGTTTTAGGCGTTGA
AGTAAATATTTTGCAGGCATTATCTAATGTAAACACCCTAAAGTTACATAACAGGTACCCTTTAGGTA
AATAAACACTAGTATGACCTTGGAGGCACAGATAGTTGAAGTAACTTGCCCAATATCACTTACATGAA
ATTGGCCCTCAAATGTGTCTGATACAACCCATGCTGCTTGTAACTATCGTTTTAAACTGCCAGGGTAA
ACTTGGACACACTTGAGCTAAGAAAAAGCTTTTAGATTTTTGCAAATTAATGTGAAAGATATGCTTTA
TGTGGATATAATATCTTCTAAATTTCGGGGATGGTAGTCCTAGAAATGTAATCCTGCCCTAGCCGAGC
TTACCCTGCCAATAATTTTTTACAGAATTGGTAAAACGGAGCACCTTTTTTTTGTCCTTGGCCACACT
GTTATCAACAGGGTGTAGATTGACATCAATCTGTAGGTGTAAACCAGAATTACTCTTTGTGACCACCA
GGAAATAGAGCAGTTCAGTTCAGGGGTTTCTTTCTGTGAATTTAGCACTGTGACCTGCATACTACAAG
TCTACTTTGTTTTCTATCCATTGTTTGTATCTGGGTATTGCAAAAGGTAGGAAAAGGACCAACCAGAT
CAGCAGAGAAGAGTTGCCTTGGAGTTTTCTTTTAGTTTTCTGCAGTTCATTAGATAGTAACTAGGCCA
TGTCATTTTACTCCCTTGTAGTGAAGATATGTTGAAGTTGTACTGGTATACTCTTCTACCTTTCTGTA
ATTTTATATTGTGTAGACTTGATAAAATTTATGTGTCAATCACCACCATTAATATCAATATTGAGCCT
CAATTCTTATTTTTCTGCCCAGTGGCTGCCAAATTACTAACATTTACAATAATTCACTACTACTAAGA
TAATCTACTAGTTCGATCACATACTTCAAATTGTTATGGAACTACTGTCTTCAGCATTGTGCTTCTGA
TAACTGATAAGTATAATTTTTTTTTTGTCCAGAGTGAACATGTCTATTCTTCCACTGTACACACTAAT
AAAAGGAAAAATTGTAATATTGGGTAAATTCATGTCCTTACACATGTAGTAGTTATGAGCCCATGTCC
CTAGAATGAGTAATAATTTATCCCTCCCTTGGTTGAATAGTCAAGAATGCTGATTTTAATTCTTCTAA
CAGCTTTATCCCTCAGAAGGGAAGGCAAGCAAGTTATATATGTAGTTTATTTGTAAGACTGATATGAA
ATTGGAAGATGAATCTACTATTAGCTTTAATTATTTTTACATTTAGGAATATTGCATCAGTAACTCAT
AATTTTGGTTTTCTGTTATCCTGAGTTAACACAAATTATCCAAGGAGATGGCGGATCATCTGCTTTGA
GGTGTTTTTTTTTGAGAATTTTAATGTATCTGAATATAAAAGGTAAAAATATGCCAACTAGCAATTTC
TGCCCATTCCAGAAGTTTGGAAATATTACTCATTACTAGGAATTAAATAAAATATGGTTTATCTATTG
TTATACCTCTTTTAATTCACATAGCTCATTTTTATCTTTTATTTTTGTTTGTTTTTTTTGAGATGGAG
TCTTGCTCTGTCACCAGGCAGGAGTGCAGTGATGCAAATCTCGGCTCACTCTAGCCACCGACTCCCTG
GTTCAAGCGATTCTCCTGCCTGAGCCTTCTGAGTAGCTGGGATTACAGGCAGGCACCACCACGCCCAG
CTAATTTTTGTAGAGACAGGATTTCACCGTGTTGGCCAGGATGGTCTCCATCTCCTGACCTCATGATC
TGCCTGCTTCGGCCTCCCAAAGTGCTGGGATTACAGGTGGGAGCCACTACGCCTGGCCCACATAGCTC
ATTTTTAGACTCACTTCCATTAAGTCTTGTTTGGACCCACGAACATTGTCTTTTTTTTTTTAAGATGG
AGTTTCACTTTTGTTGCCCAGACTGTAGTGCAATGGTGCAATCTCAGCTCACTGCAATCTCTGCCTCC
TGGGTTCTAGCAATTCTCCTGCCTCAGCCTCCCGAGTAGCTGGAATTACAGGCGCCCGCCACCACGCC
CAGCTAATTTTTGTGTTTTTAGTAGAGACGGGGTTTCACCATGTTGGGCAGGCCAGGGGTGATCCGCC
CACCTCAGCCTCCCAAAGTGCTGGGATTACAGGTGTGAGCCACCGCATCTGGCCAACATGTCTTTTTT
TTTTTTTTCCTTTTTAACCACAAAGAGACTTAAGCAGTCCTTGTCACAGATGATGAATTGATGTTGCA
AGTATTGTCTTAGCTTGGATTAATTTTCTTGCTTACTGTAATTTTAGATAATATAGCTTTGTAATTAG
AGATTTTATGTGTAAACCACAAAAATGTTTACATGAAGGCCATTATTACAGATGTGACGTGCATAATT
ATTAGTAATTTGTATGTTTACATGGGTCAGTCTGGCAAAAAATTATGAAGTTTTAAAAATTAAAAAAA
ATTATAATGCCAGTTTTACTGGAAAGTAAAATTATTTCAGTAATCGATTATAGCAAAAGTATTGATTT
TCATTCCAGACAAAAGTCAGAATGAAAGGTAATTTCTCAATACTCTTTCAGATTAATAAAAGTACCTG
TAGCGATTTTTATCATTCACAAGTATATCACAAGTAAGTTAGAATTTGAGAACTGTGTTCTAGATCTC
TGAGGAGATGCAGTCAGATTTCTGAACTGTCTCAGCAAATGGTAAGTAACTTAGAGCTAGTAATTAAT
AACCTGTCCTTTGATTTCTGATTCAGCCAAGAATGGCCATATTTGGGAAAGGCAGATCTGGAGAGTAA
CCACGTTTTCATTCATTTACCACTTCTAGGCCCCTCCAGAGCTCTCAGATATTTTGGGGTTGAGCCCT
TCCCCAAAGCCATACAGGACCTTTTTTTTGTGATCTGTTCTAGCCATTTTTATGTTGGGTGCTTGTTA
TGGACTGAGCATTTATGTCCTCCCACACCCCCCCCATACCTTTTTTGAAGTCCTAACCCCCAGTGTGA
TGGTATTTGGAGACAGGGCCTTTGGAAGGTAATTACAGTTAGAAGAAGTCGGGAGGGTTGGGCCCAGG
TCTGATTGGATTAGTGCCCTTATATGAAAAGACACCAGGACGGGCGCAGTGGCTCACACCTGTAATCC
CAGCACTTTGGGAGGCCAAGGTGGGTGGATCACGAGGTCAGGAGTTTGAGACCAGCCTGGCCAATGTA
GTGAAACACCATCTCTACTAAAAATACAAAAATTAGCTGGGTGTGGTAGCGGGCTCCTGTCATCCAAG
CTACTCGGGAGGGTGAGGCATGAGAATCACTTGAACCCGGGAGTTGGAGGTTGCAGTGAGCCCAGATT
GTGCCACTGTACTCCAGCCTGGGTGACAGAGTGAGACTCTGTCTCAAAAAAGAAAAAAAAAAAAAAAG
AGACACCAGAGAGCTTGTTAGAAGAGGTCATGTGAGCACACAGTTAGAAGACCTTCAAGCCAAAGAAG
AGGCCTGAGATTGAAACCTACCTTGCAGGTACCTTAATTTTGGACTTCCCAGCCTCCAAAACTGTGAG
AAATAAGTTTCTGTTAAGTCACTCAGTCTGTGGTATTTTGTTATGGCAGCCTGAGCAGGTAGTTGTTC
TTTCAGAAGGTGTTGATAATAACCACATGCAACACCAAGTCACAAATAATAAAACAGATGTAACTTAT
ATTCATACAGAAAGTTGGGCACTGCCATTGCCTTGTTGGTTTACACGGCTGTGCTAGTTCAGTAGCAG
AAAGGTGCTGGTCTCCTTTACTCAGTTTACAATCTAGGCAGTAGAATGTAATCACTGCTTTAAACTTG
ATACTGCTTAGGGAGAGAATCATTGGTGCTGGGTAACTTTGGGTTCTAGGTTTACTTTTTGTGTATAT
ATAACTGTTTTTGGTAAATCACAAGTTTCTGGGCTTGTCGAATTAGATTTTGTTACAGATTATGAGCT
TTATTATGCTATACAGTTAGTTGTATGTATATATGCCTTTCCCACTAGATTTTAAGCTTTTTTTTTTT
TTTTTTTTTTGTGACGGAGTCTTGCTCTTGTCGCCCAGGCTGAAGTGGAGTGCAGTGGCACAATCTCG
GCTCACTGCAGCCTCCACCTCCTAGGTTCAAGCGATTCTCCTGCCTCGGCCTCCCAAGTAACTGGGAC
TACAGGCACGTGCCACCACACCCGGCTAATTTTTGTATTTTTTGTAGAGACAGGGTTTCGCCATGTTG
GCTAGGCTGGTCTTGAACTTCTGGCCTCAGGTGATCCACCCGCCTCAGCCTCCCAAAGTGCTGGGATT
TACAGGCATGAGCCACCACGCCCAGCTATAGCTCTTTAAGGGTTGTAAATTTATAATCATTCTTTTAC
TCTCCTGCAAATTCTGTTGCACACTGCCTTAATCAAGGTAGATGCTGAATGCATTTTTGTATAATTGA
ATATGTTGCAATCCCCAACTCTCTCCAACTGTTCCTGTCAAAGCAGCCACTGGATTGTTAACTAATCC
ATATTAGATGGGGTTAATTAATATCAGATGGGACAAGTAAGGGCTAATAAGATTATAGGCCACCAAGT
AGATTTCTGTCTAGCTCTTATAGAGATTGAGTTTATTGGACCTGTTTGATAGGAAGTTTTGGTGTTTG
GGATGATTAAAACTGAAGTTCCTATTTATTGAATTATACCTATTTATATTATTTCATATCAGTGGTCC
ACATGCAAGTGAGGCTTCTGAGACAGAGTTTGAGTTCTCTCTTCAACTACCATAACACTTAACCTGTA
TCTTTTTTTTTTTTTTTTTTTTTAGACAGGAGTCTCGCTCTGTCACTCAGGCTGGAGTGTAGTGGTAT
GATCTCGGCTCACTGTAACCTCTGCCTCCTGGATTCAAGCAGTTCTCCATGTCTCAGCCTCCCTAGTA
GCTGGGATTACAGGCCTGTGCCACCATGCCTGGCTAATTTTTTTTTTGTATTTTTAGTAGAGACGGGG
TTTTACCACGTTGGCCAGGCTGGTCTCGAACTCTTGACCTCGAGCGATCAACTTGCCTTGGCCTCCCA
AAGTGCTGGGATTACAGGCATGAGCCACAGCGCCCAGCCGTCTTTTTTTTTAAATAGCAATTTAACAC
TGTTCACAGTTACTCATGTACATGTCATGCCATCTATTACACTGTAAGTTCTGTGAGGGTAGCTGTAT
CAAATTTATCTAACTCTCTCTAGTATGCATGACATAGTAAGTATTCAATAAATATTTGCATATTAGTG
ATAAGGATACAGGTTCTGAATAGTGGGTCCTTACCATTTAAGAATTAGTATTTGATGGCCGGGGGGGG
TGGCTCACGCCTGTAATCCCAGCACTTTGGGAGGCTGAGGCGGGCGGATCATGAGATCAGGAGATCGA
GACCATCCTGGCTAACATGGTGAAATCCCGTCTTTACAAAAAAAATACAAAAGAATTAACCAAGTGTG
GTGGTGGGTGCCTGTAGTCCCAGCTACTGCTTTGTGAGGCTGAGGCAGGCAGATCACCTGAGGTGGGA
AATTCAAGACCAGCCTGACCAACATGGAGAAACCCCATCTCTACTAAAAATACAAAATTAGCCGGGCG
TGGTGGCGCATGTCTGTAATCCCAGCTACTCGGGAGGCTGAGGCAGGAGAATGGCGTGAACCCGGGAG
GCGGAGCTTGCAGTGAGCCAGGATCGCGCCACTGCACTCCAGCCTGGGCGACAGAGCGAGACTCCGTC
TCAAAAAAAAAAAAAAAAAAAAAATTAGTATTTGATATTTGATCATTAAATATGAATTAAGAGGACTT
AGACTTTTTGTTAAATGTCAAGCTGGGAAAAGTTGTCATTTAAATGAATTGCCTCTTATTTAATTTCG
TCTGATGATACATTTTGTTTTTATTTTGTAAAAAATTATTTTTTTTCTTTTTGGAGACAGGGTCTTGC
TCTGTTGCCCAGGCTGGTCACAAACTCCTGACCTCAAGCAATCCTCCTGCCTTAGCCTCCCAAAATGC
TGGGATTACAGGCGTGACGACCTCGCCCGGCCTTGTATTATGATACATTTTGAACAACTACAAGTAGA
CTTGGTATAATGAACCTGCACGTACCCATTGCCAAGTTCTGACAACTGTCTGTCTATAGCCAATTATG
CATTTCTTAAATTAGAACCCCCCCAATATACCCAAATATATATATATGTGTGCATATATATAGTAAGT
TGTAACAAAGTTGTGAATTCATACCTGAAGTATCTCAAGTGATGCAAGTTTTATGAATTTTTGTTTAT
GCCTTTTGGGAAGAGTTGTATTGACAAATTTTTTATGCTTAAAGTAAACCATAAATCAAAAAAATAAA
ATCTAGGATGCAATAAAACAAAACAACTTCTTGACATAAGTATGGTATGTAAATCTGTTTTGATTGGA
AATCAATTTGTTATATTGCCAGAATTCCTGTTTTAGAATACATCTCTGCTGATCTGTCTGTATTCTTA
GACTGCATATCTGGGATGAACTCTGGGCAGAATTCACATGGGCTTCCTTTGAAATAAACAAGACTTTT
CAAATTCTTAGTCGATCTGCAGAACCTGTAGCCAGGCACTGAACCATTTTGATAGATGCAGTAATCGT
TGCAAGTGTATATTTCAAGGGAGTTCTGGCTGGGTCCTAGTTTATGCTTGTGGCAGAAGCAGTGAGTA
ACTGGGAGGAAGTTGGTGAGTAAGCTTCAAGGAAGAAGTCATTTTTAGTACTCTGGATCTTCCTGATT
TTAAAGCACTACAAAATGGTGCATTTTCATTCTTGTCAAGTGATAACAGATATATTCTGATGAGCCTG
AAATGAATATATATTGTATCATTTTTATAATATCTAGCAAGGTTTGTATTTTCCTAGAACTTGAACTA
AATTTCAGTTCATAAAATTTATAAAATACTTAGTTGTTGTAAAATATTTTTGGAATGTTCACATAGGT
GACACACAAATGTCCCATTTTCATTCTTTCTATAGTAAATATGTTCTGATATGTGAAGGTTTAGCAGA
TGCATCAGCATTTAATCCTAGAGGATCTGGCATAATCTTTTCCCCCAAGAATAGAAATTTTTTCTGCT
TATGAAAGTAGTACATGTTTCTTTAAAAACAAATCAATATTGACTTCTGCCTGCTGTATAGCACTATG
CCTCCACCTGGCCATGACCAGGGGCATGTCCTGGTCCACCTACCTGAAAATGTTTGCAACCAGCCTCC
TGGCCATGTGCACAGGGGCTGAAGTTGTCCCACAGGTATTACGGGCCAACCTGACAATACATGAAGTT
CCACCAAAGTCTGAGAACTCAGAACTGAGCTTTGGGGACTGAAAGACAGCACAAACCTCAAATTTCTC
AGCACTGGAAACCTCAAAATATAACTGAATTCCATAAATAAGATTTTAAGTCTTAAATATGTATTTTT
AAATGTATTAAAAGTCAAGCTGCTTGTATTTAAGCACCTAATACAATGCTTAGGTTGTAAAAGGAGAT
GCTCAATAGGTACTAACTGATATATTGAGATTTAATTATGGTTTGACCAATATTTATTGGAAACCGCC
AAAGCTTAAATCATCAGCTTCTTGAATGTGATTTGAAAGGTAATTTAGTATTGAATAGCATGTGAGCT
AGAGTATTTCATTCTTTCTGGTTTATTTCTTCAAATAGACTTTGAATATAATGGTGAATGGGTATTAT
AAATTAACTAATAAAAATGACATTGAAAATGAAAAAATATATATATTAAAGTGTAGAAAGTGACCAGG
CGTGGTGGCTCACACCTGTAATCCAAGCACCTTGGGAGGCTGAGGCAGGAGGATCTCTTGATCCCAGG
AGTTCAAGACCAGCCTGGGCAACATAGCGAGACTTCGTCTCTAAAAAAAAAAAAGAGAGAGAAAAAAA
TTTTTTTTATTTAAAAAAAGTGTAGAAAGTGTCAAGACCCCACTTCTTACCATTATTTGGTATATTTC
TCTATACCCACCCACCCTTCCTCCTTACTCCCTCCCTCCCTTCCCAATCTTTTTATCTTTTTGTATTC
TGATTTTTTGTTTGTATATTTTGCTTTAATTTAATGTATCCTTTAAAAATTTCCCATACATTTTATAT
GTATATATAAAAACGCATGCTGCCAAAGATAATTTATAAGAAAGACCATTGAATTTTTTTAAAAGTGA
TATATATTCATTGAAAAAAATTTAGAATATATAGCAAAGCAATAAAGAACTAAATAAAATTGCTGTAA
CTCCTCTTTCAAAGATAAGTGCTTTTATGATTTTGTTGTATTTTTTTCTGTATATAGGTACATATATA
GTATTTATAAAGCTGTACTCATAGTACATTTTCACATCACAGGTACCATATCAGTGTTATTAAATATT
TTGTATGCCAGGGGCTAGACATACCAAGACAACCAATATGTGGTTCTACTTAAATAATATTAGAGTAT
CTTTTATGATGACACTTCATGAGTTGACTATAATAATCTTAGACTTCTAAGAGTTTGGGTTTTCAAAA
GATCACTTAGCTTTTTTGGGTGATTTTTCCCCCTTACTGTGAGATGAGAGAGGCTGTTTGGATTTGGG
ATTGGGGTAGCGGGGACAGCAACTTTTCTTTTCTTTTTCTTTTTTATTTTGAGGTAGGGTATTGCTGT
GTCACCCAGGCTGGAGTGCAGTGGTGTGATCTCGGCTCACTGCAACCTCCACCTCCCGGGCTCAGGTG
ATCCTCCTGCTTCAGCCTCCCAGTAACTGGGACTACAGGCGCGTGCCACATGCCTGGCTAATTTTGTA
TTTTTAGTAGAGATGGGGTTTCACCATGTTGGCCAGGCTGGTCTCTAACTCCTGACCTCAGGTGATAC
GCCCACCTGGGCCTCCCAAAATACTGGGATTACAGGCATGAGCCGCTGCATCAGCCAGCAGTTTTTCT
TGTGGTTTTTTTTGTTTGTTTTGTTTTGTTTTGTTTTTGAGATAGGGTCTTACTCTGTTGTCCACGCT
GGAGTGCTGTGGTATGATCGTAGCTCACTGCAGCCTCAAACTCCTGGGCTCAAGTGATTCCTTCTGCC
TCCGCCTCCCGAGTAGCTGGGACTACAGGTATGCACCACCATACCTGGCAAATTTTTACAAAGTTTTT
TGTAGGGACGGGGTCTTGCTACATTCCCCATGTCGGTCTTGAACTCCTGGCCTCAAGCAACTCTCCTG
TCTCAGCCTCCCAAAGCACTGGGATTACAAGTGTGAGCCACCACACCATGCCAGTTTTTCCTGTTCAG
TGTGATATTTTATCTTGTTAGACTACAGTGTGTTAAAACTTGTTTTACTAAATTTTCAAACATACTCA
AAAGTGGAGAGAATAGTATAATGAATACCCGTATGTTCATCACCCATGTTTAGAATATTATTAAATAT
AAAGATTTTGCTGCGTTTGTCTTAGCTCTTTAAAATTTTTCTTTTTCTCTTTGTGACCTAAAGGAAAT
TCCATATCTTATCACTTTACTTCTACATTCTTGACTAAGATGACTAAGACATATAGTTACATGGTTTT
TTGTTTTGTTTTTGTTTTTTAAAGACGAAATCTCGCTCTTGTCCCCCAGGCTGGAGTGCAATGGTGCC
ATCTCAGCTCAGTGCAACCTCTGCCTTCTGGGTACAAGCGATTCTCCTGCCTCAGCCTCCCAAGTAGC
TGGGATTACAGGCTCCTGCCACCACGCCTGGCTAATTTTTGTATTTTTAGTAGAGACGGCGGGGGGAG
GTTTCACCATGTTGACAAGGCTGGTCTGGAACTCCTGACCTCAGGTGATCCACCCGCCTCGGCCTCCC
AAAGTGCTGGGATTACAGGCGTGAGCCACCGCGCCCAGCCTGTTTTTTTGTTTGTGTGTTTTGTTTTT
TTTGAGACAGAGTCTTGCTCTGTTTCCCAGGCTGGAGTGAAGTGGTGCCATCTCAGCTCAGAGACAGA
GTCTTGCTCTGTTTCCCAGGCTGGAGTGAAGTGGTGCCATCTTGGCTCACTGCAACCTTCACCTCCCA
GGTTCAAGTGATTCTCCTGCCTCAGCCTCCCAAGTAGCTGGGACTACAGGCATGTGTCACCACACCCG
GCTAATTTTTTTGTATTTTTAGTAGAGACGGGATTTCACCGTGTTGCCCAGGCTGGTCTCGAACTCCT
GAGCTCAGGCAGTCTGCCTGCCTCAGCCTCCCAAAGTGCTGGGATTACACGTGTGAACCAACCCGCCC
GGCCTGTTGTTTTCTTACATAATTCATTATCATACCTACAAAGTTAACAGTTACTAATATCATCTTAC
ACCTAAATTTCTCTGATAGACTAAGGTTATTTTTTAACATCTTAATCCAATCAAATGTTTGTATCCTG
TAATGCTCTCATTGAAACAGCTATATTTCTTTTTCAGATTAGTGATGATGAACCAGGTTATGACCTTG
ATTTATTTTGCATACCTAATCATTATGCTGAGGATTTGGAAAGGGTGTTTATTCCTCATGGACTAATT
ATGGACAGGTAAGTAAGATCTTAAAATGAGGTTTTTTACTTTTTCTTGTGTTAATTTCAAACATCAGC
AGCTGTTCTGAGTACTTGCTATTTGAACATAAACTAGGCCAACTTATTAAATAACTGATGCTTTCTAA
AATCTTCTTTATTAAAAATAAAAGAGGAGGGCCTTACTAATTACTTAGTATCAGTTGTGGTATAGTGG
GACTCTGTAGGGACCAGAACAAAGTAAACATTGAAGGGAGATGGAAGAAGGAACTCTAGCCAGAGTCT
TGCATTTCTCAGTCCTAAACAGGGTAATGGACTGGGGCTGAATCACATGAAGGCAAGGTCAGATTTTT
ATTATTATGCACATCTAGCTTGAAAATTTTCTGTTAAGTCAATTACAGTGAAAAACCTTACCTGGTAT
TGAATGCTTGCATTGTATGTCTGGCTATTCTGTGTTTTTATTTTAAAATTATAATATCAAAATATTTG
TGTTATAAAATATTCTAACTATGGAGGCCATAAACAAGAAGACTAAAGTTCTCTCCTTTCAGCCTTCT
GTACACATTTCTTCTCAAGCACTGGCCTATGCATGTATACTATATGCAAAAGTACATATATACATTTA
TATTTTAACGTATGAGTATAGTTTTAAATGTTATTGGACACTTTTAATATTAGTGTGTCTAGAGCTAT
CTAATATATTTTAAAGGTTGCATAGCATTCTGTCTTATGGAGATACCATAACTGATTTAACCAGTCCA
CTATTGATAGACACTATTTTGTTCTTACCGACTGTACTAGAAGAAACATTCTTTTACATGTTTGGTAC
TTGTTCAGCTTTATTCAAGTGGAATTTCTGGGTCAAGGGGAAAGAGTTTATTGAATATTTTGGTATTG
CCAAATTTTCCTCTAAGAAGTTGAATCATTTTATACTCCTGATGTTATATGAGAGTACCTTTCTCTTC
ACAATTTGTCTCTTTTTTTTTTTTTTTTGAGACAAGGTCTCTGTTGCCCAGGCTGGGGTGCAGTGCAG
CAGAATGATCACAGTTCACTGCAGTCTCAACCTCCTGGGTTCAAGCGATCCTTCCACCTCAGCCTCCT
GAGTAGCTGGGACTATAGGTGTGCGCCACCACTCCCAGCTAATATTTTTATTTTGTAGAAACAGGGTT
CGCCATGTTACCCAGCCTCCCAAAGTGCTGGGATTACAGGCATGAGCCACTGGCCCAGTTTCTACAGT
CTCTCTTAATATTGTATATTATCCAGAAAATTTCATTTAATCAGAACCTGCCAGTCTGATAGGTGAAA
ATGGTATCTTGTTTTTATTTGCATTTAAAAAAAATTATGATAGTGGTATGCTTGGTTTTTTTGAAGGT
ATCAAATTTTTTACCTTATGAAACATGAGGGCAAAGGATGTGATACGTGGAAGATTTAAAAAAAATTT
TTAATGCATTTTTTTGAGACAAGGTCTTGCTCTATTGTCCAGGCTGGAGTGCAGTGGCACAATCACAG
TTCACTCCAGCCTCAACATCCTGCACTAAAGTGATTTTCCCACCTCACCTCTCAAGTAGCTGGGACTA
CAGGTACATGCTACCATGCCTGGCTAATTTTTTTTTTTTTGCAGGCATGGGGTCTCACTATATTGCCC
AGGTTGGTGTGGAAGTTTAATGACTAAGAGGTGTTTGTTATAAAGTTTAATGTATGAAACTTTCTATT
AAATTCCTGATTTTATTTCTGTAGGACTGAACGTCTTGCTCGAGATGTGATGAAGGAGATGGGAGGCC
ATCACATTGTAGCCCTCTGTGTGCTCAAGGGGGGCTATAAATTCTTTGCTGACCTGCTGGATTACATC
AAAGCACTGAATAGAAATAGTGATAGATCCATTCCTATGACTGTAGATTTTATCAGACTGAAGAGCTA
TTGTGTGAGTATATTTAATATATGATTCTTTTTAGTGGCAACAGTAGGTTTTCTTATATTTTCTTTGA
ATCTCTGCAAACCATACTTGCTTTCATTTCACTTGGTTACAGTGAGATTTTTCTAACATATTCACTAG
TACTTTACATCAAAGCCAATACTGTTTTTTTAAAACTAGTCACCTTGGAGGATATATACTTATTTTAC
AGGTGTGTGTGGTTTTTTAAATAAACTCCTTTTAGGAATTGCTGTTGGGACTTGGGATACTTTTTTCA
CTATACATACTGGTGACAGATACCCTCTCTTGAGCTACATCGGTTTGTGGGGAGTCAAAAGTCCTTTG
GAGCTAGGTTTGACAAATAAGGTGGGTTAACACTTGTTTCCTAGAAAGCACATGGAGAGCTAGAGTAT
TGGCGAATTGAAGAAATCCCCCTTTTTTTTTAACACACTTAAGAAAGGGGACTGCAGGTATACTCAAG
AGAGTAAGTCGCACCAGAAACCACTTTTGATCCACAGTCTGCCTGTGTCACACAATTGAAATGCATCA
CAACATTGACACTGTGGATGAAACAAAATCAGTGTGAATTTTAGTAGTGAATTTCATTCATAATTTGA
TCGTGCAAACGTTTGATTTTTATTACTTTAGACTATTGTTTCTGATTTTATGTTGGGTTGGTATTTCC
TGTGAGTTACTGTTTTACCTTTAAAATAGGAATTTTTCATACTCTTCAAAGATTAGAACAAATGTCCA
GTTTTTGCTGTTTCATGAATGAGTCCTGTCCATCTTTGTAGAAACTCGCCTTATGTTCACATTTTTAT
TGAGAATAAGACCACTTATCTACATTTAACTATCAACCTCATCCTCTCCATTAATCATCTATTTTAGT
GACCCAAGTTTTTGACCTTTTCCATGTTTACATCAATCCTGTAGGTGATTGGGCAGCCATTTAAGTAT
TATTATAGACATTTTCACTATCCCATTAAAACCCTTTATGCCCATACATCATAACACTACTTCCTACC
CATAAGCTCCTTTTAACTTGTTAAAGTCTTGCTTGAATTAAAGACTTGTTTACGGTATCGATAAGCTT
GATATCAAAACGCCAACTTTGACCCGGAACGCGGAAAACACCTGAGAAAAACACCTGGGCGAGTCTCC
ACGTAAACGGTCAAAGTCCCCGCGGCCCTAGACAAATATTACGCGCTATGAGTAACACAAAATTATTC
AGATTTCACTTCCTCTTATTCAGTTTTCCCGCGAAAATGGCCAAATCTTACTCGGTTACGCCCAAATT
TACTACAACATCCGCCTAAAACCGCGCGAAAATTGTCACTTCCTGTGTACACCGGCGCACACCAAAAA
CGTCACTTTTGCCACATCCGTCGCTTACATGTGTTCCGCCACACTTGCAACATCACACTTCCGCCACA
CTACTACGTCACCCGCCCCGTTCCCACGCCCCGCGCCACGTCACAAACTCCACCCCCTCATTATCATA
TTGGCTTCAATCCAAAATAAGGTATATTATTGATGATGTTTAAACATTAAGAATTAATTCGATCCTGA
ATGGCGAATGGACGCGCCCTGTAGCGGCGCATTAAGCGCGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGAGCTTTACGGCAC
CTCGACCGCAAAAAACTTGATTTGGGTGATGGTTCACGTAGTGGGCCATCGCCCTGATAGACGGTTTT
TCGCCCTTTGACGTTGGAGTCCACGTTCTTTAATAGTGGACTCTTGTTCCAAACTGGAACAACACTCA
ACCCTATCGCGGTCTATTCTTTTGATTTATAAGGGATGTTGCCGATTTCGGCCTATTGGTTAAAAAAT
GAGCTGATTTAACAAAAATTTTAACAAAATTCAGAAGAACTCGTCAAGAAGGCGATAGAAGGCGATGC
GCTGCGAATCGGGAGCGGCGATACCGTAAAGCACGAGGAAGCGGTCAGCCCATTCGCCGCCAAGCTCT
TCAGCAATATCACGGGTAGCCAACGCTATGTCCTGATAGCGGTCCGCCACACCCAGCCGGCCACAGTC
GATGAATCCAGAAAAGCGGCCATTTTCCACCATGATATTCGGCAAGCAGGCATCGCCATGGGTCACGA
CGAGATCCTCGCCGTCGGGCATGCTCGCCTTGAGCCTGGCGAACAGTTCGGCTGGCGCGAGCCCCTGA
TGCTCTTCGTCCAGATCATCCTGATCGACAAGACCGGCTTCCATCCGAGTACGTGCTCGCTCGATGCG
ATGTTTCGCTTGGTGGTCGAATGGGCAGGTAGCCGGATCAAGCGTATGCAGCCGCCGCATTGCATCAG
CCATGATGGATACTTTCTCGGCAGGAGCAAGGTGAGATGACAGGAGATCCTGCCCCGGCACTTCGCCC
AATAGCAGCCAGTCCCTTCCCGCTTCAGTGACAACGTCGAGCACAGCTGCGCAAGGAACGCCCGTCGT
GGCCAGCCACGATAGCCGCGCTGCCTCGTCTTGCAGTTCATTCAGGGCACCGGACAGGTCGGTCTTGA
CAAAAAGAACCGGGCGCCCCTGCGCTGACAGCCGGAACACGGCGGCATCAGAGCAGCCGATTGTCTGT
TGTGCCCAGTCATAGCCGAATAGCCTCTCCACCCAAGCGGCCGGAGAACCTGCGTGCAATCCATCTTG
TTCAATCATGCGAAACGATCCTCATCCTGTCTCTTGATCAGAGCTTGATCCCCTGCGCCATCAGATCC
TTGGCGGCAAGAAAGCCATCCAGTTTACTTTGCAGGGCTTCCCAACCTTACCAGAGGGCGCCCCAGCT
GGCAATTCCGGTTCGCTTGCTGTCCATAAAACCGCCCAGTCTAGCTATCGCCATGTAAGCCCACTGCA
AGCTACCTGCTTTCTCTTTGCGCTTGCGTTTTCCCTTGTCCAGATAGCCCAGTAGCTGACATTCATCC
GGGGTCAGCACCGTTTCTGCGGACTGGCTTTCTACGTGAAAAGGATCTAGGTGAAGATCCTTTTTGAT
AATCTCATGGCTGCAGCAATGGCAACAACGTTGCGCAAACTATTAACTGGCGAACTACTTACTCTAGC
TTCCCGGCAACAATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACGGGGAGTCAGGCAACTAT
GGATGAACGAAATAGACAGATCGCTGAGATAGGTGCCTCACTGATTAAGCATTGGTAACTGTCAGACC
AAGTTTACTCATATATACTTTAGATTGATTTAAAACTTCATTTTTAATTTAAAAGGATCTAGGTGAAG
ATCCTTTTTGATAATCTCATGACCAAAATCCCTTAACGTGAGTTTTCGTTCCACTGAGCGTCAGAC
RightITR = first underlined and bold sequence
U6 = first underlined sequence
CMV = first bold sequence
dCas 9VP64 = second underlined sequence
HGHpA = second bold sequence
EF1α = third underlined sequence
MPH = third bold sequence
HGHpA = fourth underlined sequence
Packaging Signal = fourth bold sequence
LeftITR = second underlined and bold sequence
LV-SAM
LeftLTR-PackagingSignal-RRE-U6-sgRNA-CMV-dCas9VP64-HGHpA-EFlα-MPH-
HGHpA-RightLTR
SEQ ID NO: 9
AGACGAAAGGGCCTCGTGATACGCCTATTTTTATAGGTTAATGTCATGATAATAATGGTTTCTTAGAC
GTCAGGTGGCACTTTTCGGGGAAATGTGCGCGGAACCCCTATTTGTTTATTTTTCTAAATACATTCAA
ATATGTATCCGCTCATGAGACAATAACCCTGATAAATGCTTCAATAATATTGAAAAAGGAAGAGTATG
AGTATTCAACATTTCCGTGTCGCCCTTATTCCCTTTTTTGCGGCATTTTGCCTTCCTGTTTTTGCTCA
CCCAGAAACGCTGGTGAAAGTAAAAGATGCTGAAGATCAGTTGGGTGCACGAGTGGGTTACATCGAAC
TGGATCTCAACAGCGGTAAGATCCTTGAGAGTTTTCGCCCCGAAGAACGTTTTCCAATGATGAGCACT
TTTAAAGTTCTGCTATGTGGCGCGGTATTATCCCGTATTGACGCCGGGCAAGAGCAACTCGGTCGCCG
CATACACTATTCTCAGAATGACTTGGTTGAGTACTCACCAGTCACAGAAAAGCATCTTACGGATGGCA
TGACAGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACACCACGATGCCTGTAG
CAATGGCAACAACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTGCAGCACTGGGGCCAGATG
GTAAGCCCTCCCGTATCGTAGTTATCTACACGACGGGGAGTCAGGCAACTATGGATGAACGAAATAGA
CAGATCGCTGAGATAGGTGCCTCACTGATTAAGCATTGGTAACTGTCAGACCAAGTTTACTCATATAT
ACTTTAGATTGATTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGGTAACTGGCTTCAGCAGAG
CGCAGATACCAAATACTGTTCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAACGGGGGGTTCGT
GCACACAGCCCAGCTTGGAGCGAACGACCTACACCGAACTGAGATACCTACAGCGTGAGCTATGAGAA
AGCGCCACGCTTCCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTTTCCTGCGTTATCCCCTGA
TTCTGTGGATAACCGTATTACCGCCTTTGAGTGAGCTCAGATGGTCCCCAGATATGGCCCAACCCTCA
GCAGTTTCTTAAGACCCATCAGATGTTTCCAGGCTCCCCCAAGGACCTGAAATGACCCTGCGCCTTAT
TTGAATTAACCAATCAGCCTGCTTCTCGCTTCTGTTCGCGCGCTTCTGCTTCCCGAGCTCTATAAAAG
AGCTCACAACCCCTCACTCGGCGCGCCAGTCCTCCGACAGACTGAGTCGCCCGGGGGGGATCACCAGA
TACCGCTCGCCGCAGCCGAACGACCGAGCGCAGCGAGTCAGTGAGCGAGGAAGCGGAAGAGCGCCCAA
TACGCAAACCGCCTCTCCCCGCGCGTTGGCCGATTCATTAATGCAGCTGTGGAATGTGTGTCAGTTAG
GGTGTGGAAAGTCCCCAGGCTCCCCAGCAGGCAGAAGTATGCAAAGCATGCATCTCAATTAGTCAGCA
ACCAGGTGTGGAAAGTCCCCAGGCTCCCCAGCAGGCAGAAGTATGCAAAGCATGCATCTCAATTAGTC
AGCAACCATAGTCCCGCCCCTAACTCCGCCCATCCCGCCCCTAACTCCGCCCAGTTCCGCCCATTCTC
CGCCCCATGGCTGACTAATTTTTTTTATTTATGCAGAGGCCGAGGCCGCCTCGGCCTCTGAGCTATTC
CAGAAGTAGTGAGGAGGCTTTTTTGGAGGCCTAGGCTTTTGCAAAAAGCTTGGACACAAGACAGGCTT
GCGAGATATGTTTGAGAATACCACTTTATCCCGCGTCAGGGAGAGGCAGTGCGTAAAAAGACGCGGAC
TCATGTGAAATACTGGTTTTTAGTGCGCCAGATCTCTATAATCTCGCGCAACCTATTTTCCCCTCGAA
CACTTTTTAAGCCGTAGATAAACAGGCTGGGACACTTCACATGAGCGAAAAATACATCGTCACCTGGG
ACATGTTGCAGATCCATGCACGTAAACTCGCAAGCCGACTGATGCCTTCTGAACAATGGAAAGGCATT
ATTGCCGTAAGCCGTGGCGGTCTGTACCGGGTGCGTTACTGGCGCGTGAACTGGGTATTCGTCATGTC
GATACCGTTTGTATTTCCAGCTACGATCACGACAACCAGCGCGAGCTTAAAGTGCTGAAACGCGCAGA
AGGCGATGGCGAAGGCTTCATCGTTATTGATGACCTGGTGGATACCGGTGGTACTGCGGTTGCGATTC
GTGAAATGTATCCAAAAGCGCACTTTGTCACCATCTTCGCAAAACCGGCTGGTCGTCCGCTGGTTGAT
GACTATGTTGTTGATATCCCGCAAGATACCTGGATTGAACAGCCGTGGGATATGGGCGTCGTATTCGT
CCCGCCAATCTCCGGTCGCTAATCTTTTCAACGCCTGGCACTGCCGGGCGTTGTTCTTTTTAACTTCA
GGCGGGTTACAATAGTTTCCAGTAAGTATTCTGGAGGCTGCATCCATGACACAGGCAAACCTGAGCGA
AACCCTGTTCAAACCCCGCTTTAAACATCCTGAAACCTCGACGCTAGTCCGCCGCTTTAATCACGGCG
CACAACCGCCTGTGCAGTCGGCCCTTGATGGTAAAACCATCCCTCACTGGTATCGCATGATTAACCGT
CTGATGTGGATCTGGCGCGGCATTGACCCACGCGAAATCCTCGACGTCCAGGCACGTATTGTGATGAG
CGATGCCGAACGTACCGACGATGATTTATACGATACGGTGATTGGCTACCGTGGCGGCAACTGGATTT
ATGAGTGGGCCCCGGATCTTTGTGAAGGAACCTTACTTCTGTGGTGTGACATAATTGGACAAACTACC
TACAGAGATTTAAAGCTCTAAGGTAAATATAAAATTTTTAAGTGTATAATGTGTTAAACTACTGATTC
TAATTGTTTGTGTATTTTAGATTCCAACCTATGGAACTGATGAATGGGAGCAGTGGTGGAATGCCTTT
AATGAGGAAAACCTGTTTTGCTCAGAAGAAATGCCATCTAGTGATGATGAGGCTACTGCTGACTCTCA
ACATTCTACTCCTCCAAAAAAGAAGAGAAAGGTAGAAGACCCCAAGGACTTTCCTTCAGAATTGCTAA
GTTTTTTGAGTCATGCTGTGTTTAGTAATAGAACTCTTGCTTGCTTTGCTATTTACACCACAAAGGAA
AAAGCTGCACTGCTATACAAGAAAATTATGGAAAAATATTCTGTAACCTTTATAAGTAGGCATAACAG
TTATAATCATAACATACTGTTTTTTCTTACTCCACACAGGCATAGAGTGTCTGCTATTAATAACTATG
CTCAAAAATTGTGTACCTTTAGCTTTTTAATTTGTAAAGGGGTTAATAAGGAATATTTGATGTATAGT
GCCTTGACTAGAGATCATAATCAGCCATACCACATTTGTAGAGGTTTTACTTGCTTTAAAAAACCTCC
CACACCTCCCCCTGAACCTGAAACATAAAATGAATGCAATTGTTGTTGTTAACTTGTTTATTGCAGCT
TATAATGGTTACAAATAAAGCAATAGCATCACAAATTTCACAAATAAAGCATTTTTTTCACTGCATTC
TAGTTGTGGTTTGTCCAAACTCATCAATGTATCTTATCATGTCTGGATCAACTGGATAACTCAAGCTA
ACCAAAATCATCCCAAACTTCCCACCCCATACCCTATTACCACTGCCAATTACCTGTGGTTTCATTTA
CTCTAAACCTGTGATTCCTCTGAATTATTTTCATTTTAAAGAAATTGTATTTGTTAAATATGTACTAC
AAACTTAGTAGTTGGAAGGGCTAATTCACTCCCAAAGAAGACAAGATATCCTTGATCTGTGGATCTAC
CACACACAAGGCTACTTCCCTGATTAGCAGAACTACACACCAGGGCCAGGGGTCAGATATCCACTGAC
CTTTGGATGGTGCTACAAGCTAGTACCAGTTGAGCCAGATAAGGTAGAAGAGGCCAATAAAGGAGAGA
ACACCAGCTTGTTACACCCTGTGAGCCTGCATGGGATGGATGACCCGGAGAGAGAAGTGTTAGAGTGG
AGGTTTGACAGCCGCCTAGCATTTCATCACGTGGCCCGAGAGCTGCATCCGGAGTACTTCAAGAACTG
CTGATATCGAGCTTGCTACAAGGGACTTTCCGCTGGGGACTTTCCAGGGAGGCGTGGCCTGGGCGGGA
CTGGGGAGTGGCGAGCCCTCAGATCCTGCATATAAGCAGCTGCTTTTTGCCTGTACTGGGTCTCTCTG
GTTAGACCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGATCCCTC
AGACCCTTTTAGTCAGTGTGGAAAATCTCTAGCAGTGGCGCCCGAACAGGGACTTGAAAGCGAAAGGG
AAACCAGAGGAGCTCTCTCGACGCAGGACTCGGCTTGCTGAAGCGCGCACGGCAAGAGGCGAGGGGCG
GCGACTGGTGAGTACGCCAAAAATTTTGACTAGCGGAGGCTAGAAGGAGAGAGATGGGTGCGAGAGCG
TCAGTATTAAGCGGGGAGAATTAGATCGCGATGGGAAAAAATTCGGTTAAGGCCAGGGGGAAAGAAAA
AATATAAATTAAAACATATAGTATGGGCAAGCAGGGAGCTAGAACGATTCGCAGTTAATCCTGGCCTG
TTAGAAACATCAGAAGGCTGTAGACAAATACTGGGACAGCTACAACCATCCCTTCAGACAGGATCAGA
AGAACTTAGATCATTATATAATACAGTAGCAACCCTCTATTGTGTGCATCAAAGGATAGAGATAAAAG
ACACCAAGGAAGCTTTAGACAAGATAGAGGAAGAGCAAAACAAAAGTAAGACCACCGCACAGCAAGCG
GCCGGCCGCTGATCTTCAGACCTGGAGGAGGAGATATGAGGGACAATTGGAGAAGTGAATTATATAAA
TATAAAGTAGTAAAAATTGAACCATTAGGAGTAGCACCCACCAAGGCAAAGAGAAGAGTGGTGCAGAG
AGAAAAAAGAGCAGTGGGAATAGGAGCTTTGTTCCTTGGGTTCTTGGGAGCAGCAGGAAGCACTATGG
GCGCAGCGTCAATGACGCTGACGGTACAGGCCAGACAATTATTGTCTGGTATAGTGCAGCAGCAGAAC
AATTTGCTGAGGGCTATTGAGGCGCAACAGCATCTGTTGCAACTCACAGTCTGGGGCATCAAGCAGCT
CCAGGCAAGAATCCTGGCTGTGGAAAGATACCTAAAGGATCAACAGCTCCTGGGGATTTGGGGTTGCT
CTGGAAAACTCATTTGCACCACTGCTGTGCCTTGGAATGCTAGTTGGAGTAATAAATCTCTGGAACAG
ATTTGGAATCACACGACCTGGATGGAGTGGGACAGAGAAATTAACAATTACACAAGCTTAATACACTC
CTTAATTGAAGAATCGCAAAACCAGCAAGAAAAGAATGAACAAGAATTATTGGAATTAGATAAATGGG
CAAGTTTGTGGAATTGGTTTAACATAACAAATTGGCTGTGGTATATAAAATTATTCATAATGATAGTA
GGAGGCTTGGTAGGTTTAAGAATAGTTTTTGCTGTACTTTCTATAGTGAATAGAGTTAGGCAGGGATA
TTCACCATTATCGTTTCAGACCCACCTCCCAACCCCGAGGGGACCCGACAGGCCCGAAGGAATAGAAG
AAGAAGGTGGAGAGAGAGACAGAGACAGATCCATTCGATTAGTGAACGGATCTCGACGGTATCGCCAA
ATGGCAGTATTCATCCACAATTTTAAAAGAAAAGGGGGGATTGGGGGGTACAGTGCAGGGGAAAGAAT
AGTAGACATAATAGCAACAGACATACAAACTAAAGAATTACAAAAACAAATTACAAAAATTCAAAATT
TTCGGGTTTATTACAGGGACAGCAGAGATCCAGTTTGGATCGATAAGCTTGATATCGAATTCGTAGGG
ATAACAGGGTAATGAGGGCCTATTTCCCATGATTCCTTCATATTTGCATATACGATACAAGGCTGTTA
GAGAGATAATTGGAATTAATTTGACTGTAAACACAAAGATATTAGTACAAAATACGTGACGTAGAAAG
TAATAATTTCTTGGGTAGTTTGCAGTTTTAAAATTATGTTTTAAAATGGACTATCATATGCTTACCGT

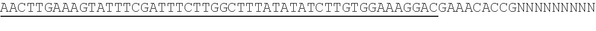

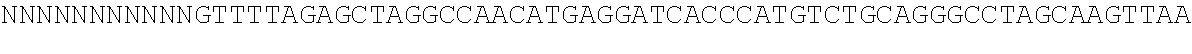

AATAAGGCTAGTCCGTTATCAACTTGGCCAACATGAGGATCACCCATGTCTGCAGGGCCAAGTGGCAC
CGAGTCGGTGCTTTTTTTGGATCCTGTTGACAATTAATCATCGGCATAGTATATCGGCATAGTATAAT
ACGACAAGGTGAGGAACTAAACCATGGCCAAGTTGACCAGTGCCGTTCCGGTGCTCACCGCGCGCGAC
GTCGCCGGAGCGGTCGAGTTCTGGACCGACCGGCTCGGGTTCTCCCGGGACTTCGTGGAGGACGACTT
CGCCGGTGTGGTCCGGGACGACGTGACCCTGTTCATCAGCGCGGTCCAGGACCAGGTGGTGCCGGACA
ACACCCTGGCCTGGGTGTGGGTGCGCGGCCTGGACGAGCTGTACGCCGAGTGGTCGGAGGTCGTGTCC
ACGAACTTCCGGGACGCCTCCGGGCCGGCCATGACCGAGATCGGCGAGCAGCCGTGGGGGCGGGAGTT
CGCCCTGCGCGACCCGGCCGGCAACTGCGTGCACTTCGTGGCCGAGGAGCAGGACTGATAGGGATAAC
AGGGTAATGCTAGCATAGTAATCAATTACGGGGTCATTAGTTCATAGCCCATATATGGAGTTCCGCGT
TACATAACTTACGGTAAATGGCCCGCCTGGCTGACCGCCCAACGACCCCCGCCCATTGACGTCAATAA
TGACGTATGTTCCCATAGTAACGTCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAATGA
CGGTAAATGGCCCGCCTGGCATTATGCCCAGTACATGACCTTATGGGACTTTCCTACTTGGCAGTACA
TCTACGTATTAGTCATCGCTATTACCATGGTGATGCGGTTTTGGCAGTACATCAATGGGCGTGGATAG
CGGTTTGACTCACGGGGATTTCCAAGTCTCCACCCCATTGACGTCAATGGGAGTTTGTTTTGCACCAA
AATCAACGGGACTTTCCAAAATGTCGTAACAACTCCGCCCCATTGACGCAAATGGGCGGTAGGCGTGT
ACGGTGGGAGGTCTATATAAGCAGAGCTCGTTTAGTGAACCGTCAGATCGCCTGGAGACGCCATCCAC
GCTGTTTTGACCTCCATAGAAGACACCGGGACCGATCCAGCCTCCGCGGATTCGAATCCCGGCCGGGA
ACGGTGCATTGGAACGCGGATTCCCCGTGCCAAGAGTGACGTAAGTACCGCCTATAGAGTCTATAGGC
CCACAAAAAATGCTTTCTTCTTTTAATATACTTTTTTGTTTATCTTATTTCTAATACTTTCCCTAATC
TCTTTCTTTCAGGGCAATAATGATACAATGTATCATGCCTCTTTGCACCATTCTAAAGAATAACAGTG
ATAATTTCTGGGTTAAGGCAATAGCAATATTTCTGCATATAAATATTTCTGCATATAAATTGTAACTG
ATGTAAGAGGTTTCATATTGCTAATAGCAGCTACAATCCAGCTACCATTCTGCTTTTATTTTATGGTT
GGGATAAGGCTGGATTATTCTGAGTCCAAGCTAGGCCCTTTTGCTAATCATGTTCATACCTCTTATCT
TCCTCCCACAGCTCCTGGGCAACGTGCTGGTCTGTGTGCTGGCCCATCACTTTGGCAAAGAATTGGGA
TCGTACGGCCACCATGAAAAGGCCGGCGGCCACGAAAAAGGCCGGCCAGGCAAAAAAGAAAAAGGACA
AGAAGTACAGCATCGGCCTGGCCATCGGCACCAACTCTGTGGGCTGGGCCGTGATCACCGACGAGTAC
AAGGTGCCCAGCAAGAAATTCAAGGTGCTGGGCAACACCGACCGGCACAGCATCAAGAAGAACCTGAT
CGGAGCCCTGCTGTTCGACAGCGGCGAAACAGCCGAGGCCACCCGGCTGAAGAGAACCGCCAGAAGAA
GATACACCAGACGGAAGAACCGGATCTGCTATCTGCAAGAGATCTTCAGCAACGAGATGGCCAAGGTG
GACGACAGCTTCTTCCACAGACTGGAAGAGTCCTTCCTGGTGGAAGAGGATAAGAAGCACGAGCGGCA
CCCCATCTTCGGCAACATCGTGGACGAGGTGGCCTACCACGAGAAGTACCCCACCATCTACCACCTGA
GAAAGAAACTGGTGGACAGCACCGACAAGGCCGACCTGCGGCTGATCTATCTGGCCCTGGCCCACATG
ATCAAGTTCCGGGGCCACTTCCTGATCGAGGGCGACCTGAACCCCGACAACAGCGACGTGGACAAGCT
GTTCATCCAGCTGGTGCAGACCTACAACCAGCTGTTCGAGGAAAACCCCATCAACGCCAGCGGCGTGG
ACGCCAAGGCCATCCTGTCTGCCAGACTGAGCAAGAGCAGACGGCTGGAAAATCTGATCGCCCAGCTG
CCCGGCGAGAAGAAGAATGGCCTGTTCGGCAACCTGATTGCCCTGAGCCTGGGCCTGACCCCCAACTT
CAAGAGCAACTTCGACCTGGCCGAGGATGCCAAACTGCAGCTGAGCAAGGACACCTACGACGACGACC
TGGACAACCTGCTGGCCCAGATCGGCGACCAGTACGCCGACCTGTTTCTGGCCGCCAAGAACCTGTCC
GACGCCATCCTGCTGAGCGACATCCTGAGAGTGAACACCGAGATCACCAAGGCCCCCCTGAGCGCCTC
TATGATCAAGAGATACGACGAGCACCACCAGGACCTGACCCTGCTGAAAGCTCTCGTGCGGCAGCAGC
TGCCTGAGAAGTACAAAGAGATTTTCTTCGACCAGAGCAAGAACGGCTACGCCGGCTACATTGACGGC
GGAGCCAGCCAGGAAGAGTTCTACAAGTTCATCAAGCCCATCCTGGAAAAGATGGACGGCACCGAGGA
ACTGCTCGTGAAGCTGAACAGAGAGGACCTGCTGCGGAAGCAGCGGACCTTCGACAACGGCAGCATCC
CCCACCAGATCCACCTGGGAGAGCTGCACGCCATTCTGCGGCGGCAGGAAGATTTTTACCCATTCCTG
AAGGACAACCGGGAAAAGATCGAGAAGATCCTGACCTTCCGCATCCCCTACTACGTGGGCCCTCTGGC
CAGGGGAAACAGCAGATTCGCCTGGATGACCAGAAAGAGCGAGGAAACCATCACCCCCTGGAACTTCG
AGGAAGTGGTGGACAAGGGCGCTTCCGCCCAGAGCTTCATCGAGCGGATGACCAACTTCGATAAGAAC
CTGCCCAACGAGAAGGTGCTGCCCAAGCACAGCCTGCTGTACGAGTACTTCACCGTGTATAACGAGCT
GACCAAAGTGAAATACGTGACCGAGGGAATGAGAAAGCCCGCCTTCCTGAGCGGCGAGCAGAAAAAGG
CCATCGTGGACCTGCTGTTCAAGACCAACCGGAAAGTGACCGTGAAGCAGCTGAAAGAGGACTACTTC
AAGAAAATCGAGTGCTTCGACTCCGTGGAAATCTCCGGCGTGGAAGATCGGTTCAACGCCTCCCTGGG
CACATACCACGATCTGCTGAAAATTATCAAGGACAAGGACTTCCTGGACAATGAGGAAAACGAGGACA
TTCTGGAAGATATCGTGCTGACCCTGACACTGTTTGAGGACAGAGAGATGATCGAGGAACGGCTGAAA
ACCTATGCCCACCTGTTCGACGACAAAGTGATGAAGCAGCTGAAGCGGCGGAGATACACCGGCTGGGG
CAGGCTGAGCCGGAAGCTGATCAACGGCATCCGGGACAAGCAGTCCGGCAAGACAATCCTGGATTTCC
TGAAGTCCGACGGCTTCGCCAACAGAAACTTCATGCAGCTGATCCACGACGACAGCCTGACCTTTAAA
GAGGACATCCAGAAAGCCCAGGTGTCCGGCCAGGGCGATAGCCTGCACGAGCACATTGCCAATCTGGC
CGGCAGCCCCGCCATTAAGAAGGGCATCCTGCAGACAGTGAAGGTGGTGGACGAGCTCGTGAAAGTGA
TGGGCCGGCACAAGCCCGAGAACATCGTGATCGAAATGGCCAGAGAGAACCAGACCACCCAGAAGGGA
CAGAAGAACAGCCGCGAGAGAATGAAGCGGATCGAAGAGGGCATCAAAGAGCTGGGCAGCCAGATCCT
GAAAGAACACCCCGTGGAAAACACCCAGCTGCAGAACGAGAAGCTGTACCTGTACTACCTGCAGAATG
GGCGGGATATGTACGTGGACCAGGAACTGGACATCAACCGGCTGTCCGACTACGATGTGGACCACATC
GTGCCTCAGAGCTTTCTGAAGGACGACTCCATCGACAACAAGGTGCTGACCAGAAGCGACAAGGCCCG
GGGCAAGAGCGACAACGTGCCCTCCGAAGAGGTCGTGAAGAAGATGAAGAACTACTGGCGGCAGCTGC
TGAACGCCAAGCTGATTACCCAGAGAAAGTTCGACAATCTGACCAAGGCCGAGAGAGGCGGCCTGAGC
GAACTGGATAAGGCCGGCTTCATCAAGAGACAGCTGGTGGAAACCCGGCAGATCACAAAGCACGTGGC
ACAGATCCTGGACTCCCGGATGAACACTAAGTACGACGAGAATGACAAGCTGATCCGGGAAGTGAAAG
TGATCACCCTGAAGTCCAAGCTGGTGTCCGATTTCCGGAAGGATTTCCAGTTTTACAAAGTGCGCGAG
ATCAACAACTACCACCACGCCCACGACGCCTACCTGAACGCCGTCGTGGGAACCGCCCTGATCAAAAA
GTACCCTAAGCTGGAAAGCGAGTTCGTGTACGGCGACTACAAGGTGTACGACGTGCGGAAGATGATCG
CCAAGAGCGAGCAGGAAATCGGCAAGGCTACCGCCAAGTACTTCTTCTACAGCAACATCATGAACTTT
TTCAAGACCGAGATTACCCTGGCCAACGGCGAGATCCGGAAGCGGCCTCTGATCGAGACAAACGGCGA
AACCGGGGAGATCGTGTGGGATAAGGGCCGGGATTTTGCCACCGTGCGGAAAGTGCTGAGCATGCCCC
AAGTGAATATCGTGAAAAAGACCGAGGTGCAGACAGGCGGCTTCAGCAAAGAGTCTATCCTGCCCAAG
AGGAACAGCGATAAGCTGATCGCCAGAAAGAAGGACTGGGACCCTAAGAAGTACGGCGGCTTCGACAG
CCCCACCGTGGCCTATTCTGTGCTGGTGGTGGCCAAAGTGGAAAAGGGCAAGTCCAAGAAACTGAAGA
GTGTGAAAGAGCTGCTGGGGATCACCATCATGGAAAGAAGCAGCTTCGAGAAGAATCCCATCGACTTT
CTGGAAGCCAAGGGCTACAAAGAAGTGAAAAAGGACCTGATCATCAAGCTGCCTAAGTACTCCCTGTT
CGAGCTGGAAAACGGCCGGAAGAGAATGCTGGCCTCTGCCGGCGAACTGCAGAAGGGAAACGAACTGG
CCCTGCCCTCCAAATATGTGAACTTCCTGTACCTGGCCAGCCACTATGAGAAGCTGAAGGGCTCCCCC
GAGGATAATGAGCAGAAACAGCTGTTTGTGGAACAGCACAAGCACTACCTGGACGAGATCATCGAGCA
GATCAGCGAGTTCTCCAAGAGAGTGATCCTGGCCGACGCTAATCTGGACAAAGTGCTGTCCGCCTACA
ACAAGCACCGGGATAAGCCCATCAGAGAGCAGGCCGAGAATATCATCCACCTGTTTACCCTGACCAAT
CTGGGAGCCCCTGCCGCCTTCAAGTACTTTGACACCACCATCGACCGGAAGAGGTACACCAGCACCAA
AGAGGTGCTGGACGCCACCCTGATCCACCAGAGCATCACCGGCCTGTACGAGACACGGATCGACCTGT
CTCAGCTGGGAGGCGACAGCGCTGGAGGAGGTGGAAGCGGAGGAGGAGGAAGCGGAGGAGGAGGTAGC
GGACCTAAGAAAAAGAGGAAGGTGGCGGCCGCTGGATCCGGACGGGCTGACGCATTGGACGATTTTGA
TCTGGATATGCTGGGAAGTGACGCCCTCGATGATTTTGACCTTGACATGCTTGGTTCGGATGCCCTTG
ATGACTTTGACCTCGACATGCTCGGCAGTGACGCCCTTGATGATTTCGACCTGGACATGCTGATTAAC
TGTACATAAACGGGTGGCATCCCTGTGACCCCTCCCCAGTGCCTCTCCTGGCCCTGGAAGTTGCCACT
CCAGTGCCCACCAGCCTTGTCCTAATAAAATTAAGTTGCATCATTTTGTCTGACTAGGTGTCCTTCTA
TAATATTATGGGGTGGAGGGGGGTGGTATGGAGCAAGGGGCAAGTTGGGAAGACAACCTGTAGGGCCT
GCGGGGTCTATTGGGAACCAAGCTGGAGTGCAGTGGCACAATCTTGGCTCACTGCAATCTCCGCCTCC
TGGGTTCAAGCGATTCTCCTGCCTCAGCCTCCCGAGTTGTTGGGATTCCAGGCATGCATGACCAGGCT
CAGCTAATTTTTGTTTTTTTGGTAGAGACGGGGTTTCACCATATTGGCCAGGCTGGTCTCCAACTCCT
AATCTCAGGTGATCTACCCACCTTGGCCTCCCAAATTGCTGGGATTACAGGCGTGAACCACTGCTCCC
TTCCCTGTCCTTGAATTCTAACTATAACGGTCCTAAGGTAGCGAAGCTAGCTGCAAAGATGGATAAAG
TTTTAAACAGAGAGGAATCTTTGCAGCTAATGGACCTTCTAGGTCTTGAAAGGAGTGGGAATTGGCTC
CGGTGCCCGTCAGTGGGCAGAGCGCACATCGCCCACAGTCCCCGAGAAGTTGGGGGGAGGGGTCGGCA
ATTGAACCGGTGCCTAGAGAAGGTGGCGCGGGGTAAACTGGGAAAGTGATGTCGTGTACTGGCTCCGC
CTTTTTCCCGAGGGTGGGGGAGAACCGTATATAAGTGCAGTAGTCGCCGTGAACGTTCTTTTTCGCAA
CGGGTTTGCCGCCAGAACACAGGTAAGTGCCGTGTGTGGTTCCCGCGGGCCTGGCCTCTTTACGGGTT
ATGGCCCTTGCGTGCCTTGAATTACTTCCACCTGGCTGCAGTACGTGATTCTTGATCCCGAGCTTCGG
GTTGGAAGTGGGTGGGAGAGTTCGAGGCCTTGCGCTTAAGGAGCCCCTTCGCCTCGTGCTTGAGTTGA
GGCCTGGCCTGGGCGCTGGGGCCGCCGCGTGCGAATCTGGTGGCACCTTCGCGCCTGTCTCGCTGCTT
TCGATAAGTCTCTAGCCATTTAAAATTTTTGATGACCTGCTGCGACGCTTTTTTTCTGGCAAGATAGT
CTTGTAAATGCGGGCCAAGATCTGCACACTGGTATTTCGGTTTTTGGGGCCGCGGGCGGCGACGGGGC
CCGTGCGTCCCAGCGCACATGTTCGGCGAGGCGGGGCCTGCGAGCGCGGCCACCGAGAATCGGACGGG
GGTAGTCTCAAGCTGGCCGGCCTGCTCTGGTGCCTGGCCTCGCGCCGCCGTGTATCGCCCCGCCCTGG
GCGGCAAGGCTGGCCCGGTCGGCACCAGTTGCGTGAGCGGAAAGATGGCCGCTTCCCGGCCCTGCTGC
AGGGAGCTCAAAATGAAGGACGCGGCGCTCGGGAGAGCGGGCGGGTGAGTCACCCACACAAAGGAAAA
GGGCCTTTCCGTCCTCAGCCGTCGCTTCATGTGACTCCACGGAGTACCGGGCGCCGTCCAGGCACCTC
GATTAGTTCTCGAGCTTTTGGAGTACGTCGTCTTTAGGTTGGGGGGAGGGGTTTTATGCGATGGAGTT
TCCCCACACTGAGTGGGTGGAGACTGAAGTTAGGCCAGCTTGGCACTTGATGTAATTCTCCTTGGAAT
TTGCCCTTTTTGAGTTTGGATCTTGGTTCATTCTCAAGCCTCAGACAGTGGTTCAAAGTTTTTTTCTT
CCATTTCAGGTGTCGTGACGTACGGCCACCATGGCTTCAAACTTTACTCAGTTCGTGCTCGTGGACAA
TGGTGGGACAGGGGATGTGACAGTGGCTCCTTCTAATTTCGCTAATGGGGTGGCAGAGTGGATCAGCT
CCAACTCACGGAGCCAGGCCTACAAGGTGACATGCAGCGTCAGGCAGTCTAGTGCCCAGAAGAGAAAG
TATACCATCAAGGTGGAGGTCCCCAAAGTGGCTACCCAGACAGTGGGCGGAGTCGAACTGCCTGTCGC
CGCTTGGAGGTCCTACCTGAACATGGAGCTCACTATCCCAATTTTCGCTACCAATTCTGACTGTGAAC
TCATCGTGAAGGCAATGCAGGGGCTCCTCAAAGACGGTAATCCTATCCCTTCCGCCATCGCCGCTAAC
TCAGGTATCTACAGCGCTGGAGGAGGTGGAAGCGGAGGAGGAGGAAGCGGAGGAGGAGGTAGCGGACC
TAAGAAAAAGAGGAAGGTGGCGGCCGCTGGATCCCCTTCAGGGCAGATCAGCAACCAGGCCCTGGCTC
TGGCCCCTAGCTCCGCTCCAGTGCTGGCCCAGACTATGGTGCCCTCTAGTGCTATGGTGCCTCTGGCC
CAGCCACCTGCTCCAGCCCCTGTGCTGACCCCAGGACCACCCCAGTCACTGAGCGCTCCAGTGCCCAA
GTCTACACAGGCCGGCGAGGGGACTCTGAGTGAAGCTCTGCTGCACCTGCAGTTCGACGCTGATGAGG
ACCTGGGAGCTCTGCTGGGGAACAGCACCGATCCCGGAGTGTTCACAGATCTGGCCTCCGTGGACAAC
TCTGAGTTTCAGCAGCTGCTGAATCAGGGCGTGTCCATGTCTCATAGTACAGCCGAACCAATGCTGAT
GGAGTACCCCGAAGCCATTACCCGGCTGGTGACCGGCAGCCAGCGGCCCCCCGACCCCGCTCCAACTC
CCCTGGGAACCAGCGGCCTGCCTAATGGGCTGTCCGGAGATGAAGACTTCTCAAGCATCGCTGATATG
GACTTTAGTGCCCTGCTGTCACAGATTTCCTCTAGTGGGCAGGGAGGAGGTGGAAGCGGCTTCAGCGT
GGACACCAGTGCCCTGCTGGACCTGTTCAGCCCCTCGGTGACCGTGCCCGACATGAGCCTGCCTGACC
TTGACAGCAGCCTGGCCAGTATCCAAGAGCTCCTGTCTCCCCAGGAGCCCCCCAGGCCTCCCGAGGCA
GAGAACAGCAGCCCGGATTCAGGGAAGCAGCTGGTGCACTACACAGCGCAGCCGCTGTTCCTGCTGGA
CCCCGGCTCCGTGGACACCGGGAGCAACGACCTGCCGGTGCTGTTTGAGCTGGGAGAGGGCTCCTACT
TCTCCGAAGGGGACGGCTTCGCCGAGGACCCCACCATCTCCCTGCTGACAGGCTCGGAGCCTCCCAAA
GCCAAGGACCCCACTGTCTCCTGTACATAAACGGGTGGCATCCCTGTGACCCCTCCCCAGTGCCTCTC
CTGGCCCTGGAAGTTGCCACTCCAGTGCCCACCAGCCTTGTCCTAATAAAATTAAGTTGCATCATTTT
GTCTGACTAGGTGTCCTTCTATAATATTATGGGGTGGAGGGGGGTGGTATGGAGCAAGGGGCAAGTTG
GGAAGACAACCTGTAGGGCCTGCGGGGTCTATTGGGAACCAAGCTGGAGTGCAGTGGCACAATCTTGG
CTCACTGCAATCTCCGCCTCCTGGGTTCAAGCGATTCTCCTGCCTCAGCCTCCCGAGTTGTTGGGATT
CCAGGCATGCATGACCAGGCTCAGCTAATTTTTGTTTTTTTGGTAGAGACGGGGTTTCACCATATTGG
CCAGGCTGGTCTCCAACTCCTAATCTCAGGTGATCTACCCACCTTGGCCTCCCAAATTGCTGGGATTA
CAGGCGTGAACCACTGCTCCCTTCCCTGTCCTTGAATTCTAACTATAACGGTCCTAAGGTAGCGAAGG
TACCTTTAAGACCAATGACTTACAAGGCAGCTGTAGATCTTAGCCACTTTTTAAAAGAAAAGGGGGGA
CTGGAAGGGCTAATTCACTCCCAACGAAGACAAGATCTGCTTTTTGCTTGTACTGGGTCTCTCTGGTT
AGACCAGATCTGAGCCTGGGAGCTCTCTGGCTAACTAGGGAACCCACTGCTTAAGCCTCAATAAAGCT
TGCCTTGAGTGCTTCAAGTAGTGTGTGCCCGTCTGTTGTGTGACTCTGGTAACTAGAGATCCCTCAGA
CCCTTTTAGTCAGTGTGGAAAATCTCTAGCAGCATCTAGAATTAATTCCGTGTATTCTATAGTGTCAC
CTAAATCGTATGTGTATGATACATAAGGTTATGTATTAATTGTAGCCGCGTTCTAACGACAATATGTA
CAAGCCTAATTGTGTAGCATCTGGCTTACTGAAGCAGACCCTATCATCTCTCTCGTAAACTGCCGTCA
GAGTCGGTTTGGTTGGACGAACCTTCTGAGTTTCTGGTAACGCCGTCCCGCACCCGGAAATGGTCAGC
GAACCAATCAGCAGGGTCATCGCTAGCCAGATCCTCTACGCCGGACGCATCGTGGCCGGCATCACCGG
CGCCACAGGTGCGGTTGCTGGCGCCTATATCGCCGACATCACCGATGGGGAAGATCGGGCTCGCCACT
TCGGGCTCATGAGCGCTTGTTTCGGCGTGGGTATGGTGGCAGGCCCCGTGGCCGGGGGACTGTTGGGC
GCCATCTCCTTGCATGCACCATTCCTTGCGGCGGCGGTGCTCAACGGCCTCAACCTACTACTGGGCTG
CTTCCTAATGCAGGAGTCGCATAAGGGAGAGCGTCGAATGGTGCACTCTCAGTACAATCTGCTCTGAT
GCCGCATAGTTAAGCCAGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAGAGGTTTTCACCGT
CATCACCGAAACGCGCG
RightITR = first underlined and bold sequence
Packaging Signal = first underlined sequence
RRE = first bold sequence
U6 = second underlined sequence
CMV = second bold sequence
dCas9VP64 = third underlined sequence
HGHpA = third bold sequence
EF1α = fourth underlined sequence
MPH = fourth bold sequence
HGHpA = fifth underlined sequence
LeftITR = second underlined and bold sequence
AAV-dCas9VP64
LeftITR-EF1α-dCas9VP64-FpA-RightITR
SEQ ID NO: 10
GTAGAAAAGATCAAAGGATCTTCTTGAGATCCTTTTTTTCTGCGCGTAATCTGCTGCTTGCAAACAAA
AAAACCACCGCTACCAGCGGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCGTAGTTAGGCCACCACTTC
AAGAACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCGGGCT
GAACGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCACATGTCCTGCAG
GCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGC
GGCCGCACGCGTGGCTCCGGTGCCCGTCAGTGGGCAGAGCGCACATCGCCCACAGTCCCCGAGAAGTT
GGGGGGAGGGGTCGGCAATTGAACCGGTGCCTAGAGAAGGTGGCGCGGGGTAAACTGGGAAAGTGATG
TCGTGTACTGGCTCCGCCTTTTTCCCGAGGGTGGGGGAGAACCGTATATAAGTGCAGTAGTCGCCGTG
AACGTTCTTTTTCGCAACGGGTTTGCCGCCAGAACACAGGAATTCGCCACCATGAAAAGGCCGGCGGC
CACGAAAAAGGCCGGCCAGGCAAAAAAGAAAAAGGACAAGAAGTACAGCATCGGCCTGGCCATCGGCA
CCAACTCTGTGGGCTGGGCCGTGATCACCGACGAGTACAAGGTGCCCAGCAAGAAATTCAAGGTGCTG
GGCAACACCGACCGGCACAGCATCAAGAAGAACCTGATCGGAGCCCTGCTGTTCGACAGCGGCGAAAC
AGCCGAGGCCACCCGGCTGAAGAGAACCGCCAGAAGAAGATACACCAGACGGAAGAACCGGATCTGCT
ATCTGCAAGAGATCTTCAGCAACGAGATGGCCAAGGTGGACGACAGCTTCTTCCACAGACTGGAAGAG
TCCTTCCTGGTGGAAGAGGATAAGAAGCACGAGCGGCACCCCATCTTCGGCAACATCGTGGACGAGGT
GGCCTACCACGAGAAGTACCCCACCATCTACCACCTGAGAAAGAAACTGGTGGACAGCACCGACAAGG
CCGACCTGCGGCTGATCTATCTGGCCCTGGCCCACATGATCAAGTTCCGGGGCCACTTCCTGATCGAG
GGCGACCTGAACCCCGACAACAGCGACGTGGACAAGCTGTTCATCCAGCTGGTGCAGACCTACAACCA
GCTGTTCGAGGAAAACCCCATCAACGCCAGCGGCGTGGACGCCAAGGCCATCCTGTCTGCCAGACTGA
GCAAGAGCAGACGGCTGGAAAATCTGATCGCCCAGCTGCCCGGCGAGAAGAAGAATGGCCTGTTCGGC
AACCTGATTGCCCTGAGCCTGGGCCTGACCCCCAACTTCAAGAGCAACTTCGACCTGGCCGAGGATGC
CAAACTGCAGCTGAGCAAGGACACCTACGACGACGACCTGGACAACCTGCTGGCCCAGATCGGCGACC
AGTACGCCGACCTGTTTCTGGCCGCCAAGAACCTGTCCGACGCCATCCTGCTGAGCGACATCCTGAGA
GTGAACACCGAGATCACCAAGGCCCCCCTGAGCGCCTCTATGATCAAGAGATACGACGAGCACCACCA
GGACCTGACCCTGCTGAAAGCTCTCGTGCGGCAGCAGCTGCCTGAGAAGTACAAAGAGATTTTCTTCG
ACCAGAGCAAGAACGGCTACGCCGGCTACATTGACGGCGGAGCCAGCCAGGAAGAGTTCTACAAGTTC
ATCAAGCCCATCCTGGAAAAGATGGACGGCACCGAGGAACTGCTCGTGAAGCTGAACAGAGAGGACCT
GCTGCGGAAGCAGCGGACCTTCGACAACGGCAGCATCCCCCACCAGATCCACCTGGGAGAGCTGCACG
CCATTCTGCGGCGGCAGGAAGATTTTTACCCATTCCTGAAGGACAACCGGGAAAAGATCGAGAAGATC
CTGACCTTCCGCATCCCCTACTACGTGGGCCCTCTGGCCAGGGGAAACAGCAGATTCGCCTGGATGAC
CAGAAAGAGCGAGGAAACCATCACCCCCTGGAACTTCGAGGAAGTGGTGGACAAGGGCGCTTCCGCCC
AGAGCTTCATCGAGCGGATGACCAACTTCGATAAGAACCTGCCCAACGAGAAGGTGCTGCCCAAGCAC
AGCCTGCTGTACGAGTACTTCACCGTGTATAACGAGCTGACCAAAGTGAAATACGTGACCGAGGGAAT
GAGAAAGCCCGCCTTCCTGAGCGGCGAGCAGAAAAAGGCCATCGTGGACCTGCTGTTCAAGACCAACC
GGAAAGTGACCGTGAAGCAGCTGAAAGAGGACTACTTCAAGAAAATCGAGTGCTTCGACTCCGTGGAA
ATCTCCGGCGTGGAAGATCGGTTCAACGCCTCCCTGGGCACATACCACGATCTGCTGAAAATTATCAA
GGACAAGGACTTCCTGGACAATGAGGAAAACGAGGACATTCTGGAAGATATCGTGCTGACCCTGACAC
TGTTTGAGGACAGAGAGATGATCGAGGAACGGCTGAAAACCTATGCCCACCTGTTCGACGACAAAGTG
ATGAAGCAGCTGAAGCGGCGGAGATACACCGGCTGGGGCAGGCTGAGCCGGAAGCTGATCAACGGCAT
CCGGGACAAGCAGTCCGGCAAGACAATCCTGGATTTCCTGAAGTCCGACGGCTTCGCCAACAGAAACT
TCATGCAGCTGATCCACGACGACAGCCTGACCTTTAAAGAGGACATCCAGAAAGCCCAGGTGTCCGGC
CAGGGCGATAGCCTGCACGAGCACATTGCCAATCTGGCCGGCAGCCCCGCCATTAAGAAGGGCATCCT
GCAGACAGTGAAGGTGGTGGACGAGCTCGTGAAAGTGATGGGCCGGCACAAGCCCGAGAACATCGTGA
TCGAAATGGCCAGAGAGAACCAGACCACCCAGAAGGGACAGAAGAACAGCCGCGAGAGAATGAAGCGG
ATCGAAGAGGGCATCAAAGAGCTGGGCAGCCAGATCCTGAAAGAACACCCCGTGGAAAACACCCAGCT
GCAGAACGAGAAGCTGTACCTGTACTACCTGCAGAATGGGCGGGATATGTACGTGGACCAGGAACTGG
ACATCAACCGGCTGTCCGACTACGATGTGGACCACATCGTGCCTCAGAGCTTTCTGAAGGACGACTCC
ATCGACAACAAGGTGCTGACCAGAAGCGACAAGGCCCGGGGCAAGAGCGACAACGTGCCCTCCGAAGA
GGTCGTGAAGAAGATGAAGAACTACTGGCGGCAGCTGCTGAACGCCAAGCTGATTACCCAGAGAAAGT
TCGACAATCTGACCAAGGCCGAGAGAGGCGGCCTGAGCGAACTGGATAAGGCCGGCTTCATCAAGAGA
CAGCTGGTGGAAACCCGGCAGATCACAAAGCACGTGGCACAGATCCTGGACTCCCGGATGAACACTAA
GTACGACGAGAATGACAAGCTGATCCGGGAAGTGAAAGTGATCACCCTGAAGTCCAAGCTGGTGTCCG
ATTTCCGGAAGGATTTCCAGTTTTACAAAGTGCGCGAGATCAACAACTACCACCACGCCCACGACGCC
TACCTGAACGCCGTCGTGGGAACCGCCCTGATCAAAAAGTACCCTAAGCTGGAAAGCGAGTTCGTGTA
CGGCGACTACAAGGTGTACGACGTGCGGAAGATGATCGCCAAGAGCGAGCAGGAAATCGGCAAGGCTA
CCGCCAAGTACTTCTTCTACAGCAACATCATGAACTTTTTCAAGACCGAGATTACCCTGGCCAACGGC
GAGATCCGGAAGCGGCCTCTGATCGAGACAAACGGCGAAACCGGGGAGATCGTGTGGGATAAGGGCCG
GGATTTTGCCACCGTGCGGAAAGTGCTGAGCATGCCCCAAGTGAATATCGTGAAAAAGACCGAGGTGC
AGACAGGCGGCTTCAGCAAAGAGTCTATCCTGCCCAAGAGGAACAGCGATAAGCTGATCGCCAGAAAG
AAGGACTGGGACCCTAAGAAGTACGGCGGCTTCGACAGCCCCACCGTGGCCTATTCTGTGCTGGTGGT
GGCCAAAGTGGAAAAGGGCAAGTCCAAGAAACTGAAGAGTGTGAAAGAGCTGCTGGGGATCACCATCA
TGGAAAGAAGCAGCTTCGAGAAGAATCCCATCGACTTTCTGGAAGCCAAGGGCTACAAAGAAGTGAAA
AAGGACCTGATCATCAAGCTGCCTAAGTACTCCCTGTTCGAGCTGGAAAACGGCCGGAAGAGAATGCT
GGCCTCTGCCGGCGAACTGCAGAAGGGAAACGAACTGGCCCTGCCCTCCAAATATGTGAACTTCCTGT
ACCTGGCCAGCCACTATGAGAAGCTGAAGGGCTCCCCCGAGGATAATGAGCAGAAACAGCTGTTTGTG
GAACAGCACAAGCACTACCTGGACGAGATCATCGAGCAGATCAGCGAGTTCTCCAAGAGAGTGATCCT
GGCCGACGCTAATCTGGACAAAGTGCTGTCCGCCTACAACAAGCACCGGGATAAGCCCATCAGAGAGC
AGGCCGAGAATATCATCCACCTGTTTACCCTGACCAATCTGGGAGCCCCTGCCGCCTTCAAGTACTTT
GACACCACCATCGACCGGAAGAGGTACACCAGCACCAAAGAGGTGCTGGACGCCACCCTGATCCACCA
GAGCATCACCGGCCTGTACGAGACACGGATCGACCTGTCTCAGCTGGGAGGCGACAGCGCTGGAGGAG
GTGGAAGCGGAGGAGGAGGAAGCGGAGGAGGAGGTAGCGGACCTAAGAAAAAGAGGAAGGTGGCGGCC
GCTGGATCCGGACGGGCTGACGCATTGGACGATTTTGATCTGGATATGCTGGGAAGTGACGCCCTCGA
TGATTTTGACCTTGACATGCTTGGTTCGGATGCCCTTGATGACTTTGACCTCGACATGCTCGGCAGTG
ACGCCCTTGATGATTTCGACCTGGACATGCTGTAACTCGAGCAATAAAGAATCGTTTGTGTTATGTTT
CAACGTGTTTATTTTTCAATTGCAGCGGACCGAGCGGCCGCAGGAACCCCTAGTGATGGAGTTGGCCA
CTCCCTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGCTGCCTGCAGGGGCGCCTGATGCGGTATTTTCT
CCTTACGCATCTGTGCGGTATTTCACACCGCATACGTCAAAGCAACCATAGTACGCGCCCTGTAGCGG
CGCATTAAGCGCGGCGGGTGTGGTGGTTACGCGCAGCGTGACCGCTACACTTGCCAGCGCCCTAGCGC
CCGCTCCTTTCGCTTTCTTCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCCCAAAAAACTTGATTTGGG
TGATGGTTCACGTAGTGGGCCATCGCCCTGATAGACGGTTTTTCGCCCTTTGACGTTGGAGTCCACGT
TCTTTAATAGTGGACTCTTGTTCCAAACTGGAACAACACTCAACCCTATCTCGGGCTATTCTTTTGAT
TTATAAGGGATTTTGCCGATTTCGGCCTATTGGTTAAAAAATGAGCTGATTTAACAAAAATTTAACGC
GAATTTTAACAAAATATTAACGTTTACAATTTTATGGTGCACTCTCAGTACAATCTGCTCTGATGCCG
CATAGTTAAGCCAGCCCCGACACCCGCCAACACCCGCTGACGCGCCCTGACGGGCTTGTCTGCTCCCG
GCATCCGCTTACAGACAAGCTGTGACCGTCTCCGGGAGCTGCATGTGTCAGAGGTTTTCACCGTCATC
ACCGAAACGCGCGAGACGAAAGGGCCTCGTGATACGCCTATTTTTATAGGTTAATGTCATGATAATAA
TGGTTTCTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCACGAGTG
GGTTACATCGAACTGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCGGGCAAGAGC
AACTCGGTCGCCGCATACACTATTCTCAGAATGACTTGGTTGAGTACTCACCAGTCACAGAAAAGCAT
CTTACGGATGGCATGACAGTAAGAGAATTATGCAGTGCTGCCATAACCATGAGTGATAACACTGCGGC
CAACTTACTTCTGACAACGATCGGAGGACCGAAGGAGCTAACCGCTTTTTTGCACAACATGGGGGATC
ATGTAACTCGCCTTGATCGTTGGGAACCGGAGCTGAATGAAGCCATACCAAACGACGAGCGTGACACC
ACGATGCCTGTAGCAATGGCAACAACGTTGCGCAAACTATTAACTGGCGAACTACTTACTCTAGCTTC
CCGGCAACAATTAATAGACTGGATGGAGGCGGATAAAGTTGCAGGACCACTTCTGCGCTCGGCCCTTC
CGGCTGGCTGGTTTATTGCTGATAAATCTGGAGCCGGTGAGCGTGGGTCTCGCGGTATCATTGCAGCA
CTGGGGCCAGATGGTAAGCCCTCCCGTATCGTAGTTATCTACACGACGGGGAGTCAGGCAACTATGGA
TGAACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGCGTCAGACCCC
RightITR = first underlined and bold sequence
EF1α = first underlined sequence
dCas 9VP64 = bold sequence
FpA = second underlined sequence
LeftITR = second underlined and bold sequence
AAV-MPH
LeftITR-CMV-MPH-HGHpA-U6-sgRNA-RightITR
SEQ ID NO: 11
AGATCAAAGGATCTTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGAAGGTAACTGGCTT
CAGCAGAGCGCAGATACCAAATACTGTCCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTCCTGCAGGCAGCTG
CGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCA
CGCGTGGAGCTAGTTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCGCCCATTGACGTC
AATAATGACGTATGTTCCCATAGTAACGTCAATAGGGACTTTCCATTGACGTCAATGGGTGGAGTATT
TACGGTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTTGGCA
GTACATCTACGTATTAGTCATCGCTATTACCATGGTGATGCGGTTTTGGCAGTACATCAATGGGCGTG
GATAGCGGTTTGACTCACGGGGATTTCCAAGTCTCCACCCCATTGACGTCAATGGGAGTTTGTTTTGC
ACCAAAATCAACGGGACTTTCCAAAATGTCGTAACAACTCCGCCCCATTGACGCAAATGGGCGGTAGG
CGTGTACGGTGGGAGGTCTATATAAGCAGAGCTCGTTTAGTGAACCGTCAGATCGCCTGGAGACGCCA
TCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTA
TAGGCCCACAAAAAATGCTTTCTTCTTTTAATATACTTTTTTGTTTATCTTATTTCTAATACTTTCCC
TAATCTCTTTCTTTCAGGGCAATAATGATACAATGTATCATGCCTCTTTGCACCATTCTAAAGAATAA
CAGTGATAATTTCTGGGTTAAGGCAATAGCAATATTTCTGCATATAAATATTTCTGCATATAAATTGT
AACTGATGTAAGAGGTTTCATATTGCTAATAGCAGCTACAATCCAGCTACCATTCTGCTTTTATTTTA
TGGTTGGGATAAGGCTGGATTATTCTGAGTCCAAGCTAGGCCCTTTTGCTAATCATGTTCATACCTCT
TATCTTCCTCCCACAGCTCCTGGGCAACGTGCTGGTCTGTGTGCTGGCCCATCACTTTGGCAAAGAAT
TGGGATTCGAACATCGATTGAATTCACCATGGCTTCAAACTTTACTCAGTTCGTGCTCGTGGACAATG
GTGGGACAGGGGATGTGACAGTGGCTCCTTCTAATTTCGCTAATGGGGTGGCAGAGTGGATCAGCTCC
AACTCACGGAGCCAGGCCTACAAGGTGACATGCAGCGTCAGGCAGTCTAGTGCCCAGAAGAGAAAGTA
TACCATCAAGGTGGAGGTCCCCAAAGTGGCTACCCAGACAGTGGGCGGAGTCGAACTGCCTGTCGCCG
CTTGGAGGTCCTACCTGAACATGGAGCTCACTATCCCAATTTTCGCTACCAATTCTGACTGTGAACTC
ATCGTGAAGGCAATGCAGGGGCTCCTCAAAGACGGTAATCCTATCCCTTCCGCCATCGCCGCTAACTC
AGGTATCTACAGCGCTGGAGGAGGTGGAAGCGGAGGAGGAGGAAGCGGAGGAGGAGGTAGCGGACCTA
AGAAAAAGAGGAAGGTGGCGGCCGCTGGATCCCCTTCAGGGCAGATCAGCAACCAGGCCCTGGCTCTG
GCCCCTAGCTCCGCTCCAGTGCTGGCCCAGACTATGGTGCCCTCTAGTGCTATGGTGCCTCTGGCCCA
GCCACCTGCTCCAGCCCCTGTGCTGACCCCAGGACCACCCCAGTCACTGAGCGCTCCAGTGCCCAAGT
CTACACAGGCCGGCGAGGGGACTCTGAGTGAAGCTCTGCTGCACCTGCAGTTCGACGCTGATGAGGAC
CTGGGAGCTCTGCTGGGGAACAGCACCGATCCCGGAGTGTTCACAGATCTGGCCTCCGTGGACAACTC
TGAGTTTCAGCAGCTGCTGAATCAGGGCGTGTCCATGTCTCATAGTACAGCCGAACCAATGCTGATGG
AGTACCCCGAAGCCATTACCCGGCTGGTGACCGGCAGCCAGCGGCCCCCCGACCCCGCTCCAACTCCC
CTGGGAACCAGCGGCCTGCCTAATGGGCTGTCCGGAGATGAAGACTTCTCAAGCATCGCTGATATGGA
CTTTAGTGCCCTGCTGTCACAGATTTCCTCTAGTGGGCAGGGAGGAGGTGGAAGCGGCTTCAGCGTGG
ACACCAGTGCCCTGCTGGACCTGTTCAGCCCCTCGGTGACCGTGCCCGACATGAGCCTGCCTGACCTT
GACAGCAGCCTGGCCAGTATCCAAGAGCTCCTGTCTCCCCAGGAGCCCCCCAGGCCTCCCGAGGCAGA
GAACAGCAGCCCGGATTCAGGGAAGCAGCTGGTGCACTACACAGCGCAGCCGCTGTTCCTGCTGGACC
CCGGCTCCGTGGACACCGGGAGCAACGACCTGCCGGTGCTGTTTGAGCTGGGAGAGGGCTCCTACTTC
TCCGAAGGGGACGGCTTCGCCGAGGACCCCACCATCTCCCTGCTGACAGGCTCGGAGCCTCCCAAAGC
CAAGGACCCCACTGTCTCCTGACCTCGAGCAGCGCTGCTCGAGAGATCTACGGGTGGCATCCCTGTGA
CCCCTCCCCAGTGCCTCTCCTGGCCCTGGAAGTTGCCACTCCAGTGCCCACCAGCCTTGTCCTAATAA
AATTAAGTTGCATCATTTTGTCTGACTAGGTGTCCTTCTATAATATTATGGGGTGGAGGGGGGTGGTA
TGGAGCAAGGGGCAAGTTGGGAAGACAACCTGTAGGGCCTGCGGGGTCTATTGGGAACCAAGCTGGAG
TGCAGTGGCACAATCTTGGCTCACTGCAATCTCCGCCTCCTGGGTTCAAGCGATTCTCCTGCCTCAGC
CTCCCGAGTTGTTGGGATTCCAGGCATGCATGACCAGGCTCAGCTAATTTTTGTTTTTTTGGTAGAGA
CGGGGTTTCACCATATTGGCCAGGCTGGTCTCCAACTCCTAATCTCAGGTGATCTACCCACCTTGGCC
TCCCAAATTGCTGGGATTACAGGCGTGAACCACTGCTCCCTTCCCTGTCCTTCTGATTTTGTAGGTAA
CCACGTGCGGACCTAGGGATAACAGGGTAATGAGGGCCTATTTCCCATGATTCCTTCATATTTGCATA
TACGATACAAGGCTGTTAGAGAGATAATTGGAATTAATTTGACTGTAAACACAAAGATATTAGTACAA
AATACGTGACGTAGAAAGTAATAATTTCTTGGGTAGTTTGCAGTTTTAAAATTATGTTTTAAAATGGA
CTATCATATGCTTACCGTAACTTGAAAGTATTTCGATTTCTTGGCTTTATATATCTTGTGGAAAGGAC

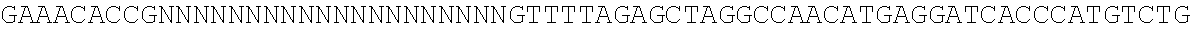

CAGGGCCTAGCAAGTTAAAATAAGGCTAGTCCGTTATCAACTTGGCCAACATGAGGATCACCCATGTC
TGCAGGGCCAAGTGGCACCGAGTCGGTGCTTTTTTTGGATCCTGTTGACAATTAATCATCGGCATAGT
ATATCGGCATAGTATAATACGACAAGGTGAGGAACTAAACCATGGCCAAGTTGACCAGTGCCGTTCCG
GTGCTCACCGCGCGCGACGTCGCCGGAGCGGTCGAGTTCTGGACCGACCGGCTCGGGTTCTCCCGGGA
CTTCGTGGAGGACGACTTCGCCGGTGTGGTCCGGGACGACGTGACCCTGTTCATCAGCGCGGTCCAGG
ACCAGGTGGTGCCGGACAACACCCTGGCCTGGGTGTGGGTGCGCGGCCTGGACGAGCTGTACGCCGAG
TGGTCGGAGGTCGTGTCCACGAACTTCCGGGACGCCTCCGGGCCGGCCATGACCGAGATCGGCGAGCA
GCCGTGGGGGCGGGAGTTCGCCCTGCGCGACCCGGCCGGCAACTGCGTGCACTTCGTGGCCGAGGAGC
AGGACTGATAGGGATAACAGGGTAATTAACTATAACGGTCCTAAGGTAGCGAAGGACCGAGCGGCCGC
AGGAACCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGCTGCCT
GCAGGGGCGCCTGATGCGGTATTTTCTCCTTACGCATCTGTGCGGTATTTCACACCGCATACGTCAAA
GCAACCATAGTACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTTGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGGCTATTCTTTTGATTTATAAGGGATTTTGCCGATTTCGGCCTATTGGTTAAAAAA
TGAGCTGATTTAACAAAAATTTAACGCGAATTTTAACAAAATATTAACGTTTACAATTTTATGGTGCA
CTCTCAGTACAATCTGCTCTGATGCCGCATAGTTAAGCCAGCCCCGACACCCGCCAACACCCGCTGAC
GCGCCCTGACGGGCTTGTCTGCTCCCGGCATCCGCTTACAGACAAGCTGTGACCGTCTCCGGGAGCTG
CATGTGTCAGAGGTTTTCACCGTCATCACCGAAACGCGCGAGACGAAAGGGCCTCGTGATACGCCTAT
TTTTATAGGTTAATGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTATGTGGCGCGGTAT
TATCCCGTATTGACGCCGGGCAAGAGCAACTCGGTCGCCGCATACACTATTCTCAGAATGACTTGGTT
GAGTACTCACCAGTCACAGAAAAGCATCTTACGGATGGCATGACAGTAAGAGAATTATGCAGTGCTGC
CATAACCATGAGTGATAACACTGCGGCCAACTTACTTCTGACAACGATCGGAGGACCGAAGGAGCTAA
CCGCTTTTTTGCACAACATGGGGGATCATGTAACTCGCCTTGATCGTTGGGAACCGGAGCTGAATGAA
GCCATACCAAACGACGAGCGTGACACCACGATGCCTGTAGCAATGGCAACAACGTTGCGCAAACTATT
AACTGGCGAACTACTTACTCTAGCTTCCCGGCAACAATTAATAGACTGGATGGAGGCGGATAAAGTTG
CAGGACCACTTCTGCGCTCGGCCCTTCCGGCTGGCTGGTTTATTGCTGATAAATCTGGAGCCGGTGAG
CGTGGGTCTCGCGGTATCATTGCAGCACTGGGGCCAGATGGTAAGCCCTCCCGTATCGTAGTTATCTA
CACGACGGGGAGTCAGGCAACTATGGATGAACGAAATAGACAGATCGCTGAGATAGGTGCCTCACTGA
TTAAGCATTGGTAACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCCCTTAACGTGAGTT
TTCGTTCCACTGAGCGTCAGACCCCGTAGAAA
RightITR = first underlined and bold sequence
CMV = first underlined sequence
MPH = first bold sequence
HGHpA = second underlined sequence
U6 = second bold sequence
LeftITR = second underlined and bold sequence
AAV-dCasΦ1-MPH-sgRNA
LeftITR-CBh-dCasΦ1-P2A-MPH-FpA-U6-sgRNA-RightITR
SEQ ID NO: 12
AGATCAAAGGATCTTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGAAGGTAACTGGCTT
CAGCAGAGCGCAGATACCAAATACTGTCCTTCTAGTGTAGCCGTAGTTAGGCCACCACTTCAAGAACT
CTGTAGCACCGCCTACATACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTCCTGCAGGCAGCTG
CGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCA
CGCGTCGTTACATAACTTACGGTAAATGGCCCGCCTGGCTGACCGCCCAACGACCCCCGCCCATTGAC
GTCAATAATGACGTATGTTCCCATAGTAACGCCAATAGGGACTTTCCATTGACGTCAATGGGTGGAGT
ATTTACGGTAAACTGCCCACTTGGCAGTACATCAAGTGTATCATATGCCAAGTACGCCCCCTATTGAC
GTCAATGACGGTAAATGGCCCGCCTGGCATTATGCCCAGTACATGACCTTATGGGACTTTCCTACTTG
GCAGTACATCTACGTATTAGTCATCGCTATTACCATGGTCGAGGTGAGCCCCACGTTCTGCTTCACTC
TCCCCATCTCCCCCCCCTCCCCACCCCCAATTTTGTATTTATTTATTTTTTAATTATTTTGTGCAGCG
ATGGGGGCGGGGGGGGGGGGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGCGACGCTGCCTTCG
CCCCGTGCCCCGCTCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCTGAGCAAGAGGTAAGGGTTTAAG
GGATGGTTGGTTGGTGGGGTATTAATGTTTAATTACCTGGAGCACCTGTCCGGAGAATTCGCCACCAT
GAAAAGGCCGGCGGCCACGAAAAAGGCCGGCCAGGCAAAAAAGAAAAAGGCCGATACCCCCACACTGT
TCACCCAATTCCTCAGACACCACCTCCCCGGCCAAAGATTTAGAAAGGACATTCTGAAGCAAGCCGGA
AGAATCCTCGCTAATAAGGGAGAGGACGCCACAATTGCCTTTCTGAGAGGCAAATCCGAGGAGAGCCC
TCCCGACTTCCAACCCCCCGTGAAGTGCCCCATCATCGCTTGCAGCAGACCTCTGACAGAATGGCCCA
TCTATCAAGCCAGCGTGGCTATCCAAGGCTACGTCTACGGCCAGTCTCTGGCCGAATTTGAGGCCAGC
GACCCCGGCTGTTCCAAGGATGGACTCCTCGGATGGTTTGACAAGACCGGCGTCTGCACCGATTATTT
CAGCGTGCAAGGACTGAACCTCATTTTCCAGAACGCTAGGAAGAGGTATATCGGCGTGCAGACCAAGG
TGACCAATAGAAACGAAAAGAGGCACAAAAAGCTGAAGAGGATCAACGCCAAGAGAATCGCTGAAGGA
CTGCCCGAGCTGACCTCCGACGAGCCCGAGAGCGCTCTGGATGAAACCGGCCATCTGATCGACCCTCC
CGGACTGAACACAAACATCTACTGCTACCAGCAAGTGAGCCCTAAGCCTCTGGCTCTCAGCGAGGTGA
ATCAGCTGCCCACCGCCTACGCTGGATACAGCACCTCCGGAGATGATCCCATCCAGCCCATGGTGACC
AAAGATAGACTGAGCATCTCCAAAGGCCAGCCCGGATATATCCCCGAGCACCAGAGGGCTCTGCTGAG
CCAAAAGAAGCATAGAAGGATGAGAGGCTACGGACTGAAGGCTAGGGCTCTGCTCGTGATCGTGAGGA
TTCAAGATGACTGGGCCGTCATCGATCTGAGGTCTCTGCTGAGGAACGCTTACTGGAGGAGGATCGTC
CAGACAAAGGAGCCCTCCACAATCACCAAGCTGCTCAAGCTCGTGACCGGCGATCCCGTGCTGGACGC
CACCAGAATGGTCGCCACCTTCACCTATAAACCCGGAATCGTGCAAGTGAGGAGCGCTAAATGTCTGA
AGAACAAGCAAGGCAGCAAGCTGTTCAGCGAAAGGTATCTGAACGAAACCGTGAGCGTGACCAGCATT
GCCCTCGGCTCCAACAATCTGGTCGCTGTGGCCACCTACAGACTGGTCAACGGAAATACCCCCGAACT
GCTGCAGAGGTTTACACTCCCTAGCCATCTGGTGAAGGATTTCGAGAGGTACAAACAAGCTCACGATA
CACTGGAGGACTCCATTCAGAAGACCGCCGTGGCTTCTCTGCCCCAAGGCCAGCAAACCGAGATTAGA
ATGTGGTCCATGTACGGCTTTAGAGAGGCCCAAGAGAGGGTCTGTCAAGAGCTGGGACTGGCCGACGG
ATCCATCCCTTGGAATGTGATGACCGCCACATCCACCATTCTGACAGATCTCTTTCTGGCCAGAGGAG
GAGACCCCAAGAAGTGCATGTTCACCAGCGAGCCCAAGAAGAAGAAGAACTCCAAGCAAGTGCTCTAT
AAGATTAGAGATAGAGCTTGGGCCAAGATGTACAGAACACTGCTGTCCAAAGAGACCAGAGAGGCTTG
GAATAAAGCTCTGTGGGGACTGAAAAGGGGCAGCCCCGACTATGCCAGACTGTCCAAGAGGAAGGAAG
AGCTGGCTAGAAGATGCGTCAACTACACCATCTCCACCGCCGAGAAGAGGGCCCAGTGTGGAAGGACC
ATTGTGGCCCTCGAAGATCTGAACATCGGCTTCTTCCACGGCAGAGGAAAACAAGAGCCCGGATGGGT
GGGACTGTTCACAAGAAAGAAGGAGAACAGATGGCTCATGCAAGCCCTCCACAAGGCTTTTCTGGAGC
TGGCTCATCATAGAGGCTACCACGTCATCGAAGTCAACCCCGCCTATACCTCCCAGACATGCCCCGTG
TGTAGACATTGCGACCCCGACAATAGAGACCAGCATAACAGAGAGGCCTTCCACTGTATCGGATGTGG
CTTCAGAGGCAACGCTGACCTCGACGTGGCCACCCACAACATTGCTATGGTGGCCATCACCGGCGAAT
CCCTCAAAAGGGCCAGAGGCTCCGTGGCTTCCAAGACACCTCAACCTCTGGCCGCCGAGGGCAGTGGA
GAGGGCAGAGGAAGTCTGCTAACATGCGGTGACGTCGAGGAGAATCCTGGCCCAGCCACCATGGCTTC
AAACTTTACTCAGTTCGTGCTCGTGGACAATGGTGGGACAGGGGATGTGACAGTGGCTCCTTCTAATT
TCGCTAATGGGGTGGCAGAGTGGATCAGCTCCAACTCACGGAGCCAGGCCTACAAGGTGACATGCAGC
GTCAGGCAGTCTAGTGCCCAGAAGAGAAAGTATACCATCAAGGTGGAGGTCCCCAAAGTGGCTACCCA
GACAGTGGGCGGAGTCGAACTGCCTGTCGCCGCTTGGAGGTCCTACCTGAACATGGAGCTCACTATCC
CAATTTTCGCTACCAATTCTGACTGTGAACTCATCGTGAAGGCAATGCAGGGGCTCCTCAAAGACGGT
AATCCTATCCCTTCCGCCATCGCCGCTAACTCAGGTATCTACAGCGCTGGAGGAGGTGGAAGCGGAGG
AGGAGGAAGCGGAGGAGGAGGTAGCGGACCTAAGAAAAAGAGGAAGGTGGCGGCCGCTGGATCCCCTT
CAGGGCAGATCAGCAACCAGGCCCTGGCTCTGGCCCCTAGCTCCGCTCCAGTGCTGGCCCAGACTATG
GTGCCCTCTAGTGCTATGGTGCCTCTGGCCCAGCCACCTGCTCCAGCCCCTGTGCTGACCCCAGGACC
ACCCCAGTCACTGAGCGCTCCAGTGCCCAAGTCTACACAGGCCGGCGAGGGGACTCTGAGTGAAGCTC
TGCTGCACCTGCAGTTCGACGCTGATGAGGACCTGGGAGCTCTGCTGGGGAACAGCACCGATCCCGGA
GTGTTCACAGATCTGGCCTCCGTGGACAACTCTGAGTTTCAGCAGCTGCTGAATCAGGGCGTGTCCAT
GTCTCATAGTACAGCCGAACCAATGCTGATGGAGTACCCCGAAGCCATTACCCGGCTGGTGACCGGCA
GCCAGCGGCCCCCCGACCCCGCTCCAACTCCCCTGGGAACCAGCGGCCTGCCTAATGGGCTGTCCGGA
GATGAAGACTTCTCAAGCATCGCTGATATGGACTTTAGTGCCCTGCTGTCACAGATTTCCTCTAGTGG
GCAGGGAGGAGGTGGAAGCGGCTTCAGCGTGGACACCAGTGCCCTGCTGGACCTGTTCAGCCCCTCGG
TGACCGTGCCCGACATGAGCCTGCCTGACCTTGACAGCAGCCTGGCCAGTATCCAAGAGCTCCTGTCT
CCCCAGGAGCCCCCCAGGCCTCCCGAGGCAGAGAACAGCAGCCCGGATTCAGGGAAGCAGCTGGTGCA
CTACACAGCGCAGCCGCTGTTCCTGCTGGACCCCGGCTCCGTGGACACCGGGAGCAACGACCTGCCGG
TGCTGTTTGAGCTGGGAGAGGGCTCCTACTTCTCCGAAGGGGACGGCTTCGCCGAGGACCCCACCATC
TCCCTGCTGACAGGCTCGGAGCCTCCCAAAGCCAAGGACCCCACTGTCTCCTGACCTCGAGCAATAAA
GAATCGTTTGTGTTATGTTTCAACGTGTTTATTTTTCAATTGCAGCGGACCTAGGGATAACAGGGTAA
TGAGGGCCTATTTCCCATGATTCCTTCATATTTGCATATACGATACAAGGCTGTTAGAGAGATAATTG
GAATTAATTTGACTGTAAACACAAAGATATTAGTACAAAATACGTGACGTAGAAAGTAATAATTTCTT
GGGTAGTTTGCAGTTTTAAAATTATGTTTTAAAATGGACTATCATATGCTTACCGTAACTTGAAAGTA
TTTCGATTTCTTGGCTTTATATATCTTGTGGAAAGGACGGCCAACATGAGGATCACCCATGTCTGCAG

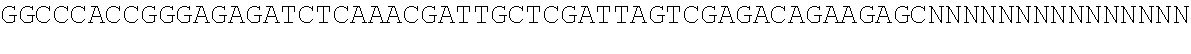

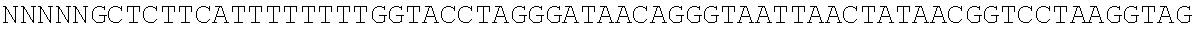

GCTCACTGAGGCCGGGCGACCAAAGGTCGCCCGACGCCCGGGCTTTGCCCGGGCGGCCTCAGTGAGCG
AGCGAGCGCGCAGCTGCCTGCAGGGGCGCCTGATGCGGTATTTTCTCCTTACGCATCTGTGCGGTATT
TCACACCGCATACGTCAAAGCAACCATAGTACGCGCCCTGTAGCGGCGCATTAAGCGCGGCGGGTGTG
GTGGTTACGCGCAGCGTGACCGCTACACTTGCCAGCGCCCTAGCGCCCGCTCCTTTCGCTTTCTTCCC
TTCCTTTCTCGCCACGTTCGCCGGCTTTCCCCGTCAAGCTCTAAATCGGGGGCTCCCTTTAGGGTTCC
GATTTAGTGCTTTACGGCACCTCGACCCCAAAAAACTTGATTTGGGTGATGGTTCACGTAGTGGGCCA
TCGCCCTGATAGACGGTTTTTCGCCCTTTGACGTTGGAGTCCACGTTCTTTAATAGTGGACTCTTGTT
CCAAACTGGAACAACACTCAACCCTATCTCGGGCTATTCTTTTGATTTATAAGGGATTTTGCCGATTT
CGGCCTATTGGTTAAAAAATGAGCTGATTTAACAAAAATTTAACGCGAATTTTAACAAAATATTAACG
TTTACAATTTTATGGTGCACTCTCAGTACAATCTGCTCTGATGCCGCATAGTTAAGCCAGCCCCGACA
CCCGCCAACACCCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCGCGAGACGAAAG
GGCCTCGTGATACGCCTATTTTTATAGGTTAATGTCATGATAATAATGGTTTCTTAGACGTCAGGTGG
CACTTTTCGGGGAAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGAGTATGAGTATTCAA
CATTTCCGTGTCGCCCTTATTCCCTTTTTTGCGGCATTTTGCCTTCCTGTTTTTGCTCACCCAGAAAC
GCTGGTGAAAGTAAAAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAGCACTTTTAAAGTT
CTGCTATGTGGCGCGGTATTATCCCGTATTGACGCCGGGCAAGAGCAACTCGGTCGCCGCATACACTA
TTCTCAGAATGACTTGGTTGAGTACTCACCAGTCACAGAAAAGCATCTTACGGATGGCATGACAGTAA
GAGAATTATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTTGATCGTTG
GGAACCGGAGCTGAATGAAGCCATACCAAACGACGAGCGTGACACCACGATGCCTGTAGCAATGGCAA
CAACGTTGCGCAAACTATTAACTGGCGAACTACTTACTCTAGCTTCCCGGCAACAATTAATAGACTGG
ATGGAGGCGGATAAAGTTGCAGGACCACTTCTGCGCTCGGCCCTTCCGGCTGGCTGGTTTATTGCTGA
TAAATCTGGAGCCGGTGAGCGTGGGTCTCGCGGTATCATTGCAGCACTGGGGCCAGATGGTAAGCCCT
CCCGTATCGTAGTTATCTACACGACGGGGAGTCAGGCAACTATGGATGAACGAAATAGACAGATCGCT
GAGATAGGTGCCTCACTGATTAAGCATTGGTAACTGTCAGACCAAGTTTACTCATATATACTTTAGAT
TGATTTAAAACTTCATTTTTAATTTAAAAGGATCTAGGTGAAGATCCTTTTTGATAATCTCATGACCA
AAATCCCTTAACGTGAGTTTTCGTTCCACTGAGCGTCAGACCCCGTAGAAA
RightITR = first underlined and bold sequence
CBh = first underlined sequence
dCasΦ1 = first bold sequence
P2A = second underlined sequence
MPH = second bold sequence
FpA = third underlined sequence
U6 = third bold sequence
LeftITR = second underlined and bold sequence

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a polycystic kidney disease (PKD), wherein said method comprises administering to said mammal nucleic acid encoding a polycystin-1 (PC-1) polypeptide or a variant of said PC-1 polypeptide, wherein said PC-1 polypeptide or said variant is expressed by kidney cells within said mammal.

2. The method of claim 1, wherein said nucleic acid encoding said PC-1 polypeptide or said variant is administered to said mammal in the form of a viral vector.

3. The method of claim 2, wherein said viral vector is a helper-dependent adenovirus (HDAd) vector.

4. The method of claim 1, wherein said nucleic acid encoding said PC-1 polypeptide or said variant is operably linked to a promoter sequence.

5. The method of claim 4, wherein said promoter sequence is selected from the group consisting of a human elongation factor 1α (EF1α) promoter sequence, a chicken β-actin hybrid (CBh) promoter sequence, a PKD1 promoter sequence, a PKD2 promoter sequence, a cytomegalovirus (CMV) promoter sequence, a Rous sarcoma virus (RSV) promoter sequence, an aquaporin 2 (AQP2) promoter sequence, a gamma-glutamyltransferase 1 (Ggt1) promoter sequence, and a Ksp-cadherin promoter sequence.

6. A method for treating a mammal having a polycystic kidney disease (PKD), wherein said method comprises administering to said mammal nucleic acid encoding a polycystin-2 (PC-2) polypeptide or a variant of said PC-2 polypeptide, wherein said PC-2 polypeptide or said variant is expressed by kidney cells within said mammal.

7. The method of claim 6, wherein said nucleic acid encoding said PC-2 polypeptide or said variant is administered to said mammal in the form of a viral vector.

8. The method of claim 7, wherein said viral vector is an adenovirus-associated virus (AAV) vector.

9. The method of claim 6, wherein said nucleic acid encoding said PC-2 polypeptide or said variant is operably linked to a promoter sequence.

10. The method of claim 9, wherein said promoter sequence is selected from the group consisting of a EF1α promoter sequence, a CBh promoter sequence, a PKD1 promoter sequence, a PKD2 promoter sequence, a CMV promoter sequence, a RSV promoter sequence, an AQP2 promoter sequence, a Ggt1 promoter sequence, and a Ksp-cadherin promoter sequence.

11. A method for treating a mammal having a polycystic kidney disease (PKD), wherein said method comprises administering to said mammal:
(a) nucleic acid encoding a PC-1 polypeptide or a variant of said PC-1 polypeptide, wherein said PC-1 polypeptide or said variant is expressed by kidney cells within said mammal; and
(b) nucleic acid encoding a PC-2 polypeptide or a variant of said PC-2 polypeptide, wherein said PC-2 polypeptide or said variant is expressed by kidney cells within said mammal.

12-19. (canceled)

20. The method of claim 11, wherein said nucleic acid encoding said PC-1 polypeptide or said variant and said nucleic acid encoding said PC-2 polypeptide or said variant are administered to said mammal in the form of a viral vector.

21. The method of claim 20, wherein said viral vector is a HDAd vector.

22. The method of claim 20, wherein said nucleic acid encoding said PC-1 polypeptide or said variant is operably linked to a first promoter sequence, and wherein said nucleic acid encoding said PC-2 polypeptide or said variant is operably linked to a second promoter sequence.

23. The method of claim 22, wherein said first promoter sequence and said second promoter sequence are each independently selected from the group consisting of a EF1α promoter sequence, a CBh promoter sequence, a PKD1 promoter sequence, a PKD2 promoter sequence, a CMV promoter sequence, a RSV promoter sequence, an AQP2 promoter sequence, a Ggt1 promoter sequence, and a Ksp-cadherin promoter sequence.

24. The method of claim 1, wherein said method comprises identifying said mammal as being in need of a treatment for said PKD.

25. The method of claim 1, wherein said mammal is a human.

26. The method of claim 1, wherein PKD is an autosomal dominant PKD (ADPKD).

27. The method of claim 1, wherein said method further comprises, prior to said administering said nucleic acid, administering a lipopolysaccharides (LPS) to said mammal.

28. (canceled)

29. (canceled)

30. A method for treating a mammal having a PKD, wherein said method comprises administering to said mammal:
(a) nucleic acid encoding a fusion polypeptide including a deactivated Cas (dCas) polypeptide and a transcriptional activator polypeptide;
(b) nucleic acid encoding a helper activator polypeptide; and
(c) nucleic acid encoding a nucleic acid molecule including (i) a nucleic acid sequence that is complementary to a target sequence within a PKD1 gene, and (ii) a nucleic acid sequence that can bind said helper activator polypeptide.

31-48. (canceled)

49. A method for delivering nucleic acid to a cell within a mammal, wherein said method comprises:
(a) administering a proteinuria-inducing agent to said mammal; and
(b) administering said nucleic acid to said mammal.

50-60. (canceled)


SEQUENCE LISTING (26 sequences)

SEQ ID NO: 1 - DNA, 12933 bp, artificial
  nucleic acid encoding a PC-1 polypeptide
atgccgcccgccgcgcccgcccgcctggcgctggccctgggcctgggcctgtggctcggggcgctggcggggggccccgggggcgcgccggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 2 - protein, 4310 aa, homo sapiens
MPPAAPARLALALGLGLWLGALAGGPGGAPGGPGRGCGPCEPPCLCGPAPGAACRVNCSGRGLRTLGPALRIPADATALDVSHNLLRALDVGLLANLSALAELDISNNKISTLEEGIFANLFNLSEINLSGNPFECDCGLAWLPRWAEEQQVRVVQPEAATCAGPGSLAGQPLLGIPLLDSGCGEEYVACLPDNSSGTVAAVSFSAAHEGLLQPEACSAFCFSTGQGLAALSEQGWCLCGAAQPSSASFACLSLCSGPPPPPAPTCRGPTLLQHVFPASPGATLVGPHGPLASGQLAAFHIAAPLPVTATRWDFGDGSAEVDAAGPAASHRYVLPGRYHVTAVLALGAGSALLGTDVQVEAAPAALELVCPSSVQSDESLDLSIQNRGGSGLEAAYSIVALGEEPARAVHPLCPSDTEIFPGNGHCYRLVVEKAAWLQAQEQCQAWAGAALAMVDSPAVQRFLVSRVTRSLDVWIGFSTVQGVEVGPAPQGEAFSLESCQNWLPGEPHPATAEHCVRLGPTGWCNTDLCSAPHSYVCELQPGGPVQDAENLLVGAPSGDLQGPLTPLAQQDGLSAPHEPVEVMVFPGLRLSREAFLTTAEFGTQELRRPAQLRLQVYRLLSTAGTPENGSEPESRSPDNRTQLAPACMPGGRWCPGANICLPLDASCHPQACANGCTSGPGLPGAPYALWREFLFSVPAGPPAQYSVTLHGQDVLMLPGDLVGLQHDAGPGALLHCSPAPGHPGPQAPYLSANASSWLPHLPAQLEGTWACPACALRLLAATEQLTVLLGLRPNPGLRLPGRYEVRAEVGNGVSRHNLSCSFDVVSPVAGLRVIYPAPRDGRLYVPTNGSALVLQVDSGANATATARWPGGSVSARFENVCPALVATFVPGCPWETNDTLFSVVALPWLSEGEHVVDVVVENSASRANLSLRVTAEEPICGLRATPSPEARVLQGVLVRYSPVVEAGSDMVFRWTINDKQSLTFQNVVFNVIYQSAAVFKLSLTASNHVSNVTVNYNVTVERMNRMQGLQVSTVPAVLSPNATLALTAGVLVDSAVEVAFLWTFGDGEQALHQFQPPYNESFPVPDPSVAQVLVEHNVMHTYAAPGEYLLTVLASNAFENLTQQVPVSVRASLPSVAVGVSDGVLVAGRPVTFYPHPLPSPGGVLYTWDFGDGSPVLTQSQPAANHTYASRGTYHVRLEVNNTVSGAAAQADVRVFEELRGLSVDMSLAVEQGAPVVVSAAVQTGDNITWTFDMGDGTVLSGPEATVEHVYLRAQNCTVTVGAASPAGHLARSLHVLVFVLEVLRVEPAACIPTQPDARLTAYVTGNPAHYLFDWTFGDGSSNTTVRGCPTVTHNFTRSGTFPLALVLSSRVNRAHYFTSICVEPEVGNVTLQPERQFVQLGDEAWLVACAWPPFPYRYTWDFGTEEAAPTRARGPEVTFIYRDPGSYLVTVTASNNISAANDSALVEVQEPVLVTSIKVNGSLGLELQQPYLFSAVGRGRPASYLWDLGDGGWLEGPEVTHAYNSTGDFTVRVAGWNEVSRSEAWLNVTVKRRVRGLVVNASRTVVPLNGSVSFSTSLEAGSDVRYSWVLCDRCTPIPGGPTISYTFRSVGTFNIIVTAENEVGSAQDSIFVYVLQLIEGLQVVGGGRYFPTNHTVQLQAVVRDGTNVSYSWTAWRDRGPALAGSGKGFSLTVLEAGTYHVQLRATNMLGSAWADCTMDFVEPVGWLMVAASPNPAAVNTSVTLSAELAGGSGVVYTWSLEEGLSWETSEPFTTHSFPTPGLHLVTMTAGNPLGSANATVEVDVQVPVSGLSIRASEPGGSFVAAGSSVPFWGQLATGTNVSWCWAVPGGSSKRGPHVTMVFPDAGTFSIRLNASNAVSWVSATYNLTAEEPIVGLVLWASSKVVAPGQLVHFQILLAAGSAVTFRLQVGGANPEVLPGPRFSHSFPRVGDHVVSVRGKNHVSWAQAQVRIVVLEAVSGLQVPNCCEPGIATGTERNFTARVQRGSRVAYAWYFSLQKVQGDSLVILSGRDVTYTPVAAGLLEIQVRAFNALGSENRTLVLEVQDAVQYVALQSGPCFTNRSAQFEAATSPSPRRVAYHWDFGDGSPGQDTDEPRAEHSYLRPGDYRVQVNASNLVSFFVAQATVTVQVLACREPEVDVVLPLQVLMRRSQRNYLEAHVDLRDCVTYQTEYRWEVYRTASCQRPGRPARVALPGVDVSRPRLVLPRLALPVGHYCFVFVVSFGDTPLTQSIQANVTVAPERLVPIIEGGSYRVWSDTRDLVLDGSESYDPNLEDGDQTPLSFHWACVASTQREAGGCALNFGPRGSSTVTIPRERLAAGVEYTFSLTVWKAGRKEEATNQTVLIRSGRVPIVSLECVSCKAQAVYEVSRSSYVYLEGRCLNCSSGSKRGRWAARTFSNKTLVLDETTTSTGSAGMRLVLRRGVLRDGEGYTFTLTVLGRSGEEEGCASIRLSPNRPPLGGSCRLFPLGAVHALTTKVHFECTGWHDAEDAGAPLVYALLLRRCRQGHCEEFCVYKGSLSSYGAVLPPGFRPHFEVGLAVVVQDQLGAAVVALNRSLAITLPEPNGSATGLTVWLHGLTASVLPGLLRQADPQHVIEYSLALVTVLNEYERALDVAAEPKHERQHRAQIRKNITETLVSLRVHTVDDIQQIAAALAQCMGPSRELVCRSCLKQTLHKLEAMMLILQAETTAGTVTPTAIGDSILNITGDLIHLASSDVRAPQPSELGAESPSRMVASQAYNLTSALMRILMRSRVLNEEPLTLAGEEIVAQGKRSDPRSLLCYGGAPGPGCHFSIPEAFSGALANLSDVVQLIFLVDSNPFPFGYISNYTVSTKVASMAFQTQAGAQIPIERLASERAITVKVPNNSDWAARGHRSSANSANSVVVQPQASVGAVVTLDSSNPAAGLHLQLNYTLLDGHYLSEEPEPYLAVYLHSEPRPNEHNCSASRRIRPESLQGADHRPYTFFISPGSRDPAGSYHLNLSSHFRWSALQVSVGLYTSLCQYFSEEDMVWRTEGLLPLEETSPRQAVCLTRHLTAFGASLFVPPSHVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 3 - DNA, 2907 bp, artificial
  nucleic acid encoding a PC-2 polypeptide
atggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatga

SEQ ID NO: 4 - protein, 968 aa, homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 5 - DNA, 34347 bp, artificial
  HDAd-PKD1 vector sequence
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggatccatgcatgttaagtttaaacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttgatgccccccctcgaggttcgacggtatcgataagcttgatttaattaaggccggcccctaggggcgcgcgcggccgctagggataacagggtaattgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaacgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgtccggagaattcgccaccatgccgcccgccgcgcccgcccgcctggcgctggccctgggcctgggcctgtggctcggggcgctggcggggggccccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcgcgccggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttaactataacggtcctaaggtagcgaagtcgaccgaatcgttgtcccttgtcacagccattgagaattttggcagggagcatgttcttagagcatttttaggctctgcgggacataacagctctgcctcagagcacatgcctttctcagctcctgaaagccactgatcaaattggaacattttgtaccttagggatgaggatatcaactctcccagccacttagagggataaatgtgatgatgcattcaattgtgactacatctgatcccaactgttgcttcagctgctctcctatagcacatggcgggaggcgtgcatcccagtagctacctccccacttttggggagatgtggttccatccatgaaacctgggtacccgcctaccaggtcctggcctatcaggtggcagggtctggtcaaagaagggcatgtgtggtcttcagcaagggagacaggacggtggtgcagagcgtctagaccctcagggcaagtctcccccacacctgctcccggggcagttgtctttgtgacctcccatccccctctgtttcatcctctataaaatgaggggctgagccccaaaataacaggcttctttgccatgatgcaaaactgctgaatctttctttctgacacacaaggcatcgagcagcctctgaaagaaccaaagccactagcaggcttcctgacttgggtttgtaggtactgaatactcccttgaaaaataaaaacatagaggcacttttctcctggctgtttattacagaacgaagaaaaaacacactggcttgaaacagacgccagatttcaaatgtagaggtgaaatacgaggtggcaattaaaatgtgattacagaaagtctggacactgagaaaagtttacaggacagtgggtgtgggttttctataacagacacttaaatatacatgacgataattgcagatagaaaccatcaaagacaaaccccaaatcaactaataatgtttacagatgttcccccccaaaccacagagccttacatcaaaacaaatactgaaaggctttaaaccaggaacagctcgccttaaccccacgagggtgcacacaagctgggctttttctctcggtctgaatggtaaagggaggaggatactctagctcctccaggtggattgctgagacagggctcggctcacacactgtctctgcgcctctcccaaatctggagaactctcccagcctcctggtaaagtgtctctgtggggcacttaacgataaaacagcttctgctgtaaagctcattaggaaagagctagcggagactgaaaggttcgcaaaagagattaagaatcacacaaggcaataggatttttagtgaacatagaaataaatggccaagtggttttctatttggcatttgtcaacttgcacaacaactcttggtcatatccacattgctcattgcattaaaaccataagcgactcagccacctagcttaacaaggtatcactggagcaaacaacacggtctgcatatttgtaacattgtataataaacacaaaacaatgcatagtaaacacaactctactgaaacaaaagccgtcgctttatttacaaagtcacaaaatgaagtataaatacttctgtcattaatgtttaggaaaaccatttacaaaattttcaaatatgtacacgtagcttgaaaaatcaccagctttccattttgtcacaggtagagagagggataagcatgggctgacaacaccactcaaattgtaacgggagacaactgcgggtatggatcgacaccacttcctagagtgatgtcaccatgggggtttctatgggcatcctgctcagatttaaagtgccccagcatcctgggtgacttgcccagaattctgggctgtggcattttgagcagcagcatgctgttccaaaatgtcgtcgatcagcctcaagttgcacacccagtcttcatctgggctcacacaggagcctttcaagagagcttcaatgaaatctacctcattgcagtcaggtgacgaaatcagatcatttagtgggggttggggctggcgcaaaaagtcggcaggtggcagctcagggggaatatccgttctgtcgaacggacctgggaactggctggcagcaacggcagaagcagcagcagcggtggcagcagcagccacatagcttggtggctcgatgccctgtatggggctcaggggactaaagctggccataccctgctggaggaacttggtggtgtttgctacaggcaccgggccctgtaccgggctctgcctgaggctctggctgcccagcaggctgaagctggggttgttggccaggggcacttgtgttcccatcgcagcgggcacttgtgcctcccaatcagatggcctctgaaggcaggcctggccagaaggtgagtgctgctgaacgctattatccacttggctgaggggtgttttccccgaaactgctgtggtcacagctgctgccgctgtgacccatgcagcattgttgaacgcagtgggcattcttggcacactaggccgtctgagctggtggggactcaaggactgggtgcccagggagctgggacagaacccaggcaggggcacttctggtggggtggccttggggctctgcatatgctggcagacagagtcaagtctgcccaggggagtctggcctgagtgtgagaggatgggacactgggggctggaggtgaaaattccttgccgcttccccagagttggtgagatcactcccatgccctcgcagctctggtgcctggtgagtgggatcattcctggactcagattgttctgaagaagcccagttctgggtggcatcaagtgcttgctagatggggggcttgccttgatccggctacacttggaggtgacttgttcttggacggctacatacagaaagagagaagtggggatgagttccaaaggcatcctcgacttcggctgtggccaccggagggtagctcctggcccaacacggacttctcacctcccgcccttggctctctactgagctcccccctgctccccaattcctcgccattcccctcatttctctgccctcagcctggactgcagttcttctgggaagctgccccaactccctaggtctgtgctcaccaagagcagatcacactggactgaaatgccagctgatttgtctcttcaagaaaattggaagctcctggaggtcagggtccatgtctgcttttacactcagtgctctgtatgcaggcctggcactgcccaccctttgacaggtggtgcatattttgtagaaggaaggaaggggccaggtggggtgggctgggctggtggcgggagctagctcagcctcttagattctctacccgatggatgtgacctgggacagcaagtgagtgtggtgagtgagtgcagacggtgctttgttcccctcttgtctcatagcctagatggcctctgagcccagatctggggctcagacaacatttgttcaactgaacggtaatgggtttcctttctgaaggctgaaatctgggagctgacattctggactccctgagttctgaagagcctggggatggagagacacggagcagaagatggaaggtagagtcccaggtgcctaagatggggaatacatctcccctcattgtcatgagagtccactctagctgatatctactgtggccaatatctaccggtacttttttggggtggacactgagtcatgcagcagtcttatggtttacccaaggtcaggtaggggagacagtgcagtcagagcacaagcccagtgtgtctgacccacccaagaatccatgctcgtatctacaaaaatgattttttctcttgtaatggtgcctaggttcttttattatcatggcatgtgtatgtttttcaactaggttacaatctggccttataaggttaacctcctggaggccaccagccttcctgaaacttgtctgtgctgtccctgcaactggagtgtgcctgatgtggcactccagcctggacaagtgggacacagactccgctgttatcaggcccaaagatgtcttccataagaccagaagagcaatggtgtagaggtgtcatgggctacaataaagatgctgacctcctgtctgagggcaagcagcctcttctggccctcagacaaatgctgagtgttcccaagactaccctcggcctggtccaatctcatcccactggtgcgtaagggttgctgaactcatgacttcttggctagcctgcaacctccacggagtgggaactacatcaggcattttgctaactgctgtatcctaggccaataaatgttgatcacatttatagctgccatggtagggtggggacccctgctatctatctgtggaggctctgggagcccctgacacaaactttctgaagcagagcctccccaaccccttttccattccctatacctgacagatggcccaggaacccattagaaatggaaggtcactgcagcagtatgtgaatgtgcgtgtgggagaagggcaggatcagagccctgggggtgtggcagcccccaagtgattctaatccagatcctagggttgtttccctgtcccattgaaatagctgctttaaggggcctgactcagggaaatcagtctcttgaattaagtggtgattttggagtcatttagaccaggccttcaattgggatccactagttctagagcggccgggcccagggaaccccgcaggcgggggcggccagtttcccgggttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgttgatttttcatctctaaacatcaacttacttaattctgccaatttcttttttttgagacagggtctcactctgtcacctaggatggagtgcagtggcacaatcactgctcactgcagcctcgacttcccgggctcgggtgattctccccaggctcaggggattctcccacttcagcctcccaagtagctgggactacaggtgcgcaccaccatccctggctaatatttgtactttattttatttatttatttatttattttttgagatggagtttcgctcttgttgcccaaatgaattgcctcttatttaatttcgtctgatgatacattttgtttttattttgtaaaaaattattttttttctttttggagacagggtcttgctctgttgcccaggctggtcacaaactcctgacctcaagcaatcctcctgccttagcctcccaaaatgctgggattacaggcgtgacgacctcgcccggccttgtattatgatacattttgaacaactacaagtagacttggtataatgaacctgcacgtacccattgccaagttctgacaactgtctgtctatagccaattatgcatttcttaaattagaacccccccaatatacccaaatatatatatatgtgtgcatatatatagtaagttgtaacaaagttgtgaattcatacctgaagtatctcaagtgatgcaagttttatgaatttttgtttatgccttttgggaagagttgtattgacaaattttttatgcttaaagtaaaccataaatcaaaaaaataaaatctaggatgcaataaaacaaaacaacttcttgacataagtatggtatgtaaatctgttttgattggaaatcaatttgttatattgccagaattcctgttttagaatacatctctgctgatctgtctgtattcttagactgcatatctgggatgaactctgggcagaattcacatgggcttcctttgaaataaacaagacttttcaaattcttagtcgatctgcagaacctgtagccaggcactgaaccattttgatagatgcagtaatcgttgcaagtgtatatttcaagggagttctggctgggtcctagtttatgcttgtggcagaagcagtgagtaactgggaggaagttggtgagtaagcttcaaggaagaagtcatttttagtactctggatcttcctgattttaaagcactacaaaatggtgcattttcattcttgtcaagtgataacagatatattctgatgagcctgaaatgaatatatattgtatcatttttataatatctagcaaggtttgtattttcctagaacttgaactaaatttcagttcataaaatttataaaatacttagttgttgtaaaatatttttggaatgttcacataggtgacacacaaatgtcccattttcattctttctatagtaaatatgttctgatatgtgaaggtttagcagatgcatcagcatttaatcctagaggatctggcataatcttttcccccaagaatagaaattttttctgcttatgaaagtagtacatgtttctttaaaaacaaatcaatattgacttctgcctgctgtatagcactatgcctccacctggccatgaccaggggcatgtcctggtccacctacctgaaaatgtttgcaaccagcctcctggccatgtgcacaggggctgaagttgtcccacaggtattacgggccaacctgacaatacatgaagttccaccaaagtctgagaactcagaactgagctttggggactgaaagacagcacaaacctcaaatttctcagcactggaaacctcaaaatataactgaattccataaataagattttaagtcttaaatatgtatttttaaatgtattaaaagtcaagctgcttgtatttaagcacctaatacaatgcttaggttgtaaaaggagatgctcaataggtactaactgatatattgagatttaattatggtttgaccaatatttattggaaaccgccaaagcttaaatcatcagcttcttgaatgtgatttgaaaggtaatttagtattgaatagcatgtgagctagagtatttcattctttctggtttatttcttcaaatagactttgaatataatggtgaatgggtattataaattaactaataaaaatgacattgaaaatgaaaaaatatatatattaaagtgtagaaagtgaccaggcgtggtggctcacacctgtaatccaagcaccttgggaggctgaggcaggaggatctcttgatcccaggagttcaagaccagcctgggcaacatagcgagacttcgtctctaaaaaaaaaaaagagagagaaaaaaattttttttatttaaaaaaagtgtagaaagtgtcaagaccccacttcttaccattatttggtatatttctctatacccacccacccttcctccttactccctccctcccttcccaatctttttatctttttgtattctgattttttgtttgtatattttgctttaatttaatgtatcctttaaaaatttcccatacattttatatgtatatataaaaacgcatgctgccaaagataatttataagaaagaccattgaatttttttaaaagtgatatatattcattgaaaaaaatttagaatatatagcaaagcaataaagaactaaataaaattgctgtaactcctctttcaaagataagtgcttttatgattttgttgtatttttttctgtatataggtacatatatagtatttataaagctgtactcatagtacattttcacatcacaggtaccatatcagtgttattaaatattttgtatgccaggggctagacataccaagacaaccaatatgtggttctacttaaataatattagagtatcttttatgatgacacttcatgagttgactataataatcttagacttctaagagtttgggttttcaaaagatcacttagcttttttgggtgatttttcccccttactgtgagatgagagaggctgtttggatttgggattggggtagcggggacagcaacttttcttttctttttcttttttattttgaggtagggtattgctgtgtcacccaggctggagtgcagtggtgtgatctcggctcactgcaacctccacctcccgggctcaggtgatcctcctgcttcagcctcccagtaactgggactacaggcgcgtgccacatgcctggctaattttgtatttttagtagagatggggtttcaccatgttggccaggctggtctctaactcctgacctcaggtgatacgcccacctgggcctcccaaaatactgggattacaggcatgagccgctgcatcagccagcagtttttcttgtggttttttttgtttgttttgttttgttttgtttttgagatagggtcttactctgttgtccacgctggagtgctgtggtatgatcgtagctcactgcagcctcaaactcctgggctcaagtgattccttctgcctccgcctcccgagtagctgggactacaggtatgcaccaccatacctggcaaatttttacaaagttttttgtagggacggggtcttgctacattccccatgtcggtcttgaactcctggcctcaagcaactctcctgtctcagcctcccaaagcactgggattacaagtgtgagccaccacaccatgccagtttttcctgttcagtgtgatattttatcttgttagactacagtgtgttaaaacttgttttactaaattttcaaacatactcaaaagtggagagaatagtataatgaatacccgtatgttcatcacccatgtttagaatattattaaatataaagattttgctgcgtttgtcttagctctttaaaatttttctttttctctttgtgacctaaaggaaattccatatcttatcactttacttctacattcttgactaagatgactaagacatatagttacatggttttttgttttgtttttgttttttaaagacgaaatctcgctcttgtcccccaggctggagtgcaatggtgccatctcagctcagtgcaacctctgccttctgggtacaagcgattctcctgcctcagcctcccaagtagctgggattacaggctcctgccaccacgcctggctaatttttgtatttttagtagagacggcggggggaggtttcaccatgttgacaaggctggtctggaactcctgacctcaggtgatccacccgcctcggcctcccaaagtgctgggattacaggcgtgagccaccgcgcccagcctgtttttttgtttgtgtgttttgttttttttgagacagagtcttgctctgtttcccaggctggagtgaagtggtgccatctcagctcagagacagagtcttgctctgtttcccaggctggagtgaagtggtgccatcttggctcactgcaaccttcacctcccaggttcaagtgattctcctgcctcagcctcccaagtagctgggactacaggcatgtgtcaccacacccggctaatttttttgtatttttagtagagacgggatttcaccgtgttgcccaggctggtctcgaactcctgagctcaggcagtctgcctgcctcagcctcccaaagtgctgggattacacgtgtgaaccaacccgcccggcctgttgttttcttacataattcattatcatacctacaaagttaacagttactaatatcatcttacacctaaatttctctgatagactaaggttattttttaacatcttaatccaatcaaatgtttgtatcctgtaatgctctcattgaaacagctatatttctttttcagattagtgatgatgaaccaggttatgaccttgatttattttgcatacctaatcattatgctgaggatttggaaagggtgtttattcctcatggactaattatggacaggtaagtaagatcttaaaatgaggttttttactttttcttgtgttaatttcaaacatcagcagctgttctgagtacttgctatttgaacataaactaggccaacttattaaataactgatgctttctaaaatcttctttattaaaaataaaagaggagggccttactaattacttagtatcagttgtggtatagtgggactctgtagggaccagaacaaagtaaacattgaagggagatggaagaaggaactctagccagagtcttgcatttctcagtcctaaacagggtaatggactggggctgaatcacatgaaggcaaggtcagatttttattattatgcacatctagcttgaaaattttctgttaagtcaattacagtgaaaaaccttacctggtattgaatgcttgcattgtatgtctggctattctgtgtttttattttaaaattataatatcaaaatatttgtgttataaaatattctaactatggaggccataaacaagaagactaaagttctctcctttcagccttctgtacacatttcttctcaagcactggcctatgcatgtatactatatgcaaaagtacatatatacatttatattttaacgtatgagtatagttttaaatgttattggacacttttaatattagtgtgtctagagctatctaatatattttaaaggttgcatagcattctgtcttatggagataccataactgatttaaccagtccactattgatagacactattttgttcttaccgactgtactagaagaaacattcttttacatgtttggtacttgttcagctttattcaagtggaatttctgggtcaaggggaaagagtttattgaatattttggtattgccaaattttcctctaagaagttgaatcattttatactcctgatgttatatgagagtacctttctcttcacaatttgtctcttttttttttttttttgagacaaggtctctgttgcccaggctggggtgcagtgcagcagaatgatcacagttcactgcagtctcaacctcctgggttcaagcgatccttccacctcagcctcctgagtagctgggactataggtgtgcgccaccactcccagctaatatttttattttgtagaaacagggttcgccatgttacccagcctcccaaagtgctgggattacaggcatgagccactggcccagtttctacagtctctcttaatattgtatattatccagaaaatttcatttaatcagaacctgccagtctgataggtgaaaatggtatcttgtttttatttgcatttaaaaaaaattatgatagtggtatgcttggtttttttgaaggtatcaaattttttaccttatgaaacatgagggcaaaggatgtgatacgtggaagatttaaaaaaaatttttaatgcatttttttgagacaaggtcttgctctattgtccaggctggagtgcagtggcacaatcacagttcactccagcctcaacatcctgcactaaagtgattttcccacctcacctctcaagtagctgggactacaggtacatgctaccatgcctggctaatttttttttttttgcaggcatggggtctcactatattgcccaggttggtgtggaagtttaatgactaagaggtgtttgttataaagtttaatgtatgaaactttctattaaattcctgattttatttctgtaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtgtgagtatatttaatatatgattctttttagtggcaacagtaggttttcttatattttctttgaatctctgcaaaccatacttgctttcatttcacttggttacagtgagatttttctaacatattcactagtactttacatcaaagccaatactgtttttttaaaactagtcaccttggaggatatatacttattttacaggtgtgtgtggttttttaaataaactccttttaggaattgctgttgggacttgggatacttttttcactatacatactggtgacagataccctctcttgagctacatcggtttgtggggagtcaaaagtcctttggagctaggtttgacaaataaggtgggttaacacttgtttcctagaaagcacatggagagctagagtattggcgaattgaagaaatccccctttttttttaacacacttaagaaaggggactgcaggtatactcaagagagtaagtcgcaccagaaaccacttttgatccacagtctgcctgtgtcacacaattgaaatgcatcacaacattgacactgtggatgaaacaaaatcagtgtgaattttagtagtgaatttcattcataatttgatcgtgcaaacgtttgatttttattactttagactattgtttctgattttatgttgggttggtatttcctgtgagttactgttttacctttaaaataggaatttttcatactcttcaaagattagaacaaatgtccagtttttgctgtttcatgaatgagtcctgtccatctttgtagaaactcgccttatgttcacatttttattgagaataagaccacttatctacatttaactatcaacctcatcctctccattaatcatctattttagtgacccaagtttttgaccttttccatgtttacatcaatcctgtaggtgattgggcagccatttaagtattattatagacattttcactatcccattaaaaccctttatgcccatacatcataacactacttcctacccataagctccttttaacttgttaaagtcttgcttgaattaaagacttgtttacggtatcgataagcttgatatcaaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgtttaaacattaagaattaattcgatcctgaatggcgaatggacgcgccctgtagcggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatggctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagac

SEQ ID NO: 6 - DNA, 7344 bp, artificial
  AAV-PKD2 vector sequence
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaactataacggtcctaaggtagcgaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatgaaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatccggaggcggcggcacgggcggcggcagcggcggcatggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgattctagagtcgacctgcagaagcttgcctcgagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaactataacggtcctaaggtagcgaacggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaa

SEQ ID NO: 7 - DNA, 38755 bp, artificial
  HDAd-PKD1-PKD2 vector sequence
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggatccatgcatgttaagtttaaacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttgatgccccccctcgaggttcgacggtatcgataagcttgatttaattaaggccggcccctaggggcgcgcgcggccgctagggataacagggtaattgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaacgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgtccggagaattcgccaccatgccgcccgccgcgcccgcccgcctggcgctggccctgggcctgggcctgtggctcggggcgctggcggggggccccgggatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcgcgccggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctttaactataacggtcctaaggtagcgaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatgaaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatccggaggcggcggcacgggcggcggcagcggcggcatggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgattctagagtcgacctgcagaagcttgcctcgagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtaactataacggtcctaaggtagcgaagtcgaccgaatcgttgtcccttgtcacagccattgagaattttggcagggagcatgttcttagagcatttttaggctctgcgggacataacagctctgcctcagagcacatgcctttctcagctcctgaaagccactgatcaaattggaacattttgtaccttagggatgaggatatcaactctcccagccacttagagggataaatgtgatgatgcattcaattgtgactacatctgatcccaactgttgcttcagctgctctcctatagcacatggcgggaggcgtgcatcccagtagctacctccccacttttggggagatgtggttccatccatgaaacctgggtacccgcctaccaggtcctggcctatcaggtggcagggtctggtcaaagaagggcatgtgtggtcttcagcaagggagacaggacggtggtgcagagcgtctagaccctcagggcaagtctcccccacacctgctcccggggcagttgtctttgtgacctcccatccccctctgtttcatcctctataaaatgaggggctgagccccaaaataacaggcttctttgccatgatgcaaaactgctgaatctttctttctgacacacaaggcatcgagcagcctctgaaagaaccaaagccactagcaggcttcctgacttgggtttgtaggtactgaatactcccttgaaaaataaaaacatagaggcacttttctcctggctgtttattacagaacgaagaaaaaacacactggcttgaaacagacgccagatttcaaatgtagaggtgaaatacgaggtggcaattaaaatgtgattacagaaagtctggacactgagaaaagtttacaggacagtgggtgtgggttttctataacagacacttaaatatacatgacgataattgcagatagaaaccatcaaagacaaaccccaaatcaactaataatgtttacagatgttcccccccaaaccacagagccttacatcaaaacaaatactgaaaggctttaaaccaggaacagctcgccttaaccccacgagggtgcacacaagctgggctttttctctcggtctgaatggtaaagggaggaggatactctagctcctccaggtggattgctgagacagggctcggctcacacactgtctctgcgcctctcccaaatctggagaactctcccagcctcctggtaaagtgtctctgtggggcacttaacgataaaacagcttctgctgtaaagctcattaggaaagagctagcggagactgaaaggttcgcaaaagagattaagaatcacacaaggcaataggatttttagtgaacatagaaataaatggccaagtggttttctatttggcatttgtcaacttgcacaacaactcttggtcatatccacattgctcattgcattaaaaccataagcgactcagccacctagcttaacaaggtatcactggagcaaacaacacggtctgcatatttgtaacattgtataataaacacaaaacaatgcatagtaaacacaactctactgaaacaaaagccgtcgctttatttacaaagtcacaaaatgaagtataaatacttctgtcattaatgtttaggaaaaccatttacaaaattttcaaatatgtacacgtagcttgaaaaatcaccagctttccattttgtcacaggtagagagagggataagcatgggctgacaacaccactcaaattgtaacgggagacaactgcgggtatggatcgacaccacttcctagagtgatgtcaccatgggggtttctatgggcatcctgctcagatttaaagtgccccagcatcctgggtgacttgcccagaattctgggctgtggcattttgagcagcagcatgctgttccaaaatgtcgtcgatcagcctcaagttgcacacccagtcttcatctgggctcacacaggagcctttcaagagagcttcaatgaaatctacctcattgcagtcaggtgacgaaatcagatcatttagtgggggttggggctggcgcaaaaagtcggcaggtggcagctcagggggaatatccgttctgtcgaacggacctgggaactggctggcagcaacggcagaagcagcagcagcggtggcagcagcagccacatagcttggtggctcgatgccctgtatggggctcaggggactaaagctggccataccctgctggaggaacttggtggtgtttgctacaggcaccgggccctgtaccgggctctgcctgaggctctggctgcccagcaggctgaagctggggttgttggccaggggcacttgtgttcccatcgcagcgggcacttgtgcctcccaatcagatggcctctgaaggcaggcctggccagaaggtgagtgctgctgaacgctattatccacttggctgaggggtgttttccccgaaactgctgtggtcacagctgctgccgctgtgacccatgcagcattgttgaacgcagtgggcattcttggcacactaggccgtctgagctggtggggactcaaggactgggtgcccagggagctgggacagaacccaggcaggggcacttctggtggggtggccttggggctctgcatatgctggcagacagagtcaagtctgcccaggggagtctggcctgagtgtgagaggatgggacactgggggctggaggtgaaaattccttgccgcttccccagagttggtgagatcactcccatgccctcgcagctctggtgcctggtgagtgggatcattcctggactcagattgttctgaagaagcccagttctgggtggcatcaagtgcttgctagatggggggcttgccttgatccggctacacttggaggtgacttgttcttggacggctacatacagaaagagagaagtggggatgagttccaaaggcatcctcgacttcggctgtggccaccggagggtagctcctggcccaacacggacttctcacctcccgcccttggctctctactgagctcccccctgctccccaattcctcgccattcccctcatttctctgccctcagcctggactgcagttcttctgggaagctgccccaactccctaggtctgtgctcaccaagagcagatcacactggactgaaatgccagctgatttgtctcttcaagaaaattggaagctcctggaggtcagggtccatgtctgcttttacactcagtgctctgtatgcaggcctggcactgcccaccctttgacaggtggtgcatattttgtagaaggaaggaaggggccaggtggggtgggctgggctggtggcgggagctagctcagcctcttagattctctacccgatggatgtgacctgggacagcaagtgagtgtggtgagtgagtgcagacggtgctttgttcccctcttgtctcatagcctagatggcctctgagcccagatctggggctcagacaacatttgttcaactgaacggtaatgggtttcctttctgaaggctgaaatctgggagctgacattctggactccctgagttctgaagagcctggggatggagagacacggagcagaagatggaaggtagagtcccaggtgcctaagatggggaatacatctcccctcattgtcatgagagtccactctagctgatatctactgtggccaatatctaccggtacttttttggggtggacactgagtcatgcagcagtcttatggtttacccaaggtcaggtaggggagacagtgcagtcagagcacaagcccagtgtgtctgacccacccaagaatccatgctcgtatctacaaaaatgattttttctcttgtaatggtgcctaggttcttttattatcatggcatgtgtatgtttttcaactaggttacaatctggccttataaggttaacctcctggaggccaccagccttcctgaaacttgtctgtgctgtccctgcaactggagtgtgcctgatgtggcactccagcctggacaagtgggacacagactccgctgttatcaggcccaaagatgtcttccataagaccagaagagcaatggtgtagaggtgtcatgggctacaataaagatgctgacctcctgtctgagggcaagcagcctcttctggccctcagacaaatgctgagtgttcccaagactaccctcggcctggtccaatctcatcccactggtgcgtaagggttgctgaactcatgacttcttggctagcctgcaacctccacggagtgggaactacatcaggcattttgctaactgctgtatcctaggccaataaatgttgatcacatttatagctgccatggtagggtggggacccctgctatctatctgtggaggctctgggagcccctgacacaaactttctgaagcagagcctccccaaccccttttccattccctatacctgacagatggcccaggaacccattagaaatggaaggtcactgcagcagtatgtgaatgtgcgtgtgggagaagggcaggatcagagccctgggggtgtggcagcccccaagtgattctaatccagatcctagggttgtttccctgtcccattgaaatagctgctttaaggggcctgactcagggaaatcagtctcttgaattaagtggtgattttggagtcatttagaccaggccttcaattgggatccactagttctagagcggccgggcccagggaaccccgcaggcgggggcggccagtttcccgggttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgttgatttttcatctctaaacatcaacttacttaattctgccaatttcttttttttgagacagggtctcactctgtcacctaggatggagtgcagtggcacaatcactgctcactgcagcctcgacttcccgggctcgggtgattctccccaggctcaggggattctcccacttcagcctcccaagtagctgggactacaggtgcgcaccaccatccctggctaatatttgtactttattttatttatttatttatttattttttgagatggagtttcgctcttgttgcccaaatgaattgcctcttatttaatttcgtctgatgatacattttgtttttattttgtaaaaaattattttttttctttttggagacagggtcttgctctgttgcccaggctggtcacaaactcctgacctcaagcaatcctcctgccttagcctcccaaaatgctgggattacaggcgtgacgacctcgcccggccttgtattatgatacattttgaacaactacaagtagacttggtataatgaacctgcacgtacccattgccaagttctgacaactgtctgtctatagccaattatgcatttcttaaattagaacccccccaatatacccaaatatatatatatgtgtgcatatatatagtaagttgtaacaaagttgtgaattcatacctgaagtatctcaagtgatgcaagttttatgaatttttgtttatgccttttgggaagagttgtattgacaaattttttatgcttaaagtaaaccataaatcaaaaaaataaaatctaggatgcaataaaacaaaacaacttcttgacataagtatggtatgtaaatctgttttgattggaaatcaatttgttatattgccagaattcctgttttagaatacatctctgctgatctgtctgtattcttagactgcatatctgggatgaactctgggcagaattcacatgggcttcctttgaaataaacaagacttttcaaattcttagtcgatctgcagaacctgtagccaggcactgaaccattttgatagatgcagtaatcgttgcaagtgtatatttcaagggagttctggctgggtcctagtttatgcttgtggcagaagcagtgagtaactgggaggaagttggtgagtaagcttcaaggaagaagtcatttttagtactctggatcttcctgattttaaagcactacaaaatggtgcattttcattcttgtcaagtgataacagatatattctgatgagcctgaaatgaatatatattgtatcatttttataatatctagcaaggtttgtattttcctagaacttgaactaaatttcagttcataaaatttataaaatacttagttgttgtaaaatatttttggaatgttcacataggtgacacacaaatgtcccattttcattctttctatagtaaatatgttctgatatgtgaaggtttagcagatgcatcagcatttaatcctagaggatctggcataatcttttcccccaagaatagaaattttttctgcttatgaaagtagtacatgtttctttaaaaacaaatcaatattgacttctgcctgctgtatagcactatgcctccacctggccatgaccaggggcatgtcctggtccacctacctgaaaatgtttgcaaccagcctcctggccatgtgcacaggggctgaagttgtcccacaggtattacgggccaacctgacaatacatgaagttccaccaaagtctgagaactcagaactgagctttggggactgaaagacagcacaaacctcaaatttctcagcactggaaacctcaaaatataactgaattccataaataagattttaagtcttaaatatgtatttttaaatgtattaaaagtcaagctgcttgtatttaagcacctaatacaatgcttaggttgtaaaaggagatgctcaataggtactaactgatatattgagatttaattatggtttgaccaatatttattggaaaccgccaaagcttaaatcatcagcttcttgaatgtgatttgaaaggtaatttagtattgaatagcatgtgagctagagtatttcattctttctggtttatttcttcaaatagactttgaatataatggtgaatgggtattataaattaactaataaaaatgacattgaaaatgaaaaaatatatatattaaagtgtagaaagtgaccaggcgtggtggctcacacctgtaatccaagcaccttgggaggctgaggcaggaggatctcttgatcccaggagttcaagaccagcctgggcaacatagcgagacttcgtctctaaaaaaaaaaaagagagagaaaaaaattttttttatttaaaaaaagtgtagaaagtgtcaagaccccacttcttaccattatttggtatatttctctatacccacccacccttcctccttactccctccctcccttcccaatctttttatctttttgtattctgattttttgtttgtatattttgctttaatttaatgtatcctttaaaaatttcccatacattttatatgtatatataaaaacgcatgctgccaaagataatttataagaaagaccattgaatttttttaaaagtgatatatattcattgaaaaaaatttagaatatatagcaaagcaataaagaactaaataaaattgctgtaactcctctttcaaagataagtgcttttatgattttgttgtatttttttctgtatataggtacatatatagtatttataaagctgtactcatagtacattttcacatcacaggtaccatatcagtgttattaaatattttgtatgccaggggctagacataccaagacaaccaatatgtggttctacttaaataatattagagtatcttttatgatgacacttcatgagttgactataataatcttagacttctaagagtttgggttttcaaaagatcacttagcttttttgggtgatttttcccccttactgtgagatgagagaggctgtttggatttgggattggggtagcggggacagcaacttttcttttctttttcttttttattttgaggtagggtattgctgtgtcacccaggctggagtgcagtggtgtgatctcggctcactgcaacctccacctcccgggctcaggtgatcctcctgcttcagcctcccagtaactgggactacaggcgcgtgccacatgcctggctaattttgtatttttagtagagatggggtttcaccatgttggccaggctggtctctaactcctgacctcaggtgatacgcccacctgggcctcccaaaatactgggattacaggcatgagccgctgcatcagccagcagtttttcttgtggttttttttgtttgttttgttttgttttgtttttgagatagggtcttactctgttgtccacgctggagtgctgtggtatgatcgtagctcactgcagcctcaaactcctgggctcaagtgattccttctgcctccgcctcccgagtagctgggactacaggtatgcaccaccatacctggcaaatttttacaaagttttttgtagggacggggtcttgctacattccccatgtcggtcttgaactcctggcctcaagcaactctcctgtctcagcctcccaaagcactgggattacaagtgtgagccaccacaccatgccagtttttcctgttcagtgtgatattttatcttgttagactacagtgtgttaaaacttgttttactaaattttcaaacatactcaaaagtggagagaatagtataatgaatacccgtatgttcatcacccatgtttagaatattattaaatataaagattttgctgcgtttgtcttagctctttaaaatttttctttttctctttgtgacctaaaggaaattccatatcttatcactttacttctacattcttgactaagatgactaagacatatagttacatggttttttgttttgtttttgttttttaaagacgaaatctcgctcttgtcccccaggctggagtgcaatggtgccatctcagctcagtgcaacctctgccttctgggtacaagcgattctcctgcctcagcctcccaagtagctgggattacaggctcctgccaccacgcctggctaatttttgtatttttagtagagacggcggggggaggtttcaccatgttgacaaggctggtctggaactcctgacctcaggtgatccacccgcctcggcctcccaaagtgctgggattacaggcgtgagccaccgcgcccagcctgtttttttgtttgtgtgttttgttttttttgagacagagtcttgctctgtttcccaggctggagtgaagtggtgccatctcagctcagagacagagtcttgctctgtttcccaggctggagtgaagtggtgccatcttggctcactgcaaccttcacctcccaggttcaagtgattctcctgcctcagcctcccaagtagctgggactacaggcatgtgtcaccacacccggctaatttttttgtatttttagtagagacgggatttcaccgtgttgcccaggctggtctcgaactcctgagctcaggcagtctgcctgcctcagcctcccaaagtgctgggattacacgtgtgaaccaacccgcccggcctgttgttttcttacataattcattatcatacctacaaagttaacagttactaatatcatcttacacctaaatttctctgatagactaaggttattttttaacatcttaatccaatcaaatgtttgtatcctgtaatgctctcattgaaacagctatatttctttttcagattagtgatgatgaaccaggttatgaccttgatttattttgcatacctaatcattatgctgaggatttggaaagggtgtttattcctcatggactaattatggacaggtaagtaagatcttaaaatgaggttttttactttttcttgtgttaatttcaaacatcagcagctgttctgagtacttgctatttgaacataaactaggccaacttattaaataactgatgctttctaaaatcttctttattaaaaataaaagaggagggccttactaattacttagtatcagttgtggtatagtgggactctgtagggaccagaacaaagtaaacattgaagggagatggaagaaggaactctagccagagtcttgcatttctcagtcctaaacagggtaatggactggggctgaatcacatgaaggcaaggtcagatttttattattatgcacatctagcttgaaaattttctgttaagtcaattacagtgaaaaaccttacctggtattgaatgcttgcattgtatgtctggctattctgtgtttttattttaaaattataatatcaaaatatttgtgttataaaatattctaactatggaggccataaacaagaagactaaagttctctcctttcagccttctgtacacatttcttctcaagcactggcctatgcatgtatactatatgcaaaagtacatatatacatttatattttaacgtatgagtatagttttaaatgttattggacacttttaatattagtgtgtctagagctatctaatatattttaaaggttgcatagcattctgtcttatggagataccataactgatttaaccagtccactattgatagacactattttgttcttaccgactgtactagaagaaacattcttttacatgtttggtacttgttcagctttattcaagtggaatttctgggtcaaggggaaagagtttattgaatattttggtattgccaaattttcctctaagaagttgaatcattttatactcctgatgttatatgagagtacctttctcttcacaatttgtctcttttttttttttttttgagacaaggtctctgttgcccaggctggggtgcagtgcagcagaatgatcacagttcactgcagtctcaacctcctgggttcaagcgatccttccacctcagcctcctgagtagctgggactataggtgtgcgccaccactcccagctaatatttttattttgtagaaacagggttcgccatgttacccagcctcccaaagtgctgggattacaggcatgagccactggcccagtttctacagtctctcttaatattgtatattatccagaaaatttcatttaatcagaacctgccagtctgataggtgaaaatggtatcttgtttttatttgcatttaaaaaaaattatgatagtggtatgcttggtttttttgaaggtatcaaattttttaccttatgaaacatgagggcaaaggatgtgatacgtggaagatttaaaaaaaatttttaatgcatttttttgagacaaggtcttgctctattgtccaggctggagtgcagtggcacaatcacagttcactccagcctcaacatcctgcactaaagtgattttcccacctcacctctcaagtagctgggactacaggtacatgctaccatgcctggctaatttttttttttttgcaggcatggggtctcactatattgcccaggttggtgtggaagtttaatgactaagaggtgtttgttataaagtttaatgtatgaaactttctattaaattcctgattttatttctgtaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtgtgagtatatttaatatatgattctttttagtggcaacagtaggttttcttatattttctttgaatctctgcaaaccatacttgctttcatttcacttggttacagtgagatttttctaacatattcactagtactttacatcaaagccaatactgtttttttaaaactagtcaccttggaggatatatacttattttacaggtgtgtgtggttttttaaataaactccttttaggaattgctgttgggacttgggatacttttttcactatacatactggtgacagataccctctcttgagctacatcggtttgtggggagtcaaaagtcctttggagctaggtttgacaaataaggtgggttaacacttgtttcctagaaagcacatggagagctagagtattggcgaattgaagaaatccccctttttttttaacacacttaagaaaggggactgcaggtatactcaagagagtaagtcgcaccagaaaccacttttgatccacagtctgcctgtgtcacacaattgaaatgcatcacaacattgacactgtggatgaaacaaaatcagtgtgaattttagtagtgaatttcattcataatttgatcgtgcaaacgtttgatttttattactttagactattgtttctgattttatgttgggttggtatttcctgtgagttactgttttacctttaaaataggaatttttcatactcttcaaagattagaacaaatgtccagtttttgctgtttcatgaatgagtcctgtccatctttgtagaaactcgccttatgttcacatttttattgagaataagaccacttatctacatttaactatcaacctcatcctctccattaatcatctattttagtgacccaagtttttgaccttttccatgtttacatcaatcctgtaggtgattgggcagccatttaagtattattatagacattttcactatcccattaaaaccctttatgcccatacatcataacactacttcctacccataagctccttttaacttgttaaagtcttgcttgaattaaagacttgtttacggtatcgataagcttgatatcaaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgtttaaacattaagaattaattcgatcctgaatggcgaatggacgcgccctgtagcggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatggctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagac

SEQ ID NO: 8 - DNA, 34814 bp, artificial
  HDAd-SAM vector sequence
  misc_feature (1599)..(1618): n is a, c, g, or t
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggatccatgcatgttaagtttaaacatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgttttttgatgccccccctcgaggttcgacggtatcgataagcttgatttaattaaggccggcccctaggggcgcgcgcggccgctagggataacagggtaatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttggatcctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatagggataacagggtaatgctagcatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggatcgtacggccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacataaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaattctaactataacggtcctaaggtagcgaagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatgaaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgtacataaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaattctaactataacggtcctaaggtagcgaagtcgaccgaatcgttgtcccttgtcacagccattgagaattttggcagggagcatgttcttagagcatttttaggctctgcgggacataacagctctgcctcagagcacatgcctttctcagctcctgaaagccactgatcaaattggaacattttgtaccttagggatgaggatatcaactctcccagccacttagagggataaatgtgatgatgcattcaattgtgactacatctgatcccaactgttgcttcagctgctctcctatagcacatggcgggaggcgtgcatcccagtagctacctccccacttttggggagatgtggttccatccatgaaacctgggtacccgcctaccaggtcctggcctatcaggtggcagggtctggtcaaagaagggcatgtgtggtcttcagcaagggagacaggacggtggtgcagagcgtctagaccctcagggcaagtctcccccacacctgctcccggggcagttgtctttgtgacctcccatccccctctgtttcatcctctataaaatgaggggctgagccccaaaataacaggcttctttgccatgatgcaaaactgctgaatctttctttctgacacacaaggcatcgagcagcctctgaaagaaccaaagccactagcaggcttcctgacttgggtttgtaggtactgaatactcccttgaaaaataaaaacatagaggcacttttctcctggctgtttattacagaacgaagaaaaaacacactggcttgaaacagacgccagatttcaaatgtagaggtgaaatacgaggtggcaattaaaatgtgattacagaaagtctggacactgagaaaagtttacaggacagtgggtgtgggttttctataacagacacttaaatatacatgacgataattgcagatagaaaccatcaaagacaaaccccaaatcaactaataatgtttacagatgttcccccccaaaccacagagccttacatcaaaacaaatactgaaaggctttaaaccaggaacagctcgccttaaccccacgagggtgcacacaagctgggctttttctctcggtctgaatggtaaagggaggaggatactctagctcctccaggtggattgctgagacagggctcggctcacacactgtctctgcgcctctcccaaatctggagaactctcccagcctcctggtaaagtgtctctgtggggcacttaacgataaaacagcttctgctgtaaagctcattaggaaagagctagcggagactgaaaggttcgcaaaagagattaagaatcacacaaggcaataggatttttagtgaacatagaaataaatggccaagtggttttctatttggcatttgtcaacttgcacaacaactcttggtcatatccacattgctcattgcattaaaaccataagcgactcagccacctagcttaacaaggtatcactggagcaaacaacacggtctgcatatttgtaacattgtataataaacacaaaacaatgcatagtaaacacaactctactgaaacaaaagccgtcgctttatttacaaagtcacaaaatgaagtataaatacttctgtcattaatgtttaggaaaaccatttacaaaattttcaaatatgtacacgtagcttgaaaaatcaccagctttccattttgtcacaggtagagagagggataagcatgggctgacaacaccactcaaattgtaacgggagacaactgcgggtatggatcgacaccacttcctagagtgatgtcaccatgggggtttctatgggcatcctgctcagatttaaagtgccccagcatcctgggtgacttgcccagaattctgggctgtggcattttgagcagcagcatgctgttccaaaatgtcgtcgatcagcctcaagttgcacacccagtcttcatctgggctcacacaggagcctttcaagagagcttcaatgaaatctacctcattgcagtcaggtgacgaaatcagatcatttagtgggggttggggctggcgcaaaaagtcggcaggtggcagctcagggggaatatccgttctgtcgaacggacctgggaactggctggcagcaacggcagaagcagcagcagcggtggcagcagcagccacatagcttggtggctcgatgccctgtatggggctcaggggactaaagctggccataccctgctggaggaacttggtggtgtttgctacaggcaccgggccctgtaccgggctctgcctgaggctctggctgcccagcaggctgaagctggggttgttggccaggggcacttgtgttcccatcgcagcgggcacttgtgcctcccaatcagatggcctctgaaggcaggcctggccagaaggtgagtgctgctgaacgctattatccacttggctgaggggtgttttccccgaaactgctgtggtcacagctgctgccgctgtgacccatgcagcattgttgaacgcagtgggcattcttggcacactaggccgtctgagctggtggggactcaaggactgggtgcccagggagctgggacagaacccaggcaggggcacttctggtggggtggccttggggctctgcatatgctggcagacagagtcaagtctgcccaggggagtctggcctgagtgtgagaggatgggacactgggggctggaggtgaaaattccttgccgcttccccagagttggtgagatcactcccatgccctcgcagctctggtgcctggtgagtgggatcattcctggactcagattgttctgaagaagcccagttctgggtggcatcaagtgcttgctagatggggggcttgccttgatccggctacacttggaggtgacttgttcttggacggctacatacagaaagagagaagtggggatgagttccaaaggcatcctcgacttcggctgtggccaccggagggtagctcctggcccaacacggacttctcacctcccgcccttggctctctactgagctcccccctgctccccaattcctcgccattcccctcatttctctgccctcagcctggactgcagttcttctgggaagctgccccaactccctaggtctgtgctcaccaagagcagatcacactggactgaaatgccagctgatttgtctcttcaagaaaattggaagctcctggaggtcagggtccatgtctgcttttacactcagtgctctgtatgcaggcctggcactgcccaccctttgacaggtggtgcatattttgtagaaggaaggaaggggccaggtggggtgggctgggctggtggcgggagctagctcagcctcttagattctctacccgatggatgtgacctgggacagcaagtgagtgtggtgagtgagtgcagacggtgctttgttcccctcttgtctcatagcctagatggcctctgagcccagatctggggctcagacaacatttgttcaactgaacggtaatgggtttcctttctgaaggctgaaatctgggagctgacattctggactccctgagttctgaagagcctggggatggagagacacggagcagaagatggaaggtagagtcccaggtgcctaagatggggaatacatctcccctcattgtcatgagagtccactctagctgatatctactgtggccaatatctaccggtacttttttggggtggacactgagtcatgcagcagtcttatggtttacccaaggtcaggtaggggagacagtgcagtcagagcacaagcccagtgtgtctgacccacccaagaatccatgctcgtatctacaaaaatgattttttctcttgtaatggtgcctaggttcttttattatcatggcatgtgtatgtttttcaactaggttacaatctggccttataaggttaacctcctggaggccaccagccttcctgaaacttgtctgtgctgtccctgcaactggagtgtgcctgatgtggcactccagcctggacaagtgggacacagactccgctgttatcaggcccaaagatgtcttccataagaccagaagagcaatggtgtagaggtgtcatgggctacaataaagatgctgacctcctgtctgagggcaagcagcctcttctggccctcagacaaatgctgagtgttcccaagactaccctcggcctggtccaatctcatcccactggtgcgtaagggttgctgaactcatgacttcttggctagcctgcaacctccacggagtgggaactacatcaggcattttgctaactgctgtatcctaggccaataaatgttgatcacatttatagctgccatggtagggtggggacccctgctatctatctgtggaggctctgggagcccctgacacaaactttctgaagcagagcctccccaaccccttttccattccctatacctgacagatggcccaggaacccattagaaatggaaggtcactgcagcagtatgtgaatgtgcgtgtgggagaagggcaggatcagagccctgggggtgtggcagcccccaagtgattctaatccagatcctagggttgtttccctgtcccattgaaatagctgctttaaggggcctgactcagggaaatcagtctcttgaattaagtggtgattttggagtcatttagaccaggccttcaattgggatccactagttctagagcggccgggcccagggaaccccgcaggcgggggcggccagtttcccgggttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgttgatttttcatctctaaacatcaacttacttaattctgccaatttcttttttttgagacagggtctcactctgtcacctaggatggagtgcagtggcacaatcactgctcactgcagcctcgacttcccgggctcgggtgattctccccaggctcaggggattctcccacttcagcctcccaagtagctgggactacaggtgcgcaccaccatccctggctaatatttgtactttattttatttatttatttatttattttttgagatggagtttcgctcttgttgcccgggctggagtacagtggcatgatctcggctcagtgcaacctctgcctcccgggttcaagcgattctcctacctcatccccctgagtagctgggattacaggcgcctgccaccatgcctggctaattttttgtatttttaatagagacgaggtttcaccatgttggccaggctactctcgaactcctgatctcaggtgatccacccgccttggcctcccaaagtgctgggattacaggcgtgagccactgcgcccggcctaatatttgtattttttgtagagatggtgttttgccatgttgtccaggctggtcttgaactcctgagctcaagcgatctgcccgcctctgcttcccaaagtgctgggattacaggcatgagccaccgtgcctggcctaggtagacgcttttagctttggggtgtgatgcctgccccagtatatagtgaatttaattattgctagagctggctgtttgttagttttctttgaacataagatactcattgtttttagtttgcaaatccctcttcctttttaaaaaatttctttcccttaaattgtttgcatgttagcaataacaaatgcttaaatggtgctatgtgctagatactcttctaagccctgttatgtatattaactaattttttaaattacacaaatcagagaggttaagtaacttgcccaagattacccaacaatactaggatttgaacctaagtttgtctcaccccagattctgctcttaatctctaaacttttaagttagtagtgacaatagtaggtatttattgaatacttaactatgttttaggcgttgaagtaaatattttgcaggcattatctaatgtaaacaccctaaagttacataacaggtaccctttaggtaaataaacactagtatgaccttggaggcacagatagttgaagtaacttgcccaatatcacttacatgaaattggccctcaaatgtgtctgatacaacccatgctgcttgtaactatcgttttaaactgccagggtaaacttggacacacttgagctaagaaaaagcttttagatttttgcaaattaatgtgaaagatatgctttatgtggatataatatcttctaaatttcggggatggtagtcctagaaatgtaatcctgccctagccgagcttaccctgccaataattttttacagaattggtaaaacggagcaccttttttttgtccttggccacactgttatcaacagggtgtagattgacatcaatctgtaggtgtaaaccagaattactctttgtgaccaccaggaaatagagcagttcagttcaggggtttctttctgtgaatttagcactgtgacctgcatactacaagtctactttgttttctatccattgtttgtatctgggtattgcaaaaggtaggaaaaggaccaaccagatcagcagagaagagttgccttggagttttcttttagttttctgcagttcattagatagtaactaggccatgtcattttactcccttgtagtgaagatatgttgaagttgtactggtatactcttctacctttctgtaattttatattgtgtagacttgataaaatttatgtgtcaatcaccaccattaatatcaatattgagcctcaattcttatttttctgcccagtggctgccaaattactaacatttacaataattcactactactaagataatctactagttcgatcacatacttcaaattgttatggaactactgtcttcagcattgtgcttctgataactgataagtataattttttttttgtccagagtgaacatgtctattcttccactgtacacactaataaaaggaaaaattgtaatattgggtaaattcatgtccttacacatgtagtagttatgagcccatgtccctagaatgagtaataatttatccctcccttggttgaatagtcaagaatgctgattttaattcttctaacagctttatccctcagaagggaaggcaagcaagttatatatgtagtttatttgtaagactgatatgaaattggaagatgaatctactattagctttaattatttttacatttaggaatattgcatcagtaactcataattttggttttctgttatcctgagttaacacaaattatccaaggagatggcggatcatctgctttgaggtgtttttttttgagaattttaatgtatctgaatataaaaggtaaaaatatgccaactagcaatttctgcccattccagaagtttggaaatattactcattactaggaattaaataaaatatggtttatctattgttatacctcttttaattcacatagctcatttttatcttttatttttgtttgttttttttgagatggagtcttgctctgtcaccaggcaggagtgcagtgatgcaaatctcggctcactctagccaccgactccctggttcaagcgattctcctgcctgagccttctgagtagctgggattacaggcaggcaccaccacgcccagctaatttttgtagagacaggatttcaccgtgttggccaggatggtctccatctcctgacctcatgatctgcctgcttcggcctcccaaagtgctgggattacaggtgggagccactacgcctggcccacatagctcatttttagactcacttccattaagtcttgtttggacccacgaacattgtctttttttttttaagatggagtttcacttttgttgcccagactgtagtgcaatggtgcaatctcagctcactgcaatctctgcctcctgggttctagcaattctcctgcctcagcctcccgagtagctggaattacaggcgcccgccaccacgcccagctaatttttgtgtttttagtagagacggggtttcaccatgttgggcaggccaggggtgatccgcccacctcagcctcccaaagtgctgggattacaggtgtgagccaccgcatctggccaacatgtcttttttttttttttcctttttaaccacaaagagacttaagcagtccttgtcacagatgatgaattgatgttgcaagtattgtcttagcttggattaattttcttgcttactgtaattttagataatatagctttgtaattagagattttatgtgtaaaccacaaaaatgtttacatgaaggccattattacagatgtgacgtgcataattattagtaatttgtatgtttacatgggtcagtctggcaaaaaattatgaagttttaaaaattaaaaaaaattataatgccagttttactggaaagtaaaattatttcagtaatcgattatagcaaaagtattgattttcattccagacaaaagtcagaatgaaaggtaatttctcaatactctttcagattaataaaagtacctgtagcgatttttatcattcacaagtatatcacaagtaagttagaatttgagaactgtgttctagatctctgaggagatgcagtcagatttctgaactgtctcagcaaatggtaagtaacttagagctagtaattaataacctgtcctttgatttctgattcagccaagaatggccatatttgggaaaggcagatctggagagtaaccacgttttcattcatttaccacttctaggcccctccagagctctcagatattttggggttgagcccttccccaaagccatacaggaccttttttttgtgatctgttctagccatttttatgttgggtgcttgttatggactgagcatttatgtcctcccacaccccccccataccttttttgaagtcctaacccccagtgtgatggtatttggagacagggcctttggaaggtaattacagttagaagaagtcgggagggttgggcccaggtctgattggattagtgcccttatatgaaaagacaccaggacgggcgcagtggctcacacctgtaatcccagcactttgggaggccaaggtgggtggatcacgaggtcaggagtttgagaccagcctggccaatgtagtgaaacaccatctctactaaaaatacaaaaattagctgggtgtggtagcgggctcctgtcatccaagctactcgggagggtgaggcatgagaatcacttgaacccgggagttggaggttgcagtgagcccagattgtgccactgtactccagcctgggtgacagagtgagactctgtctcaaaaaagaaaaaaaaaaaaaaagagacaccagagagcttgttagaagaggtcatgtgagcacacagttagaagaccttcaagccaaagaagaggcctgagattgaaacctaccttgcaggtaccttaattttggacttcccagcctccaaaactgtgagaaataagtttctgttaagtcactcagtctgtggtattttgttatggcagcctgagcaggtagttgttctttcagaaggtgttgataataaccacatgcaacaccaagtcacaaataataaaacagatgtaacttatattcatacagaaagttgggcactgccattgccttgttggtttacacggctgtgctagttcagtagcagaaaggtgctggtctcctttactcagtttacaatctaggcagtagaatgtaatcactgctttaaacttgatactgcttagggagagaatcattggtgctgggtaactttgggttctaggtttactttttgtgtatatataactgtttttggtaaatcacaagtttctgggcttgtcgaattagattttgttacagattatgagctttattatgctatacagttagttgtatgtatatatgcctttcccactagattttaagctttttttttttttttttttttgtgacggagtcttgctcttgtcgcccaggctgaagtggagtgcagtggcacaatctcggctcactgcagcctccacctcctaggttcaagcgattctcctgcctcggcctcccaagtaactgggactacaggcacgtgccaccacacccggctaatttttgtattttttgtagagacagggtttcgccatgttggctaggctggtcttgaacttctggcctcaggtgatccacccgcctcagcctcccaaagtgctgggatttacaggcatgagccaccacgcccagctatagctctttaagggttgtaaatttataatcattcttttactctcctgcaaattctgttgcacactgccttaatcaaggtagatgctgaatgcatttttgtataattgaatatgttgcaatccccaactctctccaactgttcctgtcaaagcagccactggattgttaactaatccatattagatggggttaattaatatcagatgggacaagtaagggctaataagattataggccaccaagtagatttctgtctagctcttatagagattgagtttattggacctgtttgataggaagttttggtgtttgggatgattaaaactgaagttcctatttattgaattatacctatttatattatttcatatcagtggtccacatgcaagtgaggcttctgagacagagtttgagttctctcttcaactaccataacacttaacctgtatctttttttttttttttttttttagacaggagtctcgctctgtcactcaggctggagtgtagtggtatgatctcggctcactgtaacctctgcctcctggattcaagcagttctccatgtctcagcctccctagtagctgggattacaggcctgtgccaccatgcctggctaattttttttttgtatttttagtagagacggggttttaccacgttggccaggctggtctcgaactcttgacctcgagcgatcaacttgccttggcctcccaaagtgctgggattacaggcatgagccacagcgcccagccgtctttttttttaaatagcaatttaacactgttcacagttactcatgtacatgtcatgccatctattacactgtaagttctgtgagggtagctgtatcaaatttatctaactctctctagtatgcatgacatagtaagtattcaataaatatttgcatattagtgataaggatacaggttctgaatagtgggtccttaccatttaagaattagtatttgatggccgggcggggtggctcacgcctgtaatcccagcactttgggaggctgaggcgggcggatcatgagatcaggagatcgagaccatcctggctaacatggtgaaatcccgtctttacaaaaaaaatacaaaagaattaaccaagtgtggtggtgggtgcctgtagtcccagctactgctttgtgaggctgaggcaggcagatcacctgaggtgggaaattcaagaccagcctgaccaacatggagaaaccccatctctactaaaaatacaaaattagccgggcgtggtggcgcatgtctgtaatcccagctactcgggaggctgaggcaggagaatggcgtgaacccgggaggcggagcttgcagtgagccaggatcgcgccactgcactccagcctgggcgacagagcgagactccgtctcaaaaaaaaaaaaaaaaaaaaaattagtatttgatatttgatcattaaatatgaattaagaggacttagactttttgttaaatgtcaagctgggaaaagttgtcatttaaatgaattgcctcttatttaatttcgtctgatgatacattttgtttttattttgtaaaaaattattttttttctttttggagacagggtcttgctctgttgcccaggctggtcacaaactcctgacctcaagcaatcctcctgccttagcctcccaaaatgctgggattacaggcgtgacgacctcgcccggccttgtattatgatacattttgaacaactacaagtagacttggtataatgaacctgcacgtacccattgccaagttctgacaactgtctgtctatagccaattatgcatttcttaaattagaacccccccaatatacccaaatatatatatatgtgtgcatatatatagtaagttgtaacaaagttgtgaattcatacctgaagtatctcaagtgatgcaagttttatgaatttttgtttatgccttttgggaagagttgtattgacaaattttttatgcttaaagtaaaccataaatcaaaaaaataaaatctaggatgcaataaaacaaaacaacttcttgacataagtatggtatgtaaatctgttttgattggaaatcaatttgttatattgccagaattcctgttttagaatacatctctgctgatctgtctgtattcttagactgcatatctgggatgaactctgggcagaattcacatgggcttcctttgaaataaacaagacttttcaaattcttagtcgatctgcagaacctgtagccaggcactgaaccattttgatagatgcagtaatcgttgcaagtgtatatttcaagggagttctggctgggtcctagtttatgcttgtggcagaagcagtgagtaactgggaggaagttggtgagtaagcttcaaggaagaagtcatttttagtactctggatcttcctgattttaaagcactacaaaatggtgcattttcattcttgtcaagtgataacagatatattctgatgagcctgaaatgaatatatattgtatcatttttataatatctagcaaggtttgtattttcctagaacttgaactaaatttcagttcataaaatttataaaatacttagttgttgtaaaatatttttggaatgttcacataggtgacacacaaatgtcccattttcattctttctatagtaaatatgttctgatatgtgaaggtttagcagatgcatcagcatttaatcctagaggatctggcataatcttttcccccaagaatagaaattttttctgcttatgaaagtagtacatgtttctttaaaaacaaatcaatattgacttctgcctgctgtatagcactatgcctccacctggccatgaccaggggcatgtcctggtccacctacctgaaaatgtttgcaaccagcctcctggccatgtgcacaggggctgaagttgtcccacaggtattacgggccaacctgacaatacatgaagttccaccaaagtctgagaactcagaactgagctttggggactgaaagacagcacaaacctcaaatttctcagcactggaaacctcaaaatataactgaattccataaataagattttaagtcttaaatatgtatttttaaatgtattaaaagtcaagctgcttgtatttaagcacctaatacaatgcttaggttgtaaaaggagatgctcaataggtactaactgatatattgagatttaattatggtttgaccaatatttattggaaaccgccaaagcttaaatcatcagcttcttgaatgtgatttgaaaggtaatttagtattgaatagcatgtgagctagagtatttcattctttctggtttatttcttcaaatagactttgaatataatggtgaatgggtattataaattaactaataaaaatgacattgaaaatgaaaaaatatatatattaaagtgtagaaagtgaccaggcgtggtggctcacacctgtaatccaagcaccttgggaggctgaggcaggaggatctcttgatcccaggagttcaagaccagcctgggcaacatagcgagacttcgtctctaaaaaaaaaaaagagagagaaaaaaattttttttatttaaaaaaagtgtagaaagtgtcaagaccccacttcttaccattatttggtatatttctctatacccacccacccttcctccttactccctccctcccttcccaatctttttatctttttgtattctgattttttgtttgtatattttgctttaatttaatgtatcctttaaaaatttcccatacattttatatgtatatataaaaacgcatgctgccaaagataatttataagaaagaccattgaatttttttaaaagtgatatatattcattgaaaaaaatttagaatatatagcaaagcaataaagaactaaataaaattgctgtaactcctctttcaaagataagtgcttttatgattttgttgtatttttttctgtatataggtacatatatagtatttataaagctgtactcatagtacattttcacatcacaggtaccatatcagtgttattaaatattttgtatgccaggggctagacataccaagacaaccaatatgtggttctacttaaataatattagagtatcttttatgatgacacttcatgagttgactataataatcttagacttctaagagtttgggttttcaaaagatcacttagcttttttgggtgatttttcccccttactgtgagatgagagaggctgtttggatttgggattggggtagcggggacagcaacttttcttttctttttcttttttattttgaggtagggtattgctgtgtcacccaggctggagtgcagtggtgtgatctcggctcactgcaacctccacctcccgggctcaggtgatcctcctgcttcagcctcccagtaactgggactacaggcgcgtgccacatgcctggctaattttgtatttttagtagagatggggtttcaccatgttggccaggctggtctctaactcctgacctcaggtgatacgcccacctgggcctcccaaaatactgggattacaggcatgagccgctgcatcagccagcagtttttcttgtggttttttttgtttgttttgttttgttttgtttttgagatagggtcttactctgttgtccacgctggagtgctgtggtatgatcgtagctcactgcagcctcaaactcctgggctcaagtgattccttctgcctccgcctcccgagtagctgggactacaggtatgcaccaccatacctggcaaatttttacaaagttttttgtagggacggggtcttgctacattccccatgtcggtcttgaactcctggcctcaagcaactctcctgtctcagcctcccaaagcactgggattacaagtgtgagccaccacaccatgccagtttttcctgttcagtgtgatattttatcttgttagactacagtgtgttaaaacttgttttactaaattttcaaacatactcaaaagtggagagaatagtataatgaatacccgtatgttcatcacccatgtttagaatattattaaatataaagattttgctgcgtttgtcttagctctttaaaatttttctttttctctttgtgacctaaaggaaattccatatcttatcactttacttctacattcttgactaagatgactaagacatatagttacatggttttttgttttgtttttgttttttaaagacgaaatctcgctcttgtcccccaggctggagtgcaatggtgccatctcagctcagtgcaacctctgccttctgggtacaagcgattctcctgcctcagcctcccaagtagctgggattacaggctcctgccaccacgcctggctaatttttgtatttttagtagagacggcggggggaggtttcaccatgttgacaaggctggtctggaactcctgacctcaggtgatccacccgcctcggcctcccaaagtgctgggattacaggcgtgagccaccgcgcccagcctgtttttttgtttgtgtgttttgttttttttgagacagagtcttgctctgtttcccaggctggagtgaagtggtgccatctcagctcagagacagagtcttgctctgtttcccaggctggagtgaagtggtgccatcttggctcactgcaaccttcacctcccaggttcaagtgattctcctgcctcagcctcccaagtagctgggactacaggcatgtgtcaccacacccggctaatttttttgtatttttagtagagacgggatttcaccgtgttgcccaggctggtctcgaactcctgagctcaggcagtctgcctgcctcagcctcccaaagtgctgggattacacgtgtgaaccaacccgcccggcctgttgttttcttacataattcattatcatacctacaaagttaacagttactaatatcatcttacacctaaatttctctgatagactaaggttattttttaacatcttaatccaatcaaatgtttgtatcctgtaatgctctcattgaaacagctatatttctttttcagattagtgatgatgaaccaggttatgaccttgatttattttgcatacctaatcattatgctgaggatttggaaagggtgtttattcctcatggactaattatggacaggtaagtaagatcttaaaatgaggttttttactttttcttgtgttaatttcaaacatcagcagctgttctgagtacttgctatttgaacataaactaggccaacttattaaataactgatgctttctaaaatcttctttattaaaaataaaagaggagggccttactaattacttagtatcagttgtggtatagtgggactctgtagggaccagaacaaagtaaacattgaagggagatggaagaaggaactctagccagagtcttgcatttctcagtcctaaacagggtaatggactggggctgaatcacatgaaggcaaggtcagatttttattattatgcacatctagcttgaaaattttctgttaagtcaattacagtgaaaaaccttacctggtattgaatgcttgcattgtatgtctggctattctgtgtttttattttaaaattataatatcaaaatatttgtgttataaaatattctaactatggaggccataaacaagaagactaaagttctctcctttcagccttctgtacacatttcttctcaagcactggcctatgcatgtatactatatgcaaaagtacatatatacatttatattttaacgtatgagtatagttttaaatgttattggacacttttaatattagtgtgtctagagctatctaatatattttaaaggttgcatagcattctgtcttatggagataccataactgatttaaccagtccactattgatagacactattttgttcttaccgactgtactagaagaaacattcttttacatgtttggtacttgttcagctttattcaagtggaatttctgggtcaaggggaaagagtttattgaatattttggtattgccaaattttcctctaagaagttgaatcattttatactcctgatgttatatgagagtacctttctcttcacaatttgtctcttttttttttttttttgagacaaggtctctgttgcccaggctggggtgcagtgcagcagaatgatcacagttcactgcagtctcaacctcctgggttcaagcgatccttccacctcagcctcctgagtagctgggactataggtgtgcgccaccactcccagctaatatttttattttgtagaaacagggttcgccatgttacccagcctcccaaagtgctgggattacaggcatgagccactggcccagtttctacagtctctcttaatattgtatattatccagaaaatttcatttaatcagaacctgccagtctgataggtgaaaatggtatcttgtttttatttgcatttaaaaaaaattatgatagtggtatgcttggtttttttgaaggtatcaaattttttaccttatgaaacatgagggcaaaggatgtgatacgtggaagatttaaaaaaaatttttaatgcatttttttgagacaaggtcttgctctattgtccaggctggagtgcagtggcacaatcacagttcactccagcctcaacatcctgcactaaagtgattttcccacctcacctctcaagtagctgggactacaggtacatgctaccatgcctggctaatttttttttttttgcaggcatggggtctcactatattgcccaggttggtgtggaagtttaatgactaagaggtgtttgttataaagtttaatgtatgaaactttctattaaattcctgattttatttctgtaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtgtgagtatatttaatatatgattctttttagtggcaacagtaggttttcttatattttctttgaatctctgcaaaccatacttgctttcatttcacttggttacagtgagatttttctaacatattcactagtactttacatcaaagccaatactgtttttttaaaactagtcaccttggaggatatatacttattttacaggtgtgtgtggttttttaaataaactccttttaggaattgctgttgggacttgggatacttttttcactatacatactggtgacagataccctctcttgagctacatcggtttgtggggagtcaaaagtcctttggagctaggtttgacaaataaggtgggttaacacttgtttcctagaaagcacatggagagctagagtattggcgaattgaagaaatccccctttttttttaacacacttaagaaaggggactgcaggtatactcaagagagtaagtcgcaccagaaaccacttttgatccacagtctgcctgtgtcacacaattgaaatgcatcacaacattgacactgtggatgaaacaaaatcagtgtgaattttagtagtgaatttcattcataatttgatcgtgcaaacgtttgatttttattactttagactattgtttctgattttatgttgggttggtatttcctgtgagttactgttttacctttaaaataggaatttttcatactcttcaaagattagaacaaatgtccagtttttgctgtttcatgaatgagtcctgtccatctttgtagaaactcgccttatgttcacatttttattgagaataagaccacttatctacatttaactatcaacctcatcctctccattaatcatctattttagtgacccaagtttttgaccttttccatgtttacatcaatcctgtaggtgattgggcagccatttaagtattattatagacattttcactatcccattaaaaccctttatgcccatacatcataacactacttcctacccataagctccttttaacttgttaaagtcttgcttgaattaaagacttgtttacggtatcgataagcttgatatcaaaacgccaactttgacccggaacgcggaaaacacctgagaaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataaggtatattattgatgatgtttaaacattaagaattaattcgatcctgaatggcgaatggacgcgccctgtagcggcgcattaagcgcgcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggatgttgccgatttcggcctattggttaaaaaatgagctgatttaacaaaaattttaacaaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatggctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagac

SEQ ID NO: 9 - DNA, 18173 bp, artificial
  LV-SAM vector sequence
  misc_feature (7200)..(7219): n is a, c, g, or t
agacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctcagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatcaccagataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcgtagggataacagggtaatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttggatcctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatagggataacagggtaatgctagcatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggatcgtacggccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacataaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaattctaactataacggtcctaaggtagcgaagctagctgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatgaaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgtacataaacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaattctaactataacggtcctaaggtagcgaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcg

SEQ ID NO: 10 - DNA, 7614 bp, artificial
  AAV-dCas9VP64 vector sequence
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaattcgccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtaactcgagcaataaagaatcgtttgtgttatgtttcaacgtgtttatttttcaattgcagcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 11 - DNA, 6968 bp, artificial
  AAV-MPH vector sequence
  misc_feature (4226)..(4245): n is a, c, g, or t
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgacctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggacctagggataacagggtaatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttggatcctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatagggataacagggtaattaactataacggtcctaaggtagcgaaggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaa

SEQ ID NO: 12 - DNA, 7871 bp, artificial
  AAV-dCas(phi)1-MPH-sgRNA vector sequence
  misc_feature (5698)..(5717): n is a, c, g, or t
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgtccggagaattcgccaccatgaaaaggccggcggccacgaaaaaggccggccaggcaaaaaagaaaaaggccgatacccccacactgttcacccaattcctcagacaccacctccccggccaaagatttagaaaggacattctgaagcaagccggaagaatcctcgctaataagggagaggacgccacaattgcctttctgagaggcaaatccgaggagagccctcccgacttccaaccccccgtgaagtgccccatcatcgcttgcagcagacctctgacagaatggcccatctatcaagccagcgtggctatccaaggctacgtctacggccagtctctggccgaatttgaggccagcgaccccggctgttccaaggatggactcctcggatggtttgacaagaccggcgtctgcaccgattatttcagcgtgcaaggactgaacctcattttccagaacgctaggaagaggtatatcggcgtgcagaccaaggtgaccaatagaaacgaaaagaggcacaaaaagctgaagaggatcaacgccaagagaatcgctgaaggactgcccgagctgacctccgacgagcccgagagcgctctggatgaaaccggccatctgatcgaccctcccggactgaacacaaacatctactgctaccagcaagtgagccctaagcctctggctctcagcgaggtgaatcagctgcccaccgcctacgctggatacagcacctccggagatgatcccatccagcccatggtgaccaaagatagactgagcatctccaaaggccagcccggatatatccccgagcaccagagggctctgctgagccaaaagaagcatagaaggatgagaggctacggactgaaggctagggctctgctcgtgatcgtgaggattcaagatgactgggccgtcatcgatctgaggtctctgctgaggaacgcttactggaggaggatcgtccagacaaaggagccctccacaatcaccaagctgctcaagctcgtgaccggcgatcccgtgctggacgccaccagaatggtcgccaccttcacctataaacccggaatcgtgcaagtgaggagcgctaaatgtctgaagaacaagcaaggcagcaagctgttcagcgaaaggtatctgaacgaaaccgtgagcgtgaccagcattgccctcggctccaacaatctggtcgctgtggccacctacagactggtcaacggaaatacccccgaactgctgcagaggtttacactccctagccatctggtgaaggatttcgagaggtacaaacaagctcacgatacactggaggactccattcagaagaccgccgtggcttctctgccccaaggccagcaaaccgagattagaatgtggtccatgtacggctttagagaggcccaagagagggtctgtcaagagctgggactggccgacggatccatcccttggaatgtgatgaccgccacatccaccattctgacagatctctttctggccagaggaggagaccccaagaagtgcatgttcaccagcgagcccaagaagaagaagaactccaagcaagtgctctataagattagagatagagcttgggccaagatgtacagaacactgctgtccaaagagaccagagaggcttggaataaagctctgtggggactgaaaaggggcagccccgactatgccagactgtccaagaggaaggaagagctggctagaagatgcgtcaactacaccatctccaccgccgagaagagggcccagtgtggaaggaccattgtggccctcgaagatctgaacatcggcttcttccacggcagaggaaaacaagagcccggatgggtgggactgttcacaagaaagaaggagaacagatggctcatgcaagccctccacaaggcttttctggagctggctcatcatagaggctaccacgtcatcgaagtcaaccccgcctatacctcccagacatgccccgtgtgtagacattgcgaccccgacaatagagaccagcataacagagaggccttccactgtatcggatgtggcttcagaggcaacgctgacctcgacgtggccacccacaacattgctatggtggccatcaccggcgaatccctcaaaagggccagaggctccgtggcttccaagacacctcaacctctggccgccgagggcagtggagagggcagaggaagtctgctaacatgcggtgacgtcgaggagaatcctggcccagccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgacctcgagcaataaagaatcgtttgtgttatgtttcaacgtgtttatttttcaattgcagcggacctagggataacagggtaatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacggccaacatgaggatcacccatgtctgcagggcccaccgggagagatctcaaacgattgctcgattagtcgagacagaagagcnnnnnnnnnnnnnnnnnnnngctcttcattttttttggtacctagggataacagggtaattaactataacggtcctaaggtagcgaaggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaa

SEQ ID NO: 13 - DNA, 20 bp, artificial
  nucleic acid that can encode a guide sequence that can target a PKD1 gene
tcgcgctgtggcgaaggggg

SEQ ID NO: 14 - DNA, 20 bp, artificial
  nucleic acid that can encode a guide sequence that can target a PKD1 gene
ccagtccctcatcgctggcc

SEQ ID NO: 15 - DNA, 20 bp, artificial
  nucleic acid that can encode a guide sequence that can target a PKD1 gene
ggagcggagggtgaagcctc

SEQ ID NO: 16 - DNA, 20 bp, artificial
  nucleic acid that can encode a guide sequence that can target a PKD1 gene
gaagagggcggagcctgtga

SEQ ID NO: 17 - DNA, 20 bp, artificial
  nucleic acid that can encode a guide sequence that can target a PKD1 gene
ttgcagatcctgcagtaggc

SEQ ID NO: 18 - DNA, 20 bp, artificial
  nucleic acid that can encode a guide sequence that can target a PKD1 gene
tgaaggaagggcgccctcag

SEQ ID NO: 19 - DNA, 20 bp, artificial
  nucleic acid that can encode a guide sequence that can target a PKD1 gene
cgcccagtgagcgtgagcct

SEQ ID NO: 20 - DNA, 20 bp, artificial
  nucleic acid that can encode a guide sequence that can target a PKD1 gene
ggtgggcggggtctcacggg

SEQ ID NO: 21 - DNA, 20 bp, artificial
  nucleic acid that can encode a guide sequence that can target a PKD1 gene
gcagaaggcggggcctccgg

SEQ ID NO: 22 - DNA, 20 bp, artificial
  nucleic acid that can encode a guide sequence that can target a PKD1 gene
cgctgggtctgctgcagacc

SEQ ID NO: 23 - DNA, 20 bp, artificial
  nucleic acid that can encode a guide sequence that can target a PKD2 gene
acgcggactcgggagccgcc

SEQ ID NO: 24 - DNA, 20 bp, artificial
  nucleic acid that can encode a guide sequence that can target a PKD2 gene
atccgccgcggcgcgctgag

SEQ ID NO: 25 - DNA, 20 bp, artificial
  nucleic acid that can encode a guide sequence that can target a PKD2 gene
gtgcgagggagccgcccccg

SEQ ID NO: 26 - DNA, 19 bp, artificial
  nucleic acid that can encode a sequence that can bind a MS2 polypeptide
acatgaggatcacccatgt